SEQUENCE LISTING (122 sequences)

SEQ ID NO: 1 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 112 aa, Homo sapiens
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 220 aa, Unknown
  source: /note=“Description of Unknown: CD28 sequence”
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 4 - protein, 107 aa, Unknown
  source: /note=“Description of Unknown: co-stimulatory signalling region”
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 5 - protein, 10 aa, Artificial Sequence
  source: /note=“Description of Artificial Sequence: Synthetic peptide”
EQKLISEEDL

SEQ ID NO: 6 - protein, 111 aa, Artificial Sequence
  source: /note=“Description of Artificial Sequence: Synthetic polypeptide”
IEVEQKLISEEDLLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 7 - protein, 702 aa, Artificial Sequence
  source: /note=“Description of Artificial Sequence: Synthetic polypeptide”
MGPGVLLLLLVATAWHGQGGVVSHFNDCPLSHDGYCLHDGVCMYIEALDKYACNCVVGYIGERCQYRDLKWWELRAAAPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCELRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 7 aa, Artificial Sequence
  source: /note=“Description of Artificial Sequence: Synthetic peptide”
GFTFSNY

SEQ ID NO: 9 - protein, 8 aa, Artificial Sequence
  source: /note=“Description of Artificial Sequence: Synthetic peptide”
RLKSNNYA

SEQ ID NO: 10 - protein, 6 aa, Artificial Sequence
  source: /note=“Description of Artificial Sequence: Synthetic peptide”
GNSFAY

SEQ ID NO: 11 - protein, 14 aa, Artificial Sequence
  source: /note=“Description of Artificial Sequence: Synthetic peptide”
RSSTGAVTTSNYAN

SEQ ID NO: 12 - protein, 7 aa, Artificial Sequence
  source: /note=“Description of Artificial Sequence: Synthetic peptide”
GTNNRAP

SEQ ID NO: 13 - protein, 9 aa, Artificial Sequence
  source: /note=“Description of Artificial Sequence: Synthetic peptide”
ALWYSNHWV

SEQ ID NO: 14 - protein, 117 aa, Artificial Sequence
  source: /note=“Description of Artificial Sequence: Synthetic polypeptide”
EVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSS

SEQ ID NO: 15 - protein, 112 aa, Artificial Sequence
  source: /note=“Description of Artificial Sequence: Synthetic polypeptide”
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSE

SEQ ID NO: 16 - protein, 244 aa, Artificial Sequence
  source: /note=“Description of Artificial Sequence: Synthetic polypeptide”
EVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSE

SEQ ID NO: 17 - DNA, 732 bp, Artificial Sequence
  source: /note=“Description of Artificial Sequence: Synthetic polynucleotide”
gaggtgcagctgcagcagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtacctttggtaactcctttgcttactggggccaagggaccacggtcaccgtctcctcaggtggaggcggttcaggcggaggtggctctggcggtggcggatcgcaggccgtggtcactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagcaccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtcctaggatcagag

SEQ ID NO: 18 - protein, 55 aa, Artificial Sequence
  source: /note=“Description of Artificial Sequence: Synthetic polypeptide”
VVSHFNDCPLSHDGYCLHDGVCMYIEALDKYACNCVVGYIGERCQYRDLKWWELR

SEQ ID NO: 19 - DNA, 165 bp, Artificial Sequence
  source: /note=“Description of Artificial Sequence: Synthetic polynucleotide”
gtggtgagccacttcaacgactgccctctgagccacgacggctactgcctgcacgacggcgtgtgcatgtacatcgaggccctggacaagtacgcctgcaactgcgtggtgggctacatcggcgagagatgccagtacagagacctgaagtggtgggagctgaga

SEQ ID NO: 20 - protein, 247 aa, Homo sapiens
MQPILLLLAFLLLPRADAGEIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

SEQ ID NO: 21 - protein, 277 aa, Homo sapiens
MENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETDSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYH

SEQ ID NO: 22 - protein, 479 aa, Homo sapiens
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKERSSSLEGSPDEFSNGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 23 - protein, 582 aa, Homo sapiens
MSPAPRPPRCLLLPLLTLGTALASLGSAQSSSFSPEAWLQQYGYLPPGDLRTHTQRSPQSLSAAIAAMQKFYGLQVTGKADADTMKAMRRPRCGVPDKFGAEIKANVRRKRYAIQGLKWQHNEITFCIQNYTPKVGEYATYEAIRKAFRVWESATPLRFREVPYAYIREGHEKQADIMIFFAEGFHGDSTPFDGEGGFLAHAYFPGPNIGGDTHFDSAEPWTVRNEDLNGNDIFLVAVHELGHALGLEHSSDPSAIMAPFYQWMDTENFVLPDDDRRGIQQLYGGESGFPTKMPPQPRTTSRPSVPDKPKNPTYGPNICDGNFDTVAMLRGEMFVFKERWFWRVRNNQVMDGYPMPIGQFWRGLPASINTAYERKDGKFVFFKGDKHWVFDEASLEPGYPKHIKELGRGLPTDKIDAALFWMPNGKTYFFRGNKYYRFNEELRAVDSEYPKNIKVWEGIPESPRGSFMGSDEVFTYFYKGNKYWKFNNQKLKVEPGYPKSALRDWMGCPSGGRPDEGTEEETEVIIIEVDEEGGGAVSAAAVVLPVLLLLLVLAVGLAVFFFRRHGTPRRLLYCQRSLLDKV

SEQ ID NO: 24 - protein, 157 aa, Unknown
  source: /note=“Description of Unknown: Mature IL-18 sequence”
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 25 - protein, 36 aa, Unknown
  source: /note=“Description of Unknown: Pro-peptide sequence”
MAAEPVEDNCINFVAMKFIDNTLYFIAEDDENLESD

SEQ ID NO: 26 - protein, 4 aa, Unknown
  source: /note=“Description of Unknown: GzB cleavage site sequence”
IEPD

SEQ ID NO: 27 - protein, 193 aa, Artificial Sequence
  source: /note=“Description of Artificial Sequence: Synthetic polypeptide”
MAAEPVEDNCINFVAMKFIDNTLYFIAEDDENIEPDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 28 - protein, 5 aa, Unknown
  source: /note=“Description of Unknown: Caspase-3 cleavage site sequence”
DEVDI

SEQ ID NO: 29 - protein, 194 aa, Artificial Sequence
  source: /note=“Description of Artificial Sequence: Synthetic polypeptide”
MAAEPVEDNCINFVAMKFIDNTLYFIAEDDENDEVDIYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 30 - protein, 5 aa, Unknown
  source: /note=“Description of Unknown: Caspase-8 cleavage site sequence”
IETDI

SEQ ID NO: 31 - protein, 194 aa, Artificial Sequence
  source: /note=“Description of Artificial Sequence: Synthetic polypeptide”
MAAEPVEDNCINFVAMKFIDNTLYFIAEDDENIETDIYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 32 - protein, 21 aa, Artificial Sequence
  source: /note=“Description of Artificial Sequence: Synthetic peptide”
GGGGSIPESLRAGGGGGSAAA

SEQ ID NO: 33 - protein, 210 aa, Artificial Sequence
  source: /note=“Description of Artificial Sequence: Synthetic polypeptide”
MAAEPVEDNCINFVAMKFIDNTLYFIAEDDENGGGGSIPESLRAGGGGGSAAAYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 34 - DNA, 27 bp, Artificial Sequence
  source: /note=“Description of Artificial Sequence: Synthetic oligonucleotide”
gacgacgagaacctggagagcgactac

SEQ ID NO: 35 - DNA, 27 bp, Artificial Sequence
  source: /note=“Description of Artificial Sequence: Synthetic oligonucleotide”
gacgacgagaacatcgagcccgactac

SEQ ID NO: 36 - protein, 158 aa, Homo sapiens
MEKALKIDTPQQGSIQDINHRVWVLQDQTLIAVPRKDRMSPVTIALISCRHVETLEKDRGNPIYLGLNGLNLCLMCAKVGDQPTLQLKEKDIMDLYNQPEPVKSFLFYHSQSGRNSTFESVAFPGWFIAVSSEGGCPLILTQELGKANTTDFGLTMLF

SEQ ID NO: 37 - protein, 162 aa, Artificial Sequence
  source: /note=“Description of Artificial Sequence: Synthetic polypeptide”
MEKALIEPDKIDTPQQGSIQDINHRVWVLQDQTLIAVPRKDRMSPVTIALISCRHVETLEKDRGNPIYLGLNGLNLCLMCAKVGDQPTLQLKEKDIMDLYNQPEPVKSFLFYHSQSGRNSTFESVAFPGWFIAVSSEGGCPLILTQELGKANTTDFGLTMLF

SEQ ID NO: 38 - protein, 164 aa, Homo sapiens
MNPQREAAPKSYAIRDSRQMVWVLSGNSLIAAPLSRSIKPVTLHLIACRDTEFSDKEKGNMVYLGIKGKDLCLFCAEIQGKPTLQLKLQGSQDNIGKDTCWKLVGIHTCINLDVRESCFMGTLDQWGIGVGRKKWKSSFQHHHLRKKDKDFSSMRTNIGMPGRM

SEQ ID NO: 39 - protein, 168 aa, Artificial Sequence
  source: /note=“Description of Artificial Sequence: Synthetic polypeptide”
MNPQIEPDREAAPKSYAIRDSRQMVWVLSGNSLIAAPLSRSIKPVTLHLIACRDTEFSDKEKGNMVYLGIKGKDLCLFCAEIQGKPTLQLKLQGSQDNIGKDTCWKLVGIHTCINLDVRESCFMGTLDQWGIGVGRKKWKSSFQHHHLRKKDKDFSSMRTNIGMPGRM

SEQ ID NO: 40 - protein, 169 aa, Homo sapiens
MRGTPGDADGGGRAVYQSMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 41 - protein, 169 aa, Artificial Sequence
  source: /note=“Description of Artificial Sequence: Synthetic polypeptide”
MRGTPGDADGGGRIEPDSMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 42 - protein, 153 aa, Unknown
  source: /note=“Description of Unknown: mature IL-36-alpha sequence”
KIDTPQQGSIQDINHRVWVLQDQTLIAVPRKDRMSPVTIALISCRHVETLEKDRGNPIYLGLNGLNLCLMCAKVGDQPTLQLKEKDIMDLYNQPEPVKSFLFYHSQSGRNSTFESVAFPGWFIAVSSEGGCPLILTQELGKANTTDFGLTMLF

SEQ ID NO: 43 - protein, 160 aa, Unknown
  source: /note=“Description of Unknown: mature IL-36-beta sequence”
REAAPKSYAIRDSRQMVWVLSGNSLIAAPLSRSIKPVTLHLIACRDTEFSDKEKGNMVYLGIKGKDLCLFCAEIQGKPTLQLKLQGSQDNIGKDTCWKLVGIHTCINLDVRESCFMGTLDQWGIGVGRKKWKSSFQHHHLRKKDKDFSSMRTNIGMPGRM

SEQ ID NO: 44 - protein, 152 aa, Unknown
  source: /note=“Description of Unknown: mature IL-36-gamma sequence”
SMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 45 - protein, 5 aa, Unknown
  source: /note=“Description of Unknown: cleaved pro-peptide from Pro-IL-36-alpha sequence”
MEKAL

SEQ ID NO: 46 - protein, 9 aa, Artificial Sequence
  source: /note=“Description of Artificial Sequence: Synthetic peptide”
MEKALIEPD

SEQ ID NO: 47 - protein, 4 aa, Unknown
  source: /note=“Description of Unknown: cleaved pro-peptide from Pro-IL-36-beta sequence”
MNPQ

SEQ ID NO: 48 - protein, 8 aa, Artificial Sequence
  source: /note=“Description of Artificial Sequence: Synthetic peptide”
MNPQIEPD

SEQ ID NO: 49 - protein, 17 aa, Unknown
  source: /note=“Description of Unknown: cleaved pro-peptide from Pro-IL-36-gamma sequence”
MRGTPGDADGGGRAVYQ

SEQ ID NO: 50 - protein, 17 aa, Artificial Sequence
  source: /note=“Description of Artificial Sequence: Synthetic peptide”
MRGTPGDADGGGRIEPD

SEQ ID NO: 51-100 - skipped (50 entries)

SEQ ID NO: 101 - DNA, 882 bp, Artificial Sequence
  source: /note=“Description of Artificial Sequence: Synthetic polynucleotide”
cgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccggcggaaacggtctggatctggggaaggtcggggatctttgctcacatgtggagatgtcgaagagaatcctggccctatggccgccgagcccgtggaggacaactgcatcaacttcgtggccatgaagttcatcgacaacaccctgtacttcatcgccgaggacgacgagaacctggagagcgactacttcggcaagctggagagcaagctgagcgtgatccggaacctgaacgaccaggtgctgttcatcgaccagggcaaccggcctctgttcgaggacatgaccgacagcgactgccgggacaacgctccccggaccatcttcatcatcagcatgtacaaggacagccagccccggggaatggccgtgaccatcagcgtgaagtgcgagaagatcagcaccctgagctgcgagaacaagatcatcagcttcaaggagatgaaccctcccgacaacatcaaggacaccaagagcgacatcatcttcttccagcggagcgtgcctggccacgacaacaagatgcagttcgagagcagcagctacgagggctacttcctggcctgcgagaaggagcgggacctgttcaagctgatcctgaagaaggaggacgagctgggcgaccggagcatcatgttcaccgtgcagaacgaggactaactcgag

SEQ ID NO: 102 - DNA, 9115 bp, Artificial Sequence
  source: /note=“Description of Artificial Sequence: Synthetic polynucleotide”
ccatgggccccggcgtgctgctgctgctgctggtggccaccgcctggcacggccagggcggcgtggtgagccacttcaacgactgccctctgagccacgacggctactgcctgcacgacggcgtgtgcatgtacatcgaggccctggacaagtacgcctgcaactgcgtggtgggctacatcggcgagagatgccagtacagagacctgaagtggtgggagctgagagccgccgcccccaccaccacacccgctcccagaccccctacccctgcccccaccatcgccagccagcccctgagcctgagacccgaggcctgcagacctgccgccggcggagccgtgcacaccagaggcctggacttcgcctgcgacatctacatctgggctcccctggccggcacctgcggcgtgctgctgctgagcctggtgatcaccctgtactgcaaccacaagagaggcagaaagaagctgctgtacatcttcaagcagcccttcatgagacccgtgcagaccacccaggaggaggacggctgcagctgcagattccccgaggaggaggagggcggctgcgagctgagaagaaagagaagcggcagcggcgagggcagaggcagcctgctgacctgcggcgacgtggaggagaaccccggacccatggctctcccagtgactgccctactgcttcccctagcgcttctcctgcatgcagaggtgcagctgcagcagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtacctttggtaactcctttgcttactggggccaagggaccacggtcaccgtctcctcaggtggaggcggttcaggcggaggtggctctggcggtggcggatcgcaggccgtggtcactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagcaccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtcctaggatcagaggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccggcggaaacggtctggatctggggaaggtcggggatctttgctcacatgtggagatgtcgaagagaatcctggccctatggccgccgagcccgtggaggacaactgcatcaacttcgtggccatgaagttcatcgacaacaccctgtacttcatcgccgaggacgacgagaacctggagagcgactacttcggcaagctggagagcaagctgagcgtgatccggaacctgaacgaccaggtgctgttcatcgaccagggcaaccggcctctgttcgaggacatgaccgacagcgactgccgggacaacgctccccggaccatcttcatcatcagcatgtacaaggacagccagccccggggaatggccgtgaccatcagcgtgaagtgcgagaagatcagcaccctgagctgcgagaacaagatcatcagcttcaaggagatgaaccctcccgacaacatcaaggacaccaagagcgacatcatcttcttccagcggagcgtgcctggccacgacaacaagatgcagttcgagagcagcagctacgagggctacttcctggcctgcgagaaggagcgggacctgttcaagctgatcctgaagaaggaggacgagctgggcgaccggagcatcatgttcaccgtgcagaacgaggactaactcgagggatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactg

SEQ ID NO: 103 - DNA, 9114 bp, Artificial Sequence
  source: /note=“Description of Artificial Sequence: Synthetic polynucleotide”
ccatgggccccggcgtgctgctgctgctgctggtggccaccgcctggcacggccagggcggcgtggtgagccacttcaacgactgccctctgagccacgacggctactgcctgcacgacggcgtgtgcatgtacatcgaggccctggacaagtacgcctgcaactgcgtggtgggctacatcggcgagagatgccagtacagagacctgaagtggtgggagctgagagccgccgcccccaccaccacacccgctcccagaccccctacccctgcccccaccatcgccagccagcccctgagcctgagacccgaggcctgcagacctgccgccggcggagccgtgcacaccagaggcctggacttcgcctgcgacatctacatctgggctcccctggccggcacctgcggcgtgctgctgctgagcctggtgatcaccctgtactgcaaccacaagagaggcagaaagaagctgctgtacatcttcaagcagcccttcatgagacccgtgcagaccacccaggaggaggacggctgcagctgcagattccccgaggaggaggagggcggctgcgagctgagaagaaagagaagcggcagcggcgagggcagaggcagcctgctgacctgcggcgacgtggaggagaaccccggacccatggctctcccagtgactgccctactgcttcccctagcgcttctcctgcatgcagaggtgcagctgcagcagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtacctttggtaactcctttgcttactggggccaagggaccacggtcaccgtctcctcaggtggaggcggttcaggcggaggtggctctggcggtggcggatcgcaggccgtggtcactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagcaccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtcctaggatcagaggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccggcggaaacggtctggatctggggaaggtcggggatctttgctcacatgtggagatgtcgaagagaatcctggccctatggccgccgagcccgtggaggacaactgcatcaacttcgtggccatgaagttcatcgacaacaccctgtacttcatcgccgaggacgacgagaacatcgagcccgactacttcggcaagctggagagcaagctgagcgtgatccggaacctgaacgaccaggtgctgttcatcgaccagggcaaccggcctctgttcgaggacatgaccgacagcgactgccgggacaacgctccccggaccatcttcatcatcagcatgtacaaggacagccagccccggggaatggccgtgaccatcagcgtgaagtgcgagaagatcagcaccctgagctgcgagaacaagatcatcagcttcaaggagatgaaccctcccgacaacatcaaggacaccaagagcgacatcatcttcttccagcggagcgtgcctggccacgacaacaagatgcagttcgagagcagcagctacgagggctacttcctggcctgcgagaaggagcgggacctgttcaagctgatcctgaagaaggaggacgagctgggcgaccggagcatcatgttcaccgtgcagaacgaggactaactcgaggatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactg

SEQ ID NO: 104 - DNA, 849 bp, Artificial Sequence
  source: /note=“Description of Artificial Sequence: Synthetic polynucleotide”
cgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccggcggaaacggtctggatctggggaaggtcggggatctttgctcacatgtggagatgtcgaagagaatcctggccctatgaaccggggagtgcccttccggcacctgctgctggtgctgcagctggccctgctgcctgccgctacccagggctacttcggcaagctggagagcaagctgagcgtgatccggaacctgaacgaccaggtgctgttcatcgaccagggcaaccggcctctgttcgaggacatgaccgacagcgactgccgggacaacgctccccggaccatcttcatcatcagcatgtacaaggacagccagccccggggaatggccgtgaccatcagcgtgaagtgcgagaagatcagcaccctgagctgcgagaacaagatcatcagcttcaaggagatgaaccctcccgacaacatcaaggacaccaagagcgacatcatcttcttccagcggagcgtgcctggccacgacaacaagatgcagttcgagagcagcagctacgagggctacttcctggcctgcgagaaggagcgggacctgttcaagctgatcctgaagaaggaggacgagctgggcgaccggagcatcatgttcaccgtgcagaacgaggactaactcgag

SEQ ID NO: 105 - DNA, 9082 bp, Artificial Sequence
  source: /note=“Description of Artificial Sequence: Synthetic polynucleotide”
ccatgggccccggcgtgctgctgctgctgctggtggccaccgcctggcacggccagggcggcgtggtgagccacttcaacgactgccctctgagccacgacggctactgcctgcacgacggcgtgtgcatgtacatcgaggccctggacaagtacgcctgcaactgcgtggtgggctacatcggcgagagatgccagtacagagacctgaagtggtgggagctgagagccgccgcccccaccaccacacccgctcccagaccccctacccctgcccccaccatcgccagccagcccctgagcctgagacccgaggcctgcagacctgccgccggcggagccgtgcacaccagaggcctggacttcgcctgcgacatctacatctgggctcccctggccggcacctgcggcgtgctgctgctgagcctggtgatcaccctgtactgcaaccacaagagaggcagaaagaagctgctgtacatcttcaagcagcccttcatgagacccgtgcagaccacccaggaggaggacggctgcagctgcagattccccgaggaggaggagggcggctgcgagctgagaagaaagagaagcggcagcggcgagggcagaggcagcctgctgacctgcggcgacgtggaggagaaccccggacccatggctctcccagtgactgccctactgcttcccctagcgcttctcctgcatgcagaggtgcagctgcagcagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtacctttggtaactcctttgcttactggggccaagggaccacggtcaccgtctcctcaggtggaggcggttcaggcggaggtggctctggcggtggcggatcgcaggccgtggtcactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagcaccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtcctaggatcagaggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccggcggaaacggtctggatctggggaaggtcggggatctttgctcacatgtggagatgtcgaagagaatcctggccctatgaaccggggagtgcccttccggcacctgctgctggtgctgcagctggccctgctgcctgccgctacccagggctacttcggcaagctggagagcaagctgagcgtgatccggaacctgaacgaccaggtgctgttcatcgaccagggcaaccggcctctgttcgaggacatgaccgacagcgactgccgggacaacgctccccggaccatcttcatcatcagcatgtacaaggacagccagccccggggaatggccgtgaccatcagcgtgaagtgcgagaagatcagcaccctgagctgcgagaacaagatcatcagcttcaaggagatgaaccctcccgacaacatcaaggacaccaagagcgacatcatcttcttccagcggagcgtgcctggccacgacaacaagatgcagttcgagagcagcagctacgagggctacttcctggcctgcgagaaggagcgggacctgttcaagctgatcctgaagaaggaggacgagctgggcgaccggagcatcatgttcaccgtgcagaacgaggactaactcgagggatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactg

SEQ ID NO: 106 - DNA, 885 bp, Artificial Sequence
  source: /note=“Description of Artificial Sequence: Synthetic polynucleotide”
cgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccggcggaaacggtctggatctggggaaggtcggggatctttgctcacatgtggagatgtcgaagagaatcctggccctatggccgccgagcccgtggaggacaactgcatcaacttcgtggccatgaagttcatcgacaacaccctgtacttcatcgccgaggacgacgagaacatcgagaccgacatctacttcggcaagctggagagcaagctgagcgtgatccggaacctgaacgaccaggtgctgttcatcgaccagggcaaccggcctctgttcgaggacatgaccgacagcgactgccgggacaacgctccccggaccatcttcatcatcagcatgtacaaggacagccagccccggggaatggccgtgaccatcagcgtgaagtgcgagaagatcagcaccctgagctgcgagaacaagatcatcagcttcaaggagatgaaccctcccgacaacatcaaggacaccaagagcgacatcatcttcttccagcggagcgtgcctggccacgacaacaagatgcagttcgagagcagcagctacgagggctacttcctggcctgcgagaaggagcgggacctgttcaagctgatcctgaagaaggaggacgagctgggcgaccggagcatcatgttcaccgtgcagaacgaggactaactcgag

SEQ ID NO: 107 - DNA, 9118 bp, Artificial Sequence
  source: /note=“Description of Artificial Sequence: Synthetic polynucleotide”
ccatgggccccggcgtgctgctgctgctgctggtggccaccgcctggcacggccagggcggcgtggtgagccacttcaacgactgccctctgagccacgacggctactgcctgcacgacggcgtgtgcatgtacatcgaggccctggacaagtacgcctgcaactgcgtggtgggctacatcggcgagagatgccagtacagagacctgaagtggtgggagctgagagccgccgcccccaccaccacacccgctcccagaccccctacccctgcccccaccatcgccagccagcccctgagcctgagacccgaggcctgcagacctgccgccggcggagccgtgcacaccagaggcctggacttcgcctgcgacatctacatctgggctcccctggccggcacctgcggcgtgctgctgctgagcctggtgatcaccctgtactgcaaccacaagagaggcagaaagaagctgctgtacatcttcaagcagcccttcatgagacccgtgcagaccacccaggaggaggacggctgcagctgcagattccccgaggaggaggagggcggctgcgagctgagaagaaagagaagcggcagcggcgagggcagaggcagcctgctgacctgcggcgacgtggaggagaaccccggacccatggctctcccagtgactgccctactgcttcccctagcgcttctcctgcatgcagaggtgcagctgcagcagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtacctttggtaactcctttgcttactggggccaagggaccacggtcaccgtctcctcaggtggaggcggttcaggcggaggtggctctggcggtggcggatcgcaggccgtggtcactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagcaccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtcctaggatcagaggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccggcggaaacggtctggatctggggaaggtcggggatctttgctcacatgtggagatgtcgaagagaatcctggccctatggccgccgagcccgtggaggacaactgcatcaacttcgtggccatgaagttcatcgacaacaccctgtacttcatcgccgaggacgacgagaacatcgagaccgacatctacttcggcaagctggagagcaagctgagcgtgatccggaacctgaacgaccaggtgctgttcatcgaccagggcaaccggcctctgttcgaggacatgaccgacagcgactgccgggacaacgctccccggaccatcttcatcatcagcatgtacaaggacagccagccccggggaatggccgtgaccatcagcgtgaagtgcgagaagatcagcaccctgagctgcgagaacaagatcatcagcttcaaggagatgaaccctcccgacaacatcaaggacaccaagagcgacatcatcttcttccagcggagcgtgcctggccacgacaacaagatgcagttcgagagcagcagctacgagggctacttcctggcctgcgagaaggagcgggacctgttcaagctgatcctgaagaaggaggacgagctgggcgaccggagcatcatgttcaccgtgcagaacgaggactaactcgagggatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactg

SEQ ID NO: 108 - DNA, 885 bp, Artificial Sequence
  source: /note=“Description of Artificial Sequence: Synthetic polynucleotide”
cgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccggcggaaacggtctggatctggggaaggtcggggatctttgctcacatgtggagatgtcgaagagaatcctggccctatggccgccgagcccgtggaggacaactgcatcaacttcgtggccatgaagttcatcgacaacaccctgtacttcatcgccgaggacgacgagaacgacgaggtggacatctacttcggcaagctggagagcaagctgagcgtgatccggaacctgaacgaccaggtgctgttcatcgaccagggcaaccggcctctgttcgaggacatgaccgacagcgactgccgggacaacgctccccggaccatcttcatcatcagcatgtacaaggacagccagccccggggaatggccgtgaccatcagcgtgaagtgcgagaagatcagcaccctgagctgcgagaacaagatcatcagcttcaaggagatgaaccctcccgacaacatcaaggacaccaagagcgacatcatcttcttccagcggagcgtgcctggccacgacaacaagatgcagttcgagagcagcagctacgagggctacttcctggcctgcgagaaggagcgggacctgttcaagctgatcctgaagaaggaggacgagctgggcgaccggagcatcatgttcaccgtgcagaacgaggactaactcgag

SEQ ID NO: 109 - DNA, 9118 bp, Artificial Sequence
  source: /note=“Description of Artificial Sequence: Synthetic polynucleotide”
ccatgggccccggcgtgctgctgctgctgctggtggccaccgcctggcacggccagggcggcgtggtgagccacttcaacgactgccctctgagccacgacggctactgcctgcacgacggcgtgtgcatgtacatcgaggccctggacaagtacgcctgcaactgcgtggtgggctacatcggcgagagatgccagtacagagacctgaagtggtgggagctgagagccgccgcccccaccaccacacccgctcccagaccccctacccctgcccccaccatcgccagccagcccctgagcctgagacccgaggcctgcagacctgccgccggcggagccgtgcacaccagaggcctggacttcgcctgcgacatctacatctgggctcccctggccggcacctgcggcgtgctgctgctgagcctggtgatcaccctgtactgcaaccacaagagaggcagaaagaagctgctgtacatcttcaagcagcccttcatgagacccgtgcagaccacccaggaggaggacggctgcagctgcagattccccgaggaggaggagggcggctgcgagctgagaagaaagagaagcggcagcggcgagggcagaggcagcctgctgacctgcggcgacgtggaggagaaccccggacccatggctctcccagtgactgccctactgcttcccctagcgcttctcctgcatgcagaggtgcagctgcagcagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtacctttggtaactcctttgcttactggggccaagggaccacggtcaccgtctcctcaggtggaggcggttcaggcggaggtggctctggcggtggcggatcgcaggccgtggtcactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagcaccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtcctaggatcagaggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccggcggaaacggtctggatctggggaaggtcggggatctttgctcacatgtggagatgtcgaagagaatcctggccctatggccgccgagcccgtggaggacaactgcatcaacttcgtggccatgaagttcatcgacaacaccctgtacttcatcgccgaggacgacgagaacgacgaggtggacatctacttcggcaagctggagagcaagctgagcgtgatccggaacctgaacgaccaggtgctgttcatcgaccagggcaaccggcctctgttcgaggacatgaccgacagcgactgccgggacaacgctccccggaccatcttcatcatcagcatgtacaaggacagccagccccggggaatggccgtgaccatcagcgtgaagtgcgagaagatcagcaccctgagctgcgagaacaagatcatcagcttcaaggagatgaaccctcccgacaacatcaaggacaccaagagcgacatcatcttcttccagcggagcgtgcctggccacgacaacaagatgcagttcgagagcagcagctacgagggctacttcctggcctgcgagaaggagcgggacctgttcaagctgatcctgaagaaggaggacgagctgggcgaccggagcatcatgttcaccgtgcagaacgaggactaactcgagggatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactg

SEQ ID NO: 110 - DNA, 718 bp, Artificial Sequence
  source: /note=“Description of Artificial Sequence: Synthetic polynucleotide”
caccaaggtgagcagcttcgtgcactggatcaagaagaccatgaagcggtaccgccgcaaacgctctgggtccggagaagggcggggatccttgctcacatgtggggatgttgaagagaatcctgggccaatggccgccgagcccgtggaggacaactgcatcaacttcgtggccatgaagttcatcgacaacaccctgtacttcatcgccgaggacgacgagaacatcgagcccgactacttcggcaagctggagagcaagctgagcgtgatccggaacctgaacgaccaggtgctgttcatcgaccagggcaaccggcctctgttcgaggacatgaccgacagcgactgccgggacaacgctccccggaccatcttcatcatcagcatgtacaaggacagccagccccggggaatggccgtgaccatcagcgtgaagtgcgagaagatcagcaccctgagctgcgagaacaagatcatcagcttcaaggagatgaaccctcccgacaacatcaaggacaccaagagcgacatcatcttcttccagcggagcgtgcctggccacgacaacaagatgcagttcgagagcagcagctacgagggctacttcctggcctgcgagaaggagcgggacctgttcaagctgatcctgaagaaggaggacgagctgggcgaccggagcatcatgttcaccgtgcagaacgaggactaactcgag

SEQ ID NO: 111 - DNA, 9937 bp, Artificial Sequence
  source: /note=“Description of Artificial Sequence: Synthetic polynucleotide”
ccatgggccccggcgtgctgctgctgctgctggtggccaccgcctggcacggccagggcggcgtggtgagccacttcaacgactgccctctgagccacgacggctactgcctgcacgacggcgtgtgcatgtacatcgaggccctggacaagtacgcctgcaactgcgtggtgggctacatcggcgagagatgccagtacagagacctgaagtggtgggagctgagagccgccgcccccaccaccacacccgctcccagaccccctacccctgcccccaccatcgccagccagcccctgagcctgagacccgaggcctgcagacctgccgccggcggagccgtgcacaccagaggcctggacttcgcctgcgacatctacatctgggctcccctggccggcacctgcggcgtgctgctgctgagcctggtgatcaccctgtactgcaaccacaagagaggcagaaagaagctgctgtacatcttcaagcagcccttcatgagacccgtgcagaccacccaggaggaggacggctgcagctgcagattccccgaggaggaggagggcggctgcgagctgagaagaaagagaagcggcagcggcgagggcagaggcagcctgctgacctgcggcgacgtggaggagaaccccggacccatggctctcccagtgactgccctactgcttcccctagcgcttctcctgcatgcagaggtgcagctgcagcagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtacctttggtaactcctttgcttactggggccaagggaccacggtcaccgtctcctcaggtggaggcggttcaggcggaggtggctctggcggtggcggatcgcaggccgtggtcactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagcaccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtcctaggatcagaggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccggcggaaacggtccggatctggggctacaaacttctctctcttgaagcaggccggagatgtcgaagaaaatccaggccctatgcagcccatcctgttgctcctggccttcctcttgctgcctcgggccgacgccggcgagatcatcggcggacacgaggccaagccccacagcaggccctacatggcctacctgatgatctgggaccagaagagcctgaagcggtgcggaggcttcctgatccgggacgacttcgtgctgaccgccgcccactgctggggaagcagcatcaacgtgaccctgggcgctcacaacatcaaggagcaggagcccacccagcagttcatccctgtgaagcggcccatccctcaccccgcctacaaccccaagaacttcagcaacgacatcatgctgctgcagctggagcggaaggccaagcggacccgggccgtgcagcccctgcggctgcccagcaacaaggcccaggtgaagcctggccagacctgcagcgtggccggctggggccagaccgctcccctgggcaagcacagccacaccctgcaggaggtgaagatgaccgtgcaggaggaccggaagtgcgagagcgacctgcggcactactacgacagcaccatcgagctgtgcgtgggagaccccgagatcaagaagaccagcttcaagggcgacagcggcggacccctggtgtgcaacaaggtggcccagggcatcgtgagctacggcaggaacaacggcatgccccctcgggcctgcaccaaggtgagcagcttcgtgcactggatcaagaagaccatgaagcggtaccgccgcaaacgctctgggtccggagaagggcggggatccttgctcacatgtggggatgttgaagagaatcctgggccaatggccgccgagcccgtggaggacaactgcatcaacttcgtggccatgaagttcatcgacaacaccctgtacttcatcgccgaggacgacgagaacatcgagcccgactacttcggcaagctggagagcaagctgagcgtgatccggaacctgaacgaccaggtgctgttcatcgaccagggcaaccggcctctgttcgaggacatgaccgacagcgactgccgggacaacgctccccggaccatcttcatcatcagcatgtacaaggacagccagccccggggaatggccgtgaccatcagcgtgaagtgcgagaagatcagcaccctgagctgcgagaacaagatcatcagcttcaaggagatgaaccctcccgacaacatcaaggacaccaagagcgacatcatcttcttccagcggagcgtgcctggccacgacaacaagatgcagttcgagagcagcagctacgagggctacttcctggcctgcgagaaggagcgggacctgttcaagctgatcctgaagaaggaggacgagctgggcgaccggagcatcatgttcaccgtgcagaacgaggactaactcgagggatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactg

SEQ ID NO: 112 - DNA, 11023 bp, Artificial Sequence
  source: /note=“Description of Artificial Sequence: Synthetic polynucleotide”
ccatgggccccggcgtgctgctgctgctgctggtggccaccgcctggcacggccagggcggcgtggtgagccacttcaacgactgccctctgagccacgacggctactgcctgcacgacggcgtgtgcatgtacatcgaggccctggacaagtacgcctgcaactgcgtggtgggctacatcggcgagagatgccagtacagagacctgaagtggtgggagctgagagccgccgcccccaccaccacacccgctcccagaccccctacccctgcccccaccatcgccagccagcccctgagcctgagacccgaggcctgcagacctgccgccggcggagccgtgcacaccagaggcctggacttcgcctgcgacatctacatctgggctcccctggccggcacctgcggcgtgctgctgctgagcctggtgatcaccctgtactgcaaccacaagagaggcagaaagaagctgctgtacatcttcaagcagcccttcatgagacccgtgcagaccacccaggaggaggacggctgcagctgcagattccccgaggaggaggagggcggctgcgagctgagaagaaagagaagcggcagcggcgagggcagaggcagcctgctgacctgcggcgacgtggaggagaaccccggacccatggctctcccagtgactgccctactgcttcccctagcgcttctcctgcatgcagaggtgcagctgcagcagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtacctttggtaactcctttgcttactggggccaagggaccacggtcaccgtctcctcaggtggaggcggttcaggcggaggtggctctggcggtggcggatcgcaggccgtggtcactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagcaccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtcctaggatcagaggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccggcggaaacggtccggatctggggctacaaacttctctctcttgaagcaggccggagatgtcgaagaaaatccaggccctatgcagcccatcctgttgctcctggccttcctcttgctgcctcgggccgacgccggcgagatcatcggcggacacgaggccaagccccacagcaggccctacatggcctacctgatgatctgggaccagaagagcctgaagcggtgcggaggcttcctgatccgggacgacttcgtgctgaccgccgcccactgctggggaagcagcatcaacgtgaccctgggcgctcacaacatcaaggagcaggagcccacccagcagttcatccctgtgaagcggcccatccctcaccccgcctacaaccccaagaacttcagcaacgacatcatgctgctgcagctggagcggaaggccaagcggacccgggccgtgcagcccctgcggctgcccagcaacaaggcccaggtgaagcctggccagacctgcagcgtggccggctggggccagaccgctcccctgggcaagcacagccacaccctgcaggaggtgaagatgaccgtgcaggaggaccggaagtgcgagagcgacctgcggcactactacgacagcaccatcgagctgtgcgtgggagaccccgagatcaagaagaccagcttcaagggcgacagcggcggacccctggtgtgcaacaaggtggcccagggcatcgtgagctacggcaggaacaacggcatgccccctcgggcctgcaccaaggtgagcagcttcgtgcactggatcaagaagaccatgaagcggtaccgccgcaaacgctctgggtccggagaagggcggggatccttgctcacatgtggggatgttgaagagaatcctgggccaatggccgctcggctgctgctgctgggcatcctgttgctcctgctccctctgcccgtgcccgccccctgccacaccgccgcccggagcgagtgcaagcggagccacaagttcgtgcctggcgcctggctggccggagagggcgtggacgtgaccagcctgcggaggagcggcagcttccctgtggacacccagcggttcctgcggcctgacggcacctgcaccctgtgcgagaacgccctgcaggagggcaccctgcagaggctgcccctggccctgaccaactggagggcccagggaagcggctgccagcggcacgtgaccagggccaaggtgagcagcaccgaggccgtggcccgggacgccgcccggagcatccggaacgactggaaggtgggcctggacgtgacccccaagcccaccagcaacgtgcacgtgagcgtggcaggcagccacagccaggccgccaacttcgccgcccagaagacccaccaggaccagtacagcttcagcaccgacaccgtggagtgccggttctacagcttccacgtggtgcacacaccccctctgcaccccgacttcaagcgggccctgggcgacctgccccaccacttcaacgccagcacccagcctgcctacctgcggctgatcagcaactacggcacccacttcatccgggctgtggagctgggaggcaggatcagcgccctgaccgccctgcggacctgcgagctggccctggagggcctgaccgacaacgaggtggaggactgcctgaccgtggaggcccaggtgaacatcggcatccacggcagcatcagcgccgaggccaaggcctgcgaggagaagaagaagaagcacaagatgaccgccagcttccaccagacctaccgggagcggcacagcgaggtggtgggaggccaccacaccagcatcaacgacctgctgttcggcatccaggctggacccgagcagtacagcgcctgggtgaacagcctgcctggcagccccggactggtggactacaccctggagcccctgcacgtgctgctggacagccaggaccccaggcgggaggccctgcggagggccctgagccagtacctgaccgaccgggctcggtggcgggactgcagcagaccctgcccccctggcaggcagaagagccccagggacccctgccagtgcgtgtgccacggcagcgctgtgaccacccaggactgctgccccaggcagcggggactggcccagctggaggtgaccttcatccaggcctggggcctgtggggcgactggttcaccgccaccgacgcctacgtgaagctgttcttcggaggccaggagctgcggaccagcaccgtgtgggacaacaacaaccccatctggagcgtgcggctggacttcggcgacgtgctgctggccaccggcggacccctgcggctgcaggtgtgggaccaggacagcggcagggacgacgacctgctgggcacctgcgaccaggctcccaagagcggcagccacgaggtgcggtgcaacctgaaccacggccacctgaagttccggtaccacgccaggtgcctgccccacctgggcggaggcacctgcctggactacgtgccccagatgctgctgggcgagccccctggcaaccggagcggcgctgtgtggtaactcgagggatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactg

SEQ ID NO: 113 - DNA, 9670 bp, Artificial Sequence
  source: /note=“Description of Artificial Sequence: Synthetic polynucleotide”
ggatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatggcagctgagcctgtggaggacaactgcatcaacttcgtggccatgaagttcatcgacaacaccctgtacttcatcgctgaggacgacgagaacggaggcgggggtagcatccctgagagcctgagagctggtgggggaggtggaagcgctgccgcttacttcggaaagctggagagcaagctgagcgtgatcagaaacctgaacgaccaggtgctgttcatcgaccagggcaacagacctctgttcgaggacatgaccgacagcgactgcagagacaacgctcccagaaccatcttcatcatcagcatgtacaaggacagccagcctagaggcatggccgtgaccatcagcgtgaagtgcgagaagatcagcaccctgagctgcgagaacaagatcatcagcttcaaggagatgaaccctcctgacaacatcaaggacaccaagagcgacatcatcttcttccagagaagcgtgcctggacacgacaacaagatgcagttcgagagcagcagctacgagggctacttcctggcttgcgagaaggagagagacctgttcaagctgatcctgaagaaggaggacgagctgggagacagaagcatcatgttcaccgtgcagaacgaggacagagctaagagaagcggatctggagctaccaacttcagcctgctgaagcaggctggagatgtggaggagaaccctggacccatgggctggctgtgttccggcctgctgtttcctgtgtcctgtctggtgctgctgcaggtggccagctccgggaacatgaaagtgctgcaggagcccacatgtgtgtccgactacatgtccatctctacatgtgagtggaagatgaacggccccacaaactgctctaccgagctgcggctgctgtaccagctggtgtttctgctgagcgaggcccacacctgtatcccagaaaataatggcggggccgggtgtgtgtgccacctgctgatggatgacgtggtgtctgccgacaattacaccctggacctgtgggccggacagcagctgctgtggaaggggtccttcaaaccctctgagcacgtgaagccaagggcccccggcaacctgacagtgcacaccaacgtgtctgatacactgctgctgacatggagcaatccataccctcctgacaactacctgtacaaccacctgacctacgccgtgaatatctggagcgaaaatgatcctgccgactttcggatttacaatgtgacctatctggagccctccctgagaattgccgcctctaccctgaaatctggaatctcctaccgcgccagggtgcgggcctgggcccagtgttacaacaccacctggtctgagtggagcccaagcaccaagtggcacaattcttatcgggagccttttgagcagcacctgatcccctggctgggacacctgctggtggggctgtctggcgcctttggcttcatcattctggtgtacctgctgatcaactgtaggaatacaggcccttggctgaagaaggtgctgaagtgtaacacccccgacccctctaagttcttcagccagctgtcctctgaacacgggggagatgtgcagaagtggctgtccagccctttcccatccagctcctttagccccgggggcctggcccctgagatctctccactggaagtgctggagcgggacaaggtgacccagctgctgctgcagcaggacaaggtgccagaacccgcctccctgagctccaaccacagcctgacatcttgctttacaaatcagggatacttcttcttccacctgcccgatgccctggagatcgaggcctgccaggtgtacttcacctacgatccctactctgaggaagacccagatgagggcgtggccggggccccaaccgggtccagcccacagccactgcagccactgtccggcgaagatgacgcctactgcacattcccttccagggatgacctgctgctgttcagcccatctctgctgggcggaccctctcctccaagcacagccccagggggatccggcgccggggaagagaggatgccccctagcctgcaggagcgcgtgcccagagactgggacccccagcccctgggccctccaacccctggggtgcccgacctggtggacttccagcctccacccgagctggtgctgagggaggccggcgaagaggtgcccgacgccggcccccgggagggcgtgtccttcccttggtccagacctccaggacagggcgagttccgcgccctgaacgccaggctgcctctgaacaccgatgcctacctgtctctgcaggaactgcagggccaggacccaacccacctggtgcggagaaagcgcagcggctccggcgagggccggggcagcctgctgacctgcggcgacgtggaagagaaccccggacccatgggcccaggagttctgctgctcctgctggtggccacagcttggcatggtcagggaggtgtggtgtcgcacttcaatgactgtccactgtcgcacgatggatactgcctccatgatggtgtgtgcatgtacatcgaggcattggacaagtatgcatgcaactgtgtcgtcggctacatcggagagcgatgtcagtaccgagacctgaagtggtgggaactgagagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacagccactcgag

SEQ ID NO: 114 - protein, 6 aa, Artificial Sequence
  source: /note=“Description of Artificial Sequence: Synthetic peptide”
MYPPPY

SEQ ID NO: 115 - protein, 4 aa, Artificial Sequence
  source: /note=“Description of Artificial Sequence: Synthetic peptide”
SGSG

SEQ ID NO: 116 - protein, 4 aa, Artificial Sequence
  source: /note=“Description of Artificial Sequence: Synthetic peptide”
DEVD

SEQ ID NO: 117 - protein, 8 aa, Artificial Sequence
  source: /note=“Description of Artificial Sequence: Synthetic peptide”
IPESLRAG

SEQ ID NO: 118 - protein, 31 aa, Artificial Sequence
  source: /note=“Description of Artificial Sequence: Synthetic polypeptide”
  VARIANT (6)..(22): /replace=“ ”
  VARIANT (23)..(23): /replace=“Leu”
  VARIANT (24)..(24): /replace=“Glu”
  VARIANT (25)..(25): /replace=“Ser”
  VARIANT (26)..(26): /replace=“Asp”
  SITE (1)..(31): /note=“Variant residues given in the sequence have no preference with respect to those in the annotations for variant positions”
EDDENGGGGSIPESLRAGGGGGSAAAYFGKL

SEQ ID NO: 119 - protein, 14 aa, Artificial Sequence
  source: /note=“Description of Artificial Sequence: Synthetic peptide”
EDDENLESDYFGKL

SEQ ID NO: 120 - protein, 31 aa, Artificial Sequence; identical to SEQ ID NO: 118
  source: /note=“Description of Artificial Sequence: Synthetic polypeptide”

SEQ ID NO: 121 - protein, 4 aa, Artificial Sequence
  source: /note=“Description of Artificial Sequence: Synthetic peptide”
LESD

SEQ ID NO: 122 - protein, 4 aa, Artificial Sequence
  source: /note=“Description of Artificial Sequence: Synthetic peptide”
IETD

(19) United States
(12) Patent Application Publication
(10) Pub. No.: US 2023/0000913 A1
(43) Pub. Date: Jan. 5, 2023

(54) IMMUNORESPONSIVE CELLS ARMOURED WITH SPATIOTEMPORALLY RESTRICTED ACTIVITY OF CYTOKINES OF THE IL-1 SUPERFAMILY

(71) Applicant: King's College London, Strand, London (GB)

(72) Inventors: John MAHER, Strand, London (GB); Caroline Malai HULL, Strand, London (GB)

(21) Appl. No.: 17/634,720

(22) PCT Filed: Aug. 13, 2020
(86) PCT No.: PCT/GB2020/051934

§ 371 Date: Feb. 11, 2022

Related U.S. Application Data
(60) Provisional application No. 62/886,065, filed on Aug. 13, 2019.

(51) Int. Cl. A61K 35/17; C07K 14/545; C12N 5/0783; C12N 9/64

(52) U.S. Cl. CPC A61K 35/17; C07K 14/545; C12N 5/0636; C12N 9/6467; C12N 9/6472; C12N 9/6491; C12Y 304/22061; C07K 2317/34; C07K 2317/565; C07K 2319/03; C07K 2319/02

(57) ABSTRACT

Provided herein are immunoresponsive cells having IL-1 superfamily activities with spatiotemporal restriction. The immunoresponsive cells can further express a protease for regulating the IL-1 superfamily activities, and a chimeric antigen receptor (CAR) or a parallel CAR. Also provided herein are methods of preparing the immunoresponsive cells and methods of directing T cell mediated immune response using the immunoresponsive cells.

### 1. BACKGROUND

The tumour microenvironment imposes restraints on immune effector activity, including effector activities mediated by tumour-infiltrating lymphocytes, T-cells engineered to express non-native T cell receptors (TCRs) and T-cells engineered to express chimeric antigen receptors (CARs). To address such immune suppression within the tumour stroma, there has been interest in engineering immunoresponsive cells to further express one or more proinflammatory cytokines such as interleukin (IL)-12 and/or members of the IL-1 superfamily.

The IL-1 superfamily comprises eleven members. See Baker et al., “IL-1 family members in cancer; two sides to every story,” Front. Immunol. 10: Article 1197 (2019). Pro-inflammatory members include IL-1α, IL-1β, IL-18, IL-33, IL-36α, IL-36β and IL-36γ. By contrast, antagonistic or anti-inflammatory properties have been ascribed to IL-1 receptor antagonist (IL-1Ra), IL-36Ra, IL-37 and IL-38. Importantly, some IL-1 superfamily members are synthesized in precursor forms that require proteolytic cleavage in order to demonstrate biological activity. Examples of cytokines with anti-tumour activity that are regulated in this fashion include IL-1β, IL-18 and IL-36 α-γ.

Like IL-1β and IL-36α-γ, IL-18 lacks a conventional signal or leader sequence that would direct the protein after translation to the secretory pathway involving the endoplasmic reticulum (ER) and Golgi apparatus. Instead, IL-18 is produced as a biologically inactive precursor (pro-IL-18) which is activated by cleavage of a 36 amino acid pro-peptide in the N terminal region. This cleavage reaction is mediated primarily by caspase-1, which is found in the inducible multimolecular organelle known as the inflammasome. Pro-inflammatory IL-36 family members (IL-36α, IL-36β, IL-36γ) are also synthesized as inactive precursors that undergo activation upon proteolytic cleavage of an N-terminal region. Activating enzymes of pro-IL-36 cytokines include cathepsin G, elastase and proteinase 3.

A number of laboratories have engineered CAR- or TCR-engineered T cells to express IL-18. Hu et al., “Augmentation of antitumour immunity by human and mouse CAR T cells secreting IL18,” Cell Rep. 20(13):3025-3033 (2017); Chmielewski et al. “CAR T cells releasing IL-18 convert to T-Bet^{high} FoxO1^{low} effectors that exhibit augmented activity against solid tumors,” Cell Rep. 21 (11):3205-3219 (2017); Avanzi et al., “Engineered tumor-targeted T cells mediate enhanced anti-tumor efficacy both directly and through activation of the endogenous immune system,” Cell Rep. 23(7):2130-2141 (2018); Kunert et al., “Intra-tumoral production of IL18, but not IL12, by TCR-engineered T cells is non-toxic and counteracts immune evasion of solid tumors,” Oncoimmunology 7(1): e1378842 (2017).

Hu et al. showed that the constitutive expression of mature IL-18 by CAR T-cells enhanced both their T-cell receptor dependent amplification in vivo, in addition to anti-tumour activity. In that study, details of how IL-18 was engineered for secretion are not described. Nonetheless, supplementary data demonstrate that IL-18 was both constitutively released (Fig. S1b) and constitutively active (Fig. S1c), suggesting that the mature (18 kD) form of IL-18 was fused to a conventional signal or leader peptide.

Avanzi et al. also demonstrated enhanced anti-tumour activity by IL-18-armoured CAR T cells, accompanied by autocrine CAR T-cell proliferation and persistence. Positive impact on endogenous immune surveillance was indicated by favourable modulation of the cellular infiltrate within tumours. Moreover, epitope spreading occurred, leading to enhanced anti-tumour activity of endogenous T-cells. Use of IL-18 in this manner obviated the need for lymphodepletion to achieve anti-tumour activity. Macrophage depletion significantly hindered therapeutic benefit, supporting an important role for these cells in the modulation of the tumour microenvironment. Because native IL-18 lacks a conventional signal sequence, the IL-18 construct used in the Avanzi publication was mature IL-18 expressed constitutively with an IL-2 signal peptide.

Although expression of IL-18 in CAR-T cells has been shown to improve efficacy in various experiments, safety and therapeutic benefits of constitutive expression of IL-18 have not been fully studied.

Given the strong link between IL-1 family members such as IL-18 and autoinflammatory syndromes such as macrophage-activation syndrome (Weiss et al. “Interleukin-18 diagnostically distinguishes and pathogenically promotes human and murine macrophage activation syndrome,” Blood 131(13):1442-1455 (2018)), there have been concerns that unregulated expression of mature IL-18 or other members of the IL-1 superfamily may have toxicity. Therefore, there is a need for modified strategies for “armouring” immunoresponsive cells against the repressive effects of the tumour microenvironment without causing significant toxicity to non-cancerous tissues.

Chmielewski et al. used an NFAT-responsive promoter in an attempt to restrict the release of mature IL-18 to activated CAR T-cells. They showed that IL-18 producing CAR T-cells modulate the tumour microenvironment, favouring a pro-inflammatory state that is conducive to disease elimination. Tumour-specific T-cells and NK cells were increased at that site, while immunosuppressive M2 polarized macrophages and regulatory T-cells were reduced. Moreover, the profile of costimulatory and co-inhibitory receptors expressed in the tumour were favourably altered. Broadly similar results were obtained in TCR-engineered T cells by Kunert et al. Conceptually, the restriction of mature IL-18 release to activated (NFAT-expressing) T cells should render the approach safer. However, implementation of this solution requires a cumbersome dual transduction procedure. This is because CAR expression is constitutive (achieved using the first vector) while IL-18 expression is inducible (achieved using the second vector). A single vector that contains both promoters might overcome this limitation but would be challenging to produce, given well-known issues with promoter interference. Moreover, this inducible vector demonstrated a degree of “leakiness”, indicated by toxicity seen in tumour-free mice in which IL-12 release was similarly regulated.

### 2. SUMMARY OF THE INVENTION

The present disclosure provides immunoresponsive cells having spatiotemporally restricted activity of IL-1 superfamily members with anti-tumour activity, notably IL-18, IL-36α, IL-36β and IL-36γ. Specifically, immunoresponsive cells are provided that express a modified pro-cytokine of IL-1 superfamily, wherein the modified pro-cytokine comprises, from N-terminus to C-terminus: (a) a pro-peptide; (b) a cleavage site recognized by a protease other than caspase-1, cathepsin G, elastase or proteinase 3; and (c) a biologically active cytokine fragment of the IL-1 superfamily.

CAR T-cells—both αβ CAR-T cells and γδ CAR-T cells—were generated in which an exogenous polynucleotide encoding the pro-cytokine with a cleavage site recognized by a site-specific protease other than caspase-1, cathepsin G, elastase or proteinase 3 was further introduced. In some experiments, the cells further expressed the site-specific protease. In particular, provided herein includes pro-cytokine with a cleavage site recognized by the protease, granzyme B (GzB). The applicant has found that expression of the IL-1 superfamily member with regulated activities can enhance T cell responses and anti-tumour activity of CAR T-cells in a controlled manner.

The pro-cytokine with the regulated activities can be used in combination with various CAR T-cells available in the art. For example, pCAR-T cells having parallel CAR (pCAR) constructs that bind to one or more antigens present on a target cell can be further modified to express the pro-cytokine with regulated activities.

Thus, according to some embodiments, provided herein is an immunoresponsive cell expressing: a modified pro-cytokine of IL-1 superfamily, wherein the modified pro-cytokine comprises, from N-terminus to C-terminus: (a) a pro-peptide; (b) a cleavage site recognized by a protease other than caspase-1, cathepsin G, elastase or proteinase 3; and (c) a cytokine fragment of the IL-1 superfamily.

In some embodiments, the protease is granzyme B (GzB). In some embodiments, the cleavage site has a sequence of SEQ ID NO: 26. In some embodiments, the modified pro-cytokine is a modified pro-IL-18 and has a sequence of SEQ ID NO: 27. In some embodiments, the modified pro-IL-18 was expressed from a polynucleotide of SEQ ID NO: 103 or 111.

In some embodiments, the protease is caspase-3. In some embodiments, the cleavage site has a sequence of SEQ ID NO: 28. In some embodiments, the modified pro-cytokine is a modified pro-IL-18 and has a sequence of SEQ ID NO: 29. In some embodiments, the modified pro-IL-18 was expressed from a polynucleotide of SEQ ID NO: 109.

In some embodiments, the protease is caspase-8. In some embodiments, the cleavage site has a sequence of SEQ ID NO: 30. In some embodiments, the modified pro-cytokine is a modified pro-IL-18 and has a sequence of SEQ ID NO: 31. In some embodiments, the modified pro-IL-18 was expressed from a polynucleotide of SEQ ID NO: 107.

In some embodiments, the protease is membrane-type 1 matrix metalloproteinase (MT1-MMP). In some embodiments, the cleavage site has a sequence of SEQ ID NO: 32. In some embodiments, the modified pro-cytokine is a modified pro-IL-18 and has a sequence of SEQ ID NO: 33. In some embodiments, the modified pro-IL-18 was expressed from a polynucleotide of SEQ ID NO: 113.

In some embodiments, the cytokine fragment is a polypeptide having at least 85%, 90%, 95%, 97%, 98%, 99% or 100% sequence identity to SEQ ID: 24. In some embodiments, the cytokine fragment is a polypeptide having at least about 85%, 90%, 95%, 97%, 98%, 99% or 100% sequence identity to SEQ ID: 24.

In some embodiments, the pro-peptide is a polypeptide having at least 85%, 90%, 95%, 97%, 98%, 99% or 100% sequence identity to SEQ ID: 25. In some embodiments, the pro-peptide is a polypeptide having at least about 85%, 90%, 95%, 97%, 98%, 99% or 100% sequence identity to SEQ ID: 25.

In some embodiments, the modified pro-cytokine is a modified pro-IL-36a and has a sequence of SEQ ID NO: 37. In some embodiments, the cytokine fragment is a polypeptide having at least 85%, 90%, 95%, 97%, 98%, 99% or 100% sequence identity to SEQ ID: 42. In some embodiments, the cytokine fragment is a polypeptide having at least about 85%, 90%, 95%, 97%, 98%, 99% or 100% sequence identity to SEQ ID: 42.

In some embodiments, the modified pro-cytokine is a modified pro-IL-36β and has a sequence of SEQ ID NO: 39. In some embodiments, the cytokine fragment is a polypeptide having at least 85%, 90%, 95%, 97%, 98%, 99% or 100% sequence identity to SEQ ID: 43. In some embodiments, the cytokine fragment is a polypeptide having at least about 85%, 90%, 95%, 97%, 98%, 99% or 100% sequence identity to SEQ ID: 43.

In some embodiments, the modified pro-cytokine is a modified pro-IL-36γ and has a sequence of SEQ ID NO: 41. In some embodiments, the cytokine fragment is a polypeptide having at least 85%, 90%, 95%, 97%, 98%, 99% or 100% sequence identity to SEQ ID: 44. In some embodiments, the cytokine fragment is a polypeptide having at least about 85%, 90%, 95%, 97%, 98%, 99% or 100% sequence identity to SEQ ID: 44.

In some embodiments, the immunoresponsive cell further comprises an exogenous polynucleotide encoding the protease.

In some embodiments, said immunoresponsive cell is an αβ T cell, γδ T cell, or a Natural Killer (NK) cell. In some embodiments, said T cell is an αβ T cell. In some embodiments, said T cell is a γδ T-cell.

In some embodiments, said immunoresponsive cell further comprises a chimeric antigen receptor (CAR). In some embodiments, the CAR is a second-generation chimeric antigen receptor (CAR), wherein the CAR comprises: (a) a signalling region; (b) a first co-stimulatory signalling region; (c) a transmembrane domain; and (d) a first binding element that specifically interacts with a first epitope on a first target antigen.

In some embodiments, the first epitope is an epitope on a MUC1 target antigen. In some embodiments, said first binding element comprises the CDRs of the HMFG2 antibody. In some embodiments, said first binding element comprises the V_{H} and V_{L} domains of the HMFG2 antibody. In some embodiments, said first binding element comprises an HMFG2 single-chain variable fragment (scFv).

In some embodiments, the immunoresponsive cell further comprises a chimeric co-stimulatory receptor (CCR), wherein the CCR comprises: (a) a second co-stimulatory signalling region; (b) a transmembrane domain; and (c) a second binding element that specifically interacts with a second epitope on a second target antigen.

In some embodiments, the second co-stimulatory domain is different from the first co-stimulatory domain. In some embodiments, the second target antigen comprising said second epitope is selected from the group consisting of ErbB homodimers and heterodimers. In some embodiments, said second target antigen is HER2. In some embodiments, said second target antigen is the EGF receptor. In some embodiments, said second binding element comprises T1E, the binding moiety of ICR12, or the binding moiety of ICR62.

In some embodiments, the present disclosure provides an immunoresponsive cell expressing a modified pro-IL-18, wherein the modified pro-IL-18 is a polypeptide of SEQ ID NO: 27, and wherein the cell further comprises: (a) an exogenous polynucleotide encoding GzB; (b) a chimeric antigen receptor (CAR) comprising: i. a signalling region; ii. a first co-stimulatory signalling region; iii. a transmembrane domain; and iv. a first binding element that specifically interacts with a first epitope on a MUC1 target antigen; and (c) a chimeric co-stimulatory receptor (CCR) comprising: i. a second co-stimulatory signalling region; ii. transmembrane domain; and iii. a second binding element that specifically interacts with a second epitope on a second target antigen.

In some embodiments, the present disclosure provides an immunoresponsive cell expressing a modified pro-IL-36α, pro-IL-36β or pro-IL-36γ, wherein the modified pro-IL-36α, pro-IL-36β or pro-IL-36γ is a polypeptide of SEQ ID NO: 37, 39 or 41, and wherein the cell further comprises: (a) an exogenous polynucleotide encoding GzB; (b) a chimeric antigen receptor (CAR) comprising: i. a signalling region; ii. a first co-stimulatory signalling region; iii. a transmembrane domain; and iv. a first binding element that specifically interacts with a first epitope on a MUC1 target antigen; and (c) a chimeric co-stimulatory receptor (CCR) comprising: i. a second co-stimulatory signalling region; ii. transmembrane domain; and iii. a second binding element that specifically interacts with a second epitope on a second target antigen.

In another aspect, the present disclosure provides a polynucleotide or set of polynucleotides comprising a first nucleic acid encoding a modified cytokine, wherein the modified pro-cytokine of IL-1 superfamily comprises, from N-terminus to C-terminus: (a) a pro-peptide; (b) a cleavage site recognized by a protease other than caspase-1, cathepsin G, elastase or proteinase 3; and (c) a cytokine fragment of the IL-1 superfamily.

In some embodiments, the protease is GzB. In some embodiments, the cleavage site has a sequence of SEQ ID NO: 26. In some embodiments, the modified pro-cytokine is a modified pro-IL-18 has a sequence of SEQ ID NO: 27. In some embodiments, the polynucleotide or set of polynucleotides comprise a sequence of SEQ ID NO: 103 or 111.

In some embodiments, the protease is caspase-3. In some embodiments, the cleavage site has a sequence of SEQ ID NO: 28. In some embodiments, the modified cytokine is a modified pro-IL-18 and has a sequence of SEQ ID NO: 29. In some embodiments, the polynucleotide or set of polynucleotides comprise a sequence of SEQ ID NO: 109.

In some embodiments, the protease is caspase-8. In some embodiments, the cleavage site has a sequence of SEQ ID NO: 30. In some embodiments, the modified cytokine is a modified pro-IL-18 and has a sequence of SEQ ID NO: 31. In some embodiments, the polynucleotide or set of polynucleotides comprise a sequence of SEQ ID NO: 107.

In some embodiments, the protease is MT1-MMP. In some embodiments, the cleavage site has a sequence of SEQ ID NO: 32. In some embodiments, the modified pro-cytokine is a modified pro-IL-18 and has a sequence of SEQ ID NO: 33. In some embodiments, the polynucleotide or set of polynucleotides comprise a sequence of SEQ ID NO: 113.

In some embodiments, the polynucleotide or set of polynucleotides further comprises a second nucleic acid encoding the protease.

In some embodiments, the first nucleic acid and the second nucleic acid are in a single vector.

In some embodiments, the cytokine fragment is a polypeptide having at least 85%, 90%, 95%, 97%, 98%, 99% or 100% sequence identity to SEQ ID: 24. In some embodiments, the cytokine fragment is a polypeptide having at least about 85%, 90%, 95%, 97%, 98%, 99% or 100% sequence identity to SEQ ID: 24. In some embodiments, the cytokine fragment can bind and activate an IL-18 receptor when the cleavage site is cleaved. In some embodiments, the pro-peptide is a polypeptide having at least 85%, 90%, 95%, 97%, 98%, 99% or 100% sequence identity to SEQ ID: 25. In some embodiments, the pro-peptide is a polypeptide having at least about 85%, 90%, 95%, 97%, 98%, 99% or 100% sequence identity to SEQ ID: 25.

In some embodiments, the modified pro-cytokine is a modified pro-IL-36a and has a sequence of SEQ ID NO: 37. In some embodiments, the cytokine fragment is a polypeptide having at least 85%, 90%, 95%, 97%, 98%, 99% or 100% sequence identity to SEQ ID: 42. In some embodiments, the cytokine fragment is a polypeptide having at least about 85%, 90%, 95%, 97%, 98%, 99% or 100% sequence identity to SEQ ID: 42

In some embodiments, the modified pro-cytokine is a modified pro-IL-36β and has a sequence of SEQ ID NO: 39. In some embodiments, the cytokine fragment is a polypeptide having at least 85%, 90%, 95%, 97%, 98%, 99% or 100% sequence identity to SEQ ID: 43. In some embodiments, the cytokine fragment is a polypeptide having at least about 85%, 90%, 95%, 97%, 98%, 99% or 100% sequence identity to SEQ ID: 43

In some embodiments, the modified pro-cytokine is a modified pro-IL-36γ and has a sequence of SEQ ID NO: 41. In some embodiments, the cytokine fragment is a polypeptide having at least 85%, 90%, 95%, 97%, 98%, 99% or 100% sequence identity to SEQ ID: 44. In some embodiments, the cytokine fragment is a polypeptide having at least about 85%, 90%, 95%, 97%, 98%, 99% or 100% sequence identity to SEQ ID: 44

In some embodiments, the polynucleotide or set of polynucleotides comprises a first nucleic acid encoding a modified pro-IL-36 α, β or γ, wherein the modified pro-IL-36 α, β or γ, comprises, from N-terminus to C-terminus: (a) a pro-peptide; (b) a cleavage site recognized by a protease other than cathepsin G, elastase or proteinase 3; and (c) an IL-36 α, β or γ fragment.

In some embodiments, the protease is granzyme B (GzB). In some embodiments, the cleavage site has a sequence of SEQ ID NO: 26. In some embodiments, the modified pro-IL-36 α, β or γ comprises a sequence of SEQ ID NO: 37, 39 or 41.

In some embodiments, the polynucleotide or set of polynucleotides further comprising a second nucleic acid encoding the protease. In some embodiments, the first nucleic acid and the second nucleic acid are in a single vector.

In some embodiments, the IL-36 fragment is a polypeptide having at least 85%, 90%, 95%, 97%, 98%, 99% or 100% sequence identity to SEQ ID: 42, 43 or 44. In some embodiments, the IL-36 fragment is a polypeptide having at least about 85%, 90%, 95%, 97%, 98%, 99% or 100% sequence identity to SEQ ID: 42, 43 or 44. In some embodiments, the IL-36 fragment can bind and activate an IL-36 receptor when the cleavage site is cleaved.

In some embodiments, the polynucleotide or set of polynucleotides further comprises a third nucleic acid encoding a chimeric antigen receptor (CAR). In some embodiments, the CAR is a second-generation chimeric antigen receptor (CAR), comprising: (a) a signalling region; (b) a first co-stimulatory signalling region; (c) a transmembrane domain; and (d) a first binding element that specifically interacts with a first epitope on a first target antigen.

In some embodiments, the first epitope is an epitope on a MUC1 target antigen. In some embodiments, said first binding element comprises the CDRs of the HMFG2 antibody. In some embodiments, said first binding element comprises the V_{H} and V_{L} domains of HMFG2 antibody. In some embodiments, said first binding element comprises HMFG2 single-chain variable fragment (scFv).

In some embodiments, the polynucleotide or set of polynucleotides further comprises a fourth nucleic acid encoding a chimeric co-stimulatory receptor (CCR), wherein the CCR comprises: (a) a second co-stimulatory signalling region; (b) a transmembrane domain; and (c) a second binding element that specifically interacts with a second epitope on a second target antigen.

In some embodiments, the second target antigen comprising said second epitope is selected from the group consisting of ErbB homodimers and heterodimers. In some embodiments, said second target antigen is HER2. In some embodiments, said second target antigen is EGF receptor. In some embodiments, said second binding element comprises T1E, the binding moiety of ICR12, or the binding moiety of ICR62.

In some embodiments, the third nucleic acid and the fourth nucleic acid are in a single vector.

In some embodiments, the polynucleotide or set of polynucleotides comprise: (a) a first nucleic acid encoding a modified pro-IL-18, wherein the modified pro-IL-18 is a polypeptide of SEQ ID NO: 27; (b) a second nucleic acid encoding GzB; (c) a third nucleic acid encoding a chimeric antigen receptor (CAR), wherein the CAR comprises: i. a signalling region; ii. a first co-stimulatory signalling region; iii. a transmembrane domain; and iv. a first binding element that specifically interacts with a first epitope on a MUC1 target antigen; (d) a fourth nucleic acid encoding a chimeric co-stimulatory receptor (CCR), wherein the CCR comprises: i. a second co-stimulatory signalling region; ii. transmembrane domain; and iii. a second binding element that specifically interacts with a second epitope on a second target antigen. In some embodiments, the polynucleotide or set of polynucleotides comprises the polynucleotide of SEQ ID NO: 103.

In some embodiments, the polynucleotide or set of polynucleotides comprise: (a) a first nucleic acid encoding a modified pro-IL-36, wherein the modified pro-IL-36 is a polypeptide of SEQ ID NO: 37, 39 or 41; (b) a second nucleic acid encoding GzB; (c) a third nucleic acid encoding a chimeric antigen receptor (CAR), wherein the CAR comprises: i. a signalling region; ii. a first co-stimulatory signalling region; iii. a transmembrane domain; and iv. a first binding element that specifically interacts with a first epitope on a MUC1 target antigen; (d) a fourth nucleic acid encoding a chimeric co-stimulatory receptor (CCR), wherein the CCR comprises: i. a second co-stimulatory signalling region; ii. transmembrane domain; and iii. a second binding element that specifically interacts with a second epitope on a second target antigen.

In some embodiments, said first nucleic acid and said third nucleic acid are in a single vector. In some embodiments, said first nucleic acid and said fourth nucleic acid are expressed from a single vector. In some embodiments, said first nucleic acid, said second nucleic acid, said third nucleic acid, and said fourth nucleic acid are expressed from a single vector.

In one aspect, the present invention provides a method of preparing the immunoresponsive cell, said method comprising transfecting or transducing the polynucleotide or set of polynucleotides provided herein into an immunoresponsive cell.

In another aspect, the present disclosure provides a method for directing a T cell-mediated immune response to a target cell in a patient in need thereof, said method comprising administering to the patient the immunoresponsive cell provided in this disclosure. In some embodiments, the target cell expresses MUC1.

In yet another aspect, the present disclosure provides a method of treating cancer, said method comprising administering to the patient an effective amount of the immunoresponsive cell provided in this disclosure. In some embodiments, the patient's cancer cell expresses MUC1. In some embodiments, the patient has a cancer selected from the group consisting of breast cancer, ovarian cancer, pancreatic cancer, colorectal cancer, lung cancer, gastric cancer, bladder cancer, myeloma, non-Hodgkin lymphoma, prostate cancer, esophageal cancer, endometrial cancer, hepatobiliary cancer, duodenal carcinoma, thyroid carcinoma, and renal cell carcinoma. In some embodiments, the patient has breast cancer. In some embodiments, the patient has ovarian cancer.

In one aspect, the present disclosure provides a γδ T cell expressing:

(a) a second generation chimeric antigen receptor (CAR) comprising
i. a signalling region;
ii. a co-stimulatory signalling region;
iii. a transmembrane domain;
iv. a first binding element that specifically interacts with a first epitope on a first target antigen; and
   (b) a chimeric co-stimulatory receptor (CCR) comprising
v. a co-stimulatory signalling region which is different from that of ii;
vi. a transmembrane domain; and
vii. a second binding element that specifically interacts with a second epitope on a second target antigen.

In some embodiments, the first target antigen is the same as the second target antigen.

In some embodiments, the first target antigen is a MUC antigen. In some embodiments, said first binding element comprises the CDRs of the HMFG2 antibody. In some embodiments, said first binding element comprises the V_{H} and V_{L} domains of HMFG2 antibody. In some embodiments, said first binding element comprises HMFG2 single-chain variable fragment (scFv).

In some embodiments, said second target antigen comprising said second epitope is selected from the group consisting of ErbB homodimers and heterodimers. In some embodiments, said second target antigen is HER2. In some embodiments, said second target antigen is EGF receptor. In some embodiments, said second binding element comprises T1E, ICR12, or ICR62. In some embodiments, said second binding element is T1E. In some embodiments, said second target antigen is αvβ6 integrin. In some embodiments, said second binding element is A20 peptide.

In yet another aspect, the present disclosure provides a method of making an immunoresponsive cell, comprising a step of introducing a transgene. In some embodiments, the transgene encodes a CAR or pCAR. In some embodiments, the transgene encodes a modified pro-cytokine of IL-1 superfamily, wherein the modified pro-cytokine comprises, from N-terminus to C-terminus: (a) a pro-peptide; (b) a cleavage site recognized by a protease other than caspase-1, cathepsin G, elastase or proteinase 3; and (c) a cytokine fragment of the IL-1 superfamily. In some embodiments, the method further comprises a preceding step of activating the γδ T cell with an anti-γδ TCR antibody. In some embodiments, the anti-γδ TCR antibody is immobilised.

### 3. BRIEF DESCRIPTION OF THE DRAWINGS

The drawings are not necessarily to scale, emphasis instead being placed upon illustrating the principles of various embodiments of the invention.

FIG. 1 provides schematic diagrams showing salient features of certain second generation CAR and pCAR constructs used in the experiments described herein. The cell membrane is shown as parallel horizontal lines, with the extracellular domains depicted above the membrane and intracellular domains shown below the membrane. For pCARs, the chimeric costimulatory receptor (CCR) is named first, with the CAR identified to the right of a slash or stroke mark (/).

H2 is a second generation CAR originally described in Wilkie et al., J. Immunol. 180:4901-9 (2008), incorporated herein by reference in its entirety. It comprises, from extracellular to intracellular domains, a human MUC1-targeting HMFG2 single chain antibody (scFv) domain, CD28 transmembrane and costimulatory domains, and a CD3z signalling region. Cells transduced with H2 alone are standard 2^{nd} generation CAR-T cells having specificity for the MUC1 tumour-associated glycoforms recognized by the HMFG2 scFv.

TBB/H is a pCAR. It utilizes the MUC1-targeting 2^{nd} generation “H2” CAR, but with a co-expressed chimeric costimulatory receptor (CCR). The CCR in the TBB/H pCAR has a T1E binding domain fused to CD8α transmembrane domain and a 4-1BB co-stimulatory domain. T1E is a chimeric peptide derived from transforming growth factor-α (TGF-α) and epidermal growth factor (EGF) and is a promiscuous ErbB ligand. See Wingens et al., “Structural analysis of an epidermal growth factor/transforming growth factor-alpha chimera with unique ErbB binding specificity,” J. Biol. Chem. 278:39114-23 (2003) and Davies et al., “Flexible targeting of ErbB dimers that drive tumorigenesis by using genetically engineered T cells,” the disclosures of which are incorporated herein by reference in their entireties.

FIG. 2 is a cartoon illustrating the modification of pro-IL-18 in various of the constructs used herein. IL-18 is secreted as inactive pro-IL-18. In native pro-IL-18, activation requires caspase-1 cleavage at a cleavage site between the pro-peptide and mature IL-18 protein fragment. However, caspase-1 is not expressed in T-cells. Caspase-3 and caspase-8 are upregulated in the cytoplasm of activated T-cells (Alam et al., “Early activation of caspases during T lymphocyte stimulation results in selective substrate cleavage in nonapoptotic cells,” J. Exp. Med 190(12):1879-1890 (1999); Chun et al. “Pleiotropic defects in lymphocyte activation caused by caspase-8 mutations lead to human immunodeficiency,” Nature 419(6905):395-9 (2002)). In the constructs shown at the bottom, the native caspase-1 cleavage site within pro-IL-18 has been replaced by a caspase-3 cleavage site or caspase-8 cleavage site, a GzB cleavage site or MT1-MMP cleavage site. These modified derivatives are designated pro-IL-18 (casp 3), pro-IL-18 (casp 8), pro-IL-18 (GzB) and pro-IL-18 (MT1-MMP) respectively. Comparison is made with a constitutively active form of IL-18, designated “constit IL-18” in which mature IL-18 has been placed downstream of a CD4 signal peptide.

FIG. 3 provides flow cytometry (FACS) results confirming co-expression of the second generation H2 CAR (“H28z”) and the TBB CCR (“TIE”) (together, the TBB/H pCAR) and IL-18 variants in T cells that were transfected with a retroviral vector encoding both the 2^{nd} generation TBB/H pCAR and the IL-18 variants identified along the top of the figure. Transfected T cells were analyzed for expression of the two components of the pCAR, separately measuring expression of the H28z CAR (H-2) and TIE-4-1BB CCR using FACS.

FIG. 4A shows secretion of pro-IL-18 or modified pro-IL-18 in transduced T cells as analyzed by ELISA. FIG. 4B shows functional activities of secreted IL-18 measured by an IL-18-responsive colorimetric reporter assay.

FIGS. 5A-5D provide percentage survival rates of MDA-MB-468 breast cancer cells after co-culture of the cancer cells with the pCAR T-cells expressing pro-IL-18 or modified pro-IL-18 (pro-IL-18 for FIG. 5A; constitutive (constit) IL-18 for FIG. 5B; pro-IL-18 (casp 8) for FIG. 5C; and pro-IL-18 (casp 3) for FIG. 5D) at different effector:target (T cell:tumour cell) ratios (x-axis).

FIG. 6A provides T-cell numbers and FIG. 6B provides percentage survival rates of MDA-MB-468 breast cancer cells after the indicated number of restimulation cycles with T cells expressing the TBB/H pCAR and pro-IL-18 or modified pro-IL-18 (constit IL-18, pro-IL-18 (casp 8) or pro-IL-18 (casp 3)).

FIG. 7A provides IL-18 secretion levels detected by ELISA and FIG. 7B provides IL-18 functional activities without stimulation (unstim) or with stimulation using anti-CD3/CD28 antibodies in CAR T-cells expressing the TBB/H MUC1 pCAR alone, TBB/H and pro-IL-18 (GzB), or TBB/H and constit IL-18.

FIG. 8 compares percentage survival rates of MDA-MB-468 breast cancer cells after co-culture of the cancer cells with untransduced T-cells, TBB/H pCAR T-cells, TBB/H pCAR T-cells that express pro-IL-18 or TBB/H pCAR T-cells that co-express pro-IL-18 (GzB) with additional granzyme B.

FIG. 9A provides levels of IL-18 and FIG. 9B provides levels of IFN-γ secreted from TBB/H pCAR T-cells. Comparison is made between TBB/H alone (do not express exogenous IL-18) and TBB/H pCAR T-cells that co-express pro-IL-18 or that co-express pro-IL-18 (GzB) with additional granzyme B.

FIG. 10A provides percentage survival rates of MDA-MD-468 cells and FIG. 10B provides percentage survival rates of BxPC-3 cells after restimulation cycles with T cells. Comparison is made between untransduced T cells, TBB/H pCAR T-cells (do not express exogenous IL-18) and TBB/H pCAR T-cells that either co-express pro-IL-18, constit IL-18 or the combination of pro-IL-18 (GzB) with additional granzyme B.

FIGS. 11A-11B provides the numbers of successful cycles of antigen stimulation of CAR-T cells with MDA-MD-468 tumour target cells (FIG. 11A) or BxPC-3 tumour target cells (FIG. 11B). Cells tested were TBB/H pCAR T-cells expressing no exogenous IL-18 (TBB/H) or TBB/H pCAR T-cells expressing pro-IL-18 or pro-IL-18 (GzB) together with additional granzyme B. Restimulation causing more than 20% cytotoxicity of the target tumour cells was considered to be a successful restimulation cycle.

FIG. 12 provides the number of T cells at the 4^{th} restimulation cycle for pCAR T-cells expressing no exogenous IL-18 (TBB/H) or TBB/H pCAR T-cells expressing pro-IL-18 or pro-IL-18 (GzB) together with additional granzyme B.

FIG. 13 graphs bioluminescence emission (“total flux”) in tumour-injected mice treated with PBS or pCAR T-cells expressing no exogenous IL-18 (TBB/H) or TBB/H pCAR T-cells expressing pro-IL-18, constit IL-18 or pro-IL-18 (GzB) together with additional granzyme B.

FIG. 14 provides FACS data showing T cell expression of pCAR (top) or γδ TCR (bottom) in γδ T-cells transduced with a retroviral vector encoding TBB/H pCAR alone (TBB/H) or TBB/H pCAR together with one of four IL-18 variants (pro-IL-18+pCAR; pro-IL-18 (GzB)+pCAR; constit IL-18+pCAR; or pro-IL-18 (GzB)+pCAR together with additional granzyme B).

FIG. 15A provides percentage survival rates of MDA-MD-468 cells and FIG. 15B provides percentage survival rates of BxPC-3 cells after co-culture with either untransduced T-cells or TBB/H pCAR T-cells expressing no exogenous IL-18 (TBB/H) or expressing an IL-18 variant (either pro-IL-18, constit IL-18, pro-IL-18 (GzB) or pro-IL-18 (GzB) with additional granzyme B) at different effector:target ratios.

FIG. 16 provides a diagram illustrating the structure of the construct encoding pro-IL-18 with a cleavage site recognized by MT1-MMP (MMP14).

FIGS. 17A-17C show bioluminescence emission (“total flux”) in SKOV-3 tumour-injected mice treated with 0.5 million of T4 CAR T cells (FIG. 17A), TINA CART cells (a signalling defective endodomain truncated control of T4, FIG. 17B) or T cells that co-express T4+pro-IL-18 (MT1-MMP) (FIG. 17C).

FIG. 18 provides a diagram illustrating the structure of the SFG retroviral construct encoding the TBB/H pCAR and pro-IL-18.

FIG. 19 provides a diagram illustrating the structure of the SFG retroviral construct encoding TBB/H pCAR and a modified pro-IL-18 with the GzB cleavage site, designated pro-IL-18 (GzB).

FIG. 20 provides a diagram illustrating the structure of the SFG retroviral construct encoding TBB/H pCAR and a constitutively active IL-18, designated constit IL-18.

FIG. 21 provides a diagram illustrating the structure of the SFG retroviral construct encoding TBB/H pCAR and a modified pro-IL-18 with a caspase-8 cleavage site, designated pro-IL-18 (casp 8).

FIG. 22 provides a diagram illustrating the structure of the SFG retroviral construct encoding TBB/H pCAR and a modified pro-IL-18 with a caspase-3 cleavage site, designated pro-IL-18 (casp 3).

FIG. 23 provides a diagram illustrating the structure of the SFG retroviral construct encoding TBB/H pCAR, a modified pro-IL-18 with a GzB cleavage site and additional granzyme B, designated pro-IL-18 (GzB)+granzyme B.

FIG. 24 provides a diagram illustrating the structure of the SFG retroviral construct encoding T4 pCAR and a modified pro-IL-18 with an MP1-MMP cleavage site, designated pro-IL-18 (MT1-MMP).

FIG. 25 provides illustrations of various first-generation CAR, co-stimulatory chimeric receptor, and second-generation CARs that can be used in various embodiments of the immunoresponsive cells disclosed herein.

FIG. 26 provides illustrations of various third-generation CARs and cis and trans co-stimulatory chimeric receptors that can be used in various embodiments of the immunoresponsive cells disclosed herein.

FIG. 27 provides illustrations of various dual-targeted CARs, inhibitory CARs/NOT gate, combinatorial CARs/AND gate, and TanCARs that can be used in various embodiments of the immunoresponsive cells disclosed herein.

FIG. 28 provides illustrations of Go-CART, Trucks, Armoured CARs, and CARs with engineered co-stimulation that can be used in various embodiments of the immunoresponsive cells disclosed herein.

FIG. 29 provides illustrations of SynNotch/sequential AND gate CAR and parallel (p)CAR that can be used in various embodiments of the immunoresponsive cells described herein.

FIG. 30A graphs total flux in tumour-injected mice treated with PBS or 10 million TBB/H pCAR-αβ T-cells expressing no exogenous IL-18 (TBB/H) or TBB/H pCAR-αβ T-cells expressing pro-IL-18 or pro-IL-18 (GzB) together with additional granzyme B. FIG. 30B graphs total flux in tumour-injected mice treated with PBS or 8 million TBB/H pCAR-γδ T-cells expressing no exogenous IL-18 (TBB/H) or TBB/H pCAR-γδ T-cells expressing pro-IL-18 or pro-IL-18 (GzB) together with additional granzyme B. FIG. 30C graphs total flux in tumour-injected mice treated with PBS or 4 million TBB/H pCAR-γδ T-cells expressing no exogenous IL-18 (TBB/H) or TBB/H pCAR-γδ T-cells expressing pro-IL-18 or pro-IL-18 (GzB) together with additional granzyme B. All graphs show pooled data from 3 individual mice.

FIG. 31 graphs total flux in three individual tumour-injected mice treated with PBS as a control.

FIG. 32A-32B provide total flux in individual tumour-injected mice treated with 8×10⁶ TBB/H pCAR-γδ T cells (FIG. 32A), or 4×10⁶ TBB/H pCAR-γδ T cells (FIG. 32B). In each case, T cells lacked expression of exogenous of IL-18.

FIG. 33A-33B provide total flux in individual tumour-injected mice treated with 8×10⁶ TBB/H pCAR-γδ T cells (FIG. 33A), or 4×10⁶ TBB/H pCAR-γδ T cells (FIG. 33B). In each case, T cells also produced exogenous pro-IL-18.

FIG. 34A-34B provide total flux in individual tumour-injected mice treated with 8×10⁶ TBB/H pCAR-γδ T cells (FIG. 34A), or 4×10⁶ TBB/H pCAR-γδ T cells (FIG. 34B). In each case, T cells also produced exogenous pro-IL-18 (GzB) and exogenous granzyme B.

FIG. 35 shows IL-18 activity measured in αβ T cell culture following stimulation with MUC1⁺MDA-MB-468 breast cancer cells (“+468”) or beads coated with anti-CD3 and anti-CD28 antibodies (“aCD3/28 beads”). Tested αβ T cells were untransduced or transduced to express (i) TBBH, (ii) TBBH and pro-IL-18 (GzB), (iii) TBBH and pro-IL-18 (GzB), (iv) TBBH, pro-IL-18 (GzB) and granzyme B, or (iv) TBBH and constit IL-18.

FIG. 36A-36F graph bioluminescence emission (“total flux”) in tumour-injected mice treated with or without αβ T cells. Graphs show results of mice treated with PBS (FIG. 36A), or αβ T cells expressing TBB/H (FIG. 36B), TBB/H+pro-IL-18 (FIG. 36C), TBB/H+pro-IL-18 (GzB) (FIG. 36D), TBB/H+ constit IL-18 (FIG. 36E), or TBB/H+pro-IL-18 (GzB)+granzyme B (FIG. 36F).

FIG. 37 shows the survival curves of tumor-injected mice treated with αβ TBB/H pCAR T cells or αβ TBB/H pCAR T cells that further express pro-IL-18 (GzB), constit IL-18, or pro-IL-18 (GzB) together with granzyme B.

FIG. 38 provides the numbers of successful restimulation cycles of TBB/H pCAR-T cells expressing no exogenous IL-18 (TBB/H) or TBB/H pCAR T-cells expressing pro-IL-18, pro-IL-18 (GzB), pro-IL-18 (GzB) together with additional granzyme B, or constit IL-18. The pCAR T cells were cultured with MDA-MD-468 tumour target cells (FIG. 38A) or BxPC-3 tumour target cells (FIG. 38B). Restimulation causing more than 30% cytotoxicity to the target tumour cells was considered to be a successful restimulation cycle.

FIG. 39 shows IL-18 activity measured in γδ T cell culture following stimulation with MUC1⁺MDA-MB-468 breast cancer cells (“+468”) or beads coated with anti-CD3 and anti-CD28 antibodies (“aCD3/28 beads”). The γδ T cells were untransduced or transduced to express (i) TBBH, (ii) TBBH and pro-IL-18 (GzB), (iii) TBBH and pro-IL-18 (GzB), (iv) TBBH, pro-IL-18 (GzB) and granzyme B, or (iv) TBBH and constit IL-18.

FIG. 40A-40F show bioluminescence emission (“total flux”) in tumour-injected mice treated with or without γδ T cells. Graphs show results of mice treated with PBS (FIG. 40A), or γδ T cells expressing TBB/H (FIG. 40B), TBB/H+pro-IL-18 (FIG. 40C), TBB/H+pro-IL-18 (GzB) (FIG. 40D), TBB/H+ constit IL-18 (FIG. 40E), and TBB/H+pro-IL-18 (GzB)+granzyme B (FIG. 40F).

FIG. 41 shows the survival curves of tumor-injected mice treated with γδ TBB/H pCAR T cells or γδ TBB/H pCAR T cells that further express pro-IL-18 (GzB), constit IL-18, or pro-IL-18 (GzB) together with granzyme B.

FIG. 42A provides percentage survival rates of MDA-MD-468 LT cells and FIG. 42B provides percentage survival rates of BxPC-3 LT cells after restimulation cycles with TBB/H pCAR T cells. Comparison is made between TBB/H pCAR T-cells (do not express exogenous IL-36) and TBB/H pCAR T-cells that either co-express the combination of either pro-IL-36γ together with granzyme B, or pro-IL-36γ (GzB) together with granzyme B.

FIG. 43 provides the number of T cells at each restimulation cycle in assays targeting MDA-MB-468 cells (FIG. 43A) or BxPC-3 cells (FIG. 43B) for pCAR T-cells expressing no exogenous IL-36 (TBB/H), TBB/H pCAR T-cells expressing pro-IL36γ together with granzyme B, or pro-IL-36γ (GzB) together with granzyme B.

FIG. 44A and FIG. 44B provide levels of IFN-γ secreted from TBB/H pCAR T-cells co-cultured with MDA-468-LT cells (FIG. 44A) or BxPC3-LT cells (FIG. 44B). Comparison is made between TBB/H pCAR T-cells (do not express exogenous IL-36) and TBB/H pCAR T-cells that either co-express the combination of either pro-IL-36γ together with granzyme B, or pro-IL-36γ (GzB) together with granzyme B.

FIG. 45 compares percentage survival rates of MDA-MB-468-LT cells after co-culture of the cancer cells with untransduced T-cells, TBB/H pCAR T-cells, or TBB/H pCAR T-cells that further express pro-IL-36γ and granzyme B, or pro-IL-36γ (GzB) and granzyme B at a range of initial effector to target cell ratios (E:T).

FIG. 46 compares percentage survival rates of BxPC3-LT cells after co-culture of the cancer cells with untransduced T-cells, TBB/H pCAR T-cells, or TBB/H pCAR T-cells that further express pro-IL-36γ and granzyme B, or pro-IL-36γ (GzB) and granzyme B at a range of initial effector to target cell ratios (E:T).

FIG. 47A-47D graph bioluminescence emission (“total flux”) in tumour-injected mice treated with or without αβ T cells. Graphs show results of mice treated with PBS (FIG. 47A), TBB/H (FIG. 47B), TBB/H+pro-IL-36γ+granzyme B (FIG. 47C), or TBB/H+pro-IL-36γ (GzB)+granzyme B (FIG. 47D).

FIG. 48A-48B provide flow cytometry (FACS) results confirming expression of the TBB CCR (“TIE”) (within the TBB/H pCAR) and expression of the γδ TCR in untransduced (FIG. 48A) or TBB/H pCAR γδ T cells (FIG. 48B).

FIG. 49A provides folds of cell expansion after culturing untransduced or TBB/H pCAR γδ T-cells for 15 days. FIG. 49B provides numbers of cells obtained and cultured from three individual donors at three different time points (day 1, day 8 and day 15).

FIG. 50A-50B provide viability (%) of MDA-MB-468 tumour cells (FIG. 50A) or BxPC-3 tumour cells (FIG. 50B) after culturing with untransduced or TBB/H pCAR-γδ T cells (at 1:1 ratio), compared to tumour cells cultured alone.

FIG. 51A-51B provide the numbers of successful restimulation cycles of untransduced or TBB/H pCAR γδ T cells. The T cells were cultured with MDA-MD-468 tumour target cells (FIG. 51A) or BxPC-3 tumour target cells (FIG. 51B). FIG. 51C-51D provide viability (%) of MDA-MB-468 tumour cells (FIG. 51C) or BxPC-3 tumour cells (FIG. 51D) over successive restimulation cycles with untransduced or TBB/H pCAR-γδ T cells.

FIG. 52 provides bioluminescence emission (“total flux”) in BxPC-3 tumour-injected NSG mice treated with PBS, untransduced γδ T cells (“UT”) or TBB/H pCAR γδ T cells (“TBBH”) over time.

FIG. 53 provides bioluminescence emission (“total flux”) in MDA-MB-468 tumour-injected SCID Beige mice treated with PBS or TBB/H pCAR γδ T cells (“TBBH”) over time.

### 4. DETAILED DESCRIPTION

The details of various embodiments of the invention are set forth in the description below. Other features, objects, and advantages of the invention will be apparent from the description and the drawings, and from the claims.

### 4.1. Definitions

Unless otherwise defined herein, all technical and scientific terms used herein have the meaning commonly understood by a person skilled in the art to which this invention belongs. As used herein, the following terms have the meanings ascribed to them below.

The term “IL-1 family member” refers to a member of the IL-1 family, comprising seven proteins with pro-inflammatory activity (IL-1α and IL-1β, IL-18, IL-33, IL-36α, IL-36β and IL-36γ) and four proteins with anti-inflammatory activity (IL-1 receptor antagonist (IL-1Ra), IL-36Ra, IL-37 and IL-38). In some embodiments, the IL-1 family member is IL-18, IL-36α, IL-36β or IL-36γ. IL-36α, IL-36β and IL-36γ are collectively referred to as “IL-36.”

The term “pro-cytokine” refers to an inactive precursor of a member of the IL-1 family. The pro-cytokine generally comprises (i) a pro-peptide, (ii) a cleavage site recognized by a protease, and (iii) a mature, biologically active, cytokine fragment. Activities of the cytokine fragment can be modulated by processing of the cleavage site. In preferred embodiments, the pro-cytokine is pro-IL-18, pro-IL-36α, pro-IL-36β or pro-IL-36γ.

The term “pro-IL-18” refers the native 24-kDa inactive precursor of IL-18. Pro-IL-18 comprises, from N-terminus to C-terminus, (i) a pro-peptide, (ii) a cleavage site recognized by caspase 1, and (iii) the mature, biologically active, IL-18 protein fragment. In preferred embodiments, pro-IL-18 refers to human pro-IL-18, which is a 24.2 kDa protein of 193 aa. The cDNA sequence for human pro-IL-18 is provided by GenBank/EBI Data Bank accession number AF077611 (nucleotides 1-579). The protein sequence for human pro-IL-18 is provided by GenBank accession number AAC27787.

The term “pro-IL-36α” refers the native 17.7-kDa inactive precursor of IL-36α. Pro-IL-36a comprises, from N-terminus to C-terminus, (i) a pro-peptide, (ii) a cleavage site recognized by neutrophil proteases that include cathepsin G and elastase, and (iii) the mature, biologically active, IL-36a protein fragment. In preferred embodiments, pro-IL-36a refers to human pro-IL-36α, which is a 17.7 kDa protein of 158 aa. The cDNA sequence for human pro-IL-36a is provided by GenBank/EBI Data Bank accession number AF201831.1 (nucleotides 1-477). The protein sequence for human pro-IL-36a is provided by GenBank accession number AAY14988.1 and also provided herein as SEQ ID NO: 36.

The term “pro-IL-36β” refers the native 18.5-kDa inactive precursor of IL-36β. Pro-IL-36β comprises, from N-terminus to C-terminus, (i) a pro-peptide, (ii) a cleavage site recognized by neutrophil proteases that include cathepsin G, and (iii) the mature, biologically active, IL-36β protein fragment. In preferred embodiments, pro-IL-36β refers to human pro-IL-36β, which is an 18.5 kDa protein of 164 aa. The cDNA sequence for human pro-IL-36β is provided by GenBank/EBI Data Bank accession number AF200494.1 (nucleotides 1-1190). The protein sequence for human pro-IL-36β is provided by GenBank accession number NP 055253, and also provided herein as SEQ ID NO: 38.

The term “pro-IL-36γ” refers the native 18.7-kDa inactive precursor of IL-36γ. Pro-IL-36γ comprises, from N-terminus to C-terminus, (i) a pro-peptide, (ii) a cleavage site recognized by neutrophil proteases that include proteinase 3 and elastase, and (iii) the mature, biologically active, IL-36γ protein fragment. In preferred embodiments, pro-IL-36γ refers to human pro-IL-36γ, which is an 18.7 kDa protein of 169 aa. The cDNA sequence for human pro-IL-36γ is provided by GenBank/EBI Data Bank accession number AF200492 (nucleotides 1-1183). The protein sequence for human pro-IL-36γ is provided by GenBank accession number NP 062564, and also provided herein as SEQ ID NO: 40.

The term “modified pro-cytokine” as used herein refers to a protein generated by insertion, deletion, and/or substitution of one or more amino acids of a pro-cytokine protein. In preferred embodiments, the modified pro-cytokine includes a new cleavage site recognized and cleaved by a protease other than a protease that cleaves the unmodified pro-cytokine to release a cytokine fragment.

The term “modified pro-IL-18” as used herein refers to a protein generated by insertion, deletion, and/or substitution of one or more amino acids of a pro-IL-18 protein. In preferred embodiments, the modified pro-IL-18 includes a new cleavage site recognized by a protease other than caspase-1, and the modified pro-IL-18 can be cleaved by a protease other than caspase-1 to release a biologically active IL-18 protein fragment.

The term “modified pro-IL-36” as used herein refers to a protein generated by insertion, deletion, and/or substitution of one or more amino acids of a pro-IL-36 protein. In preferred embodiments, the modified pro-IL-36 includes a new cleavage site recognized by a protease other than cathepsin G, elastase and proteinase 3 and the modified pro-IL-36 can be cleaved by a protease other than cathepsin G, elastase or proteinase 3 to release a biologically active IL-36 protein fragment.

The term “pro-IL-18 ([protease])” as used herein refers to a modified pro-IL-18 containing a cleavage site recognized by the protease identified in the bracket. For example, pro-IL-18 (GzB) refers to a modified pro-IL-18 containing a cleavage site cleavable by granzyme B (GzB), pro-IL-18 (casp 3) refers to a modified pro-IL-18 containing a cleavage site cleavable by caspase-3, and pro-IL-18 (casp 8) refers to a modified pro-IL-18 containing a cleavage site cleavable by caspase-8.

The term “pro-IL-36 (GzB)” as used herein refers to a modified pro-IL-36 containing a cleavage site recognized by GzB.

The term “cleavage site” as used herein refers to a sequence of amino acids that can be recognized by a protease. As used herein, a cleavage site “recognized by” a protease is an amino acid sequence that is cleavable by the protease under conditions present or achievable in vivo.

The terms “a biologically active cytokine fragment” and “cytokine fragment” as used herein refer to a biologically active polypeptide generated by cleavage of a pro-cytokine by a protease that recognizes a cleavage site upstream of (N-terminal to) the cytokine fragment. By biologically active is meant that the cytokine fragment can bind to and activate its corresponding receptor. The cytokine fragment can be the native cytokine protein fragment or a modification thereof. In some embodiments, the cytokine fragment has an improved biological activity as compared to native mature cytokine. In some embodiments, the cytokine fragment refers to IL-18 fragment or IL-36 fragment as defined hereunder.

The terms “IL-18 fragment” and “IL-18 protein fragment” as used herein refer to a biologically active IL-18 polypeptide generated by cleavage of a pro-IL-18 by a protease that recognizes a cleavage site upstream of (N-terminal to) the IL-18 fragment. By biologically active is meant that the IL-18 fragment can bind to and activate the IL-18 receptor. The IL-18 fragment can be the native mature IL-18 protein fragment or a modification thereof. In some embodiments, the IL-18 fragment has an improved biological activity as compared to native mature IL-18.

The terms “IL-36 fragment” and “IL-36 protein fragment” as used herein refer to a biologically active IL-36 polypeptide generated by cleavage of a pro-IL-36 by a protease that recognizes a cleavage site upstream of (N-terminal to) the IL-36 fragment. By biologically active is meant that the IL-36 fragment can bind to and activate the IL-36 receptor. The IL-36 fragment can be the native mature IL-36 protein fragment or a modification thereof. In some embodiments, the IL-36 fragment has an improved biological activity as compared to native mature IL-36. The IL-36 fragment can refer to a mature IL-36α, β or γ protein.

The term “IL-18 variant” as used herein refers collectively to pro-IL-18 proteins, modified pro-IL-18 proteins, and IL-18 fragments, including the native mature IL-18 fragment.

The term “IL-36 variant” as used herein refers collectively to pro-IL-36 proteins, modified pro-IL-36 proteins, and IL-36 fragments, including the native mature IL-36α, β or γ fragment.

As used herein with regard to the binding element of an engineered T cell receptor (TCR) or chimeric antigen receptor (CAR), and the immunoresponsive cells engineered to express such TCRs or CARs, the terms “recognize”, “specifically binds,” “specifically binds to,” “specifically interacts with,” “specific for,” “selectively binds,” “selectively interacts with,” and “selective for” a particular antigen or epitope thereof—which can be a protein antigen, a glycopeptide antigen, or a peptide-MHC complex—means binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule.

### 4.2. Other Interpretational Conventions

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### 4.3. Immunoresponsive Cells

In a first aspect, immunoresponsive cells are provided. The immunoresponsive cells express a modified pro-cytokine of IL-1 superfamily, wherein the modified pro-cytokine comprises, from N-terminus to C-terminus: (a) a pro-peptide; (b) a cleavage site recognized by a protease other than caspase-1, cathepsin G, elastase or proteinase 3; and (c) a cytokine fragment of the IL-1 superfamily.

In some embodiments, the immunoresponsive cells express a modified pro-IL-18, wherein the modified pro-IL-18 comprises, from N-terminus to C-terminus: (a) a pro-peptide; (b) a cleavage site recognized by a protease other than caspase-1; and (c) a biologically active IL-18 fragment.

In some embodiments, the immunoresponsive cells express a modified pro-IL-36, wherein the modified pro-IL-36 comprises, from N-terminus to C-terminus: (a) a pro-peptide; (b) a cleavage site recognized by a protease other than cathepsin G, elastase and proteinase 3; and (c) a biologically active IL-36 α, β or γ fragment.

### 4.3.1. Cells

In typical embodiments, the immunoresponsive cells are T cells.

In certain embodiments, the immunoresponsive cells are αβ T cells. In particular embodiments, the immunoresponsive cells are cytotoxic αβ T cells. In particular embodiments, the immunoresponsive cells are αβ helper T cells. In particular embodiments, the immunoresponsive cells are regulatory αβ T cells (Tregs).

In certain embodiments, the immunoresponsive cells are γδ T cells. In particular embodiments, the immunoresponsive cells are Vδ2⁺γδ T cells. In particular embodiments, the immunoresponsive cells are Vδ2⁻ T cells. In specific embodiments, the Vδ2⁻ T cells are Vδ1⁺ cells.

In certain embodiments, the immunoresponsive cells are Natural Killer (NK) cells.

In some embodiments, the immunoresponsive cell expresses no additional exogenous proteins. In other embodiments, the immunoresponsive cell is engineered to express additional exogenous proteins, such as an engineered T cell receptor (TCR) or chimeric antigen receptor (CAR). Immunoresponsive cells that further express engineered TCRs and CARs are described further below.

In some embodiments, the immunoresponsive cells are obtained from peripheral blood mononuclear cells (PBMCs). In some embodiments, the immunoresponsive cells are obtained from tumours. In particular embodiments, the immunoresponsive cells obtained from tumours are tumour infiltrating lymphocytes (TILs). In specific embodiments, the TILs are αβ T cells. In other specific embodiments, the TILs are γδ T cells, and in particular, Vδ2⁻γδ T cells.

### 4.3.2. Modified Pro-IL-18

In some embodiments, the immunoresponsive cell expresses a modified pro-IL-18.

The modified pro-IL-18 comprises, from N-terminus to C-terminus: (i) a pro-peptide; (ii) a cleavage site recognized by a protease other than caspase-1; and (iii) an IL-18 fragment. The modified pro-IL-18 can be cleaved by a protease that recognizes the cleavage site to release the pro-peptide and a biologically active IL-18 fragment.

### 4.3.2.1. Pro-Peptide

In typical embodiments, the pro-peptide is an unmodified native pro-peptide of a pro-IL-18 protein. In particular embodiments, the pro-peptide is an unmodified native pro-peptide of a human pro-IL-18 protein.

In other embodiments, the pro-peptide is modified from a native pro-peptide of a pro-IL-18 protein. In certain embodiments, the modified pro-peptide contains one or more amino acid modifications as compared to a native pro-IL-18 pro-peptide. In certain embodiments, the pro-peptide is a pro-peptide from a non-pro-IL-18 protein. In certain embodiments, the pro-peptide has a non-natural synthetic amino acid sequence.

In some embodiments, the pro-peptide is a polypeptide having at least 85%, 90%, 95%, 97%, 98%, 99% or 100% sequence identity to SEQ ID: 25. In some embodiments, the pro-peptide is a polypeptide having at least about 85%, 90%, 95%, 97%, 98%, 99% or 100% sequence identity to SEQ ID: 25.

### 4.3.2.2. Cleavage Site

The cleavage site in the modified pro-IL-18 is recognized by a protease other than caspase-1.

In typical embodiments, only a single cleavage site recognized by a protease other than caspase-1 is present in the modified pro-IL-18. In other embodiments, a plurality of cleavage sites recognized by a protease other than caspase-1 are introduced. In such embodiments, the plurality of cleavage sites can be cleavage sites recognized by the same or different proteases other than caspase-1.

In various embodiments, the cleavage site recognized by a protease other than caspase-1 is introduced (a) between the pro-peptide and the cleavage site for caspase-1, (b) in place of the cleavage site for caspase-1, or (c) between the cleavage site for caspase-1 and the IL-18 fragment.

In some embodiments, the cleavage site replaces the caspase-1 cleavage site of pro-IL-18. In some embodiments, the cleavage site is additional to the caspase-1 cleavage site.

In typical embodiments, the cleavage site in the modified pro-IL-18 is selected from protease cleavage sites known in the art. In typical embodiments, the protease is a protease known to be expressed in activated T cells or NK cells. In certain embodiments, the cleavage site is recognized by granzyme B (GzB), caspase-3, caspase-8, or membrane-type 1 matrix metalloproteinase (MT1-MMP, also known as MMP14), an alternative tumour-associated matrix metalloproteinase (MMP1-13), a disintegrin and metalloproteinase (ADAM) family member (notably ADAM 10 or ADAM17), cathepsin B, L or S, fibroblast-activation protein (FAP), kallikrein-related peptidases (KLK) such as KLK2, 3, 6 or 7, dipeptidyl peptidase (DPP)4, hepsin or urokinase plasminogen activator (see Dudani et al., “Harnessing protease activity to improve cancer care,” Annu. Rev. Cancer Biol., 2:353-76 (2018). In particular embodiments, the cleavage site is recognized by granzyme B (GzB). In particular embodiments, the cleavage site is recognized by caspase-3. In particular embodiments, the cleavage site is recognized by caspase-8. In particular embodiments, the cleavage site is recognized by MT1-MMP.

In some embodiments, the cleavage site comprises a sequence selected from SEQ ID Nos: 26, 28, 30, and 32. In some embodiments, the modified pro-IL-18 comprises a sequence selected from SEQ ID Nos: 27, 29, 31, and 33.

In other embodiments, the cleavage site is a non-naturally occurring synthetic cleavage site.

### 4.3.2.3. IL-18 Fragment

In various embodiments, the IL-18 fragment is a native IL-18 fragment. In preferred embodiments, the native IL-18 fragment is a human IL-18 fragment.

In other embodiments, the IL-18 fragment is modified from a native IL-18 fragment, but retains the ability to bind and activate an IL-18 receptor when cleaved from a modified pro-IL-18 by protease cleavage of the cleavage site. In various embodiments, the IL-18 fragment has a biological activity similar to, less than, or better than native mature IL-18 protein.

In some embodiments, the IL-18 fragment is a polypeptide having at least 85%, 90%, 95%, 97%, 98%, 99% or 100% sequence identity to SEQ ID: 24. In some embodiments, the IL-18 fragment is a polypeptide having at least about 85%, 90%, 95%, 97%, 98%, 99% or 100% sequence identity to SEQ ID: 24. In some embodiments, the modified pro-IL-18 protein is expressed from an exogenous sequence introduced into T cells. In some embodiments, the exogenous sequence is selected from the group consisting of SEQ ID Nos: 102, 103, 105, 107, 109, 111 and 113. In some embodiments, the exogenous sequence is a coding sequence cloned in an expression vector, for example, a viral vector or a non-viral vector.

### 4.3.3. Modified Pro-IL-36

In some embodiments, the immunoresponsive cell expresses a modified pro-IL-36 α, β or γ protein.

The modified pro-IL-36 comprises, from N-terminus to C-terminus: (i) a pro-peptide; (ii) a cleavage site recognized by a protease other than cathepsin G, elastase and proteinase 3; and (iii) an IL-36 fragment. The modified pro-IL-36 can be cleaved by a protease that recognizes the cleavage site to release the pro-peptide and a biologically active IL-36 α, β or γ fragment.

### 4.3.3.1. Pro-Peptide

In typical embodiments, the pro-peptide is an unmodified native pro-peptide of a pro-IL-36α, β or γ protein. In particular embodiments, the pro-peptide is an unmodified native pro-peptide of a human pro-IL-36 protein.

In other embodiments, the pro-peptide is modified from a native pro-peptide of a pro-IL-36 protein. In certain embodiments, the modified pro-peptide contains one or more amino acid modifications as compared to a native pro-IL-36 pro-peptide. In certain embodiments, the pro-peptide is a pro-peptide from a non-pro-IL-36 protein. In certain embodiments, the pro-peptide has a non-natural synthetic amino acid sequence.

In some embodiments, the pro-peptide is from pro-IL-36a (SEQ ID NO: 45). In some embodiments, the pro-peptide is from a modified pro-IL-36a (SEQ ID NO: 46). In some embodiments, the pro-peptide is from pro-IL-36β (SEQ ID NO: 47). In some embodiments, the pro-peptide is from a modified pro-IL-36β (SEQ ID NO: 48). In some embodiments, the pro-peptide is from pro-IL-36γ (SEQ ID NO: 49). In some embodiments, the pro-peptide is from a modified pro-IL-36γ (SEQ ID NO: 50).

### 4.3.3.2. Cleavage Site

The cleavage site in the modified pro-IL-36 is recognized by a protease other than cathepsin G, elastase and proteinase 3.

In typical embodiments, only a single cleavage site recognized by a protease other than cathepsin G, elastase and proteinase 3 is present in the modified pro-IL-36. In other embodiments, a plurality of cleavage sites recognized by a protease other than cathepsin G, elastase and proteinase 3 are introduced. In such embodiments, the plurality of cleavage sites can be cleavage sites recognized by the same or different proteases other than cathepsin G, elastase and proteinase 3.

In various embodiments, the cleavage site recognized by a protease other than cathepsin G, elastase and proteinase 3 is introduced (a) between the pro-peptide and the cleavage site for cathepsin G, elastase or proteinase 3, (b) in place of the cleavage site for cathepsin G, elastase or proteinase 3, or (c) between the cleavage site for cathepsin G, elastase or proteinase 3 and the IL-36 fragment.

In some embodiments, the cleavage site replaces the cleavage site for cathepsin G, elastase or proteinase 3, which is naturally present in pro-IL-36 α, β or γ. In some embodiments, the cleavage site is additional to the cleavage site for cathepsin G, elastase and/or proteinase 3, which is naturally present in pro-IL-36 α, β or γ.

In typical embodiments, the cleavage site in the modified pro-IL-36 is selected from protease cleavage sites known in the art. In typical embodiments, the protease is a protease known to be expressed in activated T cells or NK cells. In certain embodiments, the cleavage site is recognized by granzyme B (GzB), caspase-3, caspase-8, or membrane-type 1 matrix metalloproteinase (MT1-MMP, also known as MMP14), an alternative tumour-associated matrix metalloproteinase (MMP1-13), a disintegrin and metalloproteinase (ADAM) family member (notably ADAM 10 or ADAM17), cathepsin B, L or S, fibroblast-activation protein (FAP), kallikrein-related peptidases (KLK) such as KLK2, 3, 6 or 7, dipeptidyl peptidase (DPP)4, hepsin or urokinase plasminogen activator (see Dudani et al., “Harnessing protease activity to improve cancer care,” Annu. Rev. Cancer Biol., 2:353-76 (2018). In particular embodiments, the cleavage site is recognized by granzyme B (GzB). In particular embodiments, the cleavage site is recognized by caspase-3. In particular embodiments, the cleavage site is recognized by caspase-8. In particular embodiments, the cleavage site is recognized by MT1-MMP.

In some embodiments, the cleavage site comprises a sequence selected from SEQ ID Nos: 26, 28, 30, and 32. In some embodiments, the modified pro-IL-36 comprises a sequence selected from SEQ ID Nos: 37, 39, and 41.

In other embodiments, the cleavage site is a non-naturally occurring synthetic cleavage site.

### 4.3.3.3. IL-36 Fragment

In various embodiments, the IL-36 fragment is a native IL-36a (SEQ ID NO: 42), (3 (SEQ ID NO: 43) or γ (SEQ ID NO: 44) fragment. In preferred embodiments, the native IL-36 fragment is a human IL-36 fragment.

In other embodiments, the IL-36 fragment is modified from a native IL-36 fragment, but retains the ability to bind and activate an IL-36 receptor when cleaved from a modified pro-IL-36 by protease cleavage of the cleavage site. In various embodiments, the IL-36 fragment has a biological activity similar to, less than, or better than native mature IL-36 α, β or γ protein.

In some embodiments, the IL-36α, β or γ fragment is a polypeptide having at least 85%, 90%, 95%, 97%, 98%, 99% or 100% sequence identity to SEQ ID: 42, 43 or 44 respectively. In some embodiments, the IL-36α, β or γ fragment is a polypeptide having at least about 85%, 90%, 95%, 97%, 98%, 99% or 100% sequence identity to SEQ ID: 42, 43 or 44 respectively. In some embodiments, the modified pro-IL-36 protein is expressed from an exogenous sequence introduced into T cells. In some embodiments, the exogenous sequence is a coding sequence cloned in an expression vector, for example, a viral vector or a non-viral vector.

### 4.3.4. Expressed Protease

In some embodiments, the immunoresponsive cells are engineered to further express a protease that recognizes a cleavage site of the co-expressed modified pro-IL-18 or modified pro-IL-36.

In some embodiments, the protease is selected from the group consisting of GzB, caspase-3, caspase-8 and MT1-MMP.

In particular embodiments, the expressed protease is GzB. In preferred embodiments, the expressed protease is human GzB. In specific embodiments, the expressed protease comprises SEQ ID NO: 20 or a modification thereof.

In particular embodiments, the expressed protease is caspase-3. In preferred embodiments, the expressed protease is human caspase-3. In specific embodiments, the expressed protease comprises SEQ ID NO: 21 or a modification thereof.

In particular embodiments, the expressed protease is caspase-8. In preferred embodiments, the expressed protease in human caspase-8. In specific embodiments, the expressed protease comprises SEQ ID NO: 22 or a modification thereof.

In particular embodiments, the expressed protease is MT1-MMP. In preferred embodiments, the expressed protease is human MT1-MMP. In specific embodiments, the expressed protease comprises SEQ ID NO: 23 or a modification thereof.

In some embodiments, the expressed protease is an alternative tumour-associated matrix metalloproteinase (MMP1-13), a disintegrin and metalloproteinase (ADAM) family member (notably ADAM 10 or ADAM17), cathepsin B, L or S, fibroblast-activation protein (FAP), kallikrein-related peptidases (KLK) such as KLK2, 3, 6 or 7, dipeptidyl peptidase (DPP)4, hepsin or urokinase plasminogen activator (see Dudani et al., “Harnessing protease activity to improve cancer care,” Annu. Rev. Cancer Biol., 2:353-76 (2018).

The expressed protease is expressed from an exogenous sequence introduced into the immunoresponsive cells within an expression vector. In some embodiments, the immunoresponsive cells express a modified pro-cytokine and a protease from a single expression vector. In some embodiments, the immunoresponsive cells express a modified pro-cytokine and a protease from a plurality of expression vectors. In particular embodiments, the immunoresponsive cells express a modified pro-cytokine from a first expression vector and a protease from a second expression vector.

### 4.3.5. CAR

In typical embodiments, the immunoresponsive cells are engineered to further express a chimeric antigen receptor (CAR).

### 4.3.5.1. CAR Specificity

In typical embodiments, the CAR is specific for at least one antigen present in a cancer. In typical embodiments, the CAR is specific for at least one antigen present in a solid tumour.

In various embodiments, the antigen is a human telomerase reverse transcriptase (hTERT), survivin, mouse double minute 2 homolog (MDM2), cytochrome P450 1B1 (CYP1B), HER2/neu, Wilms' tumour gene 1 (WT1), livin, alphafetoprotein (AFP), carcinoembryonic antigen (CEA), mucin 16 (MUC16), MUC1, prostate-specific membrane antigen (PSMA), p53 or cyclin (D1). For example, the target antigen is hTERT or survivin. In some embodiments, the target antigen is CD38. In some embodiments, the target antigen is B-cell maturation antigen (BCMA, BCM). In some embodiments, the target antigen is BCMA, B-cell activating factor receptor (BAFFR, BR3), and/or transmembrane activator and CAML interactor (TACI), or a related protein thereof. For example, the target antigen in some embodiments is or is related to BAFFR or TACI. In some embodiments, the target antigen is CD33 or TIM-3. In some embodiments, it is CD26, CD30, CD53, CD92, CD100, CD148, CD150, CD200, CD261, CD262, or CD362.

In some embodiments, the CAR is specific for alpha folate receptor, 5T4, .alpha.v.beta.6 integrin, BCMA, B7-H3, B7-H6, CAIX, CD19, CD20, CD22, CD30, CD33, CD44, CD44v6, CD44v7/8, CD70, CD79a, CD79b, CD123, CD138, CD171, CEA, CSPG4, CMV, EBV, EGFR, EGFR family including ErbB2 (HER2), ErbB family homo and heterodimers, EGFRvIII, EGP2, EGP40, EPCAM, EphA2, EpCAM, FAP, fetal AchR, FR.alpha., GD2, GD3, Glypican-3 (GPC3), HLA-A1+MAGE1, HLA-A2+MAGE1, HLA-A3+MAGE1, HLA-A1+NY-ESO-1, HLA-A2+NY-ESO-1, HLA-A3+NY-ESO-1, HPV, IL-11R.alpha., IL-13R.alpha.2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, or VEGFR2.

In some embodiments, the CAR is specific for TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGSS, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-AL legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, or IGLL1.

In some embodiments, the CAR is specific to a MUC1 target antigen. In particular embodiments, the CAR is specific for a MUC1 epitope that is tumour-associated. In specific embodiments, the targeting domain of the CAR comprises CDRs of the HMFG2 antibody. See Wilkie et al., “Retargeting of human T cells to tumor-associated MUC1: the evolution of a chimeric antigen receptor,” J. Immunol. 180(7):4901-4909 (2008), incorporated herein by reference in its entirety. In some embodiments, the CAR comprises the V_{H} and V_{L} domains of the HMFG2 antibody. In some embodiments, the CAR comprises the HMFG2 single-chain variable fragment (scFv).

In some embodiments, the CAR is specific for ErbB homo- and/or heterodimers. In particular embodiments, the targeting domain of the CAR comprises the promiscuous ErbB peptide ligand, T1E. T1E is a chimeric peptide derived from transforming growth factor-α (TGF-α) and epidermal growth factor (EGF). See Wingens et al. “Structural analysis of an epidermal growth factor/transforming growth factor-alpha chimera with unique ErbB binding specificity,” J. Biol. Chem. 278:39114-23 (2003) and Davies et al., “Flexible targeting of ErbB dimers that drive tumorigenesis by using genetically engineered T cells,” Mol. Med. 18:565-576 (2012), the disclosures of which are incorporated herein by reference in their entireties.

### 4.3.5.2. CAR Format

In some embodiments, the CAR is a first-generation CAR. First-generation CARs can provide a TCR-like signal, most commonly using a CD3 zeta (CD3z or CD3) or Fcεr1γ intracellular signalling domain, and thereby elicit tumouricidal functions. However, the engagement of CD3z-chain fusion receptors may not suffice to elicit substantial IL-2 secretion and/or T-cell proliferation in the absence of a concomitant co-stimulatory signal. In physiological T-cell responses, optimal lymphocyte activation may require the engagement of one or more co-stimulatory receptors such as CD28 or 4-1BB. In some embodiments, a first-generation CAR as disclosed in Eshhar et al., “Specific activation and targeting of cytotoxic lymphocytes through chimeric single chains consisting of antibody-binding domains and the gamma or zeta subunits of the immunoglobulin and T-cell receptors,” PNAS 90(2):720-4 (1993) or a co-stimulatory chimeric receptor as disclosed in Alvarez-Vallina et al. “Antigen-specific targeting of CD28-mediated T cell co-stimulation using chimeric single-chain antibody variable fragment-CD28 receptors.” Eur. J. Immunol. 26(10):2304-9 (1996) and Krause et al., “Antigen-dependent CD28 signalling selectively enhances survival and proliferation in genetically modified activated human primary T lymphocytes,” J. Exp. Med. 188(4): 619-26 (1998), is expressed in the immunoresponsive cells described herein (FIG. 25); both references are incorporated herein by reference in their entireties.

In some embodiments, the CAR is a second-generation CAR. Second generation CARs can transduce a functional antigen-dependent co-stimulatory signal in human primary T-cells in addition to antigen-dependent TCR-like signal, permitting T-cell proliferation in addition to tumouricidal activity. Second generation CARs most commonly provide co-stimulation using co-stimulatory domains (synonymously, co-stimulatory signalling regions) derived from CD28 or 4-1BB. The combined delivery of co-stimulation plus a CD3 zeta signal can render second-generation CARs functionally superior to their first-generation counterparts. Exemplary second-generation CARs that can usefully be expressed in the immunoresponsive cells described herein are disclosed in U.S. Pat. No. 7,446,190; Finney et al., “Chimeric receptors providing both primary and costimulatory signaling in T cells from a single gene product,” J. Immunol 161(6):2791-7 (1998); Maher et al., “Human T-lymphocyte cytotoxicity and proliferation directed by a single chimeric TCRzeta/CD28 receptor,” Nat. Biotechnol. 20(1):70-5 (2002); Finney et al., “Activation of resting human primary T cells with chimeric receptors: costimulation from CD28, inducible costimulator, CD134, and CD137 in series with signals from the TCR zeta chain,” J. Immunol. 172(1):104-13 (2004); and Imai et al., “Chimeric receptors with 4-1BB signaling capacity provoke potent cytotoxicity against acute lymphoblastic leukemia,” Leukemia 18(4):676-84 (2004), incorporated herein by reference in their entireties.

Still further exemplary second-generation CARs that can usefully be expressed in the immunoresponsive cells described herein are provided in FIG. 25.

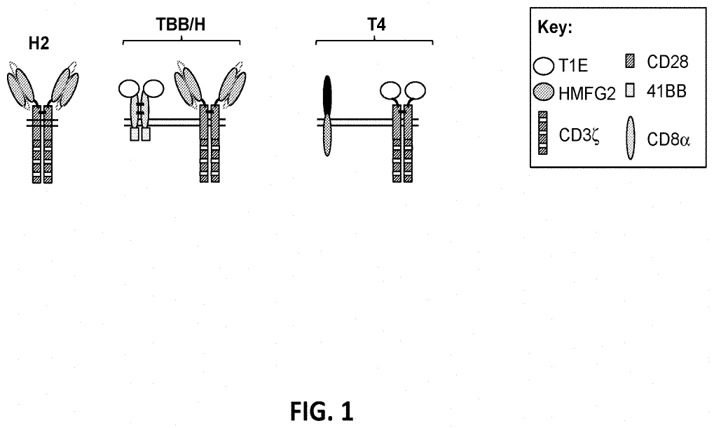

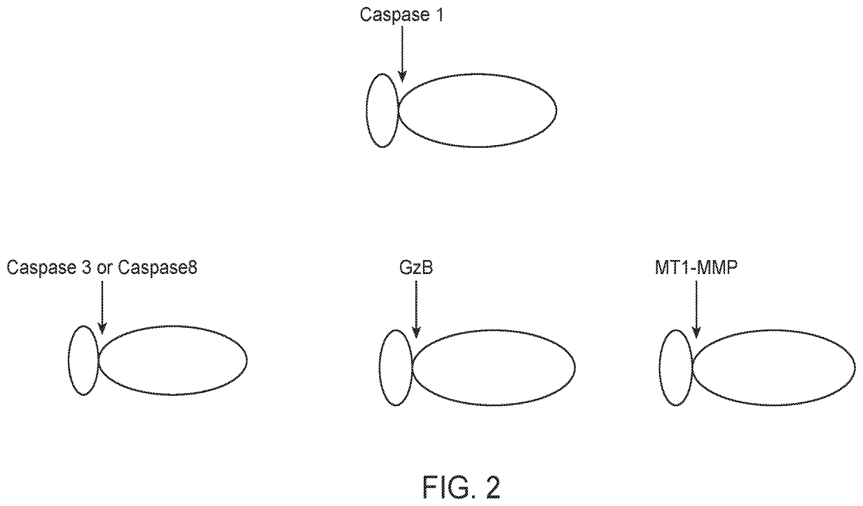

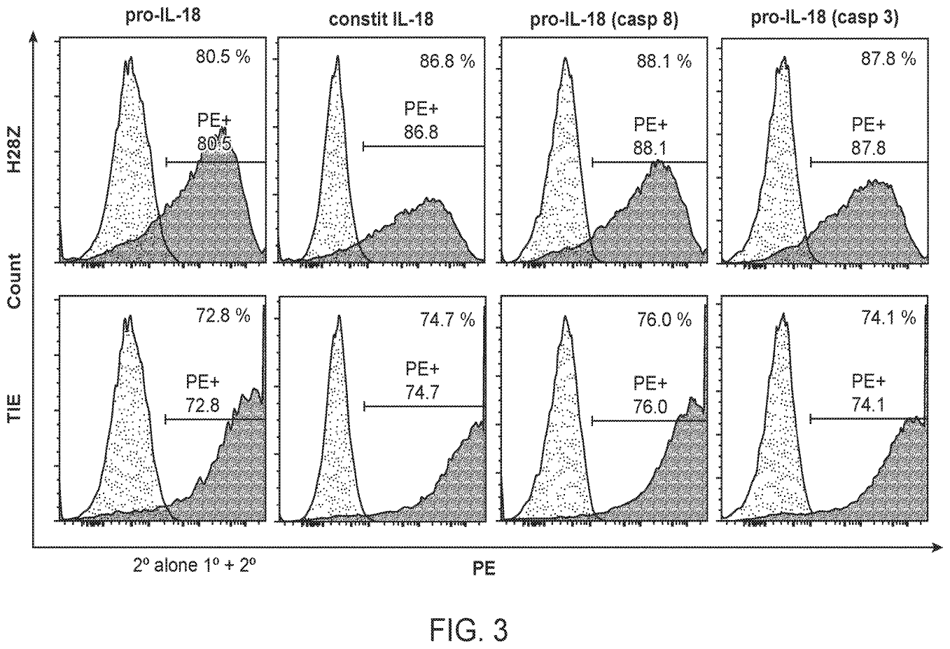

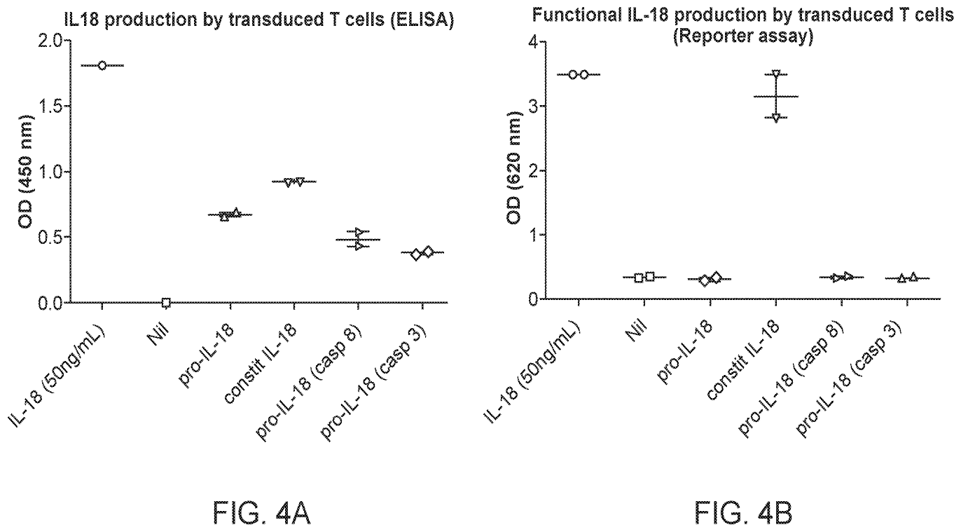

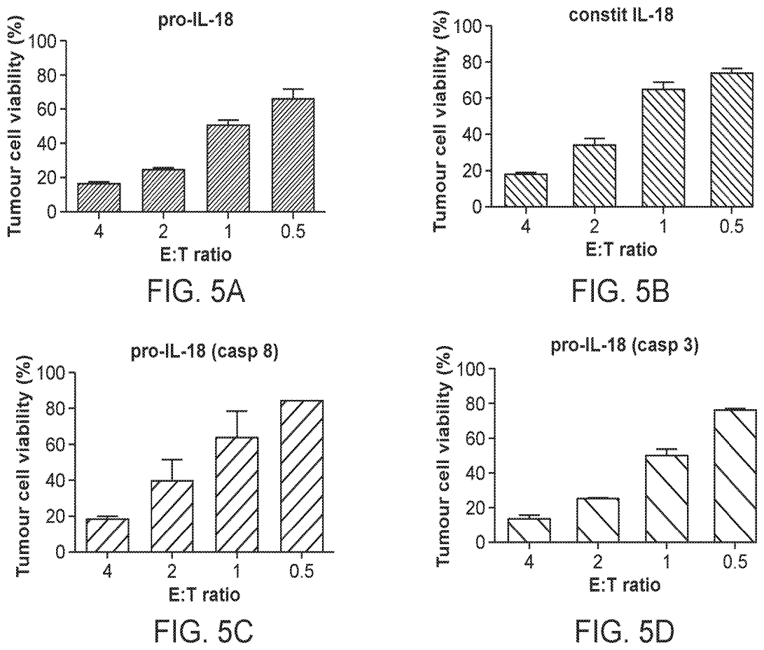

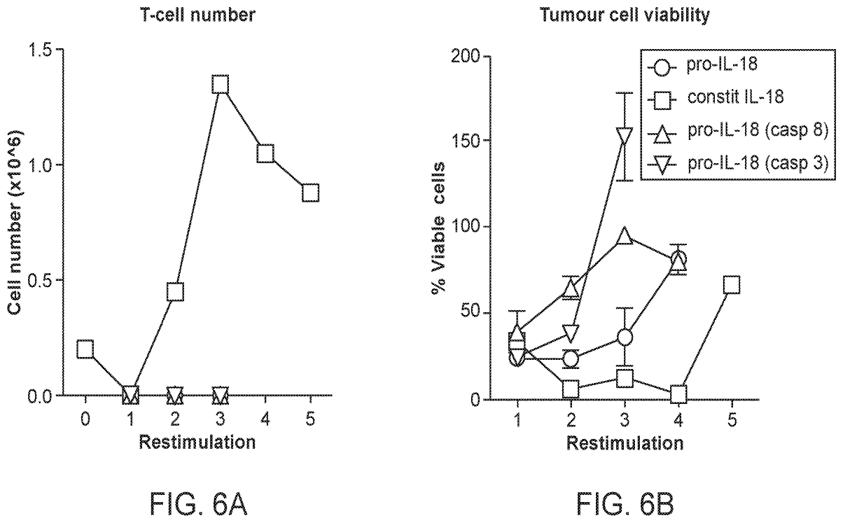

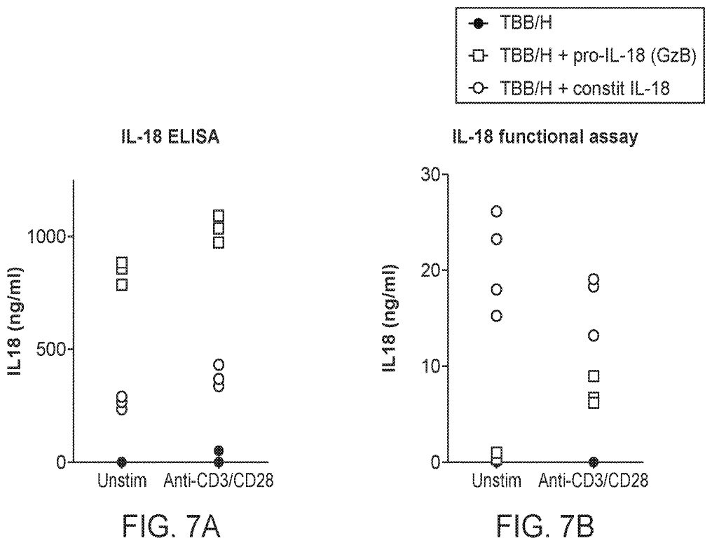

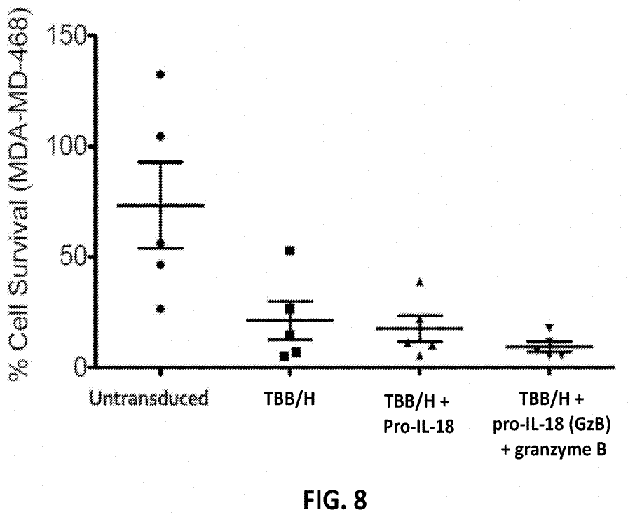

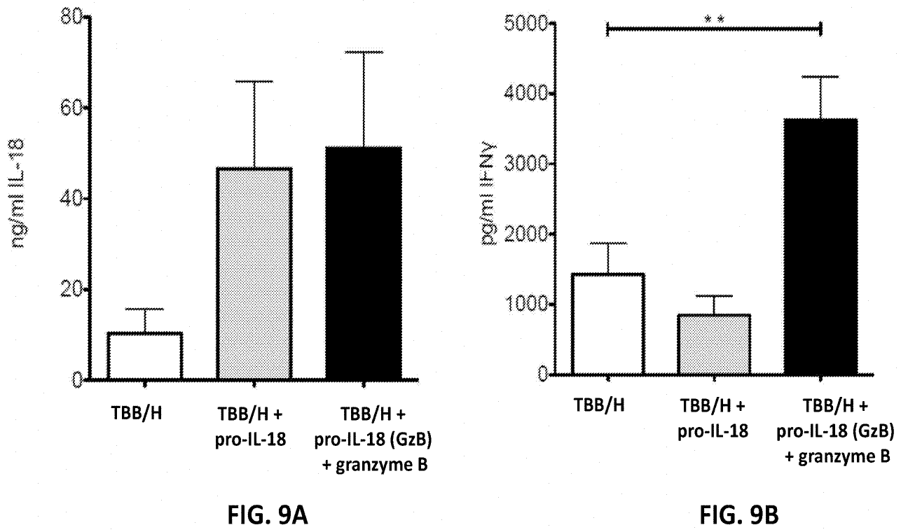

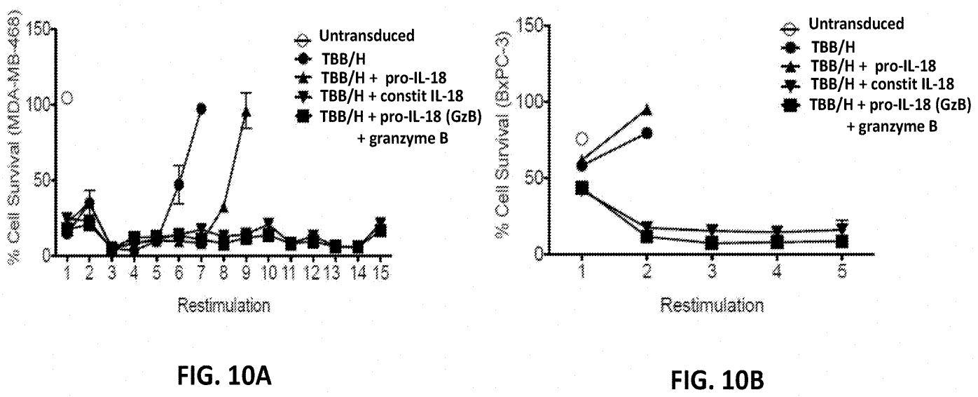

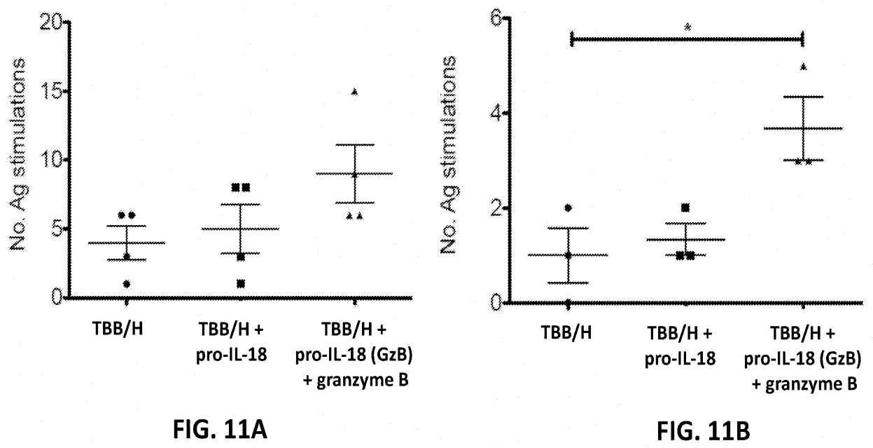

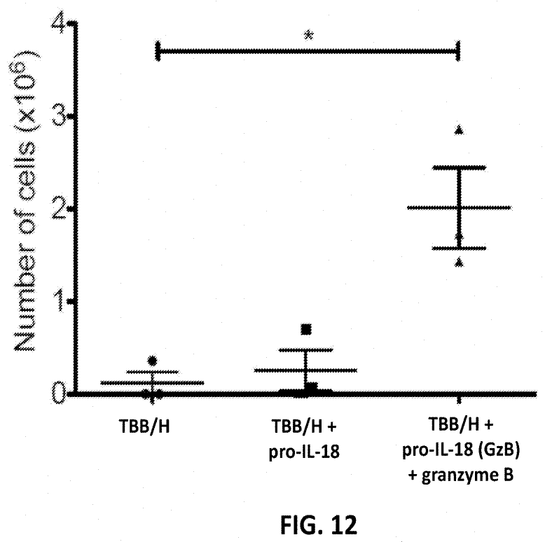

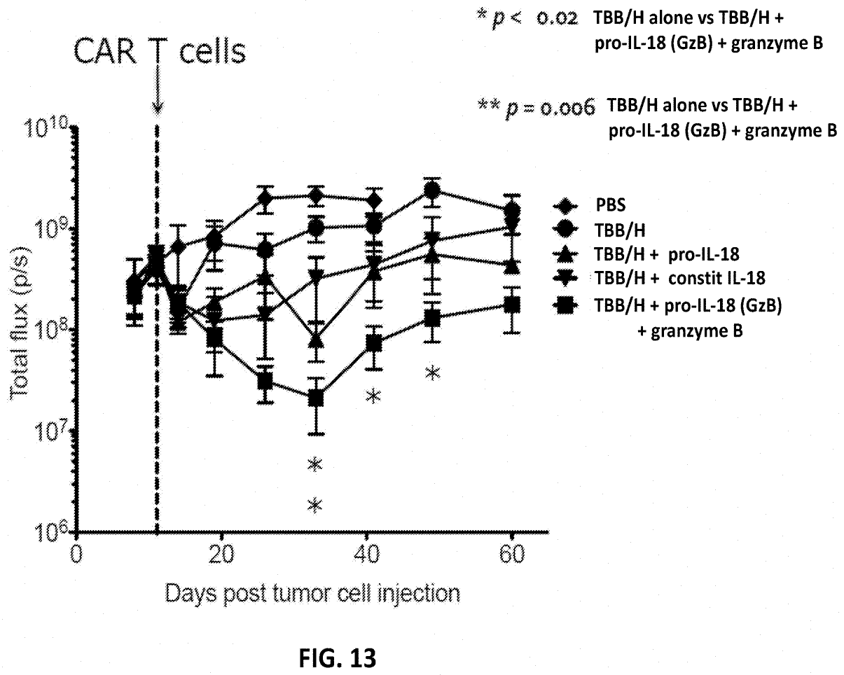

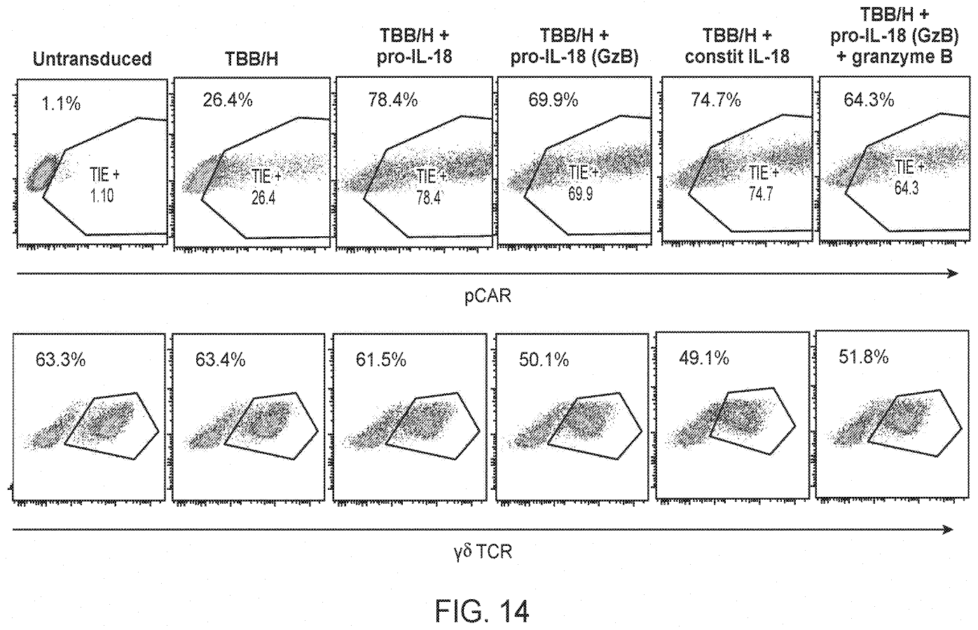

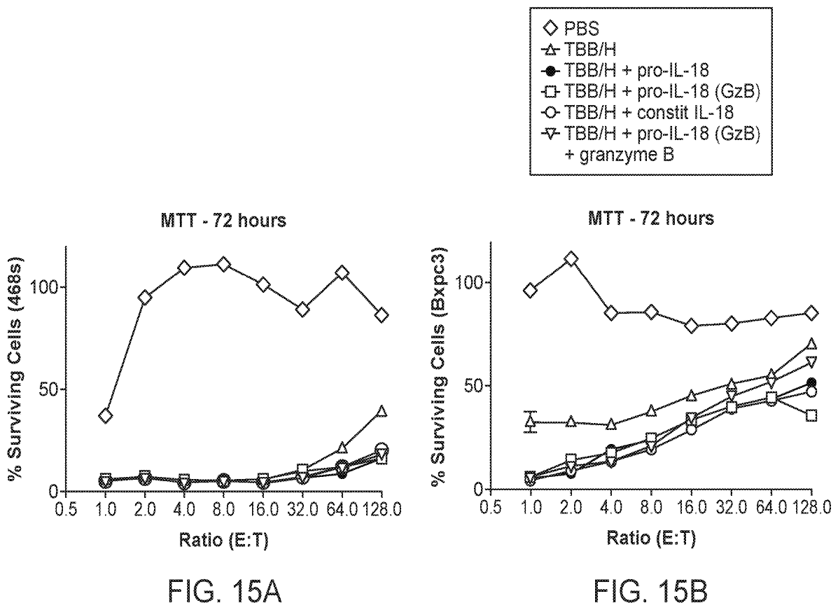

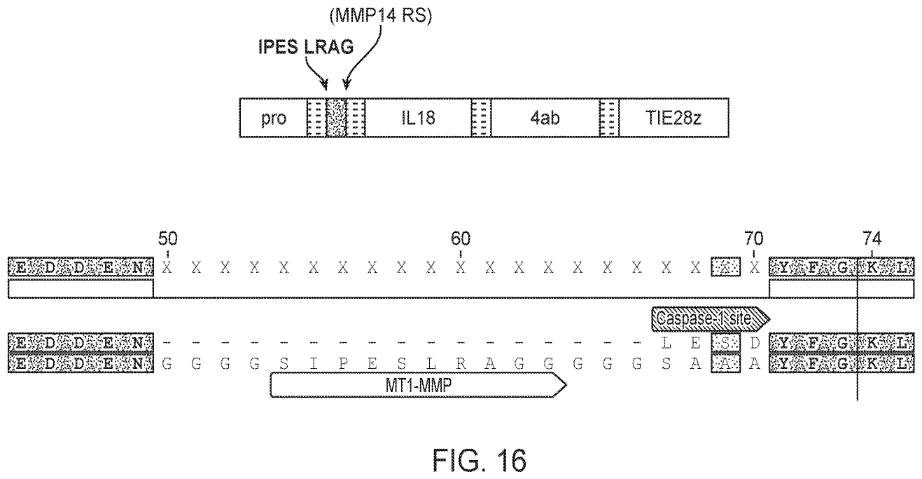

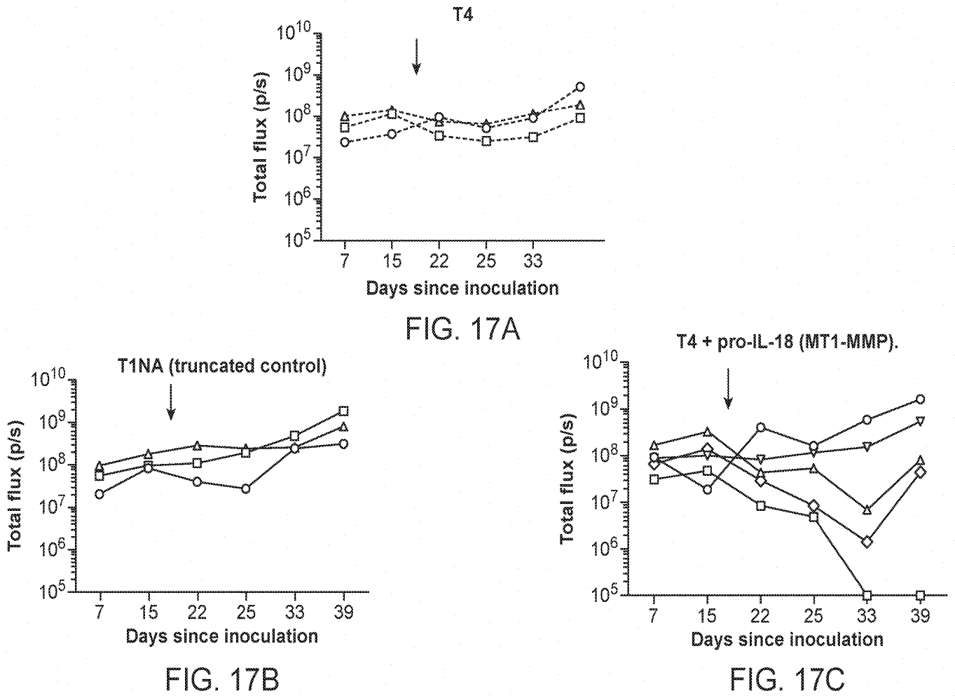

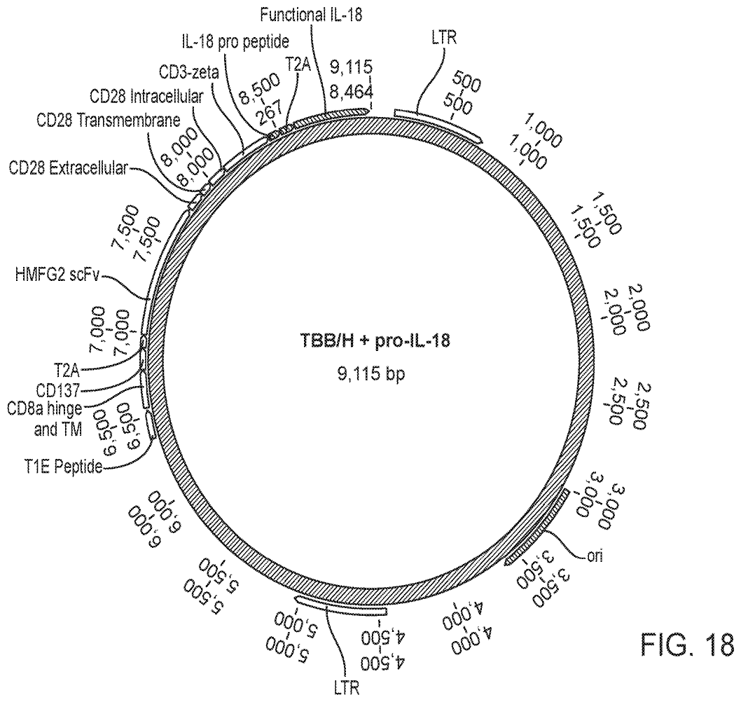

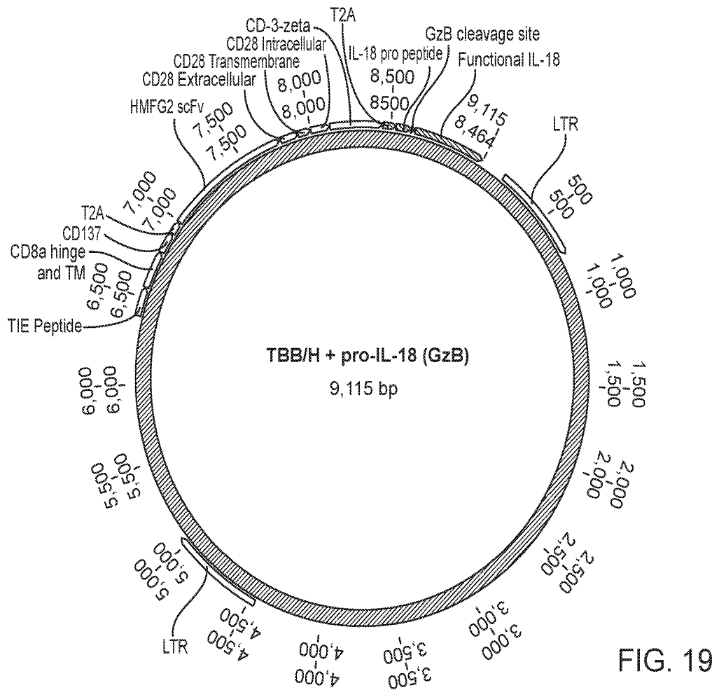

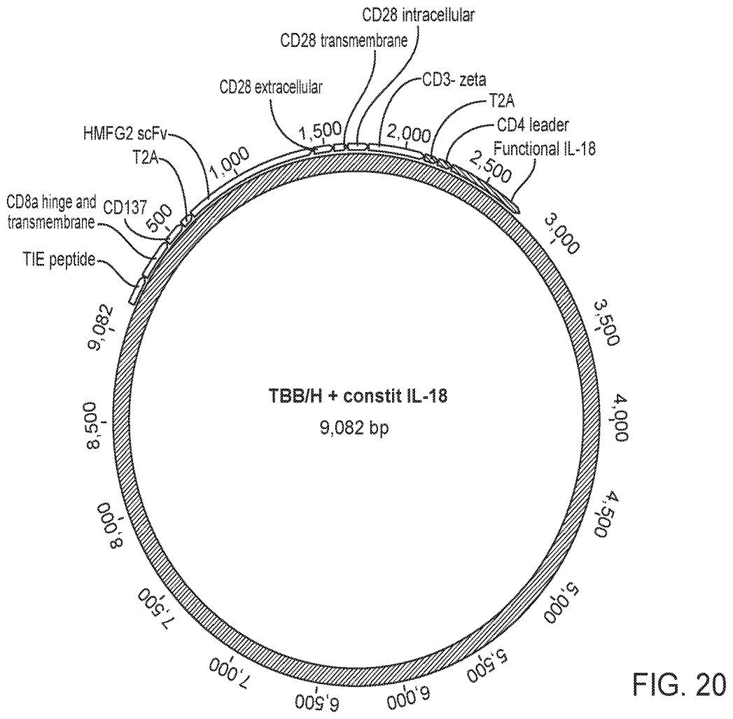

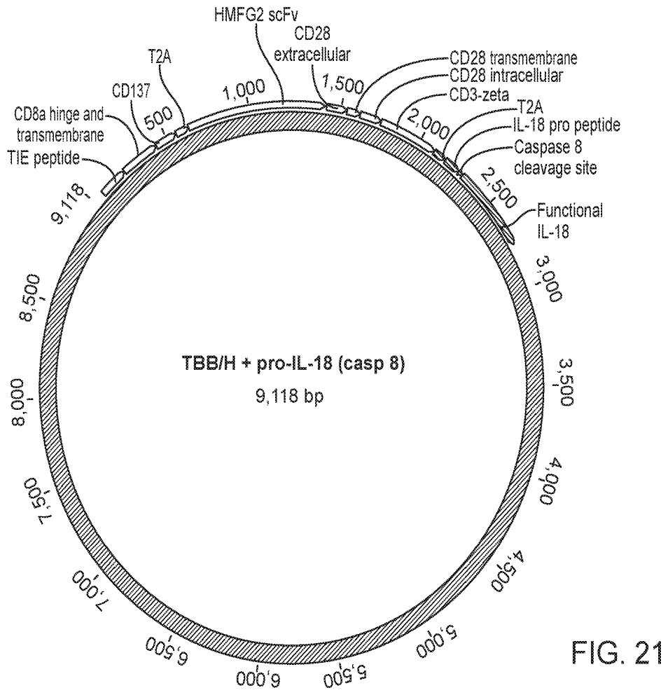

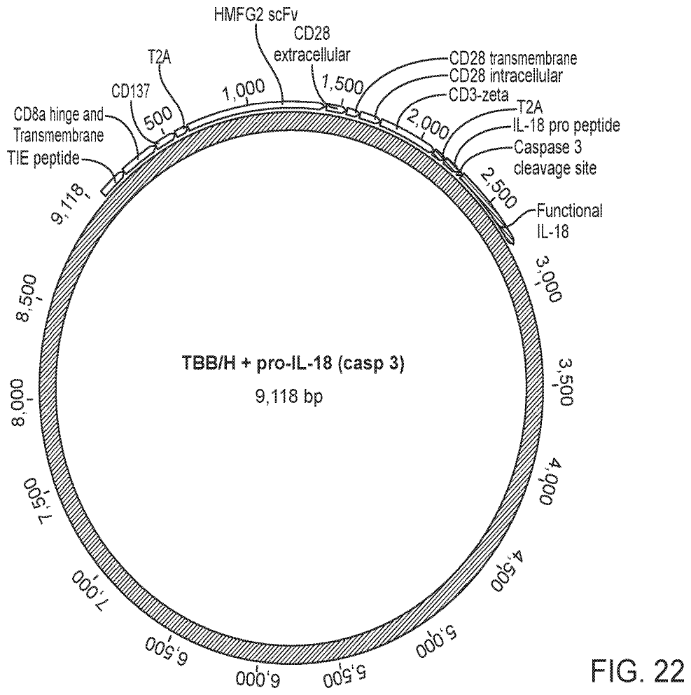

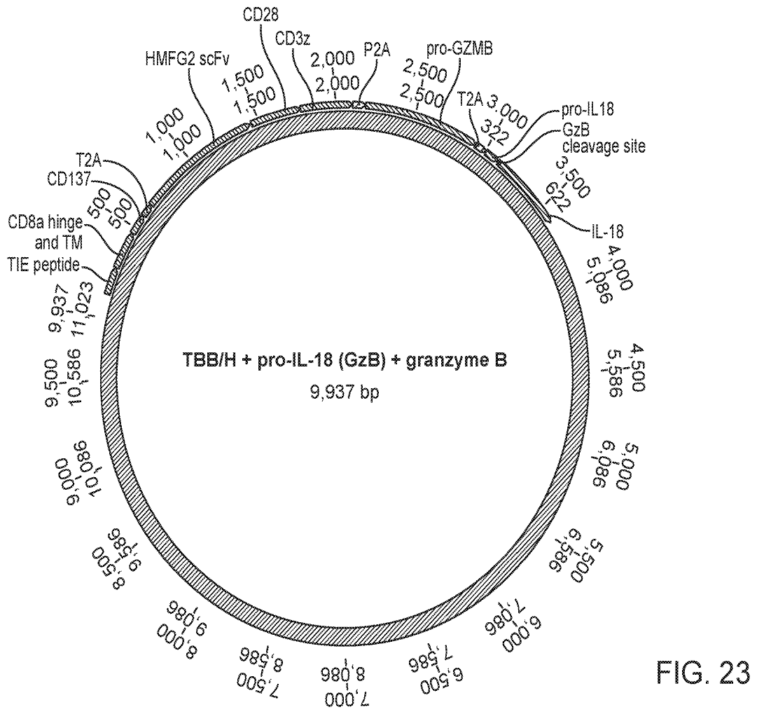

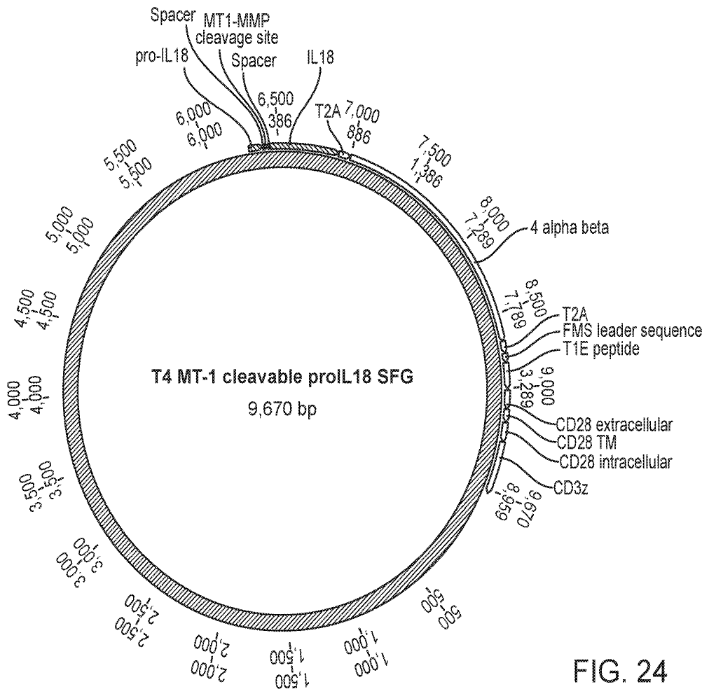

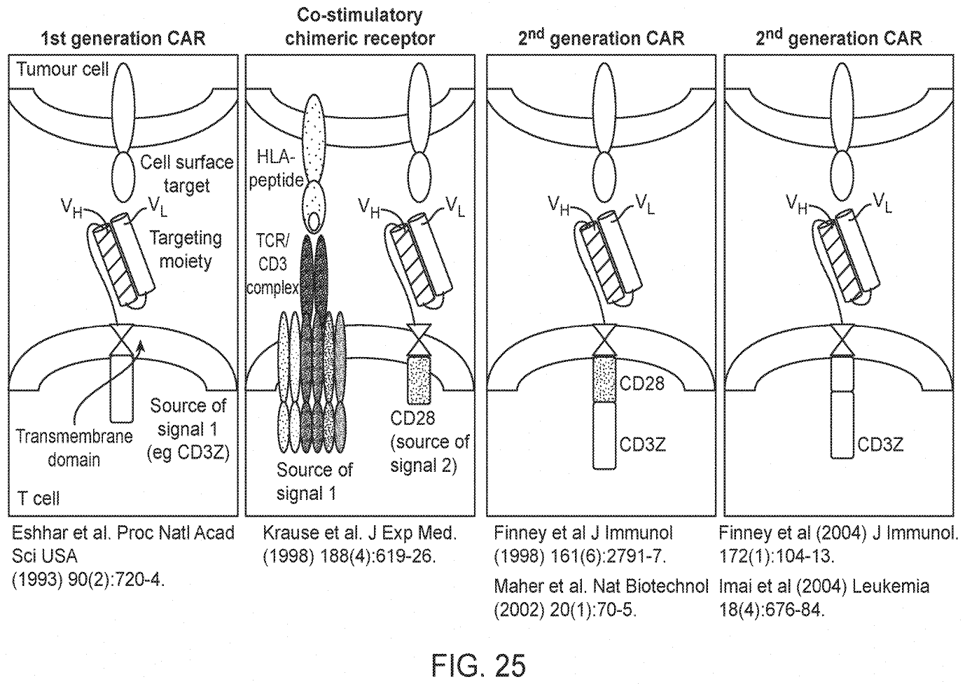

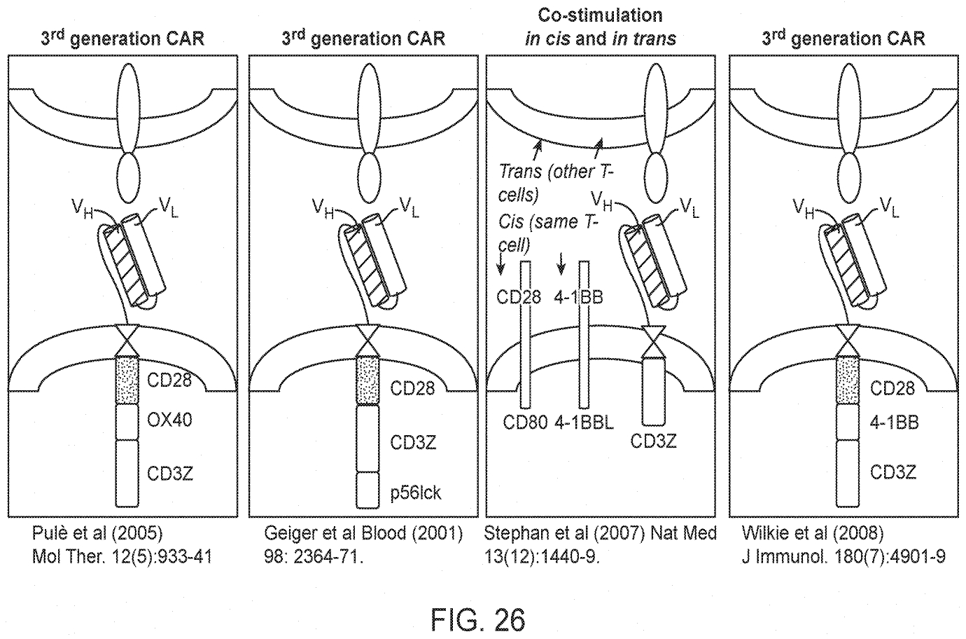

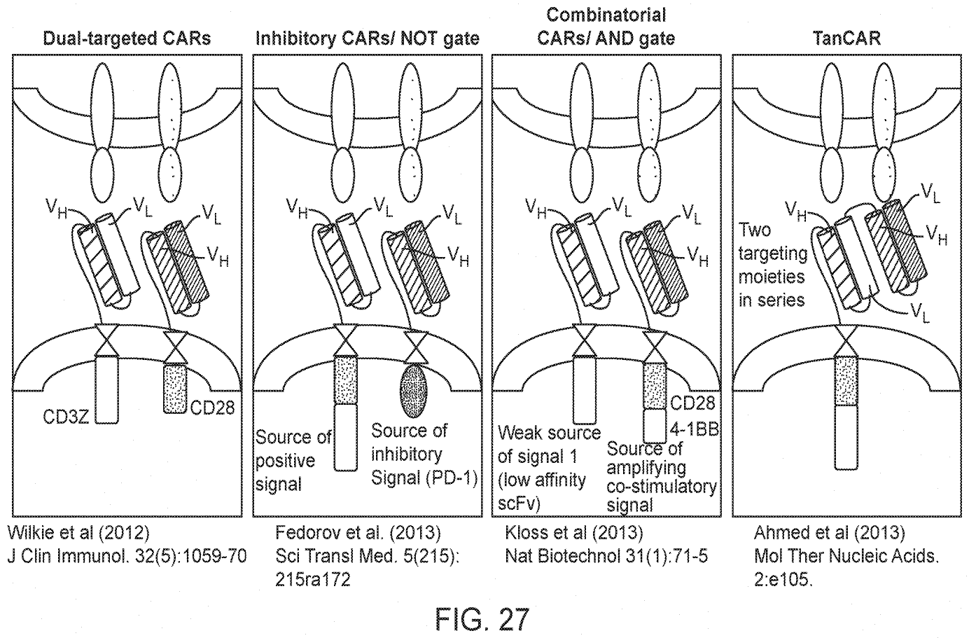

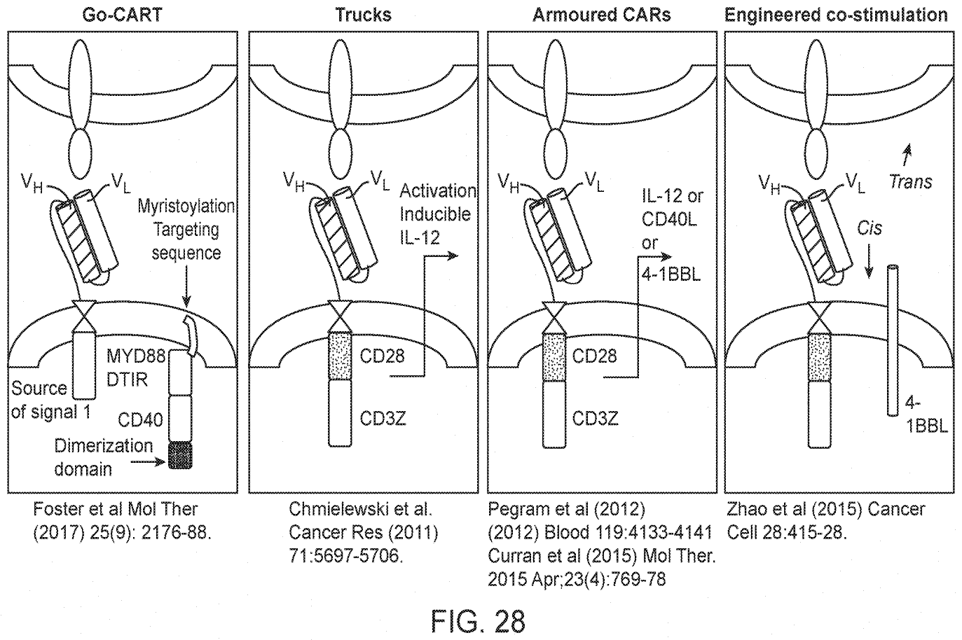

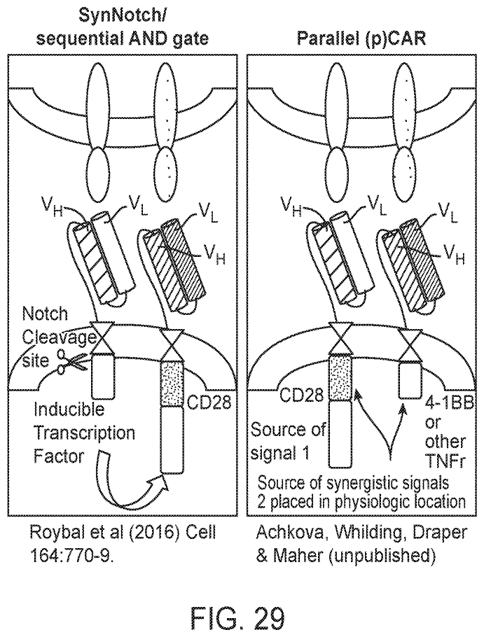

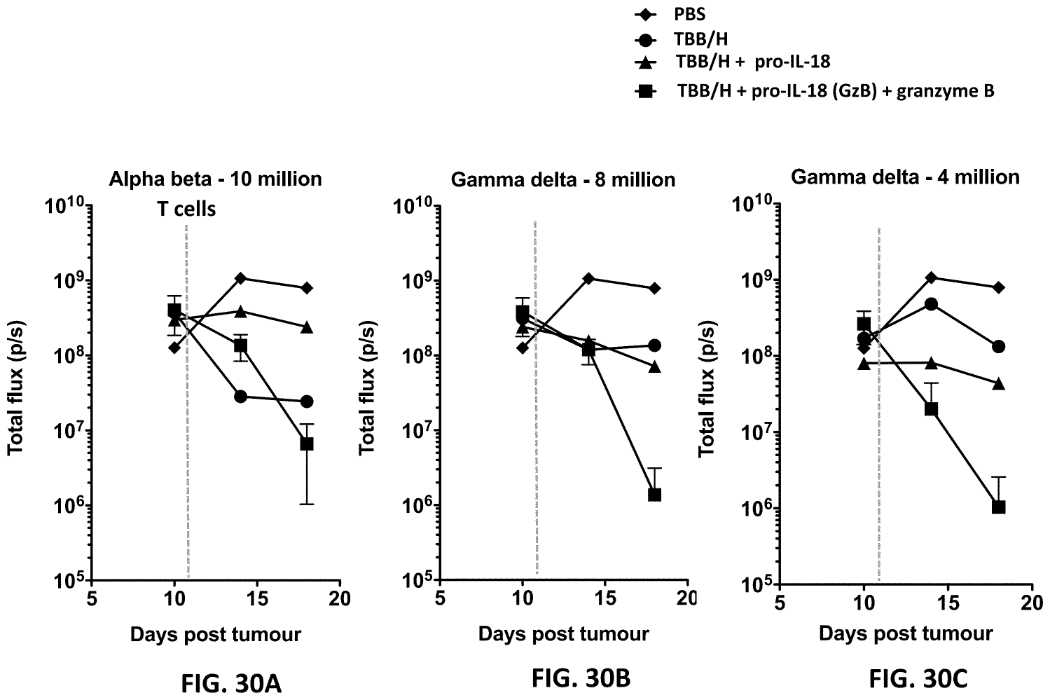

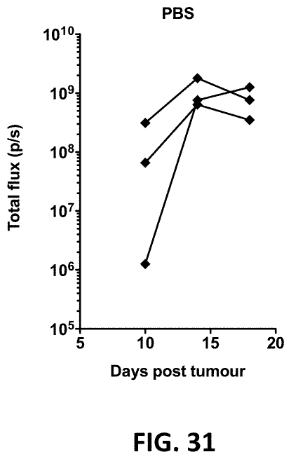

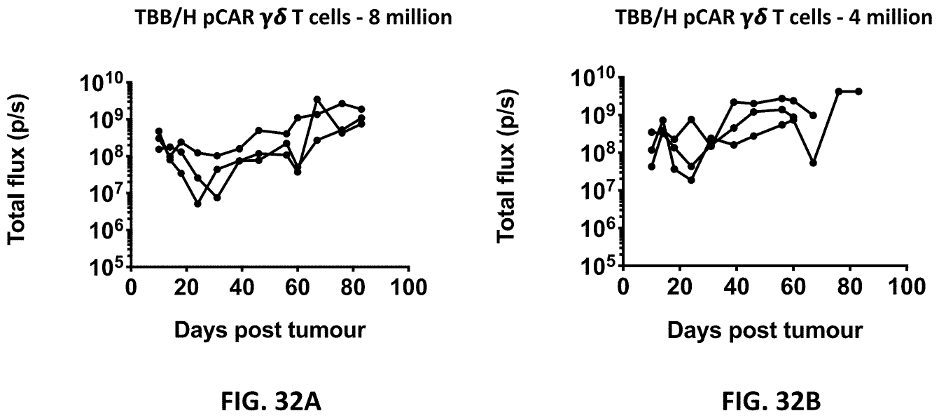

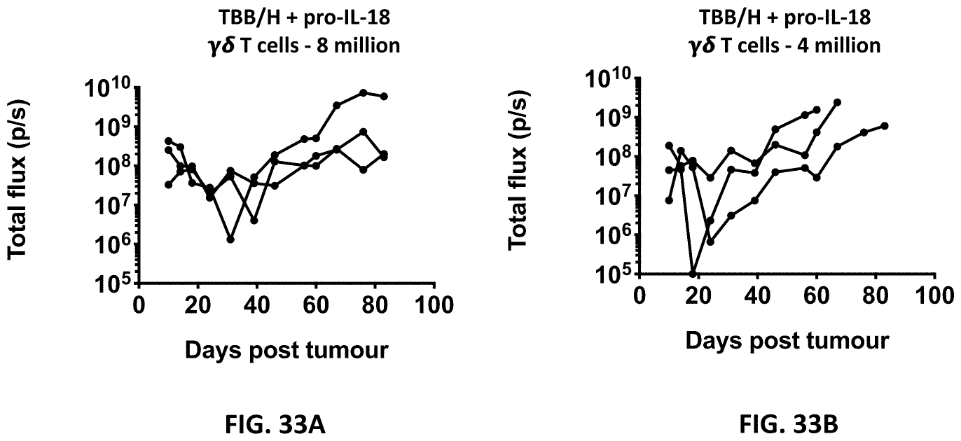

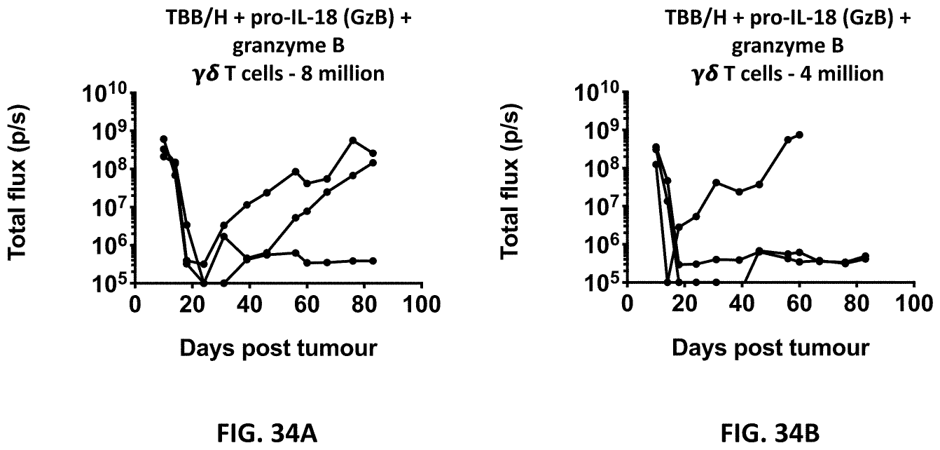

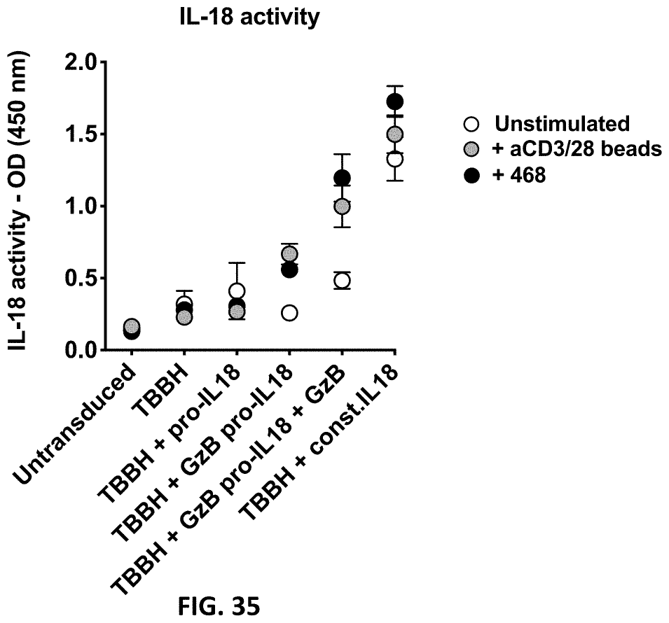

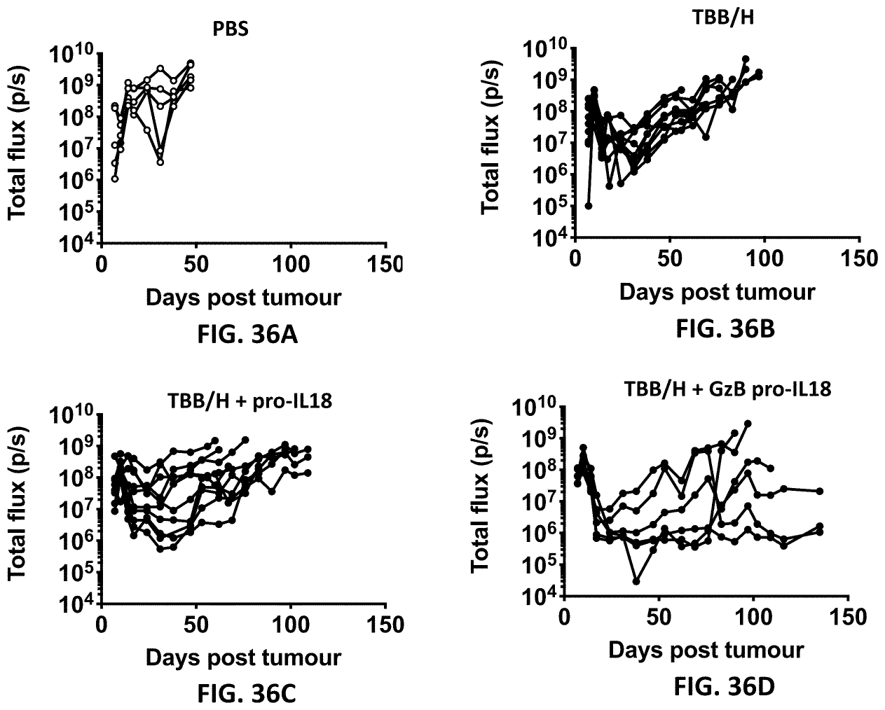

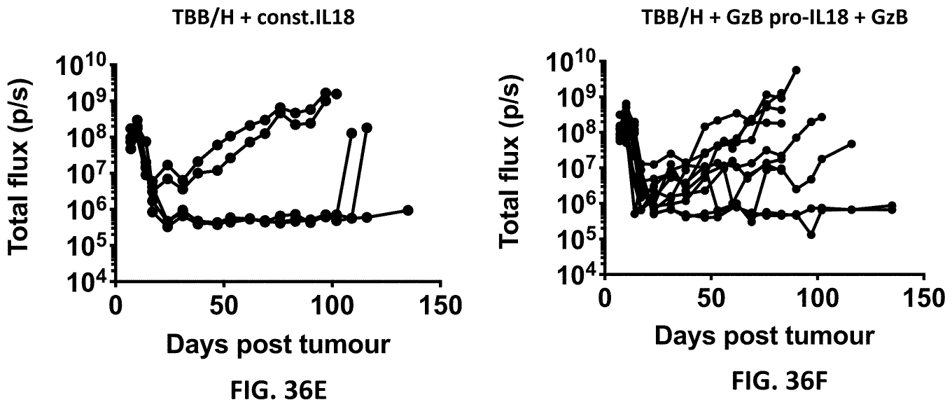

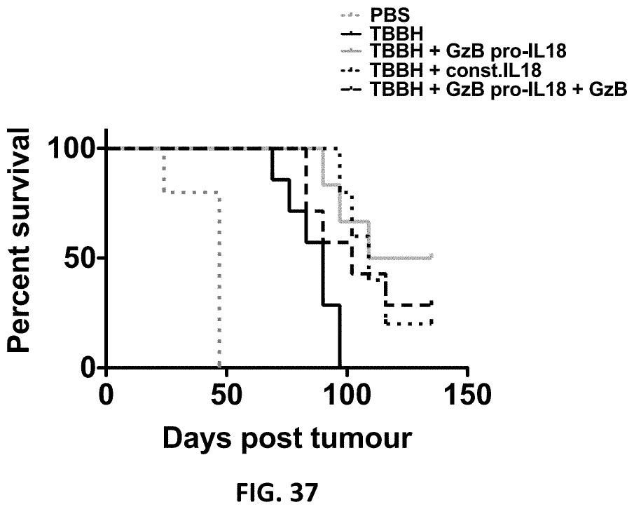

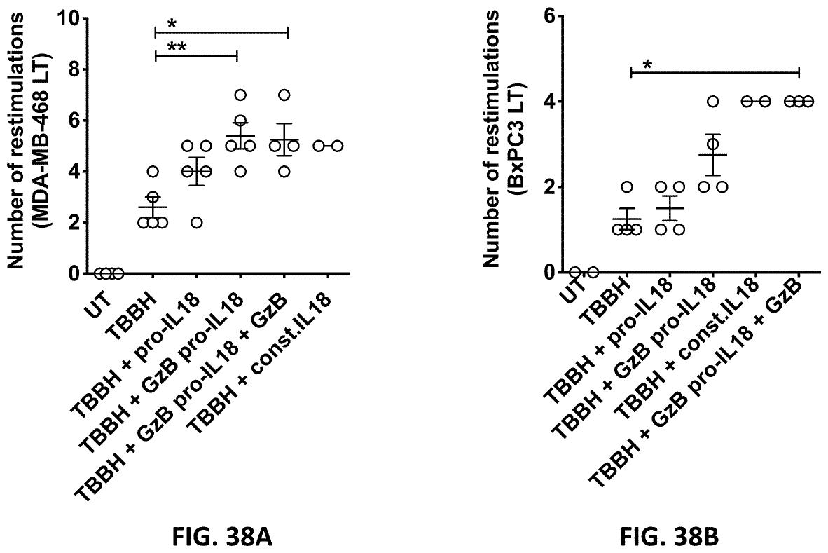

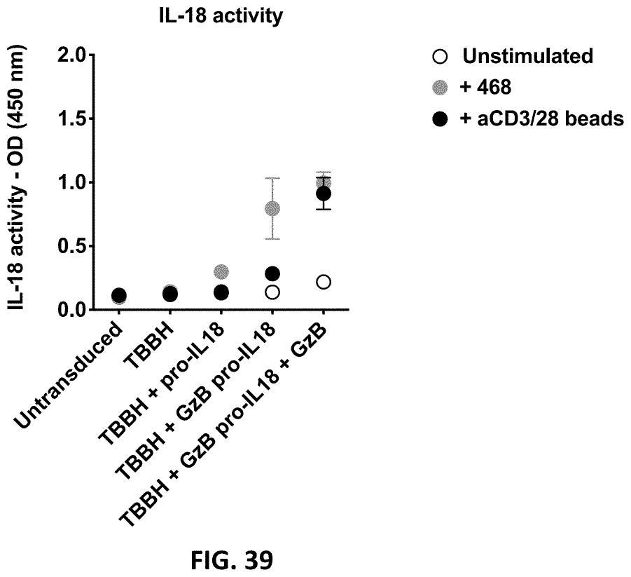

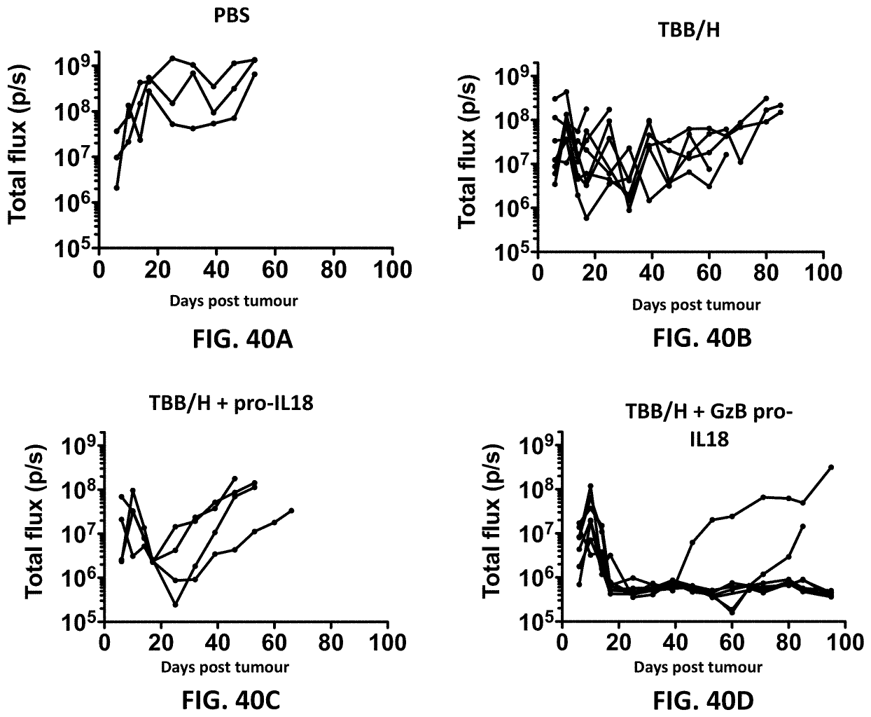

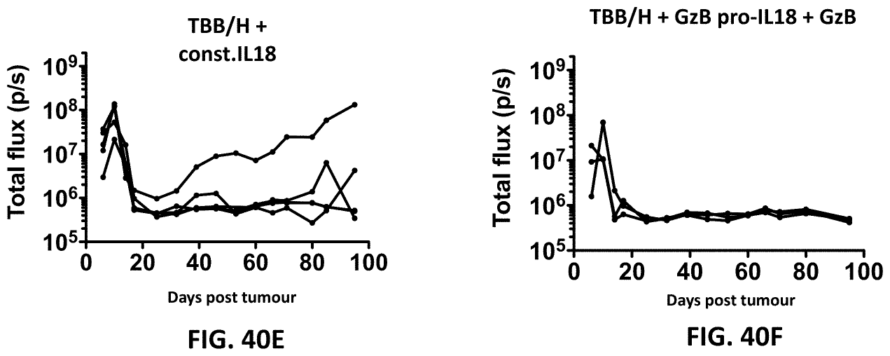

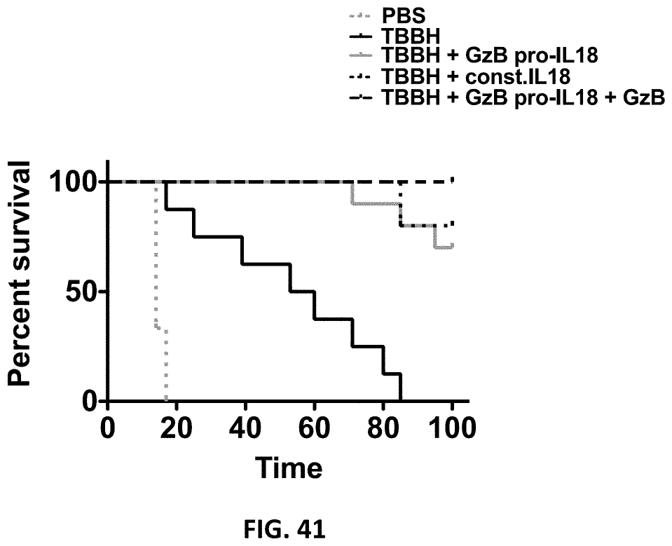

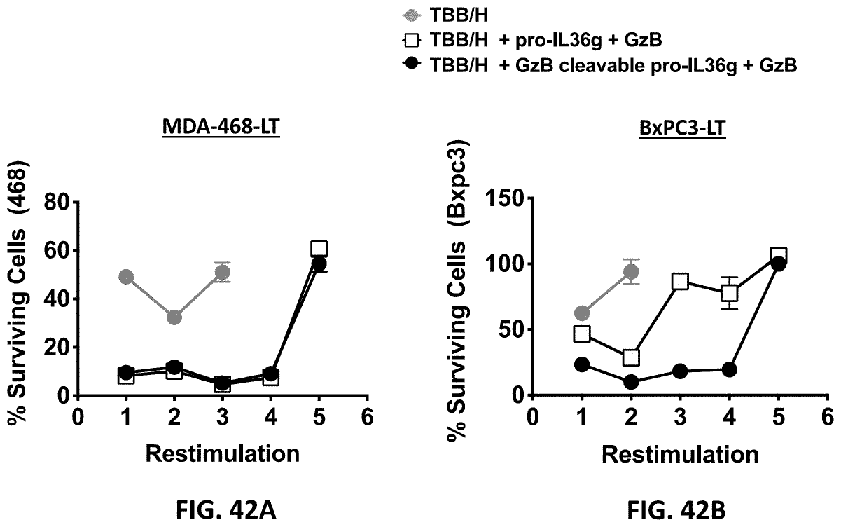

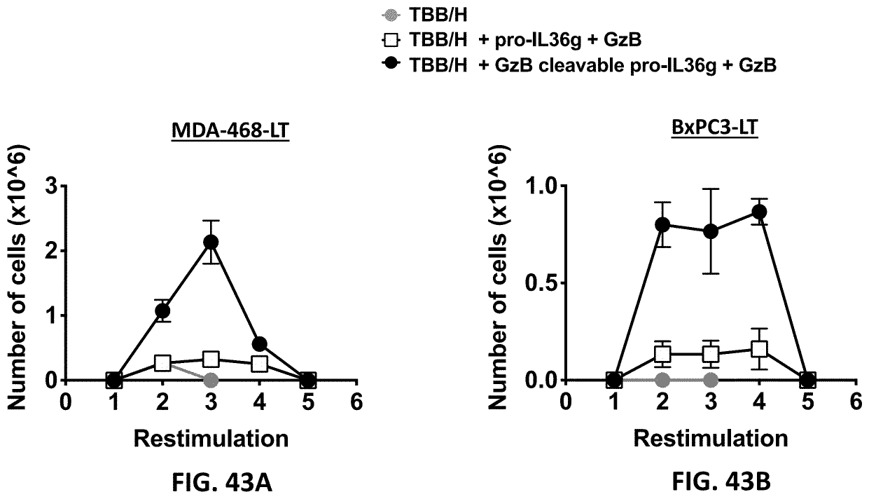

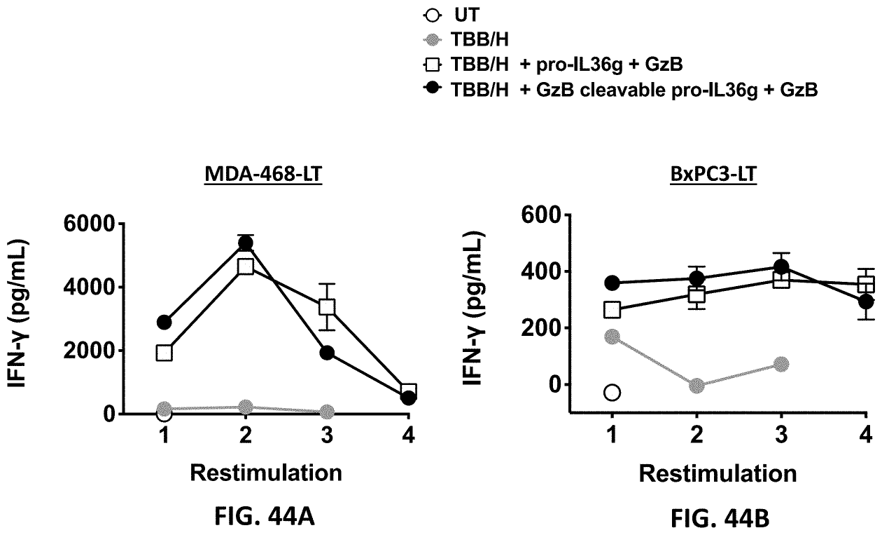

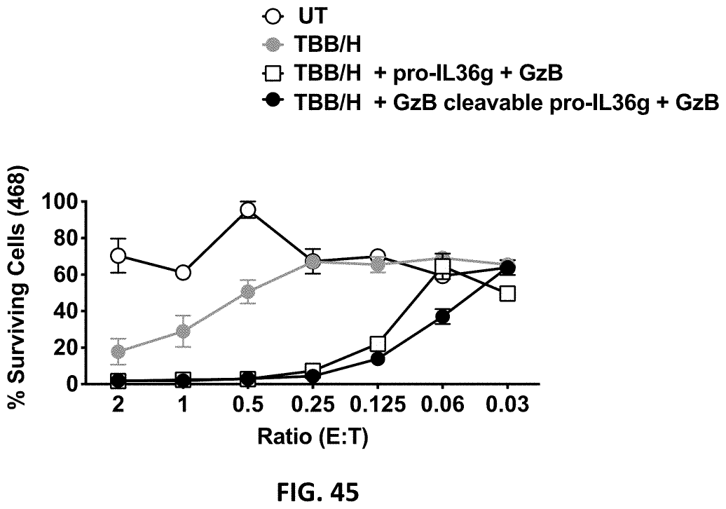

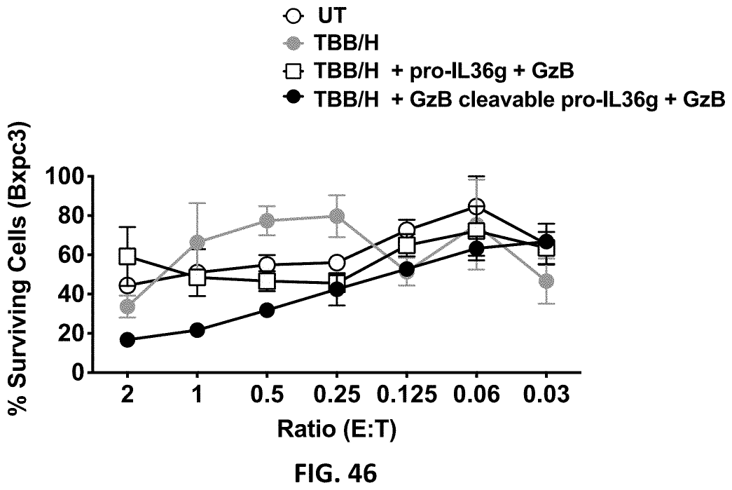

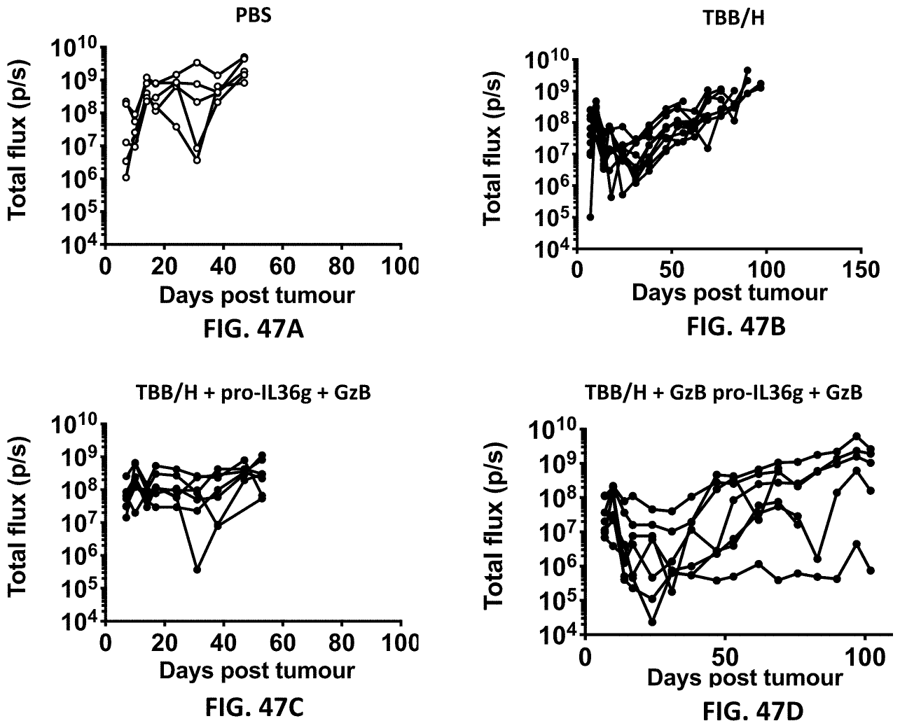

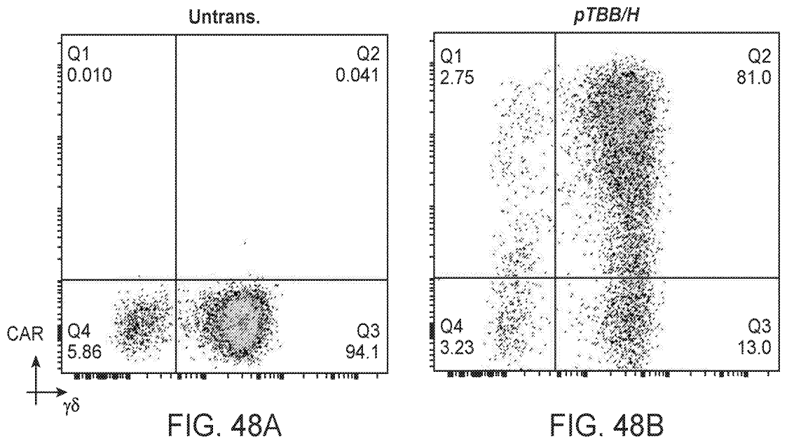

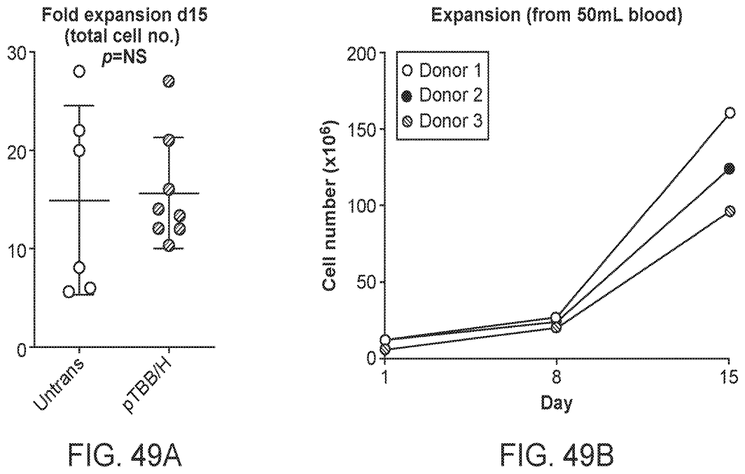

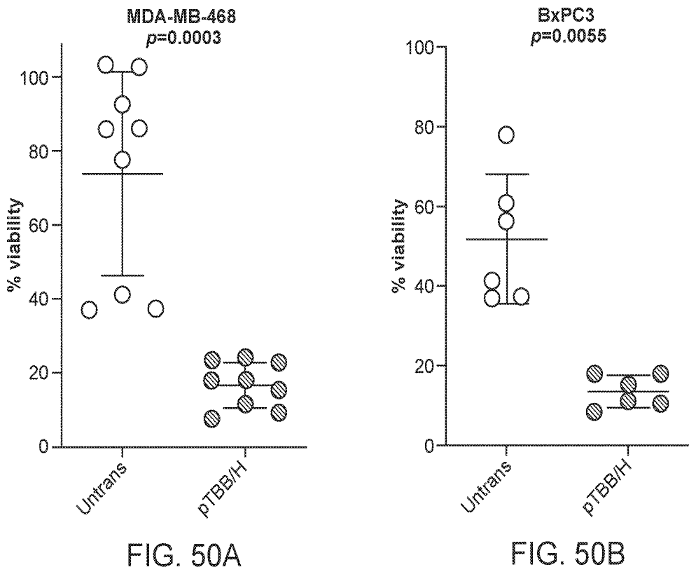

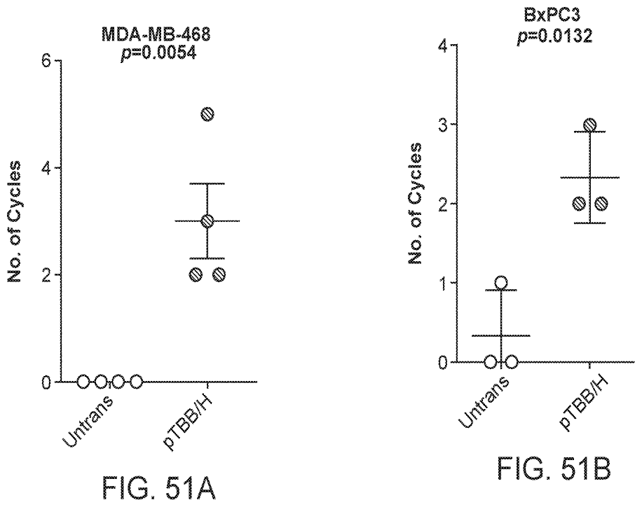

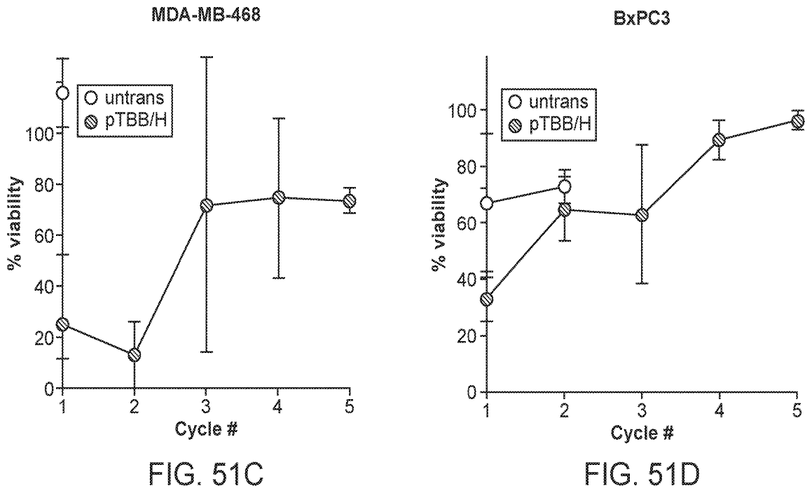

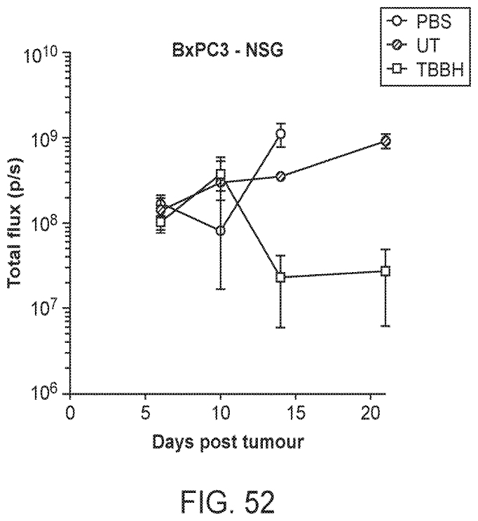

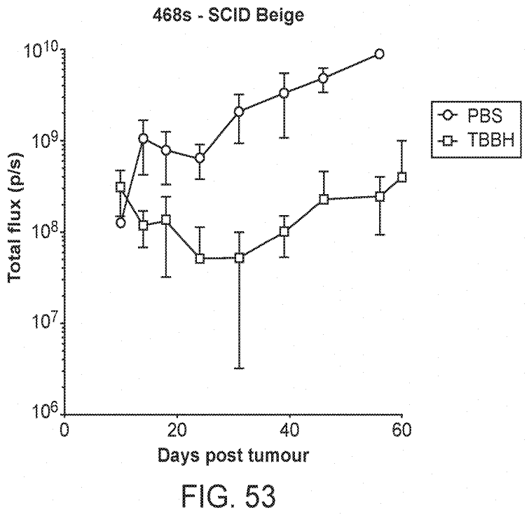

The Examples herein provide additional second generation CARs that can usefully be expressed in the immunoresponsive cells described herein. In particular embodiments, a second-generation CAR, denominated “H,” “H2”, or “H28z”, is used. The H2 CAR comprises, from extracellular to intracellular domain, a MUC-1 targeting the HMFG2 scFv, CD28 transmembrane and co-stimulatory domains, and a CD3z signalling region. See FIG. 1. The H2 CAR is described in Wilkie et al., “Retargeting of human T cells to tumor-associated MUC1: the evolution of a chimeric antigen receptor,” J. Immunol. 180:4901-9 (2008), incorporated herein by reference in its entirety. In particular embodiments, a second-generation CAR, called T1E28z, is used. The T1E28z CAR comprises, from extracellular to intracellular domain, the ErbB targeting T1E peptide, CD28 transmembrane and co-stimulatory domains, and a CD3z signalling region. See FIG. 1. The T1E28z second generation CAR is described in Davies, “Flexible targeting of ErbB dimers that drive tumourigenesis by using genetically engineered T cells,” Mol. Med. 18:565-576 (2012), incorporated herein by reference in its entirety.

In some embodiments, a third-generation CAR is used. The third-generation CAR can combine multiple co-stimulatory domains (synonymously, co-stimulatory signalling regions) with a TCR-like signalling domain (synonymously, signalling region) in cis, such as CD28+4-1BB+CD3z or CD28+OX40+CD3z, to further augment potency. In some embodiments, the third-generation CARs comprise the co-stimulatory domains aligned in series in the CAR endodomain, generally placed upstream of CD3z or its equivalent. Some exemplary third-generation CARs that can usefully be expressed in the immunoresponsive cells described herein are disclosed in Pule et al., “A chimeric T cell antigen receptor that augments cytokine release and supports clonal expansion of primary human T cells,” Mol Ther. 12(5):933-41 (2005); Geiger et al., “Integrated src kinase and costimulatory activity enhances signal transduction through single-chain chimeric receptors in T lymphocytes,” Blood 98:2364-71 (2001); and Wilkie et al., “Retargeting of human T cells to tumor-associated MUC1: the evolution of a chimeric antigen receptor,” J. Immunol. 180(7):4901-9 (2008), the disclosures of which are incorporated herein by reference in their entireties, and in FIG. 26. In some embodiments, a CAR using both cis and trans co-stimulatory signals as disclosed in Stephan et al., “T cell-encoded CD80 and 4-1BBL induce auto- and transcostimulation, resulting in potent tumor rejection,” Nat. Med. 13(12)1440-9 (2007), incorporated by reference herein, and provided in FIG. 26, is used.

Other CAR formats available and known in the art can be expressed in various embodiments of the immunoresponsive cells described herein. In particular, FIGS. 27-29 disclose additional CAR formats that can be expressed in the immunosuppressive cells of the present disclosures, including those disclosed in Wilkie et al., “Dual Targeting of ErbB2 and MUC1 in Breast Cancer Using Chimeric Antigen Receptors Engineered to Provide Complementary Signaling,” J. Clin. Immunol. 32(5)1059-70 (2012); Fedorov et al. “PD-1- and CTLA-4-based inhibitory chimeric antigen receptors (iCARs) divert off-target immunotherapy responses,” Sci. Transl. Med. 5(215)215ra172 (2013); Kloss et al. “Combinatorial antigen recognition with balanced signaling promotes selective tumor eradication by engineered T cells,” Nat. Biotechnol. 31(1):71-6 (2013); Grada et al. “TanCAR: A Novel Bispecific Chimeric Antigen Receptor for Cancer Immunotherapy,” Mol. Ther. Nucleic Acids. 2:e105 (2013); Foster et al. “Regulated Expansion and Survival of Chimeric Antigen Receptor-Modified T Cells Using Small Molecule-Dependent Inducible MyD88/CD40,” Mol Ther. 25(9):2176-2188 (2017); Chmielewski et al. “IL-12 release by engineered T cells expressing chimeric antigen receptors can effectively muster an antigen-independent macrophage response on tumor cells that have shut down tumor antigen expression,” Cancer Research, 71:5697-5706 (2011); Pegram et al., “Tumor-targeted T cells modified to secrete IL-12 eradicate systemic tumors without need for prior conditioning,” Blood 119:4133-4141 (2012); Curran et al. “Enhancing antitumor efficacy of chimeric antigen receptor T cells through constitutive CD40L expression,” Mol. Ther. 23(4):769-78 (2015); Zhao et al., “Structural design of engineered costimulation determines tumor rejection kinetics and persistence of CAR T cells,” Cancer Cell 28:415-28 (2015); Roybal et al., “Precision tumor recognition by T Cells with combinatorial antigen-sensing circuits, Cell 164:770-9 (2016); Whilding et al., “CAR T-Cells targeting the integrin alphavbeta6 and co-expressing the chemokine receptor CXCR2 demonstrate enhanced homing and efficacy against several solid malignancies,” Cancers 11(5), 674 (2019) and Kosti et al., “Perspectives on Chimeric Antigen Receptor T-Cell immunotherapy for solid tumors,” Front Immunol 9:1104, (2018) incorporated by reference in their entireties herein.

### 4.3.5.2.1. pCAR Format

In particular embodiments, a parallel CAR (pCAR) is expressed in the immunoresponsive cell.

In pCAR embodiments, immunoresponsive cells are engineered to express two constructs in parallel, a second-generation CAR and a chimeric co-stimulatory receptor (CCR). The second-generation CAR comprises, from intracellular to extracellular domain, (a) a signalling region; (b) a first co-stimulatory signalling region; (c) a transmembrane domain; and (d) a first binding element that specifically interacts with a first epitope on a first target antigen. The CCR comprises, from intracellular to extracellular domain, (a) a co-stimulatory signalling region; (b) a transmembrane domain; and (c) a second binding element that specifically interacts with a second epitope on a second target antigen. Typically, the CCR lacks a TCR-like signalling region such as CD3z. In some embodiments, the co-stimulatory domain of the CCR (the second costimulatory domain) is different from the co-stimulatory domain of the CAR (the first costimulatory domain). In some embodiments, the second epitope is different from the first epitope. Parallel CAR (pCAR)-engineered T cells have been demonstrated to have superior activity and resistance to exhaustion as compared to first generation CAR-T cells, second generation CAR-T cells, and third generation CAR-T cells. See US pre-grant publication 2019/0002521, incorporated herein by reference in its entirety.

In some embodiments, the second target antigen is different from the first target antigen. In some embodiments, the second target antigen is the same as the first target antigen.

In some embodiments, the first antigen is a MUC1 antigen. In particular embodiments, the first epitope is a tumour-associated epitope on a MUC1 target antigen. In some embodiments, the first binding element comprises the CDRs of the HMFG2 antibody. In some embodiments, the first binding element comprises the V_{H} and V_{L} domains of the HMFG2 antibody. In some embodiments, the first binding element comprises an HMFG2 single-chain variable fragment (scFv).

In particular embodiments, the CAR is the H2 second generation CAR, which comprises, from extracellular to intracellular domain, a MUC-1 targeting the HMFG2 scFv, CD28 transmembrane and co-stimulatory domains, and a CD3z signalling region. See FIG. A. The H2 CAR is described in Wilkie et al., “Retargeting of human T cells to tumor-associated MUC1: the evolution of a chimeric antigen receptor,” J. Immunol. 180:4901-9 (2008), incorporated herein by reference in its entirety.

In particular embodiments, the CAR is the T1E28z second generation CAR, which comprises, from extracellular to intracellular domain, the ErbB targeting T1E peptide, CD28 transmembrane and co-stimulatory domains, and a CD3z signalling region. See Fig A. The T1E28z second generation CAR is described in Davies, “Flexible targeting of ErbB dimers that drive tumourigenesis by using genetically engineered T cells,” Mol. Med. 18:565-576 (2012), incorporated herein by reference in its entirety.

In some embodiments, the second target antigen is selected from the group consisting of ErbB homodimers and heterodimers. In particular embodiments, the second target antigen is HER2. In particular embodiments, said second target antigen is the EGF receptor. In some embodiments, the second binding element comprises T1E, the binding moiety of ICR12, or the binding moiety of ICR62.

In some embodiments, pCARs “TBB/H” or “I12BB/H,” are expressed in the immunoresponsive cells. These pCARs utilize the MUC1-targeting 2^{nd} generation “H” (synonymously, “H2”) CAR, but with different co-expressed CCRs. The CCR in the TBB/H pCAR has a T1E binding domain fused to CD8α transmembrane domain and a 4-1BB co-stimulatory domain. T1E is a chimeric peptide derived from transforming growth factor-α (TGF-α) and epidermal growth factor (EGF) and is a promiscuous ErbB ligand. See Wingens et al., “Structural analysis of an epidermal growth factor/transforming growth factor-alpha chimera with unique ErbB binding specificity,” J. Biol. Chem. 278:39114-23 (2003) and Davies et al., “Flexible targeting of ErbB dimers that drive tumourigenesis by using genetically engineered T cells,” Mol. Med. 18:565-576 (2012), the disclosures of which are incorporated herein by reference in their entireties. The CCR in the I12BB/H pCAR has an ICR12 binding domain fused to a CD8α transmembrane domain and a 4-1BB co-stimulatory domain. ICR12 is a HER2 (ErbB2) targeting scFv domain. See Styles et al., “Rat monoclonal antibodies to the external domain of the product of the C-erbB-2 proto-oncogene,” Int. J. Cancer 45(2):320-24 (1990), incorporated herein by reference in its entirety. In some embodiments, “TBB/H” or other pCARs described in PCT/GB2020/050590, incorporated by reference in its entirety, can be used.

In some embodiments, the ABB/H and I62BB/H pCARs are used. The CAR in both ABB/H and I62BB/H is the MUC1-targeting 2^{nd} generation “H” CAR. The CCR in the ABB/H pCAR has an A20 peptide fused to CD8α transmembrane domain and a 4-1BB co-stimulatory domain. The A20 peptide binds to αvβ6 integrin. See DiCara et al., “Structure-function analysis of Arg-Gly-Asp helix motifs in alpha v beta 6 integrin ligands,” J Biol Chem. 282(13):9657-9665 (2007), incorporated herein by reference in its entirety. The CCR in the I62BB/H pCAR has an ICR62 binding domain fused to a CD8α transmembrane domain and a 4-1BB co-stimulatory domain. ICR62 is an EGFR targeting scFv domain. See Modjtahedi et al., “Antitumor activity of combinations of antibodies directed against different epitopes on the extracellular domain of the human EGF receptor,” Cell Biophys. 22(1-3):129-146 (1993), incorporated herein by reference in its entirety.

In some embodiments, the immunoresponsive cells express the modified pro-cytokine (e.g., the modified pro-IL-18 or modified pro-IL-36), optional expressed protease, and optional CAR or pCAR from a single expression construct. In some embodiments, the immunoresponsive cells express the modified pro-cytokine (e.g., the modified pro-IL-18 or modified pro-IL-36), optional protease, the CAR or pCAR from a plurality of distinct constructs.

### 4.3.5.2.2. Signalling Region

The CAR construct comprises a signalling region (i.e. a TCR-like signalling region). In some embodiments, the signalling region comprises an Immune-receptor-Tyrosine-based-Activation-Motif (ITAM), as reviewed for example by Love et al., “ITAM-mediated signaling by the T-cell antigen receptor,” Cold Spring Harbor Perspect. Biol 2(6)1 a002485 (2010). In some embodiments, the signalling region comprises the intracellular domain of human CD3 zeta chain, as described for example in U.S. Pat. No. 7,446,190, incorporated by reference herein, or a variant thereof. In particular embodiments, the signalling region comprises the domain which spans amino acid residues 52-163 of the full-length human CD3 zeta chain. The CD3 zeta chain has a number of known polymorphic forms, (e.g. Sequence ID: gb|AAF34793.1 and gb|AAA60394.1), all of which are useful herein, and shown respectively as SEQ ID NO: 1 and 2:

(SEQ ID NO: 1)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR;
(SEQ ID NO: 2)
RVKFSRSAEPPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR.

Alternative signalling regions to the CD3 zeta domain include, e.g., FceR1γ, CD3ε, and multi-ITAM. See Eshhar Z et al., “Specific activation and targeting of cytotoxic lymphocytes through chimeric single chains consisting of antibody-binding domains and the gamma or zeta subunits of the immunoglobulin and T-cell receptors,” Proc Natl Acad Sci USA 90:720-724 (1993); Nolan et al., “Bypassing immunization: optimized design of “designer T cells” against carcinoembryonic antigen (CEA)-expressing tumors, and lack of suppression by soluble CEA,” Clin Cancer Res 5: 3928-3941 (1999); Zhao et al., “A herceptin-based chimeric antigen receptor with modified signaling domains leads to enhanced survival of transduced T lymphocytes and antitumor activity,” J Immunol 183: 5563-5574 (2009); and James J R, “Tuning ITAM multiplicity on T cell receptors can control potency and selectivity to ligand density,” Sci Signal 11(531) eaan1088 (2018), the disclosures of which are incorporated herein by reference in their entireties.

### 4.3.5.2.3. Co-Stimulatory Signalling Region

In the CAR, the co-stimulatory signalling region is suitably located between the signalling region and transmembrane domain, and remote from the binding element.

In the CCR, the co-stimulatory signalling region is suitably located adjacent the transmembrane domain and remote from the binding element.

Suitable co-stimulatory signalling regions are well known in the art, and include the co-stimulatory signalling regions of members of the B7/CD28 family such as B7-1, B7-2, B7-H1, B7-H2, B7-H3, B7-H4, B7-H6, B7-H7, BTLA, CD28, CTLA-4, Gi24, ICOS, PD-1, PD-L2 or PDCD6; or ILT/CD85 family proteins such as LILRA3, LILRA4, LILRB1, LILRB2, LILRB3 or LILRB4; or tumour necrosis factor (TNF) superfamily members such as 4-1BB, BAFF, BAFF R, CD27, CD30, CD40, DR3, GITR, HVEM, LIGHT, Lymphotoxin-alpha, OX40, RELT, TACI, TL1A, TNF-alpha, or TNF RII; or members of the SLAM family such as 2B4, BLAME, CD2, CD2F-10, CD48, CD8, CD84, CD229, CRACC, NTB-A or SLAM; or members of the TIM family such as TIM-1, TIM-3 or TIM-4; or other co-stimulatory molecules such as CD7, CD96, CD160, CD200, CD300a, CRTAM, DAP12, Dectin-1, DPPIV, EphB6, Integrin alpha 4 beta 1, Integrin alpha 4 beta 7/LPAM-1, LAG-3 or TSLP R. See Mondino A et al., “Surface proteins involved in T cell costimulation,” J Leukoc Biol. 55:805-815 (1994); Thompson C B, “Distinct roles for the costimulatory ligands B7-1 and B7-2 in T helper cell differentiation?,” Cell. 81:979-982 (1995); Somoza C and Lanier L L, “T-cell costimulation via CD28-CD80/CD86 and CD40-CD40 ligand interactions,” Res Immunol. 146:171-176 (1995); Rhodes D A et al., “Regulation of immunity by butyrophilins,” Annu Rev Immunol. 34:151-172 (2016); Foell J et al., “T cell costimulatory and inhibitory receptors as therapeutic targets for inducing anti-tumor immunity”, Curr Cancer Drug Targets. 7:55-70 (2007); Greenwald R J et al., Annu Rev Immunol., “The B7 family revisited,” 23:515-548 (2005); Flem-Karlsen K et al., “B7-H3 in cancer —beyond immune regulation,” Trends Cancer. 4:401-404 (2018); Flies D B et al., “The new B7s: playing a pivotal role in tumor immunity,” J Immunother. 30:251-260 (2007); Gavrieli M et al., “BTLA abd HVEM cross talk regulates inhibition and costimulation,” Adv Immunol. 92:157-185 (2006); Zhu Y et al., “B7-H5 costimulates human T cells via CD28H,” Nat Commun. 4:2043 (2013); Omar H A et al., “Tacking molecular targets beyond PD-1/PD-L1: Novel approaches to boost patients' response to cancer immunotherapy,” Crit Rev Oncol Hematol. 135:21-29 (2019); Hashemi M et al., “Association of PDCD6 polymorphisms with the risk of cancer: Evidence from a meta-analysis,” Oncotarget. 9:24857-24868 (2018); Kang X et al., “Inhibitory leukocyte immunoglobulin-like receptors: Immune checkpoint proteins and tumor sustaining factors,” Cell Cycle. 15:25-40 (2016); Watts T H, “TNF/TNFR family members in costimulation of T cell responses,” Annu Rev Immunol. 23:23-68 (2005); Bryceson Y T et al., “Activation, coactivation, and costimulation of resting human natural killer cells,” Immunol Rev. 214:73-91 (2006); Sharpe A H, “Analysis of lymphocyte costimulation in vivo using transgenic and ‘knockout’ mice,” Curr Opin Immunol. 7:389-395 (1995); Wingren A G et al., “T cell activation pathways: B7, LFA-3, and ICAM-1 shape unique T cell profiles,” Crit Rev Immunol. 15:235-253 (1995), the disclosures of which are incorporated herein by reference in their entireties.

The co-stimulatory signalling regions may be selected depending upon the particular use intended for the immuno-responsive cell. In particular, the co-stimulatory signalling regions can be selected to work additively or synergistically together. In some embodiments, the co-stimulatory signalling regions are selected from the co-stimulatory signalling regions of CD28, CD27, ICOS, 4-1BB, OX40, CD30, GITR, HVEM, DR3 and CD40.

In a particular embodiment, one co-stimulatory signalling region of the pCAR is the co-stimulatory signalling region of CD28 and the other is the co-stimulatory signalling region of 4-1BB.

### 4.3.5.2.4. Transmembrane Domains

The transmembrane domains for the CAR and CCR constructs may be the same or different. In currently preferred embodiments, when the CAR and CCR constructs are expressed from a single vector, the transmembrane domains of the CAR and CCR are different, to ensure separation of the constructs on the surface of the cell. Selection of different transmembrane domains may also enhance stability of the expression vector since inclusion of a direct repeat nucleic acid sequence in the viral vector renders it prone to rearrangement, with deletion of sequences between the direct repeats. In embodiments in which the transmembrane domains of the CAR and CCR of the pCAR are chosen to be the same, this risk can be reduced by modifying or “wobbling” the codons selected to encode the same protein sequence.

Suitable transmembrane domains are known in the art and include for example, the transmembrane domains of CD8α, CD28, CD4 or CD3z. Selection of CD3z as transmembrane domain may lead to the association of the CAR or CCR with other elements of TCR/CD3 complex. This association may recruit more ITAMs but may also lead to the competition between the CAR/CCR and the endogenous TCR/CD3.

### 4.3.5.2.5. Co-Stimulatory Signal Domain and Transmembrane Domain

In embodiments in which the co-stimulatory signalling region of the CAR or CCR is, or comprises, the co-stimulatory signalling region of CD28, the CD28 transmembrane domain represents a suitable, often preferred, option for the transmembrane domain. The full length CD28 protein is a 220 amino acid protein of SEQ ID NO: 3, where the transmembrane domain is shown in bold type:

(SEQ ID NO: 3)
MLRLLLALNLFPSIQVTGNKILVKQSPMLVAYDNAVNLSCKYSYNLFSRE
FRASLHKGLDSAVEVCVVYGNYSQQLQVYSKTGFNCDGKLGNESVTFYLQ
NLYVNQTDIYFCKIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRS.

In some embodiments, one of the co-stimulatory signalling regions is based upon the hinge region and suitably also the transmembrane domain and endodomain of CD28. In some embodiments, the co-stimulatory signalling region comprises amino acids 114-220 of SEQ ID NO: 3, shown below as SEQ ID NO: 4:

(SEQ ID NO: 4)
IEVMYPPPYLDNEKSNGTIIHVKGKHLCPSPLFPGPSKPFWVLVVVGGVL
ACYSLLVTVAFIIFWVRSKRSRLLHSDYMNMTPRRPGPTRKHYQPYAPPR
DFAAYRS.

In a particular embodiment, one of the co-stimulatory signalling regions is a modified form of SEQ ID NO: 4 which includes a c-myc tag of SEQ ID NO: 5:

(SEQ ID NO: 5)
EQKLISEEDL.

The c-myc tag may be added to the co-stimulatory signalling region by insertion into the ectodomain or by replacement of a region in the ectodomain, which is therefore within the region of amino acids 1-152 of SEQ ID NO: 3.

In a particularly preferred embodiment, the c-myc tag replaces MYPPPY motif in the CD28 sequence. This motif represents a potentially hazardous sequence. It is responsible for interactions between CD28 and its natural ligands, CD80 and CD86, so that it provides potential for off-target toxicity when CAR-T cells or pCAR-T cells encounter a target cell that expresses either of these ligands. By replacement of this motif with a tag sequence as described above, the potential for unwanted side-effects is reduced. Thus, in a particular embodiment, the co-stimulatory signalling region of the CAR construct comprises SEQ ID NO: 6:

(SEQ ID NO: 6)
IEVEQKLISEEDLLDNEKSNGTIIHVKGKHLCPSPLFPGPSKPFWVLVVV
GGVLACYSLLVTVAFIIFWVRSKRSRLLHSDYMNMTPRRPGPTRKHYQPY
APPRDFAAYRS.

Furthermore, the inclusion of a c-myc epitope facilitates detection of the pCAR-T cells using a monoclonal antibody to the c-myc epitope. This is very useful since flow cytometric detection had proven unreliable when using some available antibodies.

In addition, the provision of a c-myc epitope tag could facilitate the antigen independent expansion of targeted CAR-T cells, for example by cross-linking of the CAR using the appropriate monoclonal antibody, either in solution or immobilised onto a solid phase (e.g., a bag).

Moreover, expression of the epitope for the anti-human c-myc antibody, 9e10, within the variable region of a TCR has previously been shown to be sufficient to enable antibody-mediated and complement mediated cytotoxicity both in vitro and in vivo (Kieback et al. Proc. Natl. Acad. Sci. USA, “A safeguard eliminates T cell receptor gene-modified autoreactive T cells after adoptive transfer,” 105(2) 623-8 (2008)). Thus, the provision of such epitope tags could also be used as a “suicide system,” whereby an antibody could be used to deplete pCAR-T cells in vivo in the event of toxicity.

### 4.3.5.2.6. Binding Elements

The binding elements of the CAR and CCR constructs of the pCAR respectively bind a first epitope and a second epitope.

In typical embodiments, the binding elements of the CAR and CCR constructs are different from one another.

In various embodiments, the binding elements of the CAR and CCR specifically bind to a first epitope and second epitope of the same antigen. In certain of these embodiments, the binding elements of the CAR and CCR specifically bind to the same, overlapping, or different epitopes of the same antigen. In embodiments in which the first and second epitopes are the same or overlapping, the binding elements on the CAR and CCR can compete in their binding.

In various embodiments, the binding elements of the CAR and CCR constructs of the pCAR bind to different antigens. In certain embodiments, the antigens are different but may be associated with the same disease, such as the same specific cancer.

Thus, suitable binding elements may be any element which provides the pCAR with the ability to recognize a target of interest. The target to which the pCARs of the invention are directed can be any target of clinical interest to which it would be desirable to direct a T cell response.

In various embodiments, the binding elements used in the CARs and CCRs of the pCARs described herein are antigen binding sites (ABS) of antibodies. In typical embodiments, the ABS used as the binding element is formatted into a single chain antibody (scFv) or is single domain antibody from a camelid, human or other species.

Alternatively, a binding element of a pCAR may comprise ligands that bind to a surface protein of interest.

In some embodiments, the binding element is associated with a leader (signal peptide) sequence which facilitates expression on the cell surface. Many leader sequences are known in the art, and these include but are not restricted to the CD8α leader sequence, immunoglobulin kappa light chain sequence, macrophage colony stimulating factor receptor (FMS) leader sequence or CD124 leader sequence.

MUC1 pCARs

In particular embodiments, at least one of the binding elements specifically interacts with an epitope on a MUC1 target antigen. In some embodiments, the binding element of the CAR specifically interacts with an epitope on a MUC1 antigen. In some embodiments, the binding element of the CCR specifically interacts with an epitope on a MUC1 target antigen, or an alternative tumour-associated molecule such as an NKG2D ligand, the αvβ6 integrin or an ErbB homo- or heterodimer. In certain embodiments, the binding element of the CAR specifically interacts with an epitope on a MUC1 antigen and the binding element of the CCR specifically interacts with the same, overlapping, or different epitope on a MUC1 target antigen.

In currently preferred embodiments, the binding element of the CAR specifically interacts with a first epitope on a MUC1 target antigen. In some embodiments, the CAR binding element comprises the antigen binding site of the HMFG2 antibody. In certain embodiments, the CAR binding element comprises the CDRs of the HMFG2 antibody. The CDR sequences of the HMFG2 antibody were determined using the tools provided on www.abysis.org and are shown below as SEQ ID NOs: 8-13:

(SEQ ID NO: 8)
VH CDR1 GFTFSNY;
(SEQ ID NO: 9)
VH CDR2 RLKSNNYA;
(SEQ ID NO: 10)
VH CDR3 GNSFAY;
(SEQ ID NO: 11)
VL CDR1 RSSTGAVTTSNYAN;
(SEQ ID NO: 12)
VL CDR2 GTNNRAP;
(SEQ ID NO: 13)
VL CDR3 ALWYSNHWV.

In certain embodiments, the CAR binding element comprises the V_{H} and V_{L} domains of the HMFG2 antibody. The V_{H} and V_{L} domain sequences of the HMFG2 antibody are shown below as SEQ ID NOs: 14-15:

(SEQ ID NO: 14)
EVQLQQSGGGLVQPGGSMKLSCVASGETFSNYWMNWVRQSPEKGLEWVAE
IRLKSNNYATHYAESVKGRFTISRDDSKSSVYLQMNNLRAEDTGIYYCTF
GNSFAYWGQGTTVTVSS
(SEQ ID NO: 15)
QAVVTQESALTTSPGETVTLTCRSSTGAVTTSNYANWVQEKPDHLFTGLI
GGTNNRAPGVPARFSGSLIGDKAALTITGAQTEDEAIYFCALWYSNHWVF
GGGTKLTVLGSE.

In particularly preferred embodiments, the CAR binding element comprises the antigen binding site of the HMFG2 antibody formatted as a scFv, either configured in the order of V_{H}-spacer-V_{L} or V_{L}-spacer V_{H}. In certain embodiments, the amino acid sequence of the scFv of the HMGF2 antibody is 70%, 75%, 80%, 85%, 90%, 95%, 97%, 99%, or 100% identical to SEQ ID NO: 16 shown below:

(SEQ ID NO: 16)
EVQLQQSGGGLVQPGGSMKLSCVASGFTFSNYWMNWVRQSPEKGLEWVAE
IRLKSNNYATHYAESVKGRFTISRDDSKSSVYLQMNNLRAEDTGIYYCTF
GNSFAYWGQGTTVTVSSGGGGSGGGGSGGGGSQAVVTQESALTTSPGETV
TLTCRSSTGAVTTSNYANWVQEKPDHLFTGLIGGTNNRAPGVPARFSGSL
IGDKAALTITGAQTEDEAIYFCALWYSNHWVFGGGTKLTVLGSE.

In certain embodiments, the nucleic acid encoding the scFv of the HMGF2 antibody is SEQ ID NO: 17 shown below:

(SEQ ID NO: 17)
GAGGTGCAGCTGCAGCAGTCTGGAGGAGGCTTGGTGCAACCTGGAGGATC
CATGAAACTCTCCTGTGTTGCCTCTGGATTCACTTTCAGTAACTACTGGA
TGAACTGGGTCCGCCAGTCTCCAGAGAAGGGGCTTGAGTGGGTTGCTGAA
ATTAGATTGAAATCTAATAATTATGCAACACATTATGCGGAGTCTGTGAA
AGGGAGGTTCACCATCTCAAGAGATGATTCCAAAAGTAGTGTCTACCTGC
AAATGAACAACTTAAGAGCTGAAGACACTGGCATTTATTACTGTACCTTT
GGTAACTCCTTTGCTTACTGGGGCCAAGGGACCACGGTCACCGTCTCCTC
AGGTGGAGGCGGTTCAGGCGGAGGTGGCTCTGGCGGTGGCGGATCGCAGG
CCGTGGTCACTCAGGAATCTGCACTCACCACATCACCTGGTGAAACAGTC
ACACTCACTTGTCGCTCAAGTACTGGGGCTGTTACAACTAGTAACTATGC
CAACTGGGTCCAAGAAAAACCAGATCATTTATTCACTGGTCTAATAGGTG
GTACCAACAACCGAGCACCAGGTGTTCCTGCCAGATTCTCAGGCTCCCTG
ATTGGAGACAAGGCTGCCCTCACCATCACAGGGGCACAGACTGAGGATGA
GGCAATATATTTCTGTGCTCTATGGTACAGCAACCATTGGGTGTTCGGTG
GAGGAACCAAACTGACTGTCCTAGGATCAGAG.

In some embodiments, the CCR binding element is ICR12, which binds to HER2. See Styles et al., “Rat monoclonal antibodies to the external domain of the product of the C-erbB-2 proto-oncogene,” Int. J. Cancer 45(2):320-24 (1990), incorporated herein by reference in its entirety. In some embodiments, the CCR binding element is ICR62, which binds to EGFR. See Modjtahedi et al., “Antitumor activity of combinations of antibodies directed against different epitopes on the extracellular domain of the human EGF receptor,” Cell Biophys. 22(1-3):129-46 (1993), incorporated herein by reference in its entirety. In some embodiments, the CCR binding element is the A20 peptide, which binds to αvβ6 integrin. See DiCara et al., “Structure-function analysis of Arg-Gly-Asp helix motifs in alpha v beta 6 integrin ligands,” J Biol Chem. 282(13):9657-9665 (2007), incorporated herein by reference in its entirety.

In some embodiments, the CCR binding element is the T1E peptide, which binds ErbB homo- and heterodimers. T1E is a chimeric peptide derived from transforming growth factor-α (TGF-α) and epidermal growth factor (EGF) and is a promiscuous ErbB ligand. The T1E peptide is a chimeric fusion protein composed of the entire mature human EGF protein, excluding the five most N-terminal amino acids (amino acids 971-975 of pro-epidermal growth factor precursor (NP 001954.2)), which have been replaced by the seven most N-terminal amino acids of the mature human TGF-α protein (amino acids 40-46 of pro-transforming growth factor alpha isoform 1 (NP 003227.1)). See Wingens et al., “Structural analysis of an epidermal growth factor/transforming growth factor-alpha chimera with unique ErbB binding specificity,” J. Biol. Chem. 278:39114-23 (2003) and Davies et al., “Flexible targeting of ErbB dimers that drive tumorigenesis by using genetically engineered T cells,” Mol. Med. 18:565-576 (2012), the disclosures of which are incorporate herein by reference in their entireties. The sequence of T1E is shown below as SEQ ID NO: 18:

(SEQ ID NO: 18)
VVSHFNDCPLSHDGYCLHDGVCMYIEALDKYACNCVVGYIGERCQYRDLK
WWELR.

In certain embodiments, the nucleic acid encoding the T1E sequence is SEQ ID NO: 19 shown below:

(SEQ ID NO: 19)
GTGGTGAGCCACTTCAACGACTGCCCTCTGAGCCACGACGGCTACTGCCT
GCACGACGGCGTGTGCATGTACATCGAGGCCCTGGACAAGTACGCCTGCA
ACTGCGTGGTGGGCTACATCGGCGAGAGATGCCAGTACAGAGACCTGAAG
TGGTGGGAGCTGAGA.

The protein sequence of TBB/H pCAR is shown below as SEQ ID NO: 7. The TBB/H pCAR comprises a CCR comprising a T1E binding domain fused to CD8α spacer and transmembrane domain and a 4-1BB co-stimulatory domain (“TBB”) and a second generation CAR comprising a human MUC1-targeting HMFG2 domain (“H”). The CCR and the CAR are linked by a furin cleavage site, Ser-Gly linker (SGSG), and T2A ribosomal skip peptide. The VH and the VL sequences of HMFG2 sequence are underlined and in bold:

(SEQ ID NO: 7)
MGPGVLLLLLVATAWHGQGGVVSHFNDCPLSHDGYCLHDGVCMYIEALDK
YACNCVVGYIGERCQYRDLKWWELRAAAPTTTPAPRPPTPAPTIASQPLS
LRPEACRPAAGGAVHTRGLDFACDIYIWAPLAGTCGVLLLSLVITLYCNH
KRGRKKLLYIFKQPFMRPVQTTQEEDGCSCRFPEEEEGGCELRRKRSGSG
EGRGSLLTCGDVEENPGPMALPVTALLLPLALLLHAEVQLQQSGGGLVQP
GGSMKLSCVASGFTFSNYWMNWVRQSPEKGLEWVAEIRLKSNNYATHYAE
SVKGRFTISRDDSKSSVYLQMNNLRAEDTGIYYCTFGNSFAYWGQGTTVT
VSSGGGGSGGGGSGGGGSQAVVTQESALTTSPGETVTLTCRSSTGAVTTS
NYANWVQEKPDHLFTGLIGGTNNRAPGVPARFSGSLIGDKAALTITGAQT
EDEAIYFCALWYSNHWVFGGGTKLTVLGSEAAAIEVMYPPPYLDNEKSNG
TIIHVKGKHLCPSPLFPGPSKPFWVLVVVGGVLACYSLLVTVAFIIFWVR
SKRSRLLHSDYMNMTPRRPGPTRKHYQPYAPPRDFAAYRSRVKFSRSADA
PAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALP
PR.

In some embodiments, one of the binding elements of the pCAR is specific for markers associated with cancers of various types, including for example, one or more ErbB homodimers or heterodimers such as EGFR and HER2. In some embodiments, the binding element binds to markers associated with prostate cancer (for example using a binding element that binds to prostate-specific membrane antigen (PSMA)), breast cancer (for example using a binding element that targets HER2 (also known as ErbB2)) or neuroblastomas (for example using a binding element that targets GD2), melanomas, small cell or non-small cell lung carcinoma, sarcomas, brain tumours, ovarian cancer, pancreatic cancer, colorectal cancer, gastric cancer, bladder cancer, myeloma, non-Hodgkin lymphoma, esophageal cancer, endometrial cancer, hepatobiliary cancer, duodenal carcinoma, thyroid carcinoma, or renal cell carcinoma.

### 4.3.5.3. Chimeric Cytokine Receptor

In a further series of embodiments, the cells expressing the CAR and CCR are engineered to co-express a chimeric cytokine receptor, in particular the 4αβ chimeric cytokine receptor (FIG. 1). In 4αβ, the ectodomain of the IL-4 receptor-α chain is joined to the transmembrane and endodomains of IL-2/15 receptor-β. This allows the selective expansion and enrichment of the genetically engineered T cells ex vivo by the culture of these cells in a suitable support medium, which, in the case of 4αβ, would comprise IL-4 as the sole cytokine support. See Wilkie et al., “Selective expansion of chimeric antigen receptor-targeted T-cells with potent effector function using interleukin-4”, J. Biol. Chem. 285(33):25538-44 (2010) and Schalkwyk et al., “Design of a Phase 1 clinical trial to evaluate intratumoural delivery of ErbB-targeted chimeric antigen receptor T-cells in locally advanced or recurrent head and neck cancer,” Human Gene Ther. Clin. Devel. 24:134-142 (2013), incorporated herein by reference in its entirety.

Similarly, the system can be used with a chimeric cytokine receptor in which the ectodomain of the IL-4 receptor-α chain is joined to the transmembrane and endodomains of another receptor that is naturally bound by a cytokine that also binds to the common γ chain.

### 4.3.6. Engineered TCRs

In some embodiments, the immunoresponsive cells are engineered to further express an engineered (non-native) T cell receptor (TCR).

Engineered TCRs that can usefully be expressed in the immunoresponsive cells described herein are described in U.S. Pat. Nos. 9,512,197; 9,822,163; and 10,344,074, the disclosures of which are incorporated herein by reference in their entireties. Engineered TCRs that can usefully be expressed in the immunoresponsive cells described herein are described in US pre-grant publication nos. 2019/0161528; 2019/0144521; 2019/0135892; 2019/0127436; 2018/0218043; 2017/0088599; 2016/0159771; and 2016/0137715, the disclosures of which are incorporated herein by reference in their entireties.

### 4.3.7. Nucleic Acids and Methods of Making pCAR-T Cells

Also provided herein is a polynucleotide or a set of polynucleotides comprising a first nucleic acid encoding a modified pro-cytokine, wherein the modified pro-cytokine comprises, from N-terminus to C-terminus: (a) a pro-peptide; (b) a cleavage site recognized by a protease other than caspase-1, cathepsin G, elastase or proteinase 3; and (c) a cytokine fragment. The cleavage site is a specific sequence recognized by a protease.

In some embodiments, the first nucleic acid encodes a modified pro-IL-18, wherein the modified pro-IL-18 comprises, from N-terminus to C-terminus: (a) a pro-peptide; (b) a cleavage site recognized by a protease other than caspase-1; and (c) an IL-18 fragment. The cleavage site is a specific sequence recognized by a protease. In some embodiments, the cleavage site is on the downstream, on the upstream, or in place of caspase-1 recognition site of pro-IL-18. In some embodiments, the cleavage site is followed by a stop codon. The cleavage site in the modified pro-IL-18 can be selected from various protease cleavage sites known in the art. For example, the cleavage site can be recognized by granzyme B (GzB), caspase-3, caspase-8, MT1-MMP (MMP14), an alternative tumour-associated matrix metalloproteinase (MMP1-13), a disintegrin and metalloproteinase (ADAM) family member (notably ADAM 10 or ADAM17), cathepsin B, L or S, fibroblast-activation protein (FAP), kallikrein-related peptidases (KLK) such as KLK2, 3, 6 or 7, dipeptidyl peptidase (DPP)4, hepsin or urokinase plasminogen activator (see Dudani et al., “Harnessing protease activity to improve cancer care,” Annu. Rev. Cancer Biol., 2:353-76 (2018). In some embodiments, the cleavage site comprises a sequence selected from SEQ ID Nos: 26, 28, 30, and 32. In some embodiments, the modified pro-IL-18 comprises the polypeptide of a sequence selected from SEQ ID Nos: 27, 29, 31, and 33. In a particular embodiment, the modified pro-IL-18 comprises the polypeptide of a sequence of SEQ ID NO: 27.

In some embodiments, the first nucleic acid is selected from the group consisting of SEQ ID Nos: 102, 103, 105, 107, 109, 111 and 113. In a particular embodiment, the first nucleic acid comprises a polynucleotide of SEQ ID NO: 103. In some embodiments, the first nucleic acid is a coding sequence cloned in an expression vector, for example, a viral vector or a non-viral vector.

Alternatively, the modified pro-cytokine is a modified pro-IL-36α, β or γ protein, wherein the modified pro-IL-36 comprises, from N-terminus to C-terminus: (a) a pro-peptide; (b) a cleavage site recognized by a protease other than cathepsin G, elastase and proteinase 3; and (c) an IL-36 fragment. The cleavage site is a specific sequence recognized by a protease. In some embodiments, the cleavage site is on the downstream, on the upstream, or in place of the cathepsin G, elastase and/or proteinase 3 recognition site of pro-IL-36 α, β or γ. In some embodiments, the cleavage site is followed by a stop codon. The cleavage site in the modified pro-IL-36 can be selected from various protease cleavage sites known in the art. For example, the cleavage site can be recognized by granzyme B (GzB), caspase-3, caspase-8, MT1-MMP (MMP14), an alternative tumour-associated matrix metalloproteinase (MMP1-13), a disintegrin and metalloproteinase (ADAM) family member (notably ADAM 10 or ADAM17), cathepsin B, L or S, fibroblast-activation protein (FAP), kallikrein-related peptidases (KLK) such as KLK2, 3, 6 or 7, dipeptidyl peptidase (DPP)4, hepsin or urokinase plasminogen activator (see Dudani et al., “Harnessing protease activity to improve cancer care,” Annu. Rev. Cancer Biol., 2:353-76 (2018). In some embodiments, the cleavage site comprises a sequence selected from SEQ ID Nos: 26, 28, 30, and 32. In some embodiments, the modified pro-IL-36α, β and γ comprises the polypeptide of a sequence selected from SEQ ID Nos: 37, 39, and 41 respectively.

In some embodiments, the polynucleotide or the set of polynucleotides further comprise a second nucleic acid encoding a protease that recognizes the cleavage site on the first nucleic acid. The protease can be granzyme B (GzB), caspase-3, caspase-8, MT1-MMP (MMP14), an alternative tumour-associated matrix metalloproteinase (MMP1-13), a disintegrin and metalloproteinase (ADAM) family member (notably ADAM 10 or ADAM17), cathepsin B, L or S, fibroblast-activation protein (FAP), kallikrein-related peptidases (KLK) such as KLK2, 3, 6 or 7, dipeptidyl peptidase (DPP)4, hepsin or urokinase plasminogen activator (see Dudani et al., “Harnessing protease activity to improve cancer care,” Annu. Rev. Cancer Biol., 2:353-76 (2018). In some embodiments, the first nucleic acid and the second nucleic acid are in a single vector or in two different vectors.

In some embodiments, the polynucleotide or the set of polynucleotides further comprise a third nucleic acid encoding a chimeric antigen receptor (CAR). In some embodiments, the CAR is a second generation CAR as described above, comprising (a) a signalling region; (b) a first co-stimulatory signalling region; (c) a transmembrane domain; and (d) a first binding element that specifically interacts with a first epitope on a first target antigen.

In some embodiments, the polynucleotide or the set of polynucleotides further comprise a fourth nucleic acid encoding a CCR as described above. In some embodiments, the CCR comprises: (a) a second co-stimulatory signalling region; (b) a transmembrane domain; and (c) a second binding element that specifically interacts with a second epitope on a second target antigen.

As indicated above, for convenience herein, the CAR and CCR combination is referred to in the singular as a pCAR, although the CAR and CCR are separate, co-expressed, proteins. The third and fourth nucleic acid can be expressed from a single vector or two or more vectors. Suitable sequences for the nucleic acids will be apparent to a skilled person based on the description of the CAR and CCR above. The sequences may be optimized for use in the required immuno-responsive cell. However, in some cases, as discussed above, codons may be varied from the optimum or “wobbled” in order to avoid repeat sequences. Particular examples of such nucleic acids will encode the preferred embodiments described above.

In order to achieve transduction, the nucleic acids encoding the pCAR are suitably introduced into one or more vectors, such as a plasmid or a retroviral or lentiviral vector. Such vectors, including plasmid vectors, or cell lines containing them, form a further aspect of the invention.

In typical embodiments, the immunoresponsive cells are subjected to genetic modification, for example by retroviral or lentiviral mediated transduction, to introduce the first, the second, the third and/or the fourth nucleic acid into the host T cell genome, thereby permitting stable expression of the modified pro-cytokine (e.g., the modified pro-IL-18 or modified pro-IL-36), the protease, CAR and/or CCR, respectively. The first, the second, the third, and/or the fourth nucleic acid can be introduced as a single vector, or as multiple vectors, each including one or more of the nucleic acids. They may then be reintroduced into the patient, optionally after expansion, to provide a beneficial therapeutic effect, as described below.

In some embodiments, the immunoresponsive cells are γδ T cells and the γδ T cells are activated by an anti-γδ TCR antibody prior to the genetic modification. In some embodiments, an immobilised anti-γδ TCR antibody is used for activation.

The first and second nucleic acids encoding the modified pro-cytokine (e.g., the modified pro-IL-18 or modified pro-IL-36) and the protease can be expressed from the same vector or a plurality of vectors. The third and fourth nucleic acids encoding the CAR and CCR can be expressed from the same vector or a plurality of vectors. In one embodiment, the first, second, third and fourth nucleic acids are expressed from the same vector. The vector or vectors containing them can be combined in a kit, which is supplied with a view to generating immuno-responsive cells of the first aspect disclosed herein.

In some embodiments, where the T cells are engineered to co-express a chimeric cytokine receptor such as 44, the expansion step may include an ex vivo culture step in a medium which comprises the cytokine, such as a medium comprising IL-4 as the sole cytokine support in the case of 44. Alternatively, the chimeric cytokine receptor may comprise the ectodomain of the IL-4 receptor-α chain joined to the endodomain used by a common γ cytokine with distinct properties, such as IL-7. Expansion of the cells in IL-4 may result in less cell differentiation than use of IL-7. In this way, selective expansion and enrichment of genetically engineered T cells with the desired state of differentiation can be ensured.

### 4.4. Methods of Treatment

As discussed above, the immunoresponsive cells expressing a modified pro-cytokine (e.g., a modified pro-IL-18 or modified IL-36) are useful in therapy to direct a T cell-mediated immune response to a target cell with reduced immune suppression. Thus, in another aspect, methods for directing a T cell-mediated immune response to a target cell in a patient in need thereof are provided. The method comprises administering to the patient a population of immuno-responsive cells as described above, wherein the binding elements are specific for the target cell. In typical embodiments, the target cell expresses MUC1.

In another aspect, methods for treating cancer in a patient in need thereof are provided. The method comprises administering to the patient a population of immuno-responsive cells as described above, wherein the binding elements are specific for the target cell. In typical embodiments, the target cell expresses MUC1. In various embodiments, the patient has breast cancer, ovarian cancer, pancreatic cancer, colorectal cancer, lung cancer, gastric cancer, bladder cancer, myeloma, non-Hodgkin lymphoma, prostate cancer, esophageal cancer, endometrial cancer, hepatobiliary cancer, duodenal carcinoma, thyroid carcinoma, or renal cell carcinoma. In some embodiments, the patient has breast cancer.

In various embodiments, a therapeutically effective number of the immunoresponsive cells is administered to the patient. In certain embodiments, the immunoresponsive cells are administered by intravenous infusion. In certain embodiments, the immunoresponsive cells are administered by intratumoural injection. In certain embodiments, the immunoresponsive cells are administered by peritumoural injection. In certain embodiments, the immunoresponsive cells are administered by intraperitoneal injection. In certain embodiments, the immunoresponsive cells are administered by a plurality of routes selected from intravenous infusion, intratumoural injection, and peritumoural injection.

In another aspect, the disclosure provides immunoresponsive cells, polynucleotides, or γδ T cells for use in therapy or as a medicament. The disclosure further provides immunoresponsive cells, polynucleotides, or γδ T cells for use in the treatment of a pathological disorder. The disclosure also provides the use of immunoresponsive cells, polynucleotides, or γδ T cells in the manufacture of a medicament for the treatment of a pathological disorder. In some embodiments, the pathological disorder is cancer.

### 5. EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### 5.1. Methods

Culture of Cell Lines

All tumour cells and 293T cells were grown in DMEM supplemented with L-glutamine and 10% FBS (D10 medium). Where indicated, tumour cells were transduced to express a firefly luciferase-tdTomato (LT) SFG vector, followed by fluorescence activated cell sorting (FACS) for red fluorescent protein (RFP) expression. MDA-MB-468-HER2⁺⁺ cells were generated by transduction of MDA-MB-468-LT cells with an SFG retroviral vector that encodes human HER2. Transduced cells were FACS sorted using the ICR12 rat anti-human HER2 antibody and goat anti-rat PE.

Retrovirus Production

293T cells were triple transfected in GeneJuice (MilliporeSigma, Merck KGaA, Darmstadt, Germany) with (i) SFG retroviral vectors encoding the indicated the modified pro-IL-18, a protease, and/or CAR/pCAR, (ii) RDF plasmid encoding the RD114 envelope and (iii) Peq-Pam plasmid encoding gag-pol, as recommended by the manufacturers. For transfection of 1.5×10⁶ 293 T cells in 100 mm plate, 4.6875 μg SFG retroviral vector, 4.6875 μg Peq-Pam plasmid, and 3.125 μg RDF plasmid were used. Viral vector containing medium was collected 48 and 72 h post-transfection, snap-frozen and stored at −80° C. In some cases, stable packaging cell lines were created by transduction of 293 VEC GALV cells with transiently produced retroviral vector encoding the modified pro-IL-18, a protease, and/or CAR/pCAR. Virus prepared from either source was used interchangeably for transduction of target cells.

α/β T Cell Culture and Transduction

Peripheral blood mononuclear cells (PBMCs) were isolated from healthy donor peripheral blood samples by density gradient centrifugation using Ficoll-Paque (Ethical approval no. 18/WS/0047). T cells were cultured in RPMI with GlutaMax supplemented with 5% human AB serum. Activation of T cells was achieved by culture in the presence of 5 μg/mL phytohemagglutinin leucoagglutinin (PHA-L) for 24-48 h after which the cells were grown in IL-2 (100 U/mL) for a further 24 h prior to gene transfer. T cell transduction was achieved using RetroNectin (Takara Bio) coated-plates according to the Manufacturer's protocol. Activated PBMCs (1×10⁶ cells) were added per well of a RetroNectin coated 6-well plate. Retrovirus-containing medium was then added at 3 mL per well with 100 U/mL IL-2.

γδ T Cell Expansion and Transduction

To produce γδ T cells 9×10⁶ PBMCs were activated per well using 6 well plates coated with 2.4 μg of activating anti-γ/δ-1 TCR antibody (BD biosciences) per well. After 24 hours, cells were grown in 100 U/mL IL-2 and 5 ng/mL TGF-β for a further 48 hours. 3×10⁶ activated PBMCs were added per well of a RetroNectin coated 6-well plate pre-coated with 3 mL of retrovirus-containing medium. Cells were grown in 100 U/mL IL-2 and 5 ng/mL TGF-β (R & D Systems) for 14 days. Fold expansion was calculated relative to starting number of PBMCs.

Cytotoxicity Assays

MDA-MB-468 tumour cells or BxPC-3 tumour cells were seeded at a density of 1×10⁴ cells/well in a 96-well plate and incubated with T cells for 72 h at range of effector:target ratios from 4 to 0.03 (e.g., FIGS. 3A-3D). Destruction of tumour cell monolayers by T cells was quantified using an MTT assay. MTT (Sigma) was added at 500 μg/ml in D10 medium for 2 hours at 37° C. and 5% CO₂. After removal of the supernatant, formazan crystals were re-suspended in 100 μL DMSO. Absorbance was measured at 560 nm. Tumour cell viability was calculated as (absorbance of monolayer cultured with T cells/absorbance of untreated monolayer alone)×100%.

Detection of IFN-γ and IL-2

Supernatant was collected at 24 h from co-cultures of MDA-MB-468 tumour cells with CAR-T/pCAR-T cells described above. Cytokine levels were quantified using a human IFN-γ (Bio-Techne) or human IL-2 ELISA kit (Invitrogen) according to the Manufacturer's protocol. Data show the mean±SEM cytokine detected from 6 independent experiments, each performed in duplicate wells.

Detection of Active Human IL-18

T cells were harvested, washed and cultured in the absence of stimulation or cytokine for 48 hours. T cells were then stimulated at either a ratio of 10:1 effector to tumour or 200:1 T cell to anti-CD3/28 bead for 24 hours. Supernatant was then harvested and cultured with 5×10⁴ HEK blue IL-18 cells/well in 96 well plates for 24 hours. 20 μl of supernatant was then taken form the co-culture and added to 180 μl QUANTI-Blue solution and absorbance measured at 620-650 nm.

Repeated Antigen Stimulation Assays

MDA-MB-468 tumour cells were co-cultured with CAR-T/pCAR-T cells at an initial effector:target ratio of 1 CAR-T/pCAR-T cell:1 tumour cell or 1 CCR+/γδ TCR+ T cell:1 tumour cell for 72-96 h. All T cells were then removed, centrifuged at 400 g for 5 mins, re-suspended in 3 ml fresh RPMI supplemented with GlutaMax and 5% human serum and added to a new tumour cell monolayer. Residual tumour cell viability was assessed by MTT assay after each co-culture. T cells were added to a fresh tumour cell monolayer if >20% (or >30% for γδ T cells) tumour cells were killed compared to untreated cells. Data show the mean±SEM number of rounds of antigen stimulation. Cell counts were performed by pooling triplicate wells and counting the total number of cells.

Alternatively, tumour cell lines were plated in triplicate at 1×10⁵ cells per well in a 24-well culture plate 24 h prior to addition of T cells. CAR-T/pCAR-T cells were added at a 1:1 effector:target ratio. Tumour cell killing was measured after 72 h using a luciferase assay, in which D-luciferin (PerkinElmer) was added at 150 mg/mL immediately prior to luminescence reading. All T cells were restimulated by adding to a new tumour cell monolayer if >20% tumour cells were killed compared to untreated cells. Tumour cell viability was calculated as (luminescence of monolayer cultured with T cells/luminescence of untreated monolayer alone)×100%.

In Vivo Studies

PBMCs from healthy donors were engineered to express the indicated CARs/pCARs or were untransduced. After 11 days (αβ T cells) or 14 days (γδ T cells) of expansion in IL-2 (100 U/mL, added every 2-3 days) or IL-2+TGF-β, cells were analyzed by flow cytometry for expression of the CCR or CCR and γδ TCR.

Female severe combined immunodeficient (SCID) Beige mice were injected via the intraperitoneal (i.p.) route with 1×10⁶ MDA-MB-468 LT cells (FIG. 13). Twelve days after tumour cell injection, mice were i.p. injected with 10×10⁶ CCR positive or CCR, γδ TCR double positive (or untransduced) T cells in 200 μl of PBS, or with PBS alone as control. Tumour status was monitored by bioluminescence imaging, performed under isoflurane anaesthesia 20 minutes after injection of StayBrite™ D-Luciferin, Potassium Salt in 2000 PBS (150 mg/kg). Image acquisition was performed at the indicated time points using an IVIS® Lumina III (PerkinElmer) with Living Image software (PerkinElmer) set for automatically optimized exposure time, binning and F/stop. Animals were humanely killed when experimental endpoints had been reached.

Female NOD SCID gamma^{null}(NSG) mice were injected via the intraperitoneal (i.p.) route with 0.5×10⁶ SKOV3 ovarian cancer cells (FIG. 15). Eighteen days after tumour cell injection respectively, mice were i.p. injected with 0.5×10⁶ CART cells in 200 μl of PBS. Tumour status was monitored by bioluminescence imaging as above. Animals were humanely killed when experimental endpoints had been reached.

Female NSG mice were injected via the intraperitoneal (i.p.) route with 1×10⁵ BxPC-3 LT cells. Nine days after tumour cell injection, mice were i.p. injected with 10×10⁶ CCR/γδ TCR double positive (or untransduced) T cells in 200 μl of PBS, or with PBS alone as control. Tumour status was monitored by bioluminescence imaging as above. Animals were humanely killed when experimental endpoints had been reached.

### 5.2. Example 1: Creation of CAR/pCAR T Cells Expressing IL-18

A vector that includes the coding sequence of the TBB/H pCAR (SEQ ID NO: 7) as described above was modified to further include the coding sequence of various human IL-18 constructs.

The construct encoding TBB/H and pro-IL-18 (FIG. 18; SEQ ID NO: 102) was generated by inserting a synthetic polynucleotide (SEQ ID NO: 101) into the unique Kfl1 and Xho1 restriction sites in the TBB/H vector, replacing the 224 bp fragment between Kfl1 and Xho1 restriction sites. The insertion site of the pro-IL-18 sequence is downstream of a second wobbled T2A, and is followed by a stop codon. This construct is predicted not to express an active IL-18 in T cells, because cleavage of the pro-peptide requires caspase-1, which is not expressed in T cells.

The construct encoding TBB/H and a modified pro-IL-18 (pro-IL-18 (GzB)) (FIG. 19; SEQ ID NO: 103) was generated by replacing GAC GAC GAG AAC CTG GAG AGC GAC TAC (SEQ ID NO: 34) of MUC1-13 to GAC GAC GAG AAC ATC GAG CCC GAC TAC (SEQ ID NO: 35; changes underlined). This modified pro-IL-18 replaces the native caspase-1 cleavage site between the IL-18 pro-peptide and the mature IL-18 protein (LESD) with a granzyme B (GzB) cleavage site (IEPD).

The construct encoding TBB/H and constitutive (constit) IL-18 (FIG. 20; SEQ ID NO: 105) was generated by inserting a synthetic polynucleotide (SEQ ID NO: 104) into the unique Kfl1 and Xho1 restriction sites in TBB/H vector, replacing the 224 bp fragment between the Kfl1 and Xho1 restriction sites. The insertion site of IL-18 is downstream of a CD4 leader, and is followed by a stop codon. The IL-18 insert encodes the mature IL-18 protein without the IL-18 pro-peptide. This construct is predicted to express constitutively active IL-18 protein in T-cells.

The construct encoding TBB/H and a modified pro-IL-18 (pro-IL-18 (casp 8)) (FIG. 19; SEQ ID NO: 107) was generated by inserting a synthetic polynucleotide (SEQ ID NO: 106) into the unique Kfl1 and Xho1 restriction sites in TBB/H construct, replacing the 224 bp fragment between Kfl1 and Xho1 restriction sites. The insertion site of the modified pro-IL-18 sequence is downstream of a second wobbled T2A, and is followed by a stop codon. This modified pro-IL-18 replaces the native caspase-1 cleavage site between the IL-18 pro-peptide and the mature IL-18 protein (LESD) with a caspase-8 cleavage site (IETD).

The construct encoding TBB/H and a modified pro-IL-18 (pro-IL-18 (casp 3)) (FIG. 22; SEQ ID NO: 109) was generated by inserting a synthetic polynucleotide (SEQ ID NO: 108) into the unique Kfl1 and Xho1 restriction sites in TBB/H construct, replacing the 224 bp fragment that was removed. The insertion site of the modified pro-IL-18 sequence is downstream of a second wobbled T2A, and is followed by a stop codon. The modified pro-IL-18 replaces the native caspase-1 cleavage site between the pro-peptide and mature protein with a caspase-3 cleavage site (DEVD).

The construct encoding TBB/H with a modified pro-IL-18 (GzB) and additional granzyme B (FIG. 23; SEQ ID NO: 111) was generated by inserting a synthetic polynucleotide (SEQ ID NO: 110) into the unique Ale1 and Xho1 restriction sites in TBB/H GzB Pfn construct (encodes granzyme B, perforin and TBBH; SEQ ID NO: 112), replacing the 1,788 bp fragment that was removed.

The construct encoding T4 and a modified pro-IL-18 (MT1-MMP) (SEQ ID NO: 113) was generated by inserting a synthetic polynucleotide of MT1-MMP cleavage site (SEQ ID NO: 32) in place of the caspase-1 site of pro-IL-18 (FIGS. 16 and 24).

SFG retroviral vectors including coding sequences of the constructs were generated as described above, and then transduced into PBMCs. T cells were expanded from PMBCs in the presence of IL-2, as described above. The T cells expressed a modified pro-IL-18. IL-18 activities depended on the expression of the protease in the T cells that recognises the cleavage site in the modified pro-IL-18.

### 5.3. Example 2: In Vitro Anti-Tumour Activity of pCAR T Cells Armoured with IL-18

T cells transfected with SFG retroviral vectors encoding the TBB/H pCAR and one of the IL-18 variants described in Example 1 were analyzed for expression of the IL-18 variant (FIG. 4A) and the pCAR, separately measuring expression of the H28z CAR (H-2) and TIE-4-1BB CCR (FIG. 3) using flow cytometry. The results provided show that the majority of transduced T cells express both components of the TBB/H pCAR.

IL-18 secretion by transfected T cells was analyzed by ELISA (FIG. 4A) and the functional activity of expressed IL-18 was tested by reporter assay (FIG. 4B) in which a commercially available reporter cell line was used to detect functional IL-18 (i.e., the active IL-18 fragment generated after pro-peptide cleavage).

Secretion of IL-18 (FIG. 4A) was detected in unstimulated T cells that had been engineered by retroviral transduction to express each of the tested IL-18 variants, namely (native) pro-IL-18; constit IL-18; pro-IL-18 (casp 8) and pro-IL-18 (casp 3). However, IL-18 activity was detected only in T cells transduced with the constitutive variant (“constit IL-18”) in which mature IL-18 fragment was placed downstream of a CD4 signal peptide (FIG. 4B). Active IL-18 was not detected in conditioned medium generated by unstimulated pCAR T-cells that express pro-IL-18 or modified pro-IL-18 in which the cleavage site has been switched to that recognised by caspase-3 (pro-IL-18 (casp3)) or caspase-8 (pro-IL-18 (casp8)).

T cells co-expressing the TBB/H pCAR and each IL-18 variant were co-cultivated in vitro for 72 hours with MDA-MB-468 breast cancer cells. The effector:target (engineered T cell:tumour cell) ratio ranged from 4 to 0, including 4, 2, 1, 0.5, 0.25, 0.125, 0.06 and 0.03. Residual viable cancer cells present after termination of the co-culture were quantified by MTT assay. The percentage survival of MDA-MB-468 breast cancer cells after co-culture with the pCAR-T cells is presented in FIGS. 5A-5D. MDA-MB-468 breast cancer cells express both MUC-1 and ErbB dimers with very low level of HER2. As shown in FIGS. 5A-5D, T cells expressing TBB/H pCAR and each IL-18 variant showed greater cytotoxic anti-tumour activity at the effector:target ratio of 4 and 2, compared to at the effector:target ratio of 1 or 0.5. There was no clear difference detected among T cells expressing different IL-18 variants.

T cells expressing the TBB/H pCAR and an IL-18 variant were then subjected to iterative restimulation with MUC1⁺MDA-MB-468 breast cancer cells (FIGS. 6A-6B). While constitutive expression of the active IL-18 fragment enabled pCAR T-cells to undergo more re-stimulation cycles with preservation of cytotoxic activity, this was not seen with pro-IL-18 or with caspase-3-cleavable (pro-IL-18 (casp 3)) or caspase-8-cleavable (pro-IL-18 (casp 8)) derivatives. Constitutive IL-18 (but not pro-IL-18 or caspase 3/8-cleavable derivatives) mediated a significant increase in CAR T-cell proliferation (FIG. 6A). Based upon these data, we concluded that neither caspase 3-cleavable or caspase 8-cleavable IL-18 muteins were being activated upon CAR T-cell stimulation. Without wishing to be bound by a theory, the most probable explanation for this is that neither protein gained access to the cytosol where active caspase 3 and caspase 8 are found in activated T-cells (Alam et al., “Early activation of caspases during T lymphocyte stimulation results in selective substrate cleavage in nonapoptotic cells,” J. Exp. Med 190(12): 1879-1890 (1999); Chun et al. “Pleiotropic defects in lymphocyte activation caused by caspase-8 mutations lead to human immunodeficiency,” Nature 419(6905):395-9 (2002)).

The GzB cleavable variant of pro-IL-18 (MUC1-13b) (hereafter referred to as “pro-IL-18 (GzB)”) was next tested as above. Unlike the caspase 3-cleavable or caspase 8-cleavable pro-IL-18 modified muteins, pro-IL-18 (GzB) was functionally active when T-cells were activated, but not in the unstimulated state (FIGS. 7A-7B). This was confirmed by stimulation of the CAR T cells using a combination of anti-CD3 and anti-CD28 antibodies (FIG. 7B). Nonetheless, when T-cells co-expressing a pCAR with IL-18 (GzB) were tested in restimulation assays, they demonstrated inferior anti-tumour activity to T-cells in which IL-18 activity was constitutive.

We reasoned that GzB itself might be a limiting factor, given that it is predominantly expressed in CD8 T-cells, whereas autocrine stimulation by IL-18 operates primarily in CD4⁺ T-cells, which naturally express much less GzB. To address this, we engineered TBB/H pCAR T-cells to co-express native GzB in addition to IL-18 (GzB). This retroviral construct was transduced into PBMC which were co-cultured with MDA-MB-468 tumour cells at an effector to target ratio of 1:1. Anti-tumour activity was measured 72 hours later.

T cells engineered to co-express TBB/H and pro-IL-18 or the combination of TBB/H, pro-IL-18 (GzB), and additional granzyme B protease elicited comparable tumour cell killing. FIG. 8 provides data from five independent donors, each performed in triplicate.

Production of IL-18 (FIG. 9A) and IFN-γ (FIG. 9B) was tested in T cells expressing TBB/H+pro-IL-18 or TBB/H+pro-IL-18 (GzB)+granzyme B. Supernatants of the T cell cultures were taken at 72 hours and IL-18 and IFN-γ concentrations were measured.

Unstimulated T cells that co-express TBB/H and pro-IL-18 or the combination of TBB/H, pro-IL-18 (GzB) and granzyme B secreted similar levels of IL-18, as detected by ELISA (FIG. 9A). However, upon activation with target-expressing tumour cells, T cells expressing TBB/H, pro-IL-18 (GzB)+granzyme B produced significantly greater amounts of IFN-γ than T cells expressing TBB/H and pro-IL-18 (FIG. 9B). Data shown is from 4 independent donors, each performed in triplicate. (**p=0.008).

Transduced T cells were further subjected to successive rounds of antigen stimulation in the absence of exogenous IL-2. Cells were cultured at an initial effector to target ratio of 1:1 using either MDA-MD-468 cells (FIG. 10A) or BxPC-3 cells (FIG. 10B) as the target population. Tumour cell survival was measured twice weekly by MTT assay after 72-96 hours. Using MDA-MD-468 cells as the target population, T cells that co-express TBB/H and constit IL-18 or the combination of TBB/H, pro-IL-18 (GzB) and granzyme B were successfully restimulated for a significantly greater number cycles than T-cells that expressed TBB/H alone or together with pro-IL-18 (FIG. 10A). A similar pattern was seen using BxPC-3 cells as the target population (FIG. 10B). Data shown is generated from 1 donor for FIG. 10A and 1 donor for FIG. 10B, each performed in triplicate.

The number of successful restimulations for each pCAR T cell population were measured and the data are provided in FIGS. 11A and 11B. pCAR T cells progressed to the next round of stimulation if more than 20% cytotoxicity was observed. Cells were cultured at an effector to target ratio of 1 using either MDA-MD-468 cells (FIG. 11A) or BxPC-3 cells (FIG. 11B) as the target population. Using MDA-MD-468 cells as the target population, T cells that co-expressed TBB/H+pro-IL-18 (GzB)+granzyme B were successfully restimulated for more cycles than T cells that co-expressed TBB/H+pro-IL-18 (FIG. 11A). A similar pattern was seen using BxPC-3 cells as the target population (FIG. 11B). Data shown is from 5 independent donors, each performed in triplicate. (* p=0.039).

The numbers of T cells in each culture were also counted at the onset of each restimulation cycle. T-cells that co-expressed TBB/H+pro-IL-18 (GzB)+granzyme B but not TBB/H+pro-IL-18 proliferated significantly more than control TBB/H pCAR T cells. Counts shown are at 4^{th} restimulation cycle and are from 3 independent donors, each performed in triplicate. (FIG. 12; * p=0.014).

### 5.4. Example 3: In Vitro Anti-Tumour Activity of pCAR αβ T Cells Armoured with IL-18

αβ T cells were engineered to express the TBB/H pCAR alone or TBB/H pCAR in combination with pro-IL-18, pro-IL-18 (GzB), constit IL-18, or pro-IL-18 (GzB) together with granzyme B, using methods described in Example 1. The αβ T cells were assayed for IL-18 activity using a reporter cell line in which a commercially available reporter cell line was used to detect functional IL-18. Results provided in FIG. 35 show that IL-18 activity was detected in TBB/H pCAR αβ T cells that co-express constit IL-18 but not in other TBB/H pCAR αβ T cells when there was no stimulation. When the αβ T cells were stimulated with MUC1⁺MDA-MB-468 breast cancer cells (“+468”) or beads coated with anti-CD3 and anti-CD28 antibodies (“aCD3/28 beads”), however, TBB/H pCAR αβ T cells that co-express pro-IL18 (GzB) and granzyme B also had IL-18 activity. TBB/H pCAR αβ T cells that co-express pro-IL18 (GzB) and granzyme B had higher IL-18 activity than stimulated TBB/H pCAR αβ T cells that express only pro-IL18 (GzB).

### 5.5. Example 4: In Vivo Anti-Tumour Activity of pCAR-αβ T-Cells Armoured with IL-18

The anti-tumour activity of the CAR-03 T and pCAR-αβ T cells was assessed in vivo in tumour xenograft mouse models.

1×10⁶ MDA-MB-468 tumour cells expressing luciferase were injected into the peritoneal cavity (i.p.) of female SCID Beige mice to develop an established xenograft model. Eleven or twelve days after the tumour injection, 1×10⁷ CAR-αβ T cells with or without IL-18 expression were injected i.p. Pooled bioluminescence emission (“total flux”) from tumours was measured for each treatment. As provided in FIG. 13 and FIGS. 36A-36F, SCID Beige mice treated with αβ T cells that co-expressed TBB/H+pro-IL-18 (GzB)+granzyme B showed a significantly greater decrease in tumour-derived total flux compared to SCID Beige mice treated with TBB/H pCAR T cells. T-cells that co-expressed TBB/H+pro-IL-18 (GzB)+granzyme B also demonstrated a trend towards improved tumour control when compared to T cells that co-expressed TBB/H with constit IL-18 (FIGS. 13, 36E, and 36F). Data shown in FIG. 13 is pooled from 6 mice. Data shown in FIG. 36B is from 10 mice, FIG. 36C from 10 mice, FIG. 36D from 6 mice, FIG. 36E from 5 mice, and FIG. 36F from 5 mice.

FIG. 37 shows survival data of mice treated with PBS, αβ T cells expressing TBB/H alone or αβ T cells expressing TBB/H in combination with const. IL-18, pro-IL-18 (GzB), or pro-IL-18 (GzB) together with granzyme B following tumor injection. Results show improved survival in mice treated with αβ T-cells co-expressing TBB/H, pro-IL-18 (GzB) and granzyme B.

### 5.6. Example 5: In Vitro Anti-Tumour Activity of pCAR-γδ T-Cells

γδ T-cells were activated using 2.4 ng of immobilised anti-γδ TCR antibody per a well of a 6 well non-TC treated plate and were engineered by retroviral transduction to express the TBB/H pCAR after 48 hours. Untransduced γδ T cells and TBB/H pCAR γδ T cells were cultured and expanded (FIG. 49A and FIG. 49B). Co-expression of the second generation H2 CAR (“H28z”) and the TBB CCR (“TIE”) (together, the TBB/H pCAR) were confirmed in untransduced (FIG. 48A) or TBB/H pCAR γδ T cells (FIG. 48B) using flow cytometry.

Anti-tumour effects of untransduced γδ T-cells and TBB/H pCAR δγ T cells were evaluated by co-culturing with MDA-MB-468 breast cancer cells (FIG. 50A) or BxPC-3 cells (FIG. 50B) at 1:1 effector:target (γδ T cell:tumour cell) ratio for 72 hours. Viability (%) of tumour cells was measured by MTT assay at the first stimulation cycle, compared to tumour cells cultured without γδ T-cells. As provided in FIG. 50A and FIG. 50B, TBB/H pCAR δγ T cells had cytotoxic effects against the tumour cells.

Untransduced γδ T-cells and TBB/H pCAR δγ T cells were further subject subjected to successive rounds of antigen stimulation. Cells were cultured at an initial effector to target ratio of 1:1 using either MDA-MD-468 cells (FIG. 51A) or BxPC-3 cells (FIG. 51B) as the target population for 72-96 hours. Cytotoxicity of γδ T cells against tumour cells was determined by MTT assay in successive mono-layer challenges and restimulation causing more than 25% cytotoxicity to the target tumour cells was considered to be a successful restimulation cycle. T cells progressed to the next round of stimulation if more than 25% cytotoxicity was observed. The number of successful restimulations for each transduced γδ T cell population were measured and the data are provided in FIGS. 51A and 51B. The results demonstrate that TBB/H pCAR δγ T cells were successfully restimulated for more cycles than k T cells.

Viability (%) of tumour cells measured over multiple stimulation cycles is provided in FIG. 51C and FIG. 51D. The data show cytotoxic activity of TBB/H pCAR δγ T cells against MDA-MD-468 tumour cells (FIG. 51C) or BxPC-3 tumour cells (FIG. 51D) over the restimulation cycles.

### 5.7. Example 6: In Vivo Anti-Tumour Activity of pCAR-γδ T-Cells

The anti-tumour activity of TBB/H pCAR δγ T cells was assessed in vivo in tumour xenograft mouse models.

For the BxPC3-NSG mouse model, 1×10⁵ BxPC3-LT tumour cells expressing luciferase were injected into the peritoneal cavity (i.p.) of NSG mice to develop an established xenograft model. For the 468s-SCID Beige mouse model, 1×10⁶ MDA-MB-468 tumour cells expressing luciferase were injected into the peritoneal cavity (i.p.) of female SCID Beige mice to develop an established xenograft model.

Eleven days after the tumour injection, 1×10⁷ untransduced δγ T cells, 1×10⁷ TBB/H pCAR δγ T cells or PBS were injected i.p. into each animal model. Pooled bioluminescence emission (“total flux”) from tumours was measured for each treatment. As provided in FIG. 52 (BxPC3-NSG) and FIG. 53 (468s-SCID Beige), in both tumour xenograft mouse models, TBB/H pCAR δγ T cells induced significant decrease in tumour-derived total flux compared to untransduced δγ T cells or PBS control, demonstrating anti-tumour activity.

### 5.8. Example 7: In Vitro Anti-Tumour Activity of pCAR-γδ T-Cells Armoured with IL-18

γδ T-cells were activated using an immobilised anti-γδ TCR antibody and were engineered by retroviral transduction to express the TBB/H pCAR, either alone, or together with pro-IL-18, pro-IL-18 (GzB), constit IL-18, or pro-IL-18 (GzB) and granzyme B. Using flow cytometry, expression of the pCAR was determined following incubation with an anti-EGF antibody (detects the CCR; FIG. 14 upper panels) while enrichment of γδ T cells was also confirmed (FIG. 14 lower panels).

Anti-tumour effects of the γδ T-cells were evaluated by co-culture with MDA-MB-468 breast cancer cells (FIG. 15A) or BxPC-3 cells (FIG. 15B) for 72 hours. The effector:target (γδ T cell:tumour cell) ratio ranged from 128 to 1, including 128, 64, 32, 16, 8, 4, 2, and 1. Residual viable cancer cells that remained after the co-culture were quantified by MTT assay. As shown in FIGS. 15A and 15B, γδ T cells expressing the TBB/H pCAR alone or the TBB/H pCAR together with any IL-18 variant (pro-IL-18; constit IL-18; pro-IL-18 (GzB) or pro-IL-18 (GzB)+granzyme B) showed greater cytotoxic effects against tumour cells compared to untransduced γδ T cells.

Transduced γδ T cells were subjected to successive rounds of antigen stimulation in the absence of exogenous IL-2. Cells were cultured at an initial effector to target ratio of 1:1 using either MDA-MD-468 cells (FIG. 38A) or BxPC-3 cells (FIG. 38B) as the target population for 72-96 hours. T cells progressed to the next round of stimulation if more than 30% cytotoxicity was observed. The number of successful restimulations for each transduced γδ T cell population were measured and the data are provided in FIGS. 38A and 38B. Using MDA-MD-468 cells as the target population, T cells that co-expressed TBB/H+pro-IL-18 (GzB)+granzyme B were successfully restimulated for more cycles than T cells that co-expressed TBB/H+pro-IL-18 (FIG. 38A). A similar pattern was seen using BxPC-3 cells as the target population (FIG. 38B). (*p<0.05 **p<0.01).

Gamma delta T cells engineered to express the TBB/H pCAR alone or in combination with pro-IL-18, pro-IL-18 (GzB), or pro-IL-18 (GzB)+granzyme B were assayed for IL-18 activity using a reporter cell line. IL-18 activity was measured without stimulation or with stimulation with MUC1⁺MDA-MB-468 breast cancer cells (“+468”) or beads coated with anti-CD3 and anti-CD28 antibodies (“aCD3/28 beads”), Results provided in FIG. 39 demonstrate that IL-18 activity is dependent on stimulation of transduced γδ T cells. Stimulation of T cells that co-express TBB/H, pro-IL-18 (GzB) and granzyme B resulted in higher IL-18 activity than stimulated T cells that co-express only TBB/H and pro-IL-18 (GzB) or TBB/H and pro-IL18 (FIG. 39).

### 5.9. Example 8: In Vivo Anti-Tumour Activity of pCAR-γδ T-Cells Armoured with IL-18

The anti-tumour activity of pCAR-γδ T cells was assessed in vivo in tumour xenograft mouse models.

1×10⁶ MDA-MB-468 tumour cells expressing luciferase were injected into the peritoneal cavity (i.p.) of female SCID Beige mice to develop an established xenograft model. Eleven days after the tumour injection, 1×10⁷ TBB/H pCAR-γδ T cells with or without IL-18 expression were injected i.p. Pooled bioluminescence emission (“total flux”) from tumours was measured for each treatment. As provided in FIGS. 40A-40F, SCID Beige mice treated with γδ T cells that co-expressed TBB/H+pro-IL-18 (GzB)+granzyme B showed a significantly greater decrease in tumour-derived total flux compared to SCID Beige mice treated with TBB/H pCAR T cells. γδT-cells that co-expressed TBB/H+pro-IL-18 (GzB)+granzyme B also demonstrated a trend towards improved tumour control when compared to γδT cells that co-expressed TBB/H with constit IL-18 (FIGS. 40E and 40F). Data shown in FIG. 40B is from 5 mice, FIG. 40C from 4 mice, FIG. 40D from 5 mice, FIG. 40E from 4 mice, and FIG. 40F from 3 mice.

FIG. 41 shows survival data of mice treated with PBS, γδ T cells expressing TBB/H alone or γδ T cells expressing TBB/H in combination with const. IL-18, pro-IL-18 (GzB), or pro-IL-18 (GzB) together with granzyme B following tumor injection. Results show that improved survival in mice treated with γδ T-cells co-expressing TBB/H, pro-IL-18 (GzB) and granzyme B.

### 5.10. Example 9: In Vivo Anti-Tumour Activity of pCAR αβ or γδ T-Cells Armoured with IL-18

The anti-tumour activity of the pCAR-T cells was assessed in vivo in tumour xenograft mouse models.

1×10⁶ MDA-MB-468 tumour cells expressing luciferase were injected into the peritoneal cavity (i.p.) of female SCID Beige mice to develop an established xenograft model. Eleven days after tumour cell injection, TBB/H pCAR T cells (1×10⁷ pCAR-αβ or -γδ T cells, or 8×10⁶ pCAR-γδ T cells, or 4×10⁶ pCAR-γδ T cells) with no exogenous IL-18 expression (“TBB/H”) or with exogenous expression of pro-IL-18 alone or pro-IL-18 (GzB) together with granzyme B were injected i.p. Pooled bioluminescence emission (“total flux”) from tumours was measured from each treatment animal.

The total fluxes measured in animals within each treatment group were pooled and provided in FIGS. 30A, 30B, and 30C. As illustrated in the graphs, SCID Beige mice treated with TBB/H pCAR-T cells that co-expressed pro-IL-18 (GzB) and granzyme B showed a significantly greater decrease in tumour-derived total flux compared to mice in other groups, those treated with PBS, TBB/H pCAR T cells or TBB/H pCAR T cells co-expressing pro-IL-18. This effect was observed with both αβ T cells (FIG. 30A) and γδ T cells (FIG. 30B and FIG. 30C).

### 5.11. Example 10: Anti-Tumour Activity of Second Generation CAR-T Cells Armoured with IL-18

5×10⁵ SKOV-3 tumour cells expressing luciferase were injected into the peritoneal cavity (i.p.) of female SCID Beige mice to develop an SKOV-3 xenograft model. 18 days after tumour cell injection, CAR-T cells were administered by i.p. injection to three groups of mice. Group one received CAR-T cells that had been engineered to co-express the T1E28z ErbB-targeted second generation CAR with the 4αβ chimeric cytokine receptor. This combination is referred to as “T4” (see Schalkwyk et al., “Design of a Phase 1 clinical trial to evaluate intratumoural delivery of ErbB-targeted chimeric antigen receptor T-cells in locally advanced or recurrent head and neck cancer,” Human Gene Ther. Clin. Devel. 24:134-142 (2013)). A second group of mice received T4-engineered T cells that co-expressed an MT1-MMP (MMP14)-cleavable pro-IL-18 variant (pro-IL18 (MT1)) (schematized in FIG. 16). Tumour cells express high levels of the MT1-MMP (MMP14) protease. A third control group received T cells that expressed an endodomain truncated and signalling inactive version of the T1E-28z CAR (termed T1NA—T1E No Activation domain).

Treatment with a low dose (0.5 million) of second generation CAR T-cells or CAR-T cells expressing T1NA (an endodomain truncated control) were ineffective in this model. By contrast, CAR T-cells that co-expressed the T4 CAR and MT1-MMP (MMP14)-cleavable pro-IL-18 caused tumour elimination in ⅕ mice with disease regression in a further 2 animals (FIG. 17C). This provides an alternative approach to restrict the activation of IL-18 to the tumour microenvironment.

### 5.12. Example 11: In Vitro Anti-Tumour Activity of pCAR-T Cells Armoured with IL-36

Constructs encoding TBB/H and a mature IL-36 fragment (pro-IL-36 γ) were generated according to methods described above. Constructs encoding TBB/H and a modified pro-IL-36 γ were then generated by adding a cleavage site recognized by granzyme B (GzB) into the construct encoding TBB/H and pro-IL-36 γ. Constructs encoding TBB/H+pro-IL-36 (GzB)+granzyme B were also generated by inserting the coding sequence for granzyme B into the constructs encoding TBB/H and a modified pro-IL-36 γ.

T cells were transfected with SFG retroviral vectors encoding the TBB/H pCAR, and pro-IL-36 γ or the modified pro-IL-36 γ (GzB).

T cells expressing TBB/H or co-expressing TBB/H, pro-IL-36 γ and granzyme B or the combination of TBB/H, pro-IL-36 γ (GzB) and granzyme B protease were subjected to iterative stimulation with MDA-MB-468 breast cancer cells or BxPC-3 pancreatic cancer cells. The effector:target (engineered T cell: tumour cell) ratio ranged from 2 to 0.03, including 1, 0.5, 0.25, 0.125, and 0.06. Residual viable cancer cells present after termination of the co-culture were quantified by MTT assay. Results shown in FIG. 42A (MDA-MB-468 cells) and FIG. 42B (BxPC-3 cells) show significant cytotoxic activity of TBB/H T cells expressing pro-IL-36 γ and granzyme B, or pro-IL-36 γ (GzB) and granzyme B. T cells co-expressing TBB/H, pro-IL-36 γ (GzB) and granzyme B significantly proliferated over the restimulation cycles (FIGS. 43A and 43B). Production of IFN-γ (FIG. 44A and FIG. 44B) was also significantly higher in T cells expressing TBB/H+pro-IL-36 γ+granzyme B or TBB/H+pro-IL-36 γ (GzB)+granzyme B compared to TBB/H T cells.

T cells engineered to co-express TBB/H+pro-IL-36 γ+granzyme B or TBB/H+pro-IL-36 γ (GrzB)+granzyme B elicited tumour cell killing of both MDA-MB-468 cells (FIG. 45) and BxPC-3 cells (FIG. 46) at effector:target (engineered T cell: tumour cell) ratios ranging from 2 to 0.03, including 1, 0.5, 0.25, 0.125, and 0.06 (all experiments performed in triplicate).

### 5.13. Example 12: In Vivo Anti-Tumour Activity of pCAR-T Cells Armoured with IL-36

Anti-tumour activity of pCAR-T cells armoured with IL-36 was further studied in vivo. 1×10⁶ MDA-MB-468 tumour cells expressing luciferase were injected into the peritoneal cavity (i.p.) of female SCID Beige mice to develop an established xenograft model. Twelve days after the tumour injection, 1×10⁷ TBB/H pCAR-T cells without IL-36 expression or TBB/H pCAR-T cells with coexpression of pro-IL36 γ and granzyme B or pro-IL36 γ (GzB) and granzyme B were injected i.p.

Pooled bioluminescence emission (“total flux”) from tumours was measured for each treatment. Mice treated with T cells co-expressing TBB/H+pro-IL-36 γ (GzB)+granzyme B show a significantly greater decrease in tumour-derived total flux compared to mice treated with TBB/H pCAR T cells (FIGS. 47A-47D).

### 6. SEQUENCES

| ID | SEQ |
|---|---|
| SEQ ID NO: 1 | RVKFSRSADAPAYQQGQNQLYNELNLGRREEYDVLDKRRGRDPEMGGKPRRKNP |
| CD3 zeta | QEGLYNELQKDKMAEAYSEIGMKGERRRGKGHDGLYQGLSTATKDTYDALHMQA |
| chain (a | LPPR |
| polymorphic | |
| form) | |
| SEQ ID NO: 2 | RVKFSRSAEPPAYQQGQNQLYNELNLGRREEYDVLDKRRGRDPEMGGKPRRKNP |
| CD3 zeta | QEGLYNELQKDKMAEAYSEIGMKGERRRGKGHDGLYQGLSTATKDTYDALHMQA |
| chain (a | LPPR |
| polymorphic | |
| form) | |
| SEQ ID NO: 3 | MLRLLLALNLFPSIQVTGNKILVKQSPMLVAYDNAVNLSCKYSYNLFSREFRASLH |
| CD28 protein | KGLDSAVEVCVVYGNYSQQLQVYSKTGFNCDGKLGNESVTFYLQNLYVNQTDIYFC |
| | KIEVMYPPPYLDNEKSNGTIIHVKGKHLCPSPLFPGPSKPFWVLVVVGGVLACYSLL |
| | VTVAFIIFWVRSKRSRLLHSDYMNMTPRRPGPTRKHYQPYAPPRDFAAYRS |
| SEQ ID NO: 4 | IEVMYPPPYLDNEKSNGTIIHVKGKHLCPSPLFPGPSKPFWVLVVVGGVLACYSLLV |
| Hinge, | TVAFIIFWVRSKRSRLLHSDYMNMTPRRPGPTRKHYQPYAPPRDFAAYRS |
| transmembrane | |
| and co- | |
| stimulatory | |
| region in CD28 | |
| protein | |
| SEQ ID NO: 5 | EQKLISEEDL |
| c-myc tag | |
| SEQ ID NO: 6 | IEVEQKLISEEDLLDNEKSNGTIIHVKGKHLCPSPLFPGPSKPFWVLVVVGGVLACYS |
| Hinge | LLVTVAFIIFWVRSKRSRLLHSDYMNMTPRRPGPTRKHYQPYAPPRDFAAYRS |
| (containing myc | |
| tag), | |
| transmembrane | |
| and co- | |
| stimulatory | |
| region in CD28 | |
| protein | |
| SEQ ID NO: 7 | MGPGVLLLLLVATAWHGQGGVVSHFNDCPLSHDGYCLHDGVCMYIEALDKYACN |
| TBB/H pCAR | CVVGYIGERCQYRDLKWWELRAAAPTTTPAPRPPTPAPTIASQPLSLRPEACRPAA |
| | GGAVHTRGLDFACDIYIWAPLAGTCGVLLLSLVITLYCNHKRGRKKLLYIFKQPFMR |
| | PVQTTQEEDGCSCRFPEEEEGGCELRRKRSGSGEGRGSLLTCGDVEENPGPMALPV |
| | TALLLPLALLLHAEVQLQQSGGGLVQPGGSMKLSCVASGFTFSNYWMNWVRQSPE |
| | KGLEWVAEIRLKSNNYATHYAESVKGRFTISRDDSKSSVYLQMNNLRAEDTGIYYC |
| | TFGNSFAYWGQGTTVTVSSGGGGSGGGGSGGGGSQAVVTQESALTTSPGETVTLTC |
| | RSSTGAVTTSNYANWVQEKPDHLFTGLIGGTNNRAPGVPARFSGSLIGDKAALTIT |
| | GAQTEDEAIYFCALWYSNHWVFGGGTKLTVLGSEAAAIEVMYPPPYLDNEKSNGT |
| | IIHVKGKHLCPSPLFPGPSKPFWVLVVVGGVLACYSLLVTVAFIIFWVRSKRSRLLHS |
| | DYMNMTPRRPGPTRKHYQPYAPPRDFAAYRSRVKFSRSADAPAYQQGQNQLYNE |
| | LNLGRREEYDVLDKRRGRDPEMGGKPRRKNPQEGLYNELQKDKMAEAYSEIGMK |
| | GERRRGKGHDGLYQGLSTATKDTYDALHMQALPPR |
| SEQ ID NO: 8 | GFTFSNY |
| V_{H} CDR1, | |
| HMFG2 | |
| SEQ ID NO: 9 | RLKSNNYA |
| V_{H} CDR2 | |
| HMFG2 | |
| SEQ ID NO: 10 | GNSFAY |
| V_{H} CDR3 | |
| HMFG2 | |
| SEQ ID NO: 11 | RSSTGAVTTSNYAN |
| V_{L} CDR1 | |
| HMFG2 | |
| SEQ ID NO: 12 | GTNNRAP |
| V_{L} CDR2 | |
| HMFG2 | |
| SEQ ID NO: 13 | ALWYSNHWV |
| V_{L} CDR3 | |
| HMFG2 | |
| SEQ ID NO: 14 | EVQLQQSGGGLVQPGGSMKLSCVASGFTFSNYWMNWVRQSPEKGLEWVAEIRLK |
| VₕHMFG2 | SNNYATHYAESVKGRFTISRDDSKSSVYLQMNNLRAEDTGIYYCTFGNSFAYWGQ |
| | GTTVTVSS |
| SEQ ID NO: 15 | QAVVTQESALTTSPGETVTLTCRSSTGAVTTSNYANWVQEKPDHLFTGLIGGTNN |
| V_{L} HMFG2 | RAPGVPARFSGSLIGDKAALTITGAQTEDEAIYFCALWYSNHWVFGGGTKLTVLGS |
| | E |
| SEQ ID NO: 16 | EVQLQQSGGGLVQPGGSMKLSCVASGFTFSNYWMNWVRQSPEKGLEWVAEIRLK |
| scFv of the | SNNYATHYAESVKGRFTISRDDSKSSVYLQMNNLRAEDTGIYYCTFGNSFAYWGQ |
| HMGF2 | GTTVTVSSGGGGSGGGGSGGGGSQAVVTQESALTTSPGETVTLTCRSSTGAVTTSNY |
| antibody | ANWVQEKPDHLFTGLIGGTNNRAPGVPARFSGSLIGDKAALTITGAQTEDEAIYFC |
| | ALWYSNHWVFGGGTKLTVLGSE |
| SEQ ID NO: 17 | GAGGTGCAGCTGCAGCAGTCTGGAGGAGGCTTGGTGCAACCTGGAGGATCCATGA |
| scFv of the | AACTCTCCTGTGTTGCCTCTGGATTCACTTTCAGTAACTACTGGATGAACTGGGT |
| HMGF2 | CCGCCAGTCTCCAGAGAAGGGGCTTGAGTGGGTTGCTGAAATTAGATTGAAATCT |
| antibody | AATAATTATGCAACACATTATGCGGAGTCTGTGAAAGGGAGGTTCACCATCTCAA |
| | GAGATGATTCCAAAAGTAGTGTCTACCTGCAAATGAACAACTTAAGAGCTGAAGA |
| | CACTGGCATTTATTACTGTACCTTTGGTAACTCCTTTGCTTACTGGGGCCAAGGG |
| | ACCACGGTCACCGTCTCCTCAGGTGGAGGCGGTTCAGGCGGAGGTGGCTCTGGCG |
| | GTGGCGGATCGCAGGCCGTGGTCACTCAGGAATCTGCACTCACCACATCACCTGGT |
| | GAAACAGTCACACTCACTTGTCGCTCAAGTACTGGGGCTGTTACAACTAGTAACT |
| | ATGCCAACTGGGTCCAAGAAAAACCAGATCATTTATTCACTGGTCTAATAGGTGG |
| | TACCAACAACCGAGCACCAGGTGTTCCTGCCAGATTCTCAGGCTCCCTGATTGGAG |
| | ACAAGGCTGCCCTCACCATCACAGGGGCACAGACTGAGGATGAGGCAATATATTT |
| | CTGTGCTCTATGGTACAGCAACCATTGGGTGTTCGGTGGAGGAACCAAACTGACT |
| | GTCCTAGGATCAGAG |
| SEQ ID NO: 18 | VVSHFNDCPLSHDGYCLHDGVCMYIEALDKYACNCVVGYIGERCQYRDLKWWELR |
| T1E | |
| SEQ ID NO: 19 | GTGGTGAGCCACTTCAACGACTGCCCTCTGAGCCACGACGGCTACTGCCTGCACGA |
| T1E | CGGCGTGTGCATGTACATCGAGGCCCTGGACAAGTACGCCTGCAACTGCGTGGTG |
| | GGCTACATCGGCGAGAGATGCCAGTACAGAGACCTGAAGTGGTGGGAGCTGAGA |
| SEQ ID NO: 20 | MQPILLLLAFLLLPRADAGEIIGGHEAKPHSRPYMAYLMIWDQKSLKRCGGFLIRD |
| GzB (aa seq) | DFVLTAAHCWGSSINVTLGAHNIKEQEPTQQFIPVKRPIPHPAYNPKNFSNDIMLL |
| | QLERKAKRTRAVQPLRLPSNKAQVKPGQTCSVAGWGQTAPLGKHSHTLQEVKMT |
| | VQEDRKCESDLRHYYDSTIELCVGDPEIKKTSFKGDSGGPLVCNKVAQGIVSYGRNN |
| | GMPPRACTKVSSFVHWIKKTMKRY |
| SEQ ID NO: 21 | MENTENSVDSKSIKNLEPKIIHGSESMDSGISLDNSYKMDYPEMGLCIIINNKNFHK |
| Caspase-3 (aa | STGMTSRSGTDVDAANLRETFRNLKYEVRNKNDLTREEIVELMRDVSKEDHSKRS |
| seq) | SFVCVLLSHGEEGIIFGTNGPVDLKKITNFFRGDRCRSLTGKPKLFIIQACRGTELDC |
| | GIETDSGVDDDMACHKIPVEADFLYAYSTAPGYYSWRNSKDGSWFIQSLCAMLKQ |
| | YADKLEFMHILTRVNRKVATEFESFSFDATFHAKKQIPCIVSMLTKELYFYH |
| SEQ ID NO: 22 | MDFSRNLYDIGEQLDSEDLASLKFLSLDYIPQRKQEPIKDALMLFQRLQEKRMLEE |
| Caspase-8 (aa | SNLSFLKELLFRINRLDLLITYLNTRKEEMERELQTPGRAQISAYRVMLYQISEEVSR |
| seq) | SELRSFKFLLQEEISKCKLDDDMNLLDIFIEMEKRVILGEGKLDILKRVCAQINKSLL |
| | KIINDYEEFSKERSSSLEGSPDEFSNGEELCGVMTISDSPREQDSESQTLDKVYQMK |
| | SKPRGYCLIINNHNFAKAREKVPKLHSIRDRNGTHLDAGALTTTFEELHFEIKPHD |
| | DCTVEQIYEILKIYQLMDHSNMDCFICCILSHGDKGIIYGTDGQEAPIYELTSQFTGL |
| | KCPSLAGKPKVFFIQACQGDNYQKGIPVETDSEEQPYLEMDLSSPQTRYIPDEADFL |
| | LGMATVNNCVSYRNPAEGTWYIQSLCQSLRERCPRGDDILTILTEVNYEVSNKDDK |
| | KNMGKQMPQPTFTLRKKLVFPSD |
| SEQ ID NO: 23 | MSPAPRPPRCLLLPLLTLGTALASLGSAQSSSFSPEAWLQQYGYLPPGDLRTHTQR |
| MT1-MMP (aa | SPQSLSAAIAAMQKFYGLQVTGKADADTMKAMRRPRCGVPDKFGAEIKANVRRK |
| seq) | RYAIQGLKWQHNEITFCIQNYTPKVGEYATYEAIRKAFRVWESATPLRFREVPYAY |
| | IREGHEKQADIMIFFAEGFHGDSTPFDGEGGFLAHAYFPGPNIGGDTHFDSAEPWT |
| | VRNEDLNGNDIFLVAVHELGHALGLEHSSDPSAIMAPFYQWMDTENFVLPDDDR |
| | RGIQQLYGGESGFPTKMPPQPRTTSRPSVPDKPKNPTYGPNICDGNFDTVAMLRG |
| | EMFVFKERWFWRVRNNQVMDGYPMPIGQFWRGLPASINTAYERKDGKFVFFKG |
| | DKHWVFDEASLEPGYPKHIKELGRGLPTDKIDAALFWMPNGKTYFFRGNKYYRF |
| | NEELRAVDSEYPKNIKVWEGIPESPRGSFMGSDEVFTYFYKGNKYWKFNNQKLKV |
| | EPGYPKSALRDWMGCPSGGRPDEGTEEETEVIIIEVDEEGGGAVSAAAVVLPVLLLL |
| | LVLAVGLAVFFFRRHGTPRRLLYCQRSLLDKV |
| SEQ ID NO: 24 | YFGKLESKLSVIRNLNDQVLFIDQGNRPLFEDMTDSDCRDNAPRTIFIISMYKDSQP |
| Mature IL-18 | RGMAVTISVKCEKISTLSCENKIISFKEMNPPDNIKDTKSDIIFFQRSVPGHDNKMQ |
| (aa seq) | FESSSYEGYFLACEKERDLFKLILKKEDELGDRSIMFTVQNED |
| SEQ ID NO: 25 | MAAEPVEDNCINFVAMKFIDNTLYFIAEDDEN(LESD) |
| Pro-peptide | |
| containing | |
| native | |
| Caspase 1 | |
| cleavage site | |
| (aa seq) | |
| SEQ ID NO: 26 | IEPD |
| GzB cleavage | |
| site (aa seq) | |
| SEQ ID NO: 27 | MAAEPVEDNCINFVAMKFIDNTLYFIAEDDENIEPDYFGKLESKLSVIRNLNDQVLF |
| Pro-IL-18 | IDQGNRPLFEDMTDSDCRDNAPRTIFIISMYKDSQPRGMAVTISVKCEKISTLSCEN |
| (GzB) (aa | KIISFKEMNPPDNIKDTKSDIIFFQRSVPGHDNKMQFESSSYEGYFLACEKERDLFK |
| seq) | LILKKEDELGDRSIMFTVQNED |
| SEQ ID NO: 28 | DEVDI |
| Caspase-3 | |
| cleavage site | |
| (aa seq) | |
| SEQ ID NO: 29 | MAAEPVEDNCINFVAMKFIDNTLYFIAEDDENDEVDIYFGKLESKLSVIRNLNDQV |
| Pro-IL-18 | LFIDQGNRPLFEDMTDSDCRDNAPRTIFIISMYKDSQPRGMAVTISVKCEKISTLSC |
| (casp 3) (aa | ENKIISFKEMNPPDNIKDTKSDIIFFQRSVPGHDNKMQFESSSYEGYFLACEKERDL |
| seq) | FKLILKKEDELGDRSIMFTVQNED |
| SEQ ID NO: 30 | IETDI |
| Caspase-8 | |
| cleavage site | |
| (aa seq) | |
| SEQ ID NO: 31 | MAAEPVEDNCINFVAMKFIDNTLYFIAEDDENIETDIYFGKLESKLSVIRNLNDQVL |
| Pro-IL-18 | FIDQGNRPLFEDMTDSDCRDNAPRTIFIISMYKDSQPRGMAVTISVKCEKISTLSCE |
| (casp 8) (aa | NKIISFKEMNPPDNIKDTKSDIIFFQRSVPGHDNKMQFESSSYEGYFLACEKERDLF |
| seq) | KLILKKEDELGDRSIMFTVQNED*LEGSGLVQFVKDRISVVQALVLTQQYHQLKPIE |
| | YEP |
| SEQ ID NO: 32 | GGGGS(IPESLRAG)GGGGSAAA |
| MT1-MMP | |
| cleavage site | |
| plus linkers | |
| (aa seq) | |
| SEQ ID NO: 33 | MAAEPVEDNCINFVAMKFIDNTLYFIAEDDENGGGGSIPESLRAGGGGGSAAAYFG |
| Pro-IL-18 | KLESKLSVIRNLNDQVLFIDQGNRPLFEDMTDSDCRDNAPRTIFIISMYKDSQPRG |
| (MT1-MMP) | MAVTISVKCEKISTLSCENKIISFKEMNPPDNIKDTKSDIIFFQRSVPGHDNKMQFES |
| (aa seq) | SSYEGYFLACEKERDLFKLILKKEDELGDRSIMFTVQNED |
| SEQ ID NO: 34 | GAC GAC GAG AAC CTG GAG AGC GAC TAC |
| (removed | |
| sequence for | |
| generation of | |
| MUC1-13b) | |
| SEQ ID NO: 35 | GAC GAC GAG AAC ATC GAG CCC GAC TAC |
| (inserted | |
| sequence for | |
| generation of | |
| MUC1-13b) | |
| SEQ ID NO: 36 | MEKALKIDTPQQGSIQDINHRVWVLQDQTLIAVPRKDRMSPVTIALISCRHVETLEKDRGNPIYLGLNG |
| Pro-IL- | LNLCLMCAKVGDQPTLQLKEKDIMDLYNQPEPVKSFLFYHSQSGRNSTFESVAFPGWFIAVSSEGGCPL |
| 36α (cleaved | ILTQELGKANTTDFGLTMLF |
| pro-peptide | |
| sequence in | |
| bold) | |
| SEQ ID NO: 37 | MEKALIEPDKIDTPQQGSIQDINHRVWVLQDQTLIAVPRKDRMSPVTIALISCRHVETLEKDRGNPIYL |
| Pro-IL- | GLNGLNLCLMCAKVGDQPTLQLKEKDIMDLYNQPEPVKSFLFYHSQSGRNSTFESVAFPGWFIAVSSE |
| 36α (GzB) | GGCPLILTQELGKANTTDFGLTMLF |
| (cleaved pro- | |
| peptide | |
| sequence in | |
| bold) | |
| SEQ ID NO: 38 | MNPQREAAPKSYAIRDSRQMVWVLSGNSLIAAPLSRSIKPVTLHLIACRDTEFSDKEKGNMVYLGIKG |
| Pro-IL- | KDLCLFCAEIQGKPTLQLKLQGSQDNIGKDTCWKLVGIHTCINLDVRESCFMGTLDQWGIGVGRKKWK |
| 36β (cleaved | SSFQHHHLRKKDKDFSSMRTNIGMPGRM |
| pro-peptide | |
| sequence in | |
| bold) | |
| SEQ ID NO: 39 | MNPQIEPDREAAPKSYAIRDSRQMVWVLSGNSLIAAPLSRSIKPVTLHLIACRDTEFSDKEKGNMVYL |
| Pro-IL- | GIKGKDLCLFCAEIQGKPTLQLKLQGSQDNIGKDTCWKLVGIHTCINLDVRESCFMGTLDQWGIGVGRK |
| 36β (GzB) | KWKSSFQHHHLRKKDKDFSSMRTNIGMPGRM |
| (cleaved pro- | |
| peptide | |
| sequence in | |
| bold) | |
| SEQ ID NO: 40 | MRGTPGDADGGGRAVYQSMCKPITGTINDLNQQVWTLQGQNLVAVPRSDSVTPVTVAVITCKYPEA |
| Pro-IL- | LEQGRGDPIYLGIQNPEMCLYCEKVGEQPTLQLKEQKIMDLYGQPEPVKPFLFYRAKTGRTSTLESVAF |
| 36γ (cleaved | PDWFIASSKRDQPIILTSELGKSYNTAFELNIND |
| pro-peptide | |
| sequence in | |
| bold) | |
| SEQ ID NO: 41 | MRGTPGDADGGGRIEPDSMCKPITGTINDLNQQVWTLQGQNLVAVPRSDSVTPVTVAVITCKYPEAL |
| Pro-IL- | EQGRGDPIYLGIQNPEMCLYCEKVGEQPTLQLKEQKIMDLYGQPEPVKPFLFYRAKTGRTSTLESVAFP |
| 36γ (GzB) | DWFIASSKRDQPIILTSELGKSYNTAFELNIND |
| (cleaved pro- | |
| peptide | |
| sequence in | |
| bold) | |
| SEQ ID NO: 42 | KIDTPQQGSIQDINHRVWVLQDQTLIAVPRKDRMSPVTIALISCRHVETLEKDRGNPIYLGLNGLNLCL |
| Mature IL- | MCAKVGDQPTLQLKEKDIMDLYNQPEPVKSFLFYHSQSGRNSTFESVAFPGWFIAVSSEGGCPLILTQE |
| 36α | LGKANTTDFGLTMLF |
| SEQ ID NO: 43 | REAAPKSYAIRDSRQMVWVLSGNSLIAAPLSRSIKPVTLHLIACRDTEFSDKEKGNMVYLGIKGKDLCL |
| Mature IL- | FCAEIQGKPTLQLKLQGSQDNIGKDTCWKLVGIHTCINLDVRESCFMGTLDQWGIGVGRKKWKSSFQH |
| 36β | HHLRKKDKDFSSMRTNIGMPGRM |
| SEQ ID NO: 44 | SMCKPITGTINDLNQQVWTLQGQNLVAVPRSDSVTPVTVAVITCKYPEALEQGRGDPIYLGIQNPEMCL |
| Mature IL-36γ | YCEKVGEQPTLQLKEQKIMDLYGQPEPVKPFLFYRAKTGRTSTLESVAFPDWFIASSKRDQPIILTSELG |
| | KSYNTAFELNIND |
| SEQ ID NO: 45 | MEKAL |
| Cleaved pro- | |
| peptide from | |
| Pro-IL-36α | |
| SEQ ID NO: 46 | MEKALIEPD |
| Cleaved pro- | |
| peptide from | |
| Pro-IL- | |
| 36α (GzB) | |
| SEQ ID NO: 47 | MNPQ |
| Cleaved pro- | |
| peptide from | |
| Pro-IL-36β | |
| SEQ ID NO: 48 | MNPQIEPD |
| Cleaved pro- | |
| peptide from | |
| Pro-IL- | |
| 36β (GzB) | |
| SEQ ID NO: 49 | MRGTPGDADGGGRAVYQ |
| Cleaved pro- | |
| peptide from | |
| Pro-IL-36γ | |
| SEQ ID NO: 50 | MRGTPGDADGGGRIEPD |
| Cleaved pro- | |
| peptide from | |
| Pro-IL- | |
| 36γ (GzB) | |
| SEQ ID NO: | CGGGACCCTGAGATGGGGGGAAAGCCGAGAAGGAAGAACCCTCAGGAAGGCCTGT |
| 101 | ACAATGAACTGCAGAAAGATAAGATGGCGGAGGCCTACAGTGAGATTGGGATGA |
| Synthesized | AAGGCGAGCGCCGGAGGGGCAAGGGGCACGATGGCCTTTACCAGGGTCTCAGTAC |
| sequence for | AGCCACCAAGGACACCTACGACGCCCTTCACATGCAGGCCCTGCCCCCTCGCCGGC |
| (TBB/H and | GGAAACGGTCTGGATCTGGGGAAGGTCGGGGATCTTTGCTCACATGTGGAGATGT |
| pro-IL-18) | CGAAGAGAATCCTGGCCCTATGGCCGCCGAGCCCGTGGAGGACAACTGCATCAAC |
| | TTCGTGGCCATGAAGTTCATCGACAACACCCTGTACTTCATCGCCGAGGACGACG |
| | AGAACCTGGAGAGCGACTACTTCGGCAAGCTGGAGAGCAAGCTGAGCGTGATCCG |
| | GAACCTGAACGACCAGGTGCTGTTCATCGACCAGGGCAACCGGCCTCTGTTCGAG |
| | GACATGACCGACAGCGACTGCCGGGACAACGCTCCCCGGACCATCTTCATCATCAG |
| | CATGTACAAGGACAGCCAGCCCCGGGGAATGGCCGTGACCATCAGCGTGAAGTGC |
| | GAGAAGATCAGCACCCTGAGCTGCGAGAACAAGATCATCAGCTTCAAGGAGATGA |
| | ACCCTCCCGACAACATCAAGGACACCAAGAGCGACATCATCTTCTTCCAGCGGAGC |
| | GTGCCTGGCCACGACAACAAGATGCAGTTCGAGAGCAGCAGCTACGAGGGCTACT |
| | TCCTGGCCTGCGAGAAGGAGCGGGACCTGTTCAAGCTGATCCTGAAGAAGGAGGA |
| | CGAGCTGGGCGACCGGAGCATCATGTTCACCGTGCAGAACGAGGACTAACTCGAG |
| SEQ ID NO: | CCATGGGCCCCGGCGTGCTGCTGCTGCTGCTGGTGGCCACCGCCTGGCACGGCCAG |
| 102 | GGCGGCGTGGTGAGCCACTTCAACGACTGCCCTCTGAGCCACGACGGCTACTGCCT |
| (TBB/H and | GCACGACGGCGTGTGCATGTACATCGAGGCCCTGGACAAGTACGCCTGCAACTGC |
| pro-IL-18) | GTGGTGGGCTACATCGGCGAGAGATGCCAGTACAGAGACCTGAAGTGGTGGGAGC |
| | TGAGAGCCGCCGCCCCCACCACCACACCCGCTCCCAGACCCCCTACCCCTGCCCCCA |
| | CCATCGCCAGCCAGCCCCTGAGCCTGAGACCCGAGGCCTGCAGACCTGCCGCCGGC |
| | GGAGCCGTGCACACCAGAGGCCTGGACTTCGCCTGCGACATCTACATCTGGGCTCC |
| | CCTGGCCGGCACCTGCGGCGTGCTGCTGCTGAGCCTGGTGATCACCCTGTACTGCA |
| | ACCACAAGAGAGGCAGAAAGAAGCTGCTGTACATCTTCAAGCAGCCCTTCATGAG |
| | ACCCGTGCAGACCACCCAGGAGGAGGACGGCTGCAGCTGCAGATTCCCCGAGGAG |
| | GAGGAGGGCGGCTGCGAGCTGAGAAGAAAGAGAAGCGGCAGCGGCGAGGGCAGA |
| | GGCAGCCTGCTGACCTGCGGCGACGTGGAGGAGAACCCCGGACCCATGGCTCTCCC |
| | AGTGACTGCCCTACTGCTTCCCCTAGCGCTTCTCCTGCATGCAGAGGTGCAGCTGC |
| | AGCAGTCTGGAGGAGGCTTGGTGCAACCTGGAGGATCCATGAAACTCTCCTGTGT |
| | TGCCTCTGGATTCACTTTCAGTAACTACTGGATGAACTGGGTCCGCCAGTCTCCAG |
| | AGAAGGGGCTTGAGTGGGTTGCTGAAATTAGATTGAAATCTAATAATTATGCAAC |
| | ACATTATGCGGAGTCTGTGAAAGGGAGGTTCACCATCTCAAGAGATGATTCCAAA |
| | AGTAGTGTCTACCTGCAAATGAACAACTTAAGAGCTGAAGACACTGGCATTTATT |
| | ACTGTACCTTTGGTAACTCCTTTGCTTACTGGGGCCAAGGGACCACGGTCACCGTC |
| | TCCTCAGGTGGAGGCGGTTCAGGCGGAGGTGGCTCTGGCGGTGGCGGATCGCAGG |
| | CCGTGGTCACTCAGGAATCTGCACTCACCACATCACCTGGTGAAACAGTCACACTC |
| | ACTTGTCGCTCAAGTACTGGGGCTGTTACAACTAGTAACTATGCCAACTGGGTCC |
| | AAGAAAAACCAGATCATTTATTCACTGGTCTAATAGGTGGTACCAACAACCGAGC |
| | ACCAGGTGTTCCTGCCAGATTCTCAGGCTCCCTGATTGGAGACAAGGCTGCCCTCA |
| | CCATCACAGGGGCACAGACTGAGGATGAGGCAATATATTTCTGTGCTCTATGGTA |
| | CAGCAACCATTGGGTGTTCGGTGGAGGAACCAAACTGACTGTCCTAGGATCAGAG |
| | GCGGCCGCAATTGAAGTTATGTATCCTCCTCCTTACCTAGACAATGAGAAGAGCA |
| | ATGGAACCATTATCCATGTGAAAGGGAAACACCTTTGTCCAAGTCCCCTATTTCC |
| | CGGACCTTCTAAGCCCTTTTGGGTGCTGGTGGTGGTTGGTGGAGTCCTGGCTTGC |
| | TATAGCTTGCTAGTAACAGTGGCCTTTATTATTTTCTGGGTGAGGAGTAAGAGGA |
| | GCAGGCTCCTGCACAGTGACTACATGAACATGACTCCCCGCCGCCCCGGGCCCACC |
| | CGCAAGCATTACCAGCCCTATGCCCCACCACGCGACTTCGCAGCCTATCGCTCCAG |
| | AGTGAAGTTCAGCAGGAGCGCAGACGCCCCCGCGTACCAGCAGGGCCAGAACCAG |
| | CTCTATAACGAGCTCAATCTAGGACGAAGAGAGGAGTACGATGTTTTGGACAAGA |
| | GACGTGGCCGGGACCCTGAGATGGGGGGAAAGCCGAGAAGGAAGAACCCTCAGGA |
| | AGGCCTGTACAATGAACTGCAGAAAGATAAGATGGCGGAGGCCTACAGTGAGATT |
| | GGGATGAAAGGCGAGCGCCGGAGGGGCAAGGGGCACGATGGCCTTTACCAGGGTC |
| | TCAGTACAGCCACCAAGGACACCTACGACGCCCTTCACATGCAGGCCCTGCCCCCT |
| | CGCCGGCGGAAACGGTCTGGATCTGGGGAAGGTCGGGGATCTTTGCTCACATGTG |
| | GAGATGTCGAAGAGAATCCTGGCCCTATGGCCGCCGAGCCCGTGGAGGACAACTG |
| | CATCAACTTCGTGGCCATGAAGTTCATCGACAACACCCTGTACTTCATCGCCGAGG |
| | ACGACGAGAACCTGGAGAGCGACTACTTCGGCAAGCTGGAGAGCAAGCTGAGCGT |
| | GATCCGGAACCTGAACGACCAGGTGCTGTTCATCGACCAGGGCAACCGGCCTCTG |
| | TTCGAGGACATGACCGACAGCGACTGCCGGGACAACGCTCCCCGGACCATCTTCAT |
| | CATCAGCATGTACAAGGACAGCCAGCCCCGGGGAATGGCCGTGACCATCAGCGTG |
| | AAGTGCGAGAAGATCAGCACCCTGAGCTGCGAGAACAAGATCATCAGCTTCAAGG |
| | AGATGAACCCTCCCGACAACATCAAGGACACCAAGAGCGACATCATCTTCTTCCA |
| | GCGGAGCGTGCCTGGCCACGACAACAAGATGCAGTTCGAGAGCAGCAGCTACGAG |
| | GGCTACTTCCTGGCCTGCGAGAAGGAGCGGGACCTGTTCAAGCTGATCCTGAAGA |
| | AGGAGGACGAGCTGGGCGACCGGAGCATCATGTTCACCGTGCAGAACGAGGACTA |
| | ACTCGAGGGATCCGGATTAGTCCAATTTGTTAAAGACAGGATATCAGTGGTCCAG |
| | GCTCTAGTTTTGACTCAACAATATCACCAGCTGAAGCCTATAGAGTACGAGCCAT |
| | AGATAAAATAAAAGATTTTATTTAGTCTCCAGAAAAAGGGGGGAATGAAAGACC |
| | CCACCTGTAGGTTTGGCAAGCTAGCTTAAGTAACGCCATTTTGCAAGGCATGGAA |
| | AAATACATAACTGAGAATAGAGAAGTTCAGATCAAGGTCAGGAACAGATGGAAC |
| | AGCTGAATATGGGCCAAACAGGATATCTGTGGTAAGCAGTTCCTGCCCCGGCTCA |
| | GGGCCAAGAACAGATGGAACAGCTGAATATGGGCCAAACAGGATATCTGTGGTAA |
| | GCAGTTCCTGCCCCGGCTCAGGGCCAAGAACAGATGGTCCCCAGATGCGGTCCAGC |
| | CCTCAGCAGTTTCTAGAGAACCATCAGATGTTTCCAGGGTGCCCCAAGGACCTGA |
| | AATGACCCTGTGCCTTATTTGAACTAACCAATCAGTTCGCTTCTCGCTTCTGTTCG |
| | CGCGCTTCTGCTCCCCGAGCTCAATAAAAGAGCCCACAACCCCTCACTCGGGGCGC |
| | CAGTCCTCCGATTGACTGAGTCGCCCGGGTACCCGTGTATCCAATAAACCCTCTTG |
| | CAGTTGCATCCGACTTGTGGTCTCGCTGTTCCTTGGGAGGGTCTCCTCTGAGTGAT |
| | TGACTACCCGTCAGCGGGGGTCTTTCACACATGCAGCATGTATCAAAATTAATTT |
| | GGTTTTTTTTCTTAAGTATTTACATTAAATGGCCATAGTACTTAAAGTTACATTG |
| | GCTTCCTTGAAATAAACATGGAGTATTCAGAATGTGTCATAAATATTTCTAATTT |
| | TAAGATAGTATCTCCATTGGCTTTCTACTTTTTCTTTTATTTTTTTTTGTCCTCTG |
| | TCTTCCATTTGTTGTTGTTGTTGTTTGTTTGTTTGTTTGTTGGTTGGTTGGTTAA |
| | TTTTTTTTTAAAGATCCTACACTATAGTTCAAGCTAGACTATTAGCTACTCTGTA |
| | ACCCAGGGTGACCTTGAAGTCATGGGTAGCCTGCTGTTTTAGCCTTCCCACATCTA |
| | AGATTACAGGTATGAGCTATCATTTTTGGTATATTGATTGATTGATTGATTGATG |
| | TGTGTGTGTGTGATTGTGTTTGTGTGTGTGACTGTGAAAATGTGTGTATGGGTGT |
| | GTGTGAATGTGTGTATGTATGTGTGTGTGTGAGTGTGTGTGTGTGTGTGTGCATG |
| | TGTGTGTGTGTGACTGTGTCTATGTGTATGACTGTGTGTGTGTGTGTGTGTGTGT |
| | GTGTGTGTGTGTGTGTGTGTGTGTTGTGAAAAAATATTCTATGGTAGTGAGAGCC |
| | AACGCTCCGGCTCAGGTGTCAGGTTGGTTTTTGAGACAGAGTCTTTCACTTAGCT |
| | TGGAATTCACTGGCCGTCGTTTTACAACGTCGTGACTGGGAAAACCCTGGCGTTA |
| | CCCAACTTAATCGCCTTGCAGCACATCCCCCTTTCGCCAGCTGGCGTAATAGCGAA |
| | GAGGCCCGCACCGATCGCCCTTCCCAACAGTTGCGCAGCCTGAATGGCGAATGGCG |
| | CCTGATGCGGTATTTTCTCCTTACGCATCTGTGCGGTATTTCACACCGCATATGGT |
| | GCACTCTCAGTACAATCTGCTCTGATGCCGCATAGTTAAGCCAGCCCCGACACCCG |
| | CCAACACCCGCTGACGCGCCCTGACGGGCTTGTCTGCTCCCGGCATCCGCTTACAG |
| | ACAAGCTGTGACCGTCTCCGGGAGCTGCATGTGTCAGAGGTTTTCACCGTCATCA |
| | CCGAAACGCGCGATGACGAAAGGGCCTCGTGATACGCCTATTTTTATAGGTTAAT |
| | GTCATGATAATAATGGTTTCTTAGACGTCAGGTGGCACTTTTCGGGGAAATGTGC |
| | GCGGAACCCCTATTTGTTTATTTTTCTAAATACATTCAAATATGTATCCGCTCAT |
| | GAGACAATAACCCTGATAAATGCTTCAATAATATTGAAAAAGGAAGAGTATGAG |
| | TATTCAACATTTCCGTGTCGCCCTTATTCCCTTTTTTGCGGCATTTTGCCTTCCTG |
| | TTTTTGCTCACCCAGAAACGCTGGTGAAAGTAAAAGATGCTGAAGATCAGTTGGG |
| | TGCACGAGTGGGTTACATCGAACTGGATCTCAACAGCGGTAAGATCCTTGAGAGT |
| | TTTCGCCCCGAAGAACGTTTTCCAATGATGAGCACTTTTAAAGTTCTGCTATGTG |
| | GCGCGGTATTATCCCGTATTGACGCCGGGCAAGAGCAACTCGGTCGCCGCATACA |
| | CTATTCTCAGAATGACTTGGTTGAGTACTCACCAGTCACAGAAAAGCATCTTACG |
| | GATGGCATGACAGTAAGAGAATTATGCAGTGCTGCCATAACCATGAGTGATAACA |
| | CTGCGGCCAACTTACTTCTGACAACGATCGGAGGACCGAAGGAGCTAACCGCTTT |
| | TTTGCACAACATGGGGGATCATGTAACTCGCCTTGATCGTTGGGAACCGGAGCTG |
| | AATGAAGCCATACCAAACGACGAGCGTGACACCACGATGCCTGTAGCAATGGCAA |
| | CAACGTTGCGCAAACTATTAACTGGCGAACTACTTACTCTAGCTTCCCGGCAACA |
| | ATTAATAGACTGGATGGAGGCGGATAAAGTTGCAGGACCACTTCTGCGCTCGGCC |
| | CTTCCGGCTGGCTGGTTTATTGCTGATAAATCTGGAGCCGGTGAGCGTGGGTCTC |
| | GCGGTATCATTGCAGCACTGGGGCCAGATGGTAAGCCCTCCCGTATCGTAGTTAT |
| | CTACACGACGGGGAGTCAGGCAACTATGGATGAACGAAATAGACAGATCGCTGAG |
| | ATAGGTGCCTCACTGATTAAGCATTGGTAACTGTCAGACCAAGTTTACTCATATA |
| | TACTTTAGATTGATTTAAAACTTCATTTTTAATTTAAAAGGATCTAGGTGAAGAT |
| | CCTTTTTGATAATCTCATGACCAAAATCCCTTAACGTGAGTTTTCGTTCCACTGA |
| | GCGTCAGACCCCGTAGAAAAGATCAAAGGATCTTCTTGAGATCCTTTTTTTCTGC |
| | GCGTAATCTGCTGCTTGCAAACAAAAAAACCACCGCTACCAGCGGTGGTTTGTTT |
| | GCCGGATCAAGAGCTACCAACTCTTTTTCCGAAGGTAACTGGCTTCAGCAGAGCG |
| | CAGATACCAAATACTGTCCTTCTAGTGTAGCCGTAGTTAGGCCACCACTTCAAGA |
| | ACTCTGTAGCACCGCCTACATACCTCGCTCTGCTAATCCTGTTACCAGTGGCTGCT |
| | GCCAGTGGCGATAAGTCGTGTCTTACCGGGTTGGACTCAAGACGATAGTTACCGG |
| | ATAAGGCGCAGCGGTCGGGCTGAACGGGGGGTTCGTGCACACAGCCCAGCTTGGA |
| | GCGAACGACCTACACCGAACTGAGATACCTACAGCGTGAGCATTGAGAAAGCGCC |
| | ACGCTTCCCGAAGGGAGAAAGGCGGACAGGTATCCGGTAAGCGGCAGGGTCGGAA |
| | CAGGAGAGCGCACGAGGGAGCTTCCAGGGGGAAACGCCTGGTATCTTTATAGTCC |
| | TGTCGGGTTTCGCCACCTCTGACTTGAGCGTCGATTTTTGTGATGCTCGTCAGGG |
| | GGGCGGAGCCTATGGAAAAACGCCAGCAACGCGGCCTTTTTACGGTTCCTGGCCT |
| | TTTGCTGGCCTTTTGCTCACATGTTCTTTCCTGCGTTATCCCCTGATTCTGTGGAT |
| | AACCGTATTACCGCCTTTGAGTGAGCTGATACCGCTCGCCGCAGCCGAACGACCGA |
| | GCGCAGCGAGTCAGTGAGCGAGGAAGCGGAAGAGCGCCCAATACGCAAACCGCCT |
| | CTCCCCGCGCGTTGGCCGATTCATTAATGCAGCTGGCACGACAGGTTTCCCGACTG |
| | GAAAGCGGGCAGTGAGCGCAACGCAATTAATGTGAGTTAGCTCACTCATTAGGCA |
| | CCCCAGGCTTTACACTTTATGCTTCCGGCTCGTATGTTGTGTGGAATTGTGAGCG |
| | GATAACAATTTCACACAGGAAACAGCTATGACCATGATTACGCCAAGCTTTGCTC |
| | TTAGGAGTTTCCTAATACATCCCAAACTCAAATATATAAAGCATTTGACTTGTTC |
| | TATGCCCTAGGGGGCGGGGGGAAGCTAAGCCAGCTTTTTTTAACATTTAAAATGT |
| | TAATTCCATTTTAAATGCACAGATGTTTTTATTTCATAAGGGTTTCAATGTGCAT |
| | GAATGCTGCAATATTCCTGTTACCAAAGCTAGTATAAATAAAAATAGATAAACGT |
| | GGAAATTACTTAGAGTTTCTGTCATTAACGTTTCCTTCCTCAGTTGACAACATAA |
| | ATGCGCTGCTGAGCAAGCCAGTTTGCATCTGTCAGGATCAATTTCCCATTATGCC |
| | AGTCATATTAATTACTAGTCAATTAGTTGATTTTTATTTTTGACATATACATGTG |
| | AATGAAAGACCCCACCTGTAGGTTTGGCAAGCTAGCTTAAGTAACGCCATTTTGC |
| | AAGGCATGGAAAAATACATAACTGAGAATAGAAAAGTTCAGATCAAGGTCAGGA |
| | ACAGATGGAACAGCTGAATATGGGCCAAACAGGATATCTGTGGTAAGCAGTTCCT |
| | GCCCCGGCTCAGGGCCAAGAACAGATGGAACAGCTGAATATGGGCCAAACAGGAT |
| | ATCTGTGGTAAGCAGTTCCTGCCCCGGCTCAGGGCCAAGAACAGATGGTCCCCAG |
| | ATGCGGTCCAGCCCTCAGCAGTTTCTAGAGAACCATCAGATGTTTCCAGGGTGCCC |
| | CAAGGACCTGAAATGACCCTGTGCCTTATTTGAACTAACCAATCAGTTCGCTTCT |
| | CGCTTCTGTTCGCGCGCTTATGCTCCCCGAGCTCAATAAAAGAGCCCACAACCCCT |
| | CACTCGGGGCGCCAGTCCTCCGATTGACTGAGTCGCCCGGGTACCCGTGTATCCAA |
| | TAAACCCTCTTGCAGTTGCATCCGACTTGTGGTCTCGCTGTTCCTTGGGAGGGTCT |
| | CCTCTGAGTGATTGACTACCCGTCAGCGGGGGTCTTTCATTTGGGGGCTCGTCCGG |
| | GATCGGGAGACCCCTGCCCAGGGACCACCGACCCACCACCGGGAGGTAAGCTGGCC |
| | AGCAACTTATCTGTGTCTGTCCGATTGTCTAGTGTCTATGACTGATTTTATGCGC |
| | CTGCGTCGGTACTAGTTAGCTAACTAGCTCTGTATCTGGCGGACCCGTGGTGGAA |
| | CTGACGAGTTCGGAACACCCGGCCGCAACCCTGGGAGACGTCCCAGGGACTTCGGG |
| | GGCCGTTTTTGTGGCCCGACCTGAGTCCTAAAATCCCGATCGTTTAGGACTCTTTG |
| | GTGCACCCCCCTTAGAGGAGGGATATGTGGTTCTGGTAGGAGACGAGAACCTAAA |
| | ACAGTTCCCGCCTCCGTCTGAATTTTTGCTTTCGGTTTGGGACCGAAGCCGCGCCG |
| | CGCGTCTTGTCTGCTGCAGCATCGTTCTGTGTTGTCTCTGTCTGACTGTGTTTCTG |
| | TATTTGTCTGAAAATATGGGCCCGGGCTAGACTGTTACCACTCCCTTAAGTTTGA |
| | CCTTAGGTCACTGGAAAGATGTCGAGCGGATCGCTCACAACCAGTCGGTAGATGT |
| | CAAGAAGAGACGTTGGGTTACCTTCTGCTCTGCAGAATGGCCAACCTTTAACGTC |
| | GGATGGCCGCGAGACGGCACCTTTAACCGAGACCTCATCACCCAGGTTAAGATCA |
| | AGGTCTTTTCACCTGGCCCGCATGGACACCCAGACCAGGTCCCCTACATCGTGACC |
| | TGGGAAGCCTTGGCTTTTGACCCCCCTCCCTGGGTCAAGCCCTTTGTACACCCTAA |
| | GCCTCCGCCTCCTCTTCCTCCATCCGCCCCGTCTCTCCCCCTTGAACCTCCTCGTTC |
| | GACCCCGCCTCGATCCTCCCTTTATCCAGCCCTCACTCCTTCTCTAGGCGCCCCCAT |
| | ATGGCCATATGAGATCTTATATGGGGCACCCCCGCCCCTTGTAAACTTCCCTGACC |
| | CTGACATGACAAGAGTTACTAACAGCCCCTCTCTCCAAGCTCACTTACAGGCTCTC |
| | TACTTAGTCCAGCACGAAGTCTGGAGACCTCTGGCGGCAGCCTACCAAGAACAAC |
| | TGGACCGACCGGTGGTACCTCACCCTTACCGAGTCGGCGACACAGTGTGGGTCCGC |
| | CGACACCAGACTAAGAACCTAGAACCTCGCTGGAAAGGACCTTACACAGTCCTGC |
| | TGACCACCCCCACCGCCCTCAAAGTAGACGGCATCGCAGCTTGGATACACGCCGCC |
| | CACGTGAAGGCTGCCGACCCCGGGGGTGGACCATCCTCTAGACTG |
| SEQ ID NO: | CCATGGGCCCCGGCGTGCTGCTGCTGCTGCTGGTGGCCACCGCCTGGCACGGCCAG |
| 103 | GGCGGCGTGGTGAGCCACTTCAACGACTGCCCTCTGAGCCACGACGGCTACTGCCT |
| TBB/H + pro- | GCACGACGGCGTGTGCATGTACATCGAGGCCCTGGACAAGTACGCCTGCAACTGC |
| IL-18 (GzB) | GTGGTGGGCTACATCGGCGAGAGATGCCAGTACAGAGACCTGAAGTGGTGGGAGC |
| | TGAGAGCCGCCGCCCCCACCACCACACCCGCTCCCAGACCCCCTACCCCTGCCCCCA |
| | CCATCGCCAGCCAGCCCCTGAGCCTGAGACCCGAGGCCTGCAGACCTGCCGCCGGC |
| | GGAGCCGTGCACACCAGAGGCCTGGACTTCGCCTGCGACATCTACATCTGGGCTCC |
| | CCTGGCCGGCACCTGCGGCGTGCTGCTGCTGAGCCTGGTGATCACCCTGTACTGCA |
| | ACCACAAGAGAGGCAGAAAGAAGCTGCTGTACATCTTCAAGCAGCCCTTCATGAG |
| | ACCCGTGCAGACCACCCAGGAGGAGGACGGCTGCAGCTGCAGATTCCCCGAGGAG |
| | GAGGAGGGCGGCTGCGAGCTGAGAAGAAAGAGAAGCGGCAGCGGCGAGGGCAGA |
| | GGCAGCCTGCTGACCTGCGGCGACGTGGAGGAGAACCCCGGACCCATGGCTCTCCC |
| | AGTGACTGCCCTACTGCTTCCCCTAGCGCTTCTCCTGCATGCAGAGGTGCAGCTGC |
| | AGCAGTCTGGAGGAGGCTTGGTGCAACCTGGAGGATCCATGAAACTCTCCTGTGT |
| | TGCCTCTGGATTCACTTTCAGTAACTACTGGATGAACTGGGTCCGCCAGTCTCCAG |
| | AGAAGGGGCTTGAGTGGGTTGCTGAAATTAGATTGAAATCTAATAATTATGCAAC |
| | ACATTATGCGGAGTCTGTGAAAGGGAGGTTCACCATCTCAAGAGATGATTCCAAA |
| | AGTAGTGTCTACCTGCAAATGAACAACTTAAGAGCTGAAGACACTGGCATTTATT |
| | ACTGTACCTTTGGTAACTCCTTTGCTTACTGGGGCCAAGGGACCACGGTCACCGTC |
| | TCCTCAGGTGGAGGCGGTTCAGGCGGAGGTGGCTCTGGCGGTGGCGGATCGCAGG |
| | CCGTGGTCACTCAGGAATCTGCACTCACCACATCACCTGGTGAAACAGTCACACTC |
| | ACTTGTCGCTCAAGTACTGGGGCTGTTACAACTAGTAACTATGCCAACTGGGTCC |
| | AAGAAAAACCAGATCATTTATTCACTGGTCTAATAGGTGGTACCAACAACCGAGC |
| | ACCAGGTGTTCCTGCCAGATTCTCAGGCTCCCTGATTGGAGACAAGGCTGCCCTCA |
| | CCATCACAGGGGCACAGACTGAGGATGAGGCAATATATTTCTGTGCTCTATGGTA |
| | CAGCAACCATTGGGTGTTCGGTGGAGGAACCAAACTGACTGTCCTAGGATCAGAG |
| | GCGGCCGCAATTGAAGTTATGTATCCTCCTCCTTACCTAGACAATGAGAAGAGCA |
| | ATGGAACCATTATCCATGTGAAAGGGAAACACCTTTGTCCAAGTCCCCTATTTCC |
| | CGGACCTTCTAAGCCCTTTTGGGTGCTGGTGGTGGTTGGTGGAGTCCTGGCTTGC |
| | TATAGCTTGCTAGTAACAGTGGCCTTTATTATTTTCTGGGTGAGGAGTAAGAGGA |
| | GCAGGCTCCTGCACAGTGACTACATGAACATGACTCCCCGCCGCCCCGGGCCCACC |
| | CGCAAGCATTACCAGCCCTATGCCCCACCACGCGACTTCGCAGCCTATCGCTCCAG |
| | AGTGAAGTTCAGCAGGAGCGCAGACGCCCCCGCGTACCAGCAGGGCCAGAACCAG |
| | CTCTATAACGAGCTCAATCTAGGACGAAGAGAGGAGTACGATGTTTTGGACAAGA |
| | GACGTGGCCGGGACCCTGAGATGGGGGGAAAGCCGAGAAGGAAGAACCCTCAGGA |
| | AGGCCTGTACAATGAACTGCAGAAAGATAAGATGGCGGAGGCCTACAGTGAGATT |
| | GGGATGAAAGGCGAGCGCCGGAGGGGCAAGGGGCACGATGGCCTTTACCAGGGTC |
| | TCAGTACAGCCACCAAGGACACCTACGACGCCCTTCACATGCAGGCCCTGCCCCCT |
| | CGCCGGCGGAAACGGTCTGGATCTGGGGAAGGTCGGGGATCTTTGCTCACATGTG |
| | GAGATGTCGAAGAGAATCCTGGCCCTATGGCCGCCGAGCCCGTGGAGGACAACTG |
| | CATCAACTTCGTGGCCATGAAGTTCATCGACAACACCCTGTACTTCATCGCCGAGG |
| | ACGACGAGAACATCGAGCCCGACTACTTCGGCAAGCTGGAGAGCAAGCTGAGCGT |
| | GATCCGGAACCTGAACGACCAGGTGCTGTTCATCGACCAGGGCAACCGGCCTCTG |
| | TTCGAGGACATGACCGACAGCGACTGCCGGGACAACGCTCCCCGGACCATCTTCAT |
| | CATCAGCATGTACAAGGACAGCCAGCCCCGGGGAATGGCCGTGACCATCAGCGTG |
| | AAGTGCGAGAAGATCAGCACCCTGAGCTGCGAGAACAAGATCATCAGCTTCAAGG |
| | AGATGAACCCTCCCGACAACATCAAGGACACCAAGAGCGACATCATCTTCTTCCA |
| | GCGGAGCGTGCCTGGCCACGACAACAAGATGCAGTTCGAGAGCAGCAGCTACGAG |
| | GGCTACTTCCTGGCCTGCGAGAAGGAGCGGGACCTGTTCAAGCTGATCCTGAAGA |
| | AGGAGGACGAGCTGGGCGACCGGAGCATCATGTTCACCGTGCAGAACGAGGACTA |
| | ACTCGAGGATCCGGATTAGTCCAATTTGTTAAAGACAGGATATCAGTGGTCCAGG |
| | CTCTAGTTTTGACTCAACAATATCACCAGCTGAAGCCTATAGAGTACGAGCCATA |
| | GATAAAATAAAAGATTTTATTTAGTCTCCAGAAAAAGGGGGGAATGAAAGACCC |
| | CACCTGTAGGTTTGGCAAGCTAGCTTAAGTAACGCCATTTTGCAAGGCATGGAAA |
| | AATACATAACTGAGAATAGAGAAGTTCAGATCAAGGTCAGGAACAGATGGAACA |
| | GCTGAATATGGGCCAAACAGGATATCTGTGGTAAGCAGTTCCTGCCCCGGCTCAG |
| | GGCCAAGAACAGATGGAACAGCTGAATATGGGCCAAACAGGATATCTGTGGTAAG |
| | CAGTTCCTGCCCCGGCTCAGGGCCAAGAACAGATGGTCCCCAGATGCGGTCCAGCC |
| | CTCAGCAGTTTCTAGAGAACCATCAGATGTTTCCAGGGTGCCCCAAGGACCTGAA |
| | ATGACCCTGTGCCTTATTTGAACTAACCAATCAGTTCGCTTCTCGCTTCTGTTCGC |
| | GCGCTTCTGCTCCCCGAGCTCAATAAAAGAGCCCACAACCCCTCACTCGGGGCGCC |
| | AGTCCTCCGATTGACTGAGTCGCCCGGGTACCCGTGTATCCAATAAACCCTCTTGC |
| | AGTTGCATCCGACTTGTGGTCTCGCTGTTCCTTGGGAGGGTCTCCTCTGAGTGATT |
| | GACTACCCGTCAGCGGGGGTCTTTCACACATGCAGCATGTATCAAAATTAATTTG |
| | GTTTTTTTTCTTAAGTATTTACATTAAATGGCCATAGTACTTAAAGTTACATTGG |
| | CTTCCTTGAAATAAACATGGAGTATTCAGAATGTGTCATAAATATTTCTAATTTT |
| | AAGATAGTATCTCCATTGGCTTTCTACTTTTTCTTTTATTTTTTTTTGTCCTCTGT |
| | CTTCCATTTGTTGTTGTTGTTGTTTGTTTGTTTGTTTGTTGGTTGGTTGGTTAAT |
| | TTTTTTTTAAAGATCCTACACTATAGTTCAAGCTAGACTATTAGCTACTCTGTAA |
| | CCCAGGGTGACCTTGAAGTCATGGGTAGCCTGCTGTTTTAGCCTTCCCACATCTAA |
| | GATTACAGGTATGAGCTATCATTTTTGGTATATTGATTGATTGATTGATTGATGT |
| | GTGTGTGTGTGATTGTGTTTGTGTGTGTGACTGTGAAAATGTGTGTATGGGTGTG |
| | TGTGAATGTGTGTATGTATGTGTGTGTGTGAGTGTGTGTGTGTGTGTGTGCATGT |
| | GTGTGTGTGTGACTGTGTCTATGTGTATGACTGTGTGTGTGTGTGTGTGTGTGTG |
| | TGTGTGTGTGTGTGTGTGTGTGTTGTGAAAAAATATTCTATGGTAGTGAGAGCCA |
| | ACGCTCCGGCTCAGGTGTCAGGTTGGTTTTTGAGACAGAGTCTTTCACTTAGCTT |
| | GGAATTCACTGGCCGTCGTTTTACAACGTCGTGACTGGGAAAACCCTGGCGTTAC |
| | CCAACTTAATCGCCTTGCAGCACATCCCCCTTTCGCCAGCTGGCGTAATAGCGAAG |
| | AGGCCCGCACCGATCGCCCTTCCCAACAGTTGCGCAGCCTGAATGGCGAATGGCGC |
| | CTGATGCGGTATTTTCTCCTTACGCATCTGTGCGGTATTTCACACCGCATATGGTG |
| | CACTCTCAGTACAATCTGCTCTGATGCCGCATAGTTAAGCCAGCCCCGACACCCGC |
| | CAACACCCGCTGACGCGCCCTGACGGGCTTGTCTGCTCCCGGCATCCGCTTACAGA |
| | CAAGCTGTGACCGTCTCCGGGAGCTGCATGTGTCAGAGGTTTTCACCGTCATCACC |
| | GAAACGCGCGATGACGAAAGGGCCTCGTGATACGCCTATTTTTATAGGTTAATGT |
| | CATGATAATAATGGTTTCTTAGACGTCAGGTGGCACTTTTCGGGGAAATGTGCGC |
| | GGAACCCCTATTTGTTTATTTTTCTAAATACATTCAAATATGTATCCGCTCATGA |
| | GACAATAACCCTGATAAATGCTTCAATAATATTGAAAAAGGAAGAGTATGAGTA |
| | TTCAACATTTCCGTGTCGCCCTTATTCCCTTTTTTGCGGCATTTTGCCTTCCTGTT |
| | TTTGCTCACCCAGAAACGCTGGTGAAAGTAAAAGATGCTGAAGATCAGTTGGGTG |
| | CACGAGTGGGTTACATCGAACTGGATCTCAACAGCGGTAAGATCCTTGAGAGTTT |
| | TCGCCCCGAAGAACGTTTTCCAATGATGAGCACTTTTAAAGTTCTGCTATGTGGC |
| | GCGGTATTATCCCGTATTGACGCCGGGCAAGAGCAACTCGGTCGCCGCATACACT |
| | ATTCTCAGAATGACTTGGTTGAGTACTCACCAGTCACAGAAAAGCATCTTACGGA |
| | TGGCATGACAGTAAGAGAATTATGCAGTGCTGCCATAACCATGAGTGATAACACT |
| | GCGGCCAACTTACTTCTGACAACGATCGGAGGACCGAAGGAGCTAACCGCTTTTT |
| | TGCACAACATGGGGGATCATGTAACTCGCCTTGATCGTTGGGAACCGGAGCTGAA |
| | TGAAGCCATACCAAACGACGAGCGTGACACCACGATGCCTGTAGCAATGGCAACA |
| | ACGTTGCGCAAACTATTAACTGGCGAACTACTTACTCTAGCTTCCCGGCAACAAT |
| | TAATAGACTGGATGGAGGCGGATAAAGTTGCAGGACCACTTCTGCGCTCGGCCCT |
| | TCCGGCTGGCTGGTTTATTGCTGATAAATCTGGAGCCGGTGAGCGTGGGTCTCGC |
| | GGTATCATTGCAGCACTGGGGCCAGATGGTAAGCCCTCCCGTATCGTAGTTATCT |
| | ACACGACGGGGAGTCAGGCAACTATGGATGAACGAAATAGACAGATCGCTGAGAT |
| | AGGTGCCTCACTGATTAAGCATTGGTAACTGTCAGACCAAGTTTACTCATATATA |
| | CTTTAGATTGATTTAAAACTTCATTTTTAATTTAAAAGGATCTAGGTGAAGATCC |
| | TTTTTGATAATCTCATGACCAAAATCCCTTAACGTGAGTTTTCGTTCCACTGAGC |
| | GTCAGACCCCGTAGAAAAGATCAAAGGATCTTCTTGAGATCCTTTTTTTCTGCGC |
| | GTAATCTGCTGCTTGCAAACAAAAAAACCACCGCTACCAGCGGTGGTTTGTTTGC |
| | CGGATCAAGAGCTACCAACTCTTTTTCCGAAGGTAACTGGCTTCAGCAGAGCGCA |
| | GATACCAAATACTGTCCTTCTAGTGTAGCCGTAGTTAGGCCACCACTTCAAGAAC |
| | TCTGTAGCACCGCCTACATACCTCGCTCTGCTAATCCTGTTACCAGTGGCTGCTGC |
| | CAGTGGCGATAAGTCGTGTCTTACCGGGTTGGACTCAAGACGATAGTTACCGGAT |
| | AAGGCGCAGCGGTCGGGCTGAACGGGGGGTTCGTGCACACAGCCCAGCTTGGAGC |
| | GAACGACCTACACCGAACTGAGATACCTACAGCGTGAGCATTGAGAAAGCGCCAC |
| | GCTTCCCGAAGGGAGAAAGGCGGACAGGTATCCGGTAAGCGGCAGGGTCGGAACA |
| | GGAGAGCGCACGAGGGAGCTTCCAGGGGGAAACGCCTGGTATCTTTATAGTCCTG |
| | TCGGGTTTCGCCACCTCTGACTTGAGCGTCGATTTTTGTGATGCTCGTCAGGGGG |
| | GCGGAGCCTATGGAAAAACGCCAGCAACGCGGCCTTTTTACGGTTCCTGGCCTTT |
| | TGCTGGCCTTTTGCTCACATGTTCTTTCCTGCGTTATCCCCTGATTCTGTGGATAA |
| | CCGTATTACCGCCTTTGAGTGAGCTGATACCGCTCGCCGCAGCCGAACGACCGAGC |
| | GCAGCGAGTCAGTGAGCGAGGAAGCGGAAGAGCGCCCAATACGCAAACCGCCTCT |
| | CCCCGCGCGTTGGCCGATTCATTAATGCAGCTGGCACGACAGGTTTCCCGACTGGA |
| | AAGCGGGCAGTGAGCGCAACGCAATTAATGTGAGTTAGCTCACTCATTAGGCACC |
| | CCAGGCTTTACACTTTATGCTTCCGGCTCGTATGTTGTGTGGAATTGTGAGCGGA |
| | TAACAATTTCACACAGGAAACAGCTATGACCATGATTACGCCAAGCTTTGCTCTT |
| | AGGAGTTTCCTAATACATCCCAAACTCAAATATATAAAGCATTTGACTTGTTCTA |
| | TGCCCTAGGGGGCGGGGGGAAGCTAAGCCAGCTTTTTTTAACATTTAAAATGTTA |
| | ATTCCATTTTAAATGCACAGATGTTTTTATTTCATAAGGGTTTCAATGTGCATGA |
| | ATGCTGCAATATTCCTGTTACCAAAGCTAGTATAAATAAAAATAGATAAACGTGG |
| | AAATTACTTAGAGTTTCTGTCATTAACGTTTCCTTCCTCAGTTGACAACATAAAT |
| | GCGCTGCTGAGCAAGCCAGTTTGCATCTGTCAGGATCAATTTCCCATTATGCCAG |
| | TCATATTAATTACTAGTCAATTAGTTGATTTTTATTTTTGACATATACATGTGAA |
| | TGAAAGACCCCACCTGTAGGTTTGGCAAGCTAGCTTAAGTAACGCCATTTTGCAA |
| | GGCATGGAAAAATACATAACTGAGAATAGAAAAGTTCAGATCAAGGTCAGGAAC |
| | AGATGGAACAGCTGAATATGGGCCAAACAGGATATCTGTGGTAAGCAGTTCCTGC |
| | CCCGGCTCAGGGCCAAGAACAGATGGAACAGCTGAATATGGGCCAAACAGGATAT |
| | CTGTGGTAAGCAGTTCCTGCCCCGGCTCAGGGCCAAGAACAGATGGTCCCCAGAT |
| | GCGGTCCAGCCCTCAGCAGTTTCTAGAGAACCATCAGATGTTTCCAGGGTGCCCCA |
| | AGGACCTGAAATGACCCTGTGCCTTATTTGAACTAACCAATCAGTTCGCTTCTCG |
| | CTTCTGTTCGCGCGCTTATGCTCCCCGAGCTCAATAAAAGAGCCCACAACCCCTCA |
| | CTCGGGGCGCCAGTCCTCCGATTGACTGAGTCGCCCGGGTACCCGTGTATCCAATA |
| | AACCCTCTTGCAGTTGCATCCGACTTGTGGTCTCGCTGTTCCTTGGGAGGGTCTCC |
| | TCTGAGTGATTGACTACCCGTCAGCGGGGGTCTTTCATTTGGGGGCTCGTCCGGG |
| | ATCGGGAGACCCCTGCCCAGGGACCACCGACCCACCACCGGGAGGTAAGCTGGCCA |
| | GCAACTTATCTGTGTCTGTCCGATTGTCTAGTGTCTATGACTGATTTTATGCGCC |
| | TGCGTCGGTACTAGTTAGCTAACTAGCTCTGTATCTGGCGGACCCGTGGTGGAAC |
| | TGACGAGTTCGGAACACCCGGCCGCAACCCTGGGAGACGTCCCAGGGACTTCGGG |
| | GGCCGTTTTTGTGGCCCGACCTGAGTCCTAAAATCCCGATCGTTTAGGACTCTTTG |
| | GTGCACCCCCCTTAGAGGAGGGATATGTGGTTCTGGTAGGAGACGAGAACCTAAA |
| | ACAGTTCCCGCCTCCGTCTGAATTTTTGCTTTCGGTTTGGGACCGAAGCCGCGCCG |
| | CGCGTCTTGTCTGCTGCAGCATCGTTCTGTGTTGTCTCTGTCTGACTGTGTTTCTG |
| | TATTTGTCTGAAAATATGGGCCCGGGCTAGACTGTTACCACTCCCTTAAGTTTGA |
| | CCTTAGGTCACTGGAAAGATGTCGAGCGGATCGCTCACAACCAGTCGGTAGATGT |
| | CAAGAAGAGACGTTGGGTTACCTTCTGCTCTGCAGAATGGCCAACCTTTAACGTC |
| | GGATGGCCGCGAGACGGCACCTTTAACCGAGACCTCATCACCCAGGTTAAGATCA |
| | AGGTCTTTTCACCTGGCCCGCATGGACACCCAGACCAGGTCCCCTACATCGTGACC |
| | TGGGAAGCCTTGGCTTTTGACCCCCCTCCCTGGGTCAAGCCCTTTGTACACCCTAA |
| | GCCTCCGCCTCCTCTTCCTCCATCCGCCCCGTCTCTCCCCCTTGAACCTCCTCGTTC |
| | GACCCCGCCTCGATCCTCCCTTTATCCAGCCCTCACTCCTTCTCTAGGCGCCCCCAT |
| | ATGGCCATATGAGATCTTATATGGGGCACCCCCGCCCCTTGTAAACTTCCCTGACC |
| | CTGACATGACAAGAGTTACTAACAGCCCCTCTCTCCAAGCTCACTTACAGGCTCTC |
| | TACTTAGTCCAGCACGAAGTCTGGAGACCTCTGGCGGCAGCCTACCAAGAACAAC |
| | TGGACCGACCGGTGGTACCTCACCCTTACCGAGTCGGCGACACAGTGTGGGTCCGC |
| | CGACACCAGACTAAGAACCTAGAACCTCGCTGGAAAGGACCTTACACAGTCCTGC |
| | TGACCACCCCCACCGCCCTCAAAGTAGACGGCATCGCAGCTTGGATACACGCCGCC |
| | CACGTGAAGGCTGCCGACCCCGGGGGTGGACCATCCTCTAGACTG |
| SEQ ID NO: | CGGGACCCTGAGATGGGGGGAAAGCCGAGAAGGAAGAACCCTCAGGAAGGCCTGT |
| 104 | ACAATGAACTGCAGAAAGATAAGATGGCGGAGGCCTACAGTGAGATTGGGATGA |
| A synthetic | AAGGCGAGCGCCGGAGGGGCAAGGGGCACGATGGCCTTTACCAGGGTCTCAGTAC |
| polynucleotide | AGCCACCAAGGACACCTACGACGCCCTTCACATGCAGGCCCTGCCCCCTCGCCGGC |
| for TBB/H | GGAAACGGTCTGGATCTGGGGAAGGTCGGGGATCTTTGCTCACATGTGGAGATGT |
| and constit | CGAAGAGAATCCTGGCCCTATGAACCGGGGAGTGCCCTTCCGGCACCTGCTGCTGG |
| IL-18 | TGCTGCAGCTGGCCCTGCTGCCTGCCGCTACCCAGGGCTACTTCGGCAAGCTGGAG |
| | AGCAAGCTGAGCGTGATCCGGAACCTGAACGACCAGGTGCTGTTCATCGACCAGG |
| | GCAACCGGCCTCTGTTCGAGGACATGACCGACAGCGACTGCCGGGACAACGCTCCC |
| | CGGACCATCTTCATCATCAGCATGTACAAGGACAGCCAGCCCCGGGGAATGGCCG |
| | TGACCATCAGCGTGAAGTGCGAGAAGATCAGCACCCTGAGCTGCGAGAACAAGAT |
| | CATCAGCTTCAAGGAGATGAACCCTCCCGACAACATCAAGGACACCAAGAGCGAC |
| | ATCATCTTCTTCCAGCGGAGCGTGCCTGGCCACGACAACAAGATGCAGTTCGAGA |
| | GCAGCAGCTACGAGGGCTACTTCCTGGCCTGCGAGAAGGAGCGGGACCTGTTCAA |
| | GCTGATCCTGAAGAAGGAGGACGAGCTGGGCGACCGGAGCATCATGTTCACCGTG |
| | CAGAACGAGGACTAACTCGAG |
| SEQ ID NO: | CCATGGGCCCCGGCGTGCTGCTGCTGCTGCTGGTGGCCACCGCCTGGCACGGCCAG |
| 105 | GGCGGCGTGGTGAGCCACTTCAACGACTGCCCTCTGAGCCACGACGGCTACTGCCT |
| TBB/H and | GCACGACGGCGTGTGCATGTACATCGAGGCCCTGGACAAGTACGCCTGCAACTGC |
| constit IL-18 | GTGGTGGGCTACATCGGCGAGAGATGCCAGTACAGAGACCTGAAGTGGTGGGAGC |
| | TGAGAGCCGCCGCCCCCACCACCACACCCGCTCCCAGACCCCCTACCCCTGCCCCCA |
| | CCATCGCCAGCCAGCCCCTGAGCCTGAGACCCGAGGCCTGCAGACCTGCCGCCGGC |
| | GGAGCCGTGCACACCAGAGGCCTGGACTTCGCCTGCGACATCTACATCTGGGCTCC |
| | CCTGGCCGGCACCTGCGGCGTGCTGCTGCTGAGCCTGGTGATCACCCTGTACTGCA |
| | ACCACAAGAGAGGCAGAAAGAAGCTGCTGTACATCTTCAAGCAGCCCTTCATGAG |
| | ACCCGTGCAGACCACCCAGGAGGAGGACGGCTGCAGCTGCAGATTCCCCGAGGAG |
| | GAGGAGGGCGGCTGCGAGCTGAGAAGAAAGAGAAGCGGCAGCGGCGAGGGCAGA |
| | GGCAGCCTGCTGACCTGCGGCGACGTGGAGGAGAACCCCGGACCCATGGCTCTCCC |
| | AGTGACTGCCCTACTGCTTCCCCTAGCGCTTCTCCTGCATGCAGAGGTGCAGCTGC |
| | AGCAGTCTGGAGGAGGCTTGGTGCAACCTGGAGGATCCATGAAACTCTCCTGTGT |
| | TGCCTCTGGATTCACTTTCAGTAACTACTGGATGAACTGGGTCCGCCAGTCTCCAG |
| | AGAAGGGGCTTGAGTGGGTTGCTGAAATTAGATTGAAATCTAATAATTATGCAAC |
| | ACATTATGCGGAGTCTGTGAAAGGGAGGTTCACCATCTCAAGAGATGATTCCAAA |
| | AGTAGTGTCTACCTGCAAATGAACAACTTAAGAGCTGAAGACACTGGCATTTATT |
| | ACTGTACCTTTGGTAACTCCTTTGCTTACTGGGGCCAAGGGACCACGGTCACCGTC |
| | TCCTCAGGTGGAGGCGGTTCAGGCGGAGGTGGCTCTGGCGGTGGCGGATCGCAGG |
| | CCGTGGTCACTCAGGAATCTGCACTCACCACATCACCTGGTGAAACAGTCACACTC |
| | ACTTGTCGCTCAAGTACTGGGGCTGTTACAACTAGTAACTATGCCAACTGGGTCC |
| | AAGAAAAACCAGATCATTTATTCACTGGTCTAATAGGTGGTACCAACAACCGAGC |
| | ACCAGGTGTTCCTGCCAGATTCTCAGGCTCCCTGATTGGAGACAAGGCTGCCCTCA |
| | CCATCACAGGGGCACAGACTGAGGATGAGGCAATATATTTCTGTGCTCTATGGTA |
| | CAGCAACCATTGGGTGTTCGGTGGAGGAACCAAACTGACTGTCCTAGGATCAGAG |
| | GCGGCCGCAATTGAAGTTATGTATCCTCCTCCTTACCTAGACAATGAGAAGAGCA |
| | ATGGAACCATTATCCATGTGAAAGGGAAACACCTTTGTCCAAGTCCCCTATTTCC |
| | CGGACCTTCTAAGCCCTTTTGGGTGCTGGTGGTGGTTGGTGGAGTCCTGGCTTGC |
| | TATAGCTTGCTAGTAACAGTGGCCTTTATTATTTTCTGGGTGAGGAGTAAGAGGA |
| | GCAGGCTCCTGCACAGTGACTACATGAACATGACTCCCCGCCGCCCCGGGCCCACC |
| | CGCAAGCATTACCAGCCCTATGCCCCACCACGCGACTTCGCAGCCTATCGCTCCAG |
| | AGTGAAGTTCAGCAGGAGCGCAGACGCCCCCGCGTACCAGCAGGGCCAGAACCAG |
| | CTCTATAACGAGCTCAATCTAGGACGAAGAGAGGAGTACGATGTTTTGGACAAGA |
| | GACGTGGCCGGGACCCTGAGATGGGGGGAAAGCCGAGAAGGAAGAACCCTCAGGA |
| | AGGCCTGTACAATGAACTGCAGAAAGATAAGATGGCGGAGGCCTACAGTGAGATT |
| | GGGATGAAAGGCGAGCGCCGGAGGGGCAAGGGGCACGATGGCCTTTACCAGGGTC |
| | TCAGTACAGCCACCAAGGACACCTACGACGCCCTTCACATGCAGGCCCTGCCCCCT |
| | CGCCGGCGGAAACGGTCTGGATCTGGGGAAGGTCGGGGATCTTTGCTCACATGTG |
| | GAGATGTCGAAGAGAATCCTGGCCCTATGAACCGGGGAGTGCCCTTCCGGCACCT |
| | GCTGCTGGTGCTGCAGCTGGCCCTGCTGCCTGCCGCTACCCAGGGCTACTTCGGCA |
| | AGCTGGAGAGCAAGCTGAGCGTGATCCGGAACCTGAACGACCAGGTGCTGTTCAT |
| | CGACCAGGGCAACCGGCCTCTGTTCGAGGACATGACCGACAGCGACTGCCGGGAC |
| | AACGCTCCCCGGACCATCTTCATCATCAGCATGTACAAGGACAGCCAGCCCCGGGG |
| | AATGGCCGTGACCATCAGCGTGAAGTGCGAGAAGATCAGCACCCTGAGCTGCGAG |
| | AACAAGATCATCAGCTTCAAGGAGATGAACCCTCCCGACAACATCAAGGACACCA |
| | AGAGCGACATCATCTTCTTCCAGCGGAGCGTGCCTGGCCACGACAACAAGATGCA |
| | GTTCGAGAGCAGCAGCTACGAGGGCTACTTCCTGGCCTGCGAGAAGGAGCGGGAC |
| | CTGTTCAAGCTGATCCTGAAGAAGGAGGACGAGCTGGGCGACCGGAGCATCATGT |
| | TCACCGTGCAGAACGAGGACTAACTCGAGGGATCCGGATTAGTCCAATTTGTTAA |
| | AGACAGGATATCAGTGGTCCAGGCTCTAGTTTTGACTCAACAATATCACCAGCTG |
| | AAGCCTATAGAGTACGAGCCATAGATAAAATAAAAGATTTTATTTAGTCTCCAGA |
| | AAAAGGGGGGAATGAAAGACCCCACCTGTAGGTTTGGCAAGCTAGCTTAAGTAAC |
| | GCCATTTTGCAAGGCATGGAAAAATACATAACTGAGAATAGAGAAGTTCAGATCA |
| | AGGTCAGGAACAGATGGAACAGCTGAATATGGGCCAAACAGGATATCTGTGGTAA |
| | GCAGTTCCTGCCCCGGCTCAGGGCCAAGAACAGATGGAACAGCTGAATATGGGCC |
| | AAACAGGATATCTGTGGTAAGCAGTTCCTGCCCCGGCTCAGGGCCAAGAACAGAT |
| | GGTCCCCAGATGCGGTCCAGCCCTCAGCAGTTTCTAGAGAACCATCAGATGTTTCC |
| | AGGGTGCCCCAAGGACCTGAAATGACCCTGTGCCTTATTTGAACTAACCAATCAG |
| | TTCGCTTCTCGCTTCTGTTCGCGCGCTTCTGCTCCCCGAGCTCAATAAAAGAGCCC |
| | ACAACCCCTCACTCGGGGCGCCAGTCCTCCGATTGACTGAGTCGCCCGGGTACCCG |
| | TGTATCCAATAAACCCTCTTGCAGTTGCATCCGACTTGTGGTCTCGCTGTTCCTTG |
| | GGAGGGTCTCCTCTGAGTGATTGACTACCCGTCAGCGGGGGTCTTTCACACATGC |
| | AGCATGTATCAAAATTAATTTGGTTTTTTTTCTTAAGTATTTACATTAAATGGCC |
| | ATAGTACTTAAAGTTACATTGGCTTCCTTGAAATAAACATGGAGTATTCAGAATG |
| | TGTCATAAATATTTCTAATTTTAAGATAGTATCTCCATTGGCTTTCTACTTTTTC |
| | TTTTATTTTTTTTTGTCCTCTGTCTTCCATTTGTTGTTGTTGTTGTTTGTTTGTTT |
| | GTTTGTTGGTTGGTTGGTTAATTTTTTTTTAAAGATCCTACACTATAGTTCAAGC |
| | TAGACTATTAGCTACTCTGTAACCCAGGGTGACCTTGAAGTCATGGGTAGCCTGC |
| | TGTTTTAGCCTTCCCACATCTAAGATTACAGGTATGAGCTATCATTTTTGGTATA |
| | TTGATTGATTGATTGATTGATGTGTGTGTGTGTGATTGTGTTTGTGTGTGTGACT |
| | GTGAAAATGTGTGTATGGGTGTGTGTGAATGTGTGTATGTATGTGTGTGTGTGA |
| | GTGTGTGTGTGTGTGTGTGCATGTGTGTGTGTGTGACTGTGTCTATGTGTATGAC |
| | TGTGTGTGTGTGTGTGTGTGTGTGTGTGTGTGTGTGTGTGTGTGTGTTGTGAAAA |
| | AATATTCTATGGTAGTGAGAGCCAACGCTCCGGCTCAGGTGTCAGGTTGGTTTTT |
| | GAGACAGAGTCTTTCACTTAGCTTGGAATTCACTGGCCGTCGTTTTACAACGTCG |
| | TGACTGGGAAAACCCTGGCGTTACCCAACTTAATCGCCTTGCAGCACATCCCCCTT |
| | TCGCCAGCTGGCGTAATAGCGAAGAGGCCCGCACCGATCGCCCTTCCCAACAGTTG |
| | CGCAGCCTGAATGGCGAATGGCGCCTGATGCGGTATTTTCTCCTTACGCATCTGT |
| | GCGGTATTTCACACCGCATATGGTGCACTCTCAGTACAATCTGCTCTGATGCCGCA |
| | TAGTTAAGCCAGCCCCGACACCCGCCAACACCCGCTGACGCGCCCTGACGGGCTTG |
| | TCTGCTCCCGGCATCCGCTTACAGACAAGCTGTGACCGTCTCCGGGAGCTGCATGT |
| | GTCAGAGGTTTTCACCGTCATCACCGAAACGCGCGATGACGAAAGGGCCTCGTGA |
| | TACGCCTATTTTTATAGGTTAATGTCATGATAATAATGGTTTCTTAGACGTCAGG |
| | TGGCACTTTTCGGGGAAATGTGCGCGGAACCCCTATTTGTTTATTTTTCTAAATA |
| | CATTCAAATATGTATCCGCTCATGAGACAATAACCCTGATAAATGCTTCAATAAT |
| | ATTGAAAAAGGAAGAGTATGAGTATTCAACATTTCCGTGTCGCCCTTATTCCCTT |
| | TTTTGCGGCATTTTGCCTTCCTGTTTTTGCTCACCCAGAAACGCTGGTGAAAGTA |
| | AAAGATGCTGAAGATCAGTTGGGTGCACGAGTGGGTTACATCGAACTGGATCTCA |
| | ACAGCGGTAAGATCCTTGAGAGTTTTCGCCCCGAAGAACGTTTTCCAATGATGAG |
| | CACTTTTAAAGTTCTGCTATGTGGCGCGGTATTATCCCGTATTGACGCCGGGCAA |
| | GAGCAACTCGGTCGCCGCATACACTATTCTCAGAATGACTTGGTTGAGTACTCAC |
| | CAGTCACAGAAAAGCATCTTACGGATGGCATGACAGTAAGAGAATTATGCAGTGC |
| | TGCCATAACCATGAGTGATAACACTGCGGCCAACTTACTTCTGACAACGATCGGA |
| | GGACCGAAGGAGCTAACCGCTTTTTTGCACAACATGGGGGATCATGTAACTCGCC |
| | TTGATCGTTGGGAACCGGAGCTGAATGAAGCCATACCAAACGACGAGCGTGACAC |
| | CACGATGCCTGTAGCAATGGCAACAACGTTGCGCAAACTATTAACTGGCGAACTA |
| | CTTACTCTAGCTTCCCGGCAACAATTAATAGACTGGATGGAGGCGGATAAAGTTG |
| | CAGGACCACTTCTGCGCTCGGCCCTTCCGGCTGGCTGGTTTATTGCTGATAAATCT |
| | GGAGCCGGTGAGCGTGGGTCTCGCGGTATCATTGCAGCACTGGGGCCAGATGGTA |
| | AGCCCTCCCGTATCGTAGTTATCTACACGACGGGGAGTCAGGCAACTATGGATGA |
| | ACGAAATAGACAGATCGCTGAGATAGGTGCCTCACTGATTAAGCATTGGTAACTG |
| | TCAGACCAAGTTTACTCATATATACTTTAGATTGATTTAAAACTTCATTTTTAAT |
| | TTAAAAGGATCTAGGTGAAGATCCTTTTTGATAATCTCATGACCAAAATCCCTTA |
| | ACGTGAGTTTTCGTTCCACTGAGCGTCAGACCCCGTAGAAAAGATCAAAGGATCT |
| | TCTTGAGATCCTTTTTTTCTGCGCGTAATCTGCTGCTTGCAAACAAAAAAACCAC |
| | CGCTACCAGCGGTGGTTTGTTTGCCGGATCAAGAGCTACCAACTCTTTTTCCGAA |
| | GGTAACTGGCTTCAGCAGAGCGCAGATACCAAATACTGTCCTTCTAGTGTAGCCG |
| | TAGTTAGGCCACCACTTCAAGAACTCTGTAGCACCGCCTACATACCTCGCTCTGCT |
| | AATCCTGTTACCAGTGGCTGCTGCCAGTGGCGATAAGTCGTGTCTTACCGGGTTG |
| | GACTCAAGACGATAGTTACCGGATAAGGCGCAGCGGTCGGGCTGAACGGGGGGTT |
| | CGTGCACACAGCCCAGCTTGGAGCGAACGACCTACACCGAACTGAGATACCTACA |
| | GCGTGAGCATTGAGAAAGCGCCACGCTTCCCGAAGGGAGAAAGGCGGACAGGTAT |
| | CCGGTAAGCGGCAGGGTCGGAACAGGAGAGCGCACGAGGGAGCTTCCAGGGGGAA |
| | ACGCCTGGTATCTTTATAGTCCTGTCGGGTTTCGCCACCTCTGACTTGAGCGTCGA |
| | TTTTTGTGATGCTCGTCAGGGGGGCGGAGCCTATGGAAAAACGCCAGCAACGCGG |
| | CCTTTTTACGGTTCCTGGCCTTTTGCTGGCCTTTTGCTCACATGTTCTTTCCTGCG |
| | TTATCCCCTGATTCTGTGGATAACCGTATTACCGCCTTTGAGTGAGCTGATACCGC |
| | TCGCCGCAGCCGAACGACCGAGCGCAGCGAGTCAGTGAGCGAGGAAGCGGAAGAG |
| | CGCCCAATACGCAAACCGCCTCTCCCCGCGCGTTGGCCGATTCATTAATGCAGCTG |
| | GCACGACAGGTTTCCCGACTGGAAAGCGGGCAGTGAGCGCAACGCAATTAATGTG |
| | AGTTAGCTCACTCATTAGGCACCCCAGGCTTTACACTTTATGCTTCCGGCTCGTAT |
| | GTTGTGTGGAATTGTGAGCGGATAACAATTTCACACAGGAAACAGCTATGACCAT |
| | GATTACGCCAAGCTTTGCTCTTAGGAGTTTCCTAATACATCCCAAACTCAAATAT |
| | ATAAAGCATTTGACTTGTTCTATGCCCTAGGGGGCGGGGGGAAGCTAAGCCAGCT |
| | TTTTTTAACATTTAAAATGTTAATTCCATTTTAAATGCACAGATGTTTTTATTTC |
| | ATAAGGGTTTCAATGTGCATGAATGCTGCAATATTCCTGTTACCAAAGCTAGTAT |
| | AAATAAAAATAGATAAACGTGGAAATTACTTAGAGTTTCTGTCATTAACGTTTCC |
| | TTCCTCAGTTGACAACATAAATGCGCTGCTGAGCAAGCCAGTTTGCATCTGTCAG |
| | GATCAATTTCCCATTATGCCAGTCATATTAATTACTAGTCAATTAGTTGATTTTT |
| | ATTTTTGACATATACATGTGAATGAAAGACCCCACCTGTAGGTTTGGCAAGCTAG |
| | CTTAAGTAACGCCATTTTGCAAGGCATGGAAAAATACATAACTGAGAATAGAAAA |
| | GTTCAGATCAAGGTCAGGAACAGATGGAACAGCTGAATATGGGCCAAACAGGATA |
| | TCTGTGGTAAGCAGTTCCTGCCCCGGCTCAGGGCCAAGAACAGATGGAACAGCTG |
| | AATATGGGCCAAACAGGATATCTGTGGTAAGCAGTTCCTGCCCCGGCTCAGGGCC |
| | AAGAACAGATGGTCCCCAGATGCGGTCCAGCCCTCAGCAGTTTCTAGAGAACCAT |
| | CAGATGTTTCCAGGGTGCCCCAAGGACCTGAAATGACCCTGTGCCTTATTTGAAC |
| | TAACCAATCAGTTCGCTTCTCGCTTCTGTTCGCGCGCTTATGCTCCCCGAGCTCAA |
| | TAAAAGAGCCCACAACCCCTCACTCGGGGCGCCAGTCCTCCGATTGACTGAGTCGC |
| | CCGGGTACCCGTGTATCCAATAAACCCTCTTGCAGTTGCATCCGACTTGTGGTCTC |
| | GCTGTTCCTTGGGAGGGTCTCCTCTGAGTGATTGACTACCCGTCAGCGGGGGTCTT |
| | TCATTTGGGGGCTCGTCCGGGATCGGGAGACCCCTGCCCAGGGACCACCGACCCAC |
| | CACCGGGAGGTAAGCTGGCCAGCAACTTATCTGTGTCTGTCCGATTGTCTAGTGT |
| | CTATGACTGATTTTATGCGCCTGCGTCGGTACTAGTTAGCTAACTAGCTCTGTAT |
| | CTGGCGGACCCGTGGTGGAACTGACGAGTTCGGAACACCCGGCCGCAACCCTGGG |
| | AGACGTCCCAGGGACTTCGGGGGCCGTTTTTGTGGCCCGACCTGAGTCCTAAAAT |
| | CCCGATCGTTTAGGACTCTTTGGTGCACCCCCCTTAGAGGAGGGATATGTGGTTC |
| | TGGTAGGAGACGAGAACCTAAAACAGTTCCCGCCTCCGTCTGAATTTTTGCTTTC |
| | GGTTTGGGACCGAAGCCGCGCCGCGCGTCTTGTCTGCTGCAGCATCGTTCTGTGTT |
| | GTCTCTGTCTGACTGTGTTTCTGTATTTGTCTGAAAATATGGGCCCGGGCTAGAC |
| | TGTTACCACTCCCTTAAGTTTGACCTTAGGTCACTGGAAAGATGTCGAGCGGATC |
| | GCTCACAACCAGTCGGTAGATGTCAAGAAGAGACGTTGGGTTACCTTCTGCTCTG |
| | CAGAATGGCCAACCTTTAACGTCGGATGGCCGCGAGACGGCACCTTTAACCGAGA |
| | CCTCATCACCCAGGTTAAGATCAAGGTCTTTTCACCTGGCCCGCATGGACACCCAG |
| | ACCAGGTCCCCTACATCGTGACCTGGGAAGCCTTGGCTTTTGACCCCCCTCCCTGG |
| | GTCAAGCCCTTTGTACACCCTAAGCCTCCGCCTCCTCTTCCTCCATCCGCCCCGTCT |
| | CTCCCCCTTGAACCTCCTCGTTCGACCCCGCCTCGATCCTCCCTTTATCCAGCCCTC |
| | ACTCCTTCTCTAGGCGCCCCCATATGGCCATATGAGATCTTATATGGGGCACCCCC |
| | GCCCCTTGTAAACTTCCCTGACCCTGACATGACAAGAGTTACTAACAGCCCCTCTC |
| | TCCAAGCTCACTTACAGGCTCTCTACTTAGTCCAGCACGAAGTCTGGAGACCTCTG |
| | GCGGCAGCCTACCAAGAACAACTGGACCGACCGGTGGTACCTCACCCTTACCGAGT |
| | CGGCGACACAGTGTGGGTCCGCCGACACCAGACTAAGAACCTAGAACCTCGCTGG |
| | AAAGGACCTTACACAGTCCTGCTGACCACCCCCACCGCCCTCAAAGTAGACGGCAT |
| | CGCAGCTTGGATACACGCCGCCCACGTGAAGGCTGCCGACCCCGGGGGTGGACCAT |
| | CCTCTAGACTG |
| SEQ ID NO: | CGGGACCCTGAGATGGGGGGAAAGCCGAGAAGGAAGAACCCTCAGGAAGGCCTGT |
| 106 | ACAATGAACTGCAGAAAGATAAGATGGCGGAGGCCTACAGTGAGATTGGGATGA |
| Synthesized | AAGGCGAGCGCCGGAGGGGCAAGGGGCACGATGGCCTTTACCAGGGTCTCAGTAC |
| sequence for | AGCCACCAAGGACACCTACGACGCCCTTCACATGCAGGCCCTGCCCCCTCGCCGGC |
| T/BBH + pro- | GGAAACGGTCTGGATCTGGGGAAGGTCGGGGATCTTTGCTCACATGTGGAGATGT |
| IL-18 (Casp 8) | CGAAGAGAATCCTGGCCCTATGGCCGCCGAGCCCGTGGAGGACAACTGCATCAAC |
| | TTCGTGGCCATGAAGTTCATCGACAACACCCTGTACTTCATCGCCGAGGACGACG |
| | AGAACATCGAGACCGACATCTACTTCGGCAAGCTGGAGAGCAAGCTGAGCGTGAT |
| | CCGGAACCTGAACGACCAGGTGCTGTTCATCGACCAGGGCAACCGGCCTCTGTTCG |
| | AGGACATGACCGACAGCGACTGCCGGGACAACGCTCCCCGGACCATCTTCATCATC |
| | AGCATGTACAAGGACAGCCAGCCCCGGGGAATGGCCGTGACCATCAGCGTGAAGT |
| | GCGAGAAGATCAGCACCCTGAGCTGCGAGAACAAGATCATCAGCTTCAAGGAGAT |
| | GAACCCTCCCGACAACATCAAGGACACCAAGAGCGACATCATCTTCTTCCAGCGG |
| | AGCGTGCCTGGCCACGACAACAAGATGCAGTTCGAGAGCAGCAGCTACGAGGGCT |
| | ACTTCCTGGCCTGCGAGAAGGAGCGGGACCTGTTCAAGCTGATCCTGAAGAAGGA |
| | GGACGAGCTGGGCGACCGGAGCATCATGTTCACCGTGCAGAACGAGGACTAACTC |
| | GAG |
| SEQ ID NO: | CCATGGGCCCCGGCGTGCTGCTGCTGCTGCTGGTGGCCACCGCCTGGCACGGCCAG |
| 107 | GGCGGCGTGGTGAGCCACTTCAACGACTGCCCTCTGAGCCACGACGGCTACTGCCT |
| T/BBH + pro- | GCACGACGGCGTGTGCATGTACATCGAGGCCCTGGACAAGTACGCCTGCAACTGC |
| IL-18 (Casp 8) | GTGGTGGGCTACATCGGCGAGAGATGCCAGTACAGAGACCTGAAGTGGTGGGAGC |
| | TGAGAGCCGCCGCCCCCACCACCACACCCGCTCCCAGACCCCCTACCCCTGCCCCCA |
| | CCATCGCCAGCCAGCCCCTGAGCCTGAGACCCGAGGCCTGCAGACCTGCCGCCGGC |
| | GGAGCCGTGCACACCAGAGGCCTGGACTTCGCCTGCGACATCTACATCTGGGCTCC |
| | CCTGGCCGGCACCTGCGGCGTGCTGCTGCTGAGCCTGGTGATCACCCTGTACTGCA |
| | ACCACAAGAGAGGCAGAAAGAAGCTGCTGTACATCTTCAAGCAGCCCTTCATGAG |
| | ACCCGTGCAGACCACCCAGGAGGAGGACGGCTGCAGCTGCAGATTCCCCGAGGAG |
| | GAGGAGGGCGGCTGCGAGCTGAGAAGAAAGAGAAGCGGCAGCGGCGAGGGCAGA |
| | GGCAGCCTGCTGACCTGCGGCGACGTGGAGGAGAACCCCGGACCCATGGCTCTCCC |
| | AGTGACTGCCCTACTGCTTCCCCTAGCGCTTCTCCTGCATGCAGAGGTGCAGCTGC |
| | AGCAGTCTGGAGGAGGCTTGGTGCAACCTGGAGGATCCATGAAACTCTCCTGTGT |
| | TGCCTCTGGATTCACTTTCAGTAACTACTGGATGAACTGGGTCCGCCAGTCTCCAG |
| | AGAAGGGGCTTGAGTGGGTTGCTGAAATTAGATTGAAATCTAATAATTATGCAAC |
| | ACATTATGCGGAGTCTGTGAAAGGGAGGTTCACCATCTCAAGAGATGATTCCAAA |
| | AGTAGTGTCTACCTGCAAATGAACAACTTAAGAGCTGAAGACACTGGCATTTATT |
| | ACTGTACCTTTGGTAACTCCTTTGCTTACTGGGGCCAAGGGACCACGGTCACCGTC |
| | TCCTCAGGTGGAGGCGGTTCAGGCGGAGGTGGCTCTGGCGGTGGCGGATCGCAGG |
| | CCGTGGTCACTCAGGAATCTGCACTCACCACATCACCTGGTGAAACAGTCACACTC |
| | ACTTGTCGCTCAAGTACTGGGGCTGTTACAACTAGTAACTATGCCAACTGGGTCC |
| | AAGAAAAACCAGATCATTTATTCACTGGTCTAATAGGTGGTACCAACAACCGAGC |
| | ACCAGGTGTTCCTGCCAGATTCTCAGGCTCCCTGATTGGAGACAAGGCTGCCCTCA |
| | CCATCACAGGGGCACAGACTGAGGATGAGGCAATATATTTCTGTGCTCTATGGTA |
| | CAGCAACCATTGGGTGTTCGGTGGAGGAACCAAACTGACTGTCCTAGGATCAGAG |
| | GCGGCCGCAATTGAAGTTATGTATCCTCCTCCTTACCTAGACAATGAGAAGAGCA |
| | ATGGAACCATTATCCATGTGAAAGGGAAACACCTTTGTCCAAGTCCCCTATTTCC |
| | CGGACCTTCTAAGCCCTTTTGGGTGCTGGTGGTGGTTGGTGGAGTCCTGGCTTGC |
| | TATAGCTTGCTAGTAACAGTGGCCTTTATTATTTTCTGGGTGAGGAGTAAGAGGA |
| | GCAGGCTCCTGCACAGTGACTACATGAACATGACTCCCCGCCGCCCCGGGCCCACC |
| | CGCAAGCATTACCAGCCCTATGCCCCACCACGCGACTTCGCAGCCTATCGCTCCAG |
| | AGTGAAGTTCAGCAGGAGCGCAGACGCCCCCGCGTACCAGCAGGGCCAGAACCAG |
| | CTCTATAACGAGCTCAATCTAGGACGAAGAGAGGAGTACGATGTTTTGGACAAGA |
| | GACGTGGCCGGGACCCTGAGATGGGGGGAAAGCCGAGAAGGAAGAACCCTCAGGA |
| | AGGCCTGTACAATGAACTGCAGAAAGATAAGATGGCGGAGGCCTACAGTGAGATT |
| | GGGATGAAAGGCGAGCGCCGGAGGGGCAAGGGGCACGATGGCCTTTACCAGGGTC |
| | TCAGTACAGCCACCAAGGACACCTACGACGCCCTTCACATGCAGGCCCTGCCCCCT |
| | CGCCGGCGGAAACGGTCTGGATCTGGGGAAGGTCGGGGATCTTTGCTCACATGTG |
| | GAGATGTCGAAGAGAATCCTGGCCCTATGGCCGCCGAGCCCGTGGAGGACAACTG |
| | CATCAACTTCGTGGCCATGAAGTTCATCGACAACACCCTGTACTTCATCGCCGAGG |
| | ACGACGAGAACATCGAGACCGACATCTACTTCGGCAAGCTGGAGAGCAAGCTGAG |
| | CGTGATCCGGAACCTGAACGACCAGGTGCTGTTCATCGACCAGGGCAACCGGCCTC |
| | TGTTCGAGGACATGACCGACAGCGACTGCCGGGACAACGCTCCCCGGACCATCTTC |
| | ATCATCAGCATGTACAAGGACAGCCAGCCCCGGGGAATGGCCGTGACCATCAGCG |
| | TGAAGTGCGAGAAGATCAGCACCCTGAGCTGCGAGAACAAGATCATCAGCTTCAA |
| | GGAGATGAACCCTCCCGACAACATCAAGGACACCAAGAGCGACATCATCTTCTTC |
| | CAGCGGAGCGTGCCTGGCCACGACAACAAGATGCAGTTCGAGAGCAGCAGCTACG |
| | AGGGCTACTTCCTGGCCTGCGAGAAGGAGCGGGACCTGTTCAAGCTGATCCTGAA |
| | GAAGGAGGACGAGCTGGGCGACCGGAGCATCATGTTCACCGTGCAGAACGAGGAC |
| | TAACTCGAGGGATCCGGATTAGTCCAATTTGTTAAAGACAGGATATCAGTGGTCC |
| | AGGCTCTAGTTTTGACTCAACAATATCACCAGCTGAAGCCTATAGAGTACGAGCC |
| | ATAGATAAAATAAAAGATTTTATTTAGTCTCCAGAAAAAGGGGGGAATGAAAGA |
| | CCCCACCTGTAGGTTTGGCAAGCTAGCTTAAGTAACGCCATTTTGCAAGGCATGG |
| | AAAAATACATAACTGAGAATAGAGAAGTTCAGATCAAGGTCAGGAACAGATGGA |
| | ACAGCTGAATATGGGCCAAACAGGATATCTGTGGTAAGCAGTTCCTGCCCCGGCT |
| | CAGGGCCAAGAACAGATGGAACAGCTGAATATGGGCCAAACAGGATATCTGTGGT |
| | AAGCAGTTCCTGCCCCGGCTCAGGGCCAAGAACAGATGGTCCCCAGATGCGGTCC |
| | AGCCCTCAGCAGTTTCTAGAGAACCATCAGATGTTTCCAGGGTGCCCCAAGGACC |
| | TGAAATGACCCTGTGCCTTATTTGAACTAACCAATCAGTTCGCTTCTCGCTTCTGT |
| | TCGCGCGCTTCTGCTCCCCGAGCTCAATAAAAGAGCCCACAACCCCTCACTCGGGG |
| | CGCCAGTCCTCCGATTGACTGAGTCGCCCGGGTACCCGTGTATCCAATAAACCCTC |
| | TTGCAGTTGCATCCGACTTGTGGTCTCGCTGTTCCTTGGGAGGGTCTCCTCTGAGT |
| | GATTGACTACCCGTCAGCGGGGGTCTTTCACACATGCAGCATGTATCAAAATTAA |
| | TTTGGTTTTTTTTCTTAAGTATTTACATTAAATGGCCATAGTACTTAAAGTTACA |
| | TTGGCTTCCTTGAAATAAACATGGAGTATTCAGAATGTGTCATAAATATTTCTAA |
| | TTTTAAGATAGTATCTCCATTGGCTTTCTACTTTTTCTTTTATTTTTTTTTGTCCT |
| | CTGTCTTCCATTTGTTGTTGTTGTTGTTTGTTTGTTTGTTTGTTGGTTGGTTGGT |
| | TAATTTTTTTTTAAAGATCCTACACTATAGTTCAAGCTAGACTATTAGCTACTCT |
| | GTAACCCAGGGTGACCTTGAAGTCATGGGTAGCCTGCTGTTTTAGCCTTCCCACA |
| | TCTAAGATTACAGGTATGAGCTATCATTTTTGGTATATTGATTGATTGATTGATT |
| | GATGTGTGTGTGTGTGATTGTGTTTGTGTGTGTGACTGTGAAAATGTGTGTATGG |
| | GTGTGTGTGAATGTGTGTATGTATGTGTGTGTGTGAGTGTGTGTGTGTGTGTGTG |
| | CATGTGTGTGTGTGTGACTGTGTCTATGTGTATGACTGTGTGTGTGTGTGTGTGT |
| | GTGTGTGTGTGTGTGTGTGTGTGTGTGTTGTGAAAAAATATTCTATGGTAGTGAG |
| | AGCCAACGCTCCGGCTCAGGTGTCAGGTTGGTTTTTGAGACAGAGTCTTTCACTT |
| | AGCTTGGAATTCACTGGCCGTCGTTTTACAACGTCGTGACTGGGAAAACCCTGGC |
| | GTTACCCAACTTAATCGCCTTGCAGCACATCCCCCTTTCGCCAGCTGGCGTAATAG |
| | CGAAGAGGCCCGCACCGATCGCCCTTCCCAACAGTTGCGCAGCCTGAATGGCGAAT |
| | GGCGCCTGATGCGGTATTTTCTCCTTACGCATCTGTGCGGTATTTCACACCGCATA |
| | TGGTGCACTCTCAGTACAATCTGCTCTGATGCCGCATAGTTAAGCCAGCCCCGACA |
| | CCCGCCAACACCCGCTGACGCGCCCTGACGGGCTTGTCTGCTCCCGGCATCCGCTT |
| | ACAGACAAGCTGTGACCGTCTCCGGGAGCTGCATGTGTCAGAGGTTTTCACCGTC |
| | ATCACCGAAACGCGCGATGACGAAAGGGCCTCGTGATACGCCTATTTTTATAGGT |
| | TAATGTCATGATAATAATGGTTTCTTAGACGTCAGGTGGCACTTTTCGGGGAAAT |
| | GTGCGCGGAACCCCTATTTGTTTATTTTTCTAAATACATTCAAATATGTATCCGC |
| | TCATGAGACAATAACCCTGATAAATGCTTCAATAATATTGAAAAAGGAAGAGTA |
| | TGAGTATTCAACATTTCCGTGTCGCCCTTATTCCCTTTTTTGCGGCATTTTGCCTT |
| | CCTGTTTTTGCTCACCCAGAAACGCTGGTGAAAGTAAAAGATGCTGAAGATCAGT |
| | TGGGTGCACGAGTGGGTTACATCGAACTGGATCTCAACAGCGGTAAGATCCTTGA |
| | GAGTTTTCGCCCCGAAGAACGTTTTCCAATGATGAGCACTTTTAAAGTTCTGCTA |
| | TGTGGCGCGGTATTATCCCGTATTGACGCCGGGCAAGAGCAACTCGGTCGCCGCA |
| | TACACTATTCTCAGAATGACTTGGTTGAGTACTCACCAGTCACAGAAAAGCATCT |
| | TACGGATGGCATGACAGTAAGAGAATTATGCAGTGCTGCCATAACCATGAGTGAT |
| | AACACTGCGGCCAACTTACTTCTGACAACGATCGGAGGACCGAAGGAGCTAACCG |
| | CTTTTTTGCACAACATGGGGGATCATGTAACTCGCCTTGATCGTTGGGAACCGGA |
| | GCTGAATGAAGCCATACCAAACGACGAGCGTGACACCACGATGCCTGTAGCAATG |
| | GCAACAACGTTGCGCAAACTATTAACTGGCGAACTACTTACTCTAGCTTCCCGGC |
| | AACAATTAATAGACTGGATGGAGGCGGATAAAGTTGCAGGACCACTTCTGCGCTC |
| | GGCCCTTCCGGCTGGCTGGTTTATTGCTGATAAATCTGGAGCCGGTGAGCGTGGG |
| | TCTCGCGGTATCATTGCAGCACTGGGGCCAGATGGTAAGCCCTCCCGTATCGTAG |
| | TTATCTACACGACGGGGAGTCAGGCAACTATGGATGAACGAAATAGACAGATCGC |
| | TGAGATAGGTGCCTCACTGATTAAGCATTGGTAACTGTCAGACCAAGTTTACTCA |
| | TATATACTTTAGATTGATTTAAAACTTCATTTTTAATTTAAAAGGATCTAGGTGA |
| | AGATCCTTTTTGATAATCTCATGACCAAAATCCCTTAACGTGAGTTTTCGTTCCA |
| | CTGAGCGTCAGACCCCGTAGAAAAGATCAAAGGATCTTCTTGAGATCCTTTTTTT |
| | CTGCGCGTAATCTGCTGCTTGCAAACAAAAAAACCACCGCTACCAGCGGTGGTTT |
| | GTTTGCCGGATCAAGAGCTACCAACTCTTTTTCCGAAGGTAACTGGCTTCAGCAG |
| | AGCGCAGATACCAAATACTGTCCTTCTAGTGTAGCCGTAGTTAGGCCACCACTTC |
| | AAGAACTCTGTAGCACCGCCTACATACCTCGCTCTGCTAATCCTGTTACCAGTGGC |
| | TGCTGCCAGTGGCGATAAGTCGTGTCTTACCGGGTTGGACTCAAGACGATAGTTA |
| | CCGGATAAGGCGCAGCGGTCGGGCTGAACGGGGGGTTCGTGCACACAGCCCAGCT |
| | TGGAGCGAACGACCTACACCGAACTGAGATACCTACAGCGTGAGCATTGAGAAAG |
| | CGCCACGCTTCCCGAAGGGAGAAAGGCGGACAGGTATCCGGTAAGCGGCAGGGTC |
| | GGAACAGGAGAGCGCACGAGGGAGCTTCCAGGGGGAAACGCCTGGTATCTTTATA |
| | GTCCTGTCGGGTTTCGCCACCTCTGACTTGAGCGTCGATTTTTGTGATGCTCGTCA |
| | GGGGGGCGGAGCCTATGGAAAAACGCCAGCAACGCGGCCTTTTTACGGTTCCTGG |
| | CCTTTTGCTGGCCTTTTGCTCACATGTTCTTTCCTGCGTTATCCCCTGATTCTGTG |
| | GATAACCGTATTACCGCCTTTGAGTGAGCTGATACCGCTCGCCGCAGCCGAACGAC |
| | CGAGCGCAGCGAGTCAGTGAGCGAGGAAGCGGAAGAGCGCCCAATACGCAAACCG |
| | CCTCTCCCCGCGCGTTGGCCGATTCATTAATGCAGCTGGCACGACAGGTTTCCCGA |
| | CTGGAAAGCGGGCAGTGAGCGCAACGCAATTAATGTGAGTTAGCTCACTCATTAG |
| | GCACCCCAGGCTTTACACTTTATGCTTCCGGCTCGTATGTTGTGTGGAATTGTGA |
| | GCGGATAACAATTTCACACAGGAAACAGCTATGACCATGATTACGCCAAGCTTTG |
| | CTCTTAGGAGTTTCCTAATACATCCCAAACTCAAATATATAAAGCATTTGACTTG |
| | TTCTATGCCCTAGGGGGCGGGGGGAAGCTAAGCCAGCTTTTTTTAACATTTAAAA |
| | TGTTAATTCCATTTTAAATGCACAGATGTTTTTATTTCATAAGGGTTTCAATGTG |
| | CATGAATGCTGCAATATTCCTGTTACCAAAGCTAGTATAAATAAAAATAGATAAA |
| | CGTGGAAATTACTTAGAGTTTCTGTCATTAACGTTTCCTTCCTCAGTTGACAACA |
| | TAAATGCGCTGCTGAGCAAGCCAGTTTGCATCTGTCAGGATCAATTTCCCATTAT |
| | GCCAGTCATATTAATTACTAGTCAATTAGTTGATTTTTATTTTTGACATATACAT |
| | GTGAATGAAAGACCCCACCTGTAGGTTTGGCAAGCTAGCTTAAGTAACGCCATTT |
| | TGCAAGGCATGGAAAAATACATAACTGAGAATAGAAAAGTTCAGATCAAGGTCA |
| | GGAACAGATGGAACAGCTGAATATGGGCCAAACAGGATATCTGTGGTAAGCAGTT |
| | CCTGCCCCGGCTCAGGGCCAAGAACAGATGGAACAGCTGAATATGGGCCAAACAG |
| | GATATCTGTGGTAAGCAGTTCCTGCCCCGGCTCAGGGCCAAGAACAGATGGTCCC |
| | CAGATGCGGTCCAGCCCTCAGCAGTTTCTAGAGAACCATCAGATGTTTCCAGGGT |
| | GCCCCAAGGACCTGAAATGACCCTGTGCCTTATTTGAACTAACCAATCAGTTCGC |
| | TTCTCGCTTCTGTTCGCGCGCTTATGCTCCCCGAGCTCAATAAAAGAGCCCACAAC |
| | CCCTCACTCGGGGCGCCAGTCCTCCGATTGACTGAGTCGCCCGGGTACCCGTGTAT |
| | CCAATAAACCCTCTTGCAGTTGCATCCGACTTGTGGTCTCGCTGTTCCTTGGGAGG |
| | GTCTCCTCTGAGTGATTGACTACCCGTCAGCGGGGGTCTTTCATTTGGGGGCTCGT |
| | CCGGGATCGGGAGACCCCTGCCCAGGGACCACCGACCCACCACCGGGAGGTAAGCT |
| | GGCCAGCAACTTATCTGTGTCTGTCCGATTGTCTAGTGTCTATGACTGATTTTAT |
| | GCGCCTGCGTCGGTACTAGTTAGCTAACTAGCTCTGTATCTGGCGGACCCGTGGT |
| | GGAACTGACGAGTTCGGAACACCCGGCCGCAACCCTGGGAGACGTCCCAGGGACT |
| | TCGGGGGCCGTTTTTGTGGCCCGACCTGAGTCCTAAAATCCCGATCGTTTAGGAC |
| | TCTTTGGTGCACCCCCCTTAGAGGAGGGATATGTGGTTCTGGTAGGAGACGAGAA |
| | CCTAAAACAGTTCCCGCCTCCGTCTGAATTTTTGCTTTCGGTTTGGGACCGAAGCC |
| | GCGCCGCGCGTCTTGTCTGCTGCAGCATCGTTCTGTGTTGTCTCTGTCTGACTGTG |
| | TTTCTGTATTTGTCTGAAAATATGGGCCCGGGCTAGACTGTTACCACTCCCTTAA |
| | GTTTGACCTTAGGTCACTGGAAAGATGTCGAGCGGATCGCTCACAACCAGTCGGT |
| | AGATGTCAAGAAGAGACGTTGGGTTACCTTCTGCTCTGCAGAATGGCCAACCTTT |
| | AACGTCGGATGGCCGCGAGACGGCACCTTTAACCGAGACCTCATCACCCAGGTTA |
| | AGATCAAGGTCTTTTCACCTGGCCCGCATGGACACCCAGACCAGGTCCCCTACATC |
| | GTGACCTGGGAAGCCTTGGCTTTTGACCCCCCTCCCTGGGTCAAGCCCTTTGTACA |
| | CCCTAAGCCTCCGCCTCCTCTTCCTCCATCCGCCCCGTCTCTCCCCCTTGAACCTCC |
| | TCGTTCGACCCCGCCTCGATCCTCCCTTTATCCAGCCCTCACTCCTTCTCTAGGCGC |
| | CCCCATATGGCCATATGAGATCTTATATGGGGCACCCCCGCCCCTTGTAAACTTCC |
| | CTGACCCTGACATGACAAGAGTTACTAACAGCCCCTCTCTCCAAGCTCACTTACAG |
| | GCTCTCTACTTAGTCCAGCACGAAGTCTGGAGACCTCTGGCGGCAGCCTACCAAGA |
| | ACAACTGGACCGACCGGTGGTACCTCACCCTTACCGAGTCGGCGACACAGTGTGG |
| | GTCCGCCGACACCAGACTAAGAACCTAGAACCTCGCTGGAAAGGACCTTACACAG |
| | TCCTGCTGACCACCCCCACCGCCCTCAAAGTAGACGGCATCGCAGCTTGGATACAC |
| | GCCGCCCACGTGAAGGCTGCCGACCCCGGGGGTGGACCATCCTCTAGACTG |
| SEQ ID NO: | CGGGACCCTGAGATGGGGGGAAAGCCGAGAAGGAAGAACCCTCAGGAAGGCCTGT |
| 108 | ACAATGAACTGCAGAAAGATAAGATGGCGGAGGCCTACAGTGAGATTGGGATGA |
| Synthetic | AAGGCGAGCGCCGGAGGGGCAAGGGGCACGATGGCCTTTACCAGGGTCTCAGTAC |
| polynucleotide | AGCCACCAAGGACACCTACGACGCCCTTCACATGCAGGCCCTGCCCCCTCGCCGGC |
| for T/BBH + | GGAAACGGTCTGGATCTGGGGAAGGTCGGGGATCTTTGCTCACATGTGGAGATGT |
| pro-IL-18 | CGAAGAGAATCCTGGCCCTATGGCCGCCGAGCCCGTGGAGGACAACTGCATCAAC |
| (Casp 3) | TTCGTGGCCATGAAGTTCATCGACAACACCCTGTACTTCATCGCCGAGGACGACG |
| | AGAACGACGAGGTGGACATCTACTTCGGCAAGCTGGAGAGCAAGCTGAGCGTGAT |
| | CCGGAACCTGAACGACCAGGTGCTGTTCATCGACCAGGGCAACCGGCCTCTGTTCG |
| | AGGACATGACCGACAGCGACTGCCGGGACAACGCTCCCCGGACCATCTTCATCATC |
| | AGCATGTACAAGGACAGCCAGCCCCGGGGAATGGCCGTGACCATCAGCGTGAAGT |
| | GCGAGAAGATCAGCACCCTGAGCTGCGAGAACAAGATCATCAGCTTCAAGGAGAT |
| | GAACCCTCCCGACAACATCAAGGACACCAAGAGCGACATCATCTTCTTCCAGCGG |
| | AGCGTGCCTGGCCACGACAACAAGATGCAGTTCGAGAGCAGCAGCTACGAGGGCT |
| | ACTTCCTGGCCTGCGAGAAGGAGCGGGACCTGTTCAAGCTGATCCTGAAGAAGGA |
| | GGACGAGCTGGGCGACCGGAGCATCATGTTCACCGTGCAGAACGAGGACTAACTC |
| | GAG |
| SEQ ID NO: | CCATGGGCCCCGGCGTGCTGCTGCTGCTGCTGGTGGCCACCGCCTGGCACGGCCAG |
| 109 | GGCGGCGTGGTGAGCCACTTCAACGACTGCCCTCTGAGCCACGACGGCTACTGCCT |
| T/BBH + pro- | GCACGACGGCGTGTGCATGTACATCGAGGCCCTGGACAAGTACGCCTGCAACTGC |
| IL-18 (Casp 3) | GTGGTGGGCTACATCGGCGAGAGATGCCAGTACAGAGACCTGAAGTGGTGGGAGC |
| | TGAGAGCCGCCGCCCCCACCACCACACCCGCTCCCAGACCCCCTACCCCTGCCCCCA |
| | CCATCGCCAGCCAGCCCCTGAGCCTGAGACCCGAGGCCTGCAGACCTGCCGCCGGC |
| | GGAGCCGTGCACACCAGAGGCCTGGACTTCGCCTGCGACATCTACATCTGGGCTCC |
| | CCTGGCCGGCACCTGCGGCGTGCTGCTGCTGAGCCTGGTGATCACCCTGTACTGCA |
| | ACCACAAGAGAGGCAGAAAGAAGCTGCTGTACATCTTCAAGCAGCCCTTCATGAG |
| | ACCCGTGCAGACCACCCAGGAGGAGGACGGCTGCAGCTGCAGATTCCCCGAGGAG |
| | GAGGAGGGCGGCTGCGAGCTGAGAAGAAAGAGAAGCGGCAGCGGCGAGGGCAGA |
| | GGCAGCCTGCTGACCTGCGGCGACGTGGAGGAGAACCCCGGACCCATGGCTCTCCC |
| | AGTGACTGCCCTACTGCTTCCCCTAGCGCTTCTCCTGCATGCAGAGGTGCAGCTGC |
| | AGCAGTCTGGAGGAGGCTTGGTGCAACCTGGAGGATCCATGAAACTCTCCTGTGT |
| | TGCCTCTGGATTCACTTTCAGTAACTACTGGATGAACTGGGTCCGCCAGTCTCCAG |
| | AGAAGGGGCTTGAGTGGGTTGCTGAAATTAGATTGAAATCTAATAATTATGCAAC |
| | ACATTATGCGGAGTCTGTGAAAGGGAGGTTCACCATCTCAAGAGATGATTCCAAA |
| | AGTAGTGTCTACCTGCAAATGAACAACTTAAGAGCTGAAGACACTGGCATTTATT |
| | ACTGTACCTTTGGTAACTCCTTTGCTTACTGGGGCCAAGGGACCACGGTCACCGTC |
| | TCCTCAGGTGGAGGCGGTTCAGGCGGAGGTGGCTCTGGCGGTGGCGGATCGCAGG |
| | CCGTGGTCACTCAGGAATCTGCACTCACCACATCACCTGGTGAAACAGTCACACTC |
| | ACTTGTCGCTCAAGTACTGGGGCTGTTACAACTAGTAACTATGCCAACTGGGTCC |
| | AAGAAAAACCAGATCATTTATTCACTGGTCTAATAGGTGGTACCAACAACCGAGC |
| | ACCAGGTGTTCCTGCCAGATTCTCAGGCTCCCTGATTGGAGACAAGGCTGCCCTCA |
| | CCATCACAGGGGCACAGACTGAGGATGAGGCAATATATTTCTGTGCTCTATGGTA |
| | CAGCAACCATTGGGTGTTCGGTGGAGGAACCAAACTGACTGTCCTAGGATCAGAG |
| | GCGGCCGCAATTGAAGTTATGTATCCTCCTCCTTACCTAGACAATGAGAAGAGCA |
| | ATGGAACCATTATCCATGTGAAAGGGAAACACCTTTGTCCAAGTCCCCTATTTCC |
| | CGGACCTTCTAAGCCCTTTTGGGTGCTGGTGGTGGTTGGTGGAGTCCTGGCTTGC |
| | TATAGCTTGCTAGTAACAGTGGCCTTTATTATTTTCTGGGTGAGGAGTAAGAGGA |
| | GCAGGCTCCTGCACAGTGACTACATGAACATGACTCCCCGCCGCCCCGGGCCCACC |
| | CGCAAGCATTACCAGCCCTATGCCCCACCACGCGACTTCGCAGCCTATCGCTCCAG |
| | AGTGAAGTTCAGCAGGAGCGCAGACGCCCCCGCGTACCAGCAGGGCCAGAACCAG |
| | CTCTATAACGAGCTCAATCTAGGACGAAGAGAGGAGTACGATGTTTTGGACAAGA |
| | GACGTGGCCGGGACCCTGAGATGGGGGGAAAGCCGAGAAGGAAGAACCCTCAGGA |
| | AGGCCTGTACAATGAACTGCAGAAAGATAAGATGGCGGAGGCCTACAGTGAGATT |
| | GGGATGAAAGGCGAGCGCCGGAGGGGCAAGGGGCACGATGGCCTTTACCAGGGTC |
| | TCAGTACAGCCACCAAGGACACCTACGACGCCCTTCACATGCAGGCCCTGCCCCCT |
| | CGCCGGCGGAAACGGTCTGGATCTGGGGAAGGTCGGGGATCTTTGCTCACATGTG |
| | GAGATGTCGAAGAGAATCCTGGCCCTATGGCCGCCGAGCCCGTGGAGGACAACTG |
| | CATCAACTTCGTGGCCATGAAGTTCATCGACAACACCCTGTACTTCATCGCCGAGG |
| | ACGACGAGAACGACGAGGTGGACATCTACTTCGGCAAGCTGGAGAGCAAGCTGAG |
| | CGTGATCCGGAACCTGAACGACCAGGTGCTGTTCATCGACCAGGGCAACCGGCCTC |
| | TGTTCGAGGACATGACCGACAGCGACTGCCGGGACAACGCTCCCCGGACCATCTTC |
| | ATCATCAGCATGTACAAGGACAGCCAGCCCCGGGGAATGGCCGTGACCATCAGCG |
| | TGAAGTGCGAGAAGATCAGCACCCTGAGCTGCGAGAACAAGATCATCAGCTTCAA |
| | GGAGATGAACCCTCCCGACAACATCAAGGACACCAAGAGCGACATCATCTTCTTC |
| | CAGCGGAGCGTGCCTGGCCACGACAACAAGATGCAGTTCGAGAGCAGCAGCTACG |
| | AGGGCTACTTCCTGGCCTGCGAGAAGGAGCGGGACCTGTTCAAGCTGATCCTGAA |
| | GAAGGAGGACGAGCTGGGCGACCGGAGCATCATGTTCACCGTGCAGAACGAGGAC |
| | TAACTCGAGGGATCCGGATTAGTCCAATTTGTTAAAGACAGGATATCAGTGGTCC |
| | AGGCTCTAGTTTTGACTCAACAATATCACCAGCTGAAGCCTATAGAGTACGAGCC |
| | ATAGATAAAATAAAAGATTTTATTTAGTCTCCAGAAAAAGGGGGGAATGAAAGA |
| | CCCCACCTGTAGGTTTGGCAAGCTAGCTTAAGTAACGCCATTTTGCAAGGCATGG |
| | AAAAATACATAACTGAGAATAGAGAAGTTCAGATCAAGGTCAGGAACAGATGGA |
| | ACAGCTGAATATGGGCCAAACAGGATATCTGTGGTAAGCAGTTCCTGCCCCGGCT |
| | CAGGGCCAAGAACAGATGGAACAGCTGAATATGGGCCAAACAGGATATCTGTGGT |
| | AAGCAGTTCCTGCCCCGGCTCAGGGCCAAGAACAGATGGTCCCCAGATGCGGTCC |
| | AGCCCTCAGCAGTTTCTAGAGAACCATCAGATGTTTCCAGGGTGCCCCAAGGACC |
| | TGAAATGACCCTGTGCCTTATTTGAACTAACCAATCAGTTCGCTTCTCGCTTCTGT |
| | TCGCGCGCTTCTGCTCCCCGAGCTCAATAAAAGAGCCCACAACCCCTCACTCGGGG |
| | CGCCAGTCCTCCGATTGACTGAGTCGCCCGGGTACCCGTGTATCCAATAAACCCTC |
| | TTGCAGTTGCATCCGACTTGTGGTCTCGCTGTTCCTTGGGAGGGTCTCCTCTGAGT |
| | GATTGACTACCCGTCAGCGGGGGTCTTTCACACATGCAGCATGTATCAAAATTAA |
| | TTTGGTTTTTTTTCTTAAGTATTTACATTAAATGGCCATAGTACTTAAAGTTACA |
| | TTGGCTTCCTTGAAATAAACATGGAGTATTCAGAATGTGTCATAAATATTTCTAA |
| | TTTTAAGATAGTATCTCCATTGGCTTTCTACTTTTTCTTTTATTTTTTTTTGTCCT |
| | CTGTCTTCCATTTGTTGTTGTTGTTGTTTGTTTGTTTGTTTGTTGGTTGGTTGGT |
| | TAATTTTTTTTTAAAGATCCTACACTATAGTTCAAGCTAGACTATTAGCTACTCT |
| | GTAACCCAGGGTGACCTTGAAGTCATGGGTAGCCTGCTGTTTTAGCCTTCCCACA |
| | TCTAAGATTACAGGTATGAGCTATCATTTTTGGTATATTGATTGATTGATTGATT |
| | GATGTGTGTGTGTGTGATTGTGTTTGTGTGTGTGACTGTGAAAATGTGTGTATGG |
| | GTGTGTGTGAATGTGTGTATGTATGTGTGTGTGTGAGTGTGTGTGTGTGTGTGTG |
| | CATGTGTGTGTGTGTGACTGTGTCTATGTGTATGACTGTGTGTGTGTGTGTGTGT |
| | GTGTGTGTGTGTGTGTGTGTGTGTGTGTTGTGAAAAAATATTCTATGGTAGTGAG |
| | AGCCAACGCTCCGGCTCAGGTGTCAGGTTGGTTTTTGAGACAGAGTCTTTCACTT |
| | AGCTTGGAATTCACTGGCCGTCGTTTTACAACGTCGTGACTGGGAAAACCCTGGC |
| | GTTACCCAACTTAATCGCCTTGCAGCACATCCCCCTTTCGCCAGCTGGCGTAATAG |
| | CGAAGAGGCCCGCACCGATCGCCCTTCCCAACAGTTGCGCAGCCTGAATGGCGAAT |
| | GGCGCCTGATGCGGTATTTTCTCCTTACGCATCTGTGCGGTATTTCACACCGCATA |
| | TGGTGCACTCTCAGTACAATCTGCTCTGATGCCGCATAGTTAAGCCAGCCCCGACA |
| | CCCGCCAACACCCGCTGACGCGCCCTGACGGGCTTGTCTGCTCCCGGCATCCGCTT |
| | ACAGACAAGCTGTGACCGTCTCCGGGAGCTGCATGTGTCAGAGGTTTTCACCGTC |
| | ATCACCGAAACGCGCGATGACGAAAGGGCCTCGTGATACGCCTATTTTTATAGGT |
| | TAATGTCATGATAATAATGGTTTCTTAGACGTCAGGTGGCACTTTTCGGGGAAAT |
| | GTGCGCGGAACCCCTATTTGTTTATTTTTCTAAATACATTCAAATATGTATCCGC |
| | TCATGAGACAATAACCCTGATAAATGCTTCAATAATATTGAAAAAGGAAGAGTA |
| | TGAGTATTCAACATTTCCGTGTCGCCCTTATTCCCTTTTTTGCGGCATTTTGCCTT |
| | CCTGTTTTTGCTCACCCAGAAACGCTGGTGAAAGTAAAAGATGCTGAAGATCAGT |
| | TGGGTGCACGAGTGGGTTACATCGAACTGGATCTCAACAGCGGTAAGATCCTTGA |
| | GAGTTTTCGCCCCGAAGAACGTTTTCCAATGATGAGCACTTTTAAAGTTCTGCTA |
| | TGTGGCGCGGTATTATCCCGTATTGACGCCGGGCAAGAGCAACTCGGTCGCCGCA |
| | TACACTATTCTCAGAATGACTTGGTTGAGTACTCACCAGTCACAGAAAAGCATCT |
| | TACGGATGGCATGACAGTAAGAGAATTATGCAGTGCTGCCATAACCATGAGTGAT |
| | AACACTGCGGCCAACTTACTTCTGACAACGATCGGAGGACCGAAGGAGCTAACCG |
| | CTTTTTTGCACAACATGGGGGATCATGTAACTCGCCTTGATCGTTGGGAACCGGA |
| | GCTGAATGAAGCCATACCAAACGACGAGCGTGACACCACGATGCCTGTAGCAATG |
| | GCAACAACGTTGCGCAAACTATTAACTGGCGAACTACTTACTCTAGCTTCCCGGC |
| | AACAATTAATAGACTGGATGGAGGCGGATAAAGTTGCAGGACCACTTCTGCGCTC |
| | GGCCCTTCCGGCTGGCTGGTTTATTGCTGATAAATCTGGAGCCGGTGAGCGTGGG |
| | TCTCGCGGTATCATTGCAGCACTGGGGCCAGATGGTAAGCCCTCCCGTATCGTAG |
| | TTATCTACACGACGGGGAGTCAGGCAACTATGGATGAACGAAATAGACAGATCGC |
| | TGAGATAGGTGCCTCACTGATTAAGCATTGGTAACTGTCAGACCAAGTTTACTCA |
| | TATATACTTTAGATTGATTTAAAACTTCATTTTTAATTTAAAAGGATCTAGGTGA |
| | AGATCCTTTTTGATAATCTCATGACCAAAATCCCTTAACGTGAGTTTTCGTTCCA |
| | CTGAGCGTCAGACCCCGTAGAAAAGATCAAAGGATCTTCTTGAGATCCTTTTTTT |
| | CTGCGCGTAATCTGCTGCTTGCAAACAAAAAAACCACCGCTACCAGCGGTGGTTT |
| | GTTTGCCGGATCAAGAGCTACCAACTCTTTTTCCGAAGGTAACTGGCTTCAGCAG |
| | AGCGCAGATACCAAATACTGTCCTTCTAGTGTAGCCGTAGTTAGGCCACCACTTC |
| | AAGAACTCTGTAGCACCGCCTACATACCTCGCTCTGCTAATCCTGTTACCAGTGGC |
| | TGCTGCCAGTGGCGATAAGTCGTGTCTTACCGGGTTGGACTCAAGACGATAGTTA |
| | CCGGATAAGGCGCAGCGGTCGGGCTGAACGGGGGGTTCGTGCACACAGCCCAGCT |
| | TGGAGCGAACGACCTACACCGAACTGAGATACCTACAGCGTGAGCATTGAGAAAG |
| | CGCCACGCTTCCCGAAGGGAGAAAGGCGGACAGGTATCCGGTAAGCGGCAGGGTC |
| | GGAACAGGAGAGCGCACGAGGGAGCTTCCAGGGGGAAACGCCTGGTATCTTTATA |
| | GTCCTGTCGGGTTTCGCCACCTCTGACTTGAGCGTCGATTTTTGTGATGCTCGTCA |
| | GGGGGGCGGAGCCTATGGAAAAACGCCAGCAACGCGGCCTTTTTACGGTTCCTGG |
| | CCTTTTGCTGGCCTTTTGCTCACATGTTCTTTCCTGCGTTATCCCCTGATTCTGTG |
| | GATAACCGTATTACCGCCTTTGAGTGAGCTGATACCGCTCGCCGCAGCCGAACGAC |
| | CGAGCGCAGCGAGTCAGTGAGCGAGGAAGCGGAAGAGCGCCCAATACGCAAACCG |
| | CCTCTCCCCGCGCGTTGGCCGATTCATTAATGCAGCTGGCACGACAGGTTTCCCGA |
| | CTGGAAAGCGGGCAGTGAGCGCAACGCAATTAATGTGAGTTAGCTCACTCATTAG |
| | GCACCCCAGGCTTTACACTTTATGCTTCCGGCTCGTATGTTGTGTGGAATTGTGA |
| | GCGGATAACAATTTCACACAGGAAACAGCTATGACCATGATTACGCCAAGCTTTG |
| | CTCTTAGGAGTTTCCTAATACATCCCAAACTCAAATATATAAAGCATTTGACTTG |
| | TTCTATGCCCTAGGGGGCGGGGGGAAGCTAAGCCAGCTTTTTTTAACATTTAAAA |
| | TGTTAATTCCATTTTAAATGCACAGATGTTTTTATTTCATAAGGGTTTCAATGTG |
| | CATGAATGCTGCAATATTCCTGTTACCAAAGCTAGTATAAATAAAAATAGATAAA |
| | CGTGGAAATTACTTAGAGTTTCTGTCATTAACGTTTCCTTCCTCAGTTGACAACA |
| | TAAATGCGCTGCTGAGCAAGCCAGTTTGCATCTGTCAGGATCAATTTCCCATTAT |
| | GCCAGTCATATTAATTACTAGTCAATTAGTTGATTTTTATTTTTGACATATACAT |
| | GTGAATGAAAGACCCCACCTGTAGGTTTGGCAAGCTAGCTTAAGTAACGCCATTT |
| | TGCAAGGCATGGAAAAATACATAACTGAGAATAGAAAAGTTCAGATCAAGGTCA |
| | GGAACAGATGGAACAGCTGAATATGGGCCAAACAGGATATCTGTGGTAAGCAGTT |
| | CCTGCCCCGGCTCAGGGCCAAGAACAGATGGAACAGCTGAATATGGGCCAAACAG |
| | GATATCTGTGGTAAGCAGTTCCTGCCCCGGCTCAGGGCCAAGAACAGATGGTCCC |
| | CAGATGCGGTCCAGCCCTCAGCAGTTTCTAGAGAACCATCAGATGTTTCCAGGGT |
| | GCCCCAAGGACCTGAAATGACCCTGTGCCTTATTTGAACTAACCAATCAGTTCGC |
| | TTCTCGCTTCTGTTCGCGCGCTTATGCTCCCCGAGCTCAATAAAAGAGCCCACAAC |
| | CCCTCACTCGGGGCGCCAGTCCTCCGATTGACTGAGTCGCCCGGGTACCCGTGTAT |
| | CCAATAAACCCTCTTGCAGTTGCATCCGACTTGTGGTCTCGCTGTTCCTTGGGAGG |
| | GTCTCCTCTGAGTGATTGACTACCCGTCAGCGGGGGTCTTTCATTTGGGGGCTCGT |
| | CCGGGATCGGGAGACCCCTGCCCAGGGACCACCGACCCACCACCGGGAGGTAAGCT |
| | GGCCAGCAACTTATCTGTGTCTGTCCGATTGTCTAGTGTCTATGACTGATTTTAT |
| | GCGCCTGCGTCGGTACTAGTTAGCTAACTAGCTCTGTATCTGGCGGACCCGTGGT |
| | GGAACTGACGAGTTCGGAACACCCGGCCGCAACCCTGGGAGACGTCCCAGGGACT |
| | TCGGGGGCCGTTTTTGTGGCCCGACCTGAGTCCTAAAATCCCGATCGTTTAGGAC |
| | TCTTTGGTGCACCCCCCTTAGAGGAGGGATATGTGGTTCTGGTAGGAGACGAGAA |
| | CCTAAAACAGTTCCCGCCTCCGTCTGAATTTTTGCTTTCGGTTTGGGACCGAAGCC |
| | GCGCCGCGCGTCTTGTCTGCTGCAGCATCGTTCTGTGTTGTCTCTGTCTGACTGTG |
| | TTTCTGTATTTGTCTGAAAATATGGGCCCGGGCTAGACTGTTACCACTCCCTTAA |
| | GTTTGACCTTAGGTCACTGGAAAGATGTCGAGCGGATCGCTCACAACCAGTCGGT |
| | AGATGTCAAGAAGAGACGTTGGGTTACCTTCTGCTCTGCAGAATGGCCAACCTTT |
| | AACGTCGGATGGCCGCGAGACGGCACCTTTAACCGAGACCTCATCACCCAGGTTA |
| | AGATCAAGGTCTTTTCACCTGGCCCGCATGGACACCCAGACCAGGTCCCCTACATC |
| | GTGACCTGGGAAGCCTTGGCTTTTGACCCCCCTCCCTGGGTCAAGCCCTTTGTACA |
| | CCCTAAGCCTCCGCCTCCTCTTCCTCCATCCGCCCCGTCTCTCCCCCTTGAACCTCC |
| | TCGTTCGACCCCGCCTCGATCCTCCCTTTATCCAGCCCTCACTCCTTCTCTAGGCGC |
| | CCCCATATGGCCATATGAGATCTTATATGGGGCACCCCCGCCCCTTGTAAACTTCC |
| | CTGACCCTGACATGACAAGAGTTACTAACAGCCCCTCTCTCCAAGCTCACTTACAG |
| | GCTCTCTACTTAGTCCAGCACGAAGTCTGGAGACCTCTGGCGGCAGCCTACCAAGA |
| | ACAACTGGACCGACCGGTGGTACCTCACCCTTACCGAGTCGGCGACACAGTGTGG |
| | GTCCGCCGACACCAGACTAAGAACCTAGAACCTCGCTGGAAAGGACCTTACACAG |
| | TCCTGCTGACCACCCCCACCGCCCTCAAAGTAGACGGCATCGCAGCTTGGATACAC |
| | GCCGCCCACGTGAAGGCTGCCGACCCCGGGGGTGGACCATCCTCTAGACTG |
| SEQ ID NO: | CACCAAGGTGAGCAGCTTCGTGCACTGGATCAAGAAGACCATGAAGCGGTACCGC |
| 110 | CGCAAACGCTCTGGGTCCGGAGAAGGGCGGGGATCCTTGCTCACATGTGGGGATG |
| Synthesized | TTGAAGAGAATCCTGGGCCAATGGCCGCCGAGCCCGTGGAGGACAACTGCATCAA |
| sequence for | CTTCGTGGCCATGAAGTTCATCGACAACACCCTGTACTTCATCGCCGAGGACGACG |
| TBBH + GZB + | AGAACATCGAGCCCGACTACTTCGGCAAGCTGGAGAGCAAGCTGAGCGTGATCCG |
| pro-IL-18 | GAACCTGAACGACCAGGTGCTGTTCATCGACCAGGGCAACCGGCCTCTGTTCGAG |
| (GZB) | GACATGACCGACAGCGACTGCCGGGACAACGCTCCCCGGACCATCTTCATCATCAG |
| | CATGTACAAGGACAGCCAGCCCCGGGGAATGGCCGTGACCATCAGCGTGAAGTGC |
| | GAGAAGATCAGCACCCTGAGCTGCGAGAACAAGATCATCAGCTTCAAGGAGATGA |
| | ACCCTCCCGACAACATCAAGGACACCAAGAGCGACATCATCTTCTTCCAGCGGAGC |
| | GTGCCTGGCCACGACAACAAGATGCAGTTCGAGAGCAGCAGCTACGAGGGCTACT |
| | TCCTGGCCTGCGAGAAGGAGCGGGACCTGTTCAAGCTGATCCTGAAGAAGGAGGA |
| | CGAGCTGGGCGACCGGAGCATCATGTTCACCGTGCAGAACGAGGACTAACTCGAG |
| SEQ ID NO: | CCATGGGCCCCGGCGTGCTGCTGCTGCTGCTGGTGGCCACCGCCTGGCACGGCCAG |
| 111 | GGCGGCGTGGTGAGCCACTTCAACGACTGCCCTCTGAGCCACGACGGCTACTGCCT |
| TBBH + GZB + | GCACGACGGCGTGTGCATGTACATCGAGGCCCTGGACAAGTACGCCTGCAACTGC |
| pro-IL-18 | GTGGTGGGCTACATCGGCGAGAGATGCCAGTACAGAGACCTGAAGTGGTGGGAGC |
| (GZB) | TGAGAGCCGCCGCCCCCACCACCACACCCGCTCCCAGACCCCCTACCCCTGCCCCCA |
| | CCATCGCCAGCCAGCCCCTGAGCCTGAGACCCGAGGCCTGCAGACCTGCCGCCGGC |
| | GGAGCCGTGCACACCAGAGGCCTGGACTTCGCCTGCGACATCTACATCTGGGCTCC |
| | CCTGGCCGGCACCTGCGGCGTGCTGCTGCTGAGCCTGGTGATCACCCTGTACTGCA |
| | ACCACAAGAGAGGCAGAAAGAAGCTGCTGTACATCTTCAAGCAGCCCTTCATGAG |
| | ACCCGTGCAGACCACCCAGGAGGAGGACGGCTGCAGCTGCAGATTCCCCGAGGAG |
| | GAGGAGGGCGGCTGCGAGCTGAGAAGAAAGAGAAGCGGCAGCGGCGAGGGCAGA |
| | GGCAGCCTGCTGACCTGCGGCGACGTGGAGGAGAACCCCGGACCCATGGCTCTCCC |
| | AGTGACTGCCCTACTGCTTCCCCTAGCGCTTCTCCTGCATGCAGAGGTGCAGCTGC |
| | AGCAGTCTGGAGGAGGCTTGGTGCAACCTGGAGGATCCATGAAACTCTCCTGTGT |
| | TGCCTCTGGATTCACTTTCAGTAACTACTGGATGAACTGGGTCCGCCAGTCTCCAG |
| | AGAAGGGGCTTGAGTGGGTTGCTGAAATTAGATTGAAATCTAATAATTATGCAAC |
| | ACATTATGCGGAGTCTGTGAAAGGGAGGTTCACCATCTCAAGAGATGATTCCAAA |
| | AGTAGTGTCTACCTGCAAATGAACAACTTAAGAGCTGAAGACACTGGCATTTATT |
| | ACTGTACCTTTGGTAACTCCTTTGCTTACTGGGGCCAAGGGACCACGGTCACCGTC |
| | TCCTCAGGTGGAGGCGGTTCAGGCGGAGGTGGCTCTGGCGGTGGCGGATCGCAGG |
| | CCGTGGTCACTCAGGAATCTGCACTCACCACATCACCTGGTGAAACAGTCACACTC |
| | ACTTGTCGCTCAAGTACTGGGGCTGTTACAACTAGTAACTATGCCAACTGGGTCC |
| | AAGAAAAACCAGATCATTTATTCACTGGTCTAATAGGTGGTACCAACAACCGAGC |
| | ACCAGGTGTTCCTGCCAGATTCTCAGGCTCCCTGATTGGAGACAAGGCTGCCCTCA |
| | CCATCACAGGGGCACAGACTGAGGATGAGGCAATATATTTCTGTGCTCTATGGTA |
| | CAGCAACCATTGGGTGTTCGGTGGAGGAACCAAACTGACTGTCCTAGGATCAGAG |
| | GCGGCCGCAATTGAAGTTATGTATCCTCCTCCTTACCTAGACAATGAGAAGAGCA |
| | ATGGAACCATTATCCATGTGAAAGGGAAACACCTTTGTCCAAGTCCCCTATTTCC |
| | CGGACCTTCTAAGCCCTTTTGGGTGCTGGTGGTGGTTGGTGGAGTCCTGGCTTGC |
| | TATAGCTTGCTAGTAACAGTGGCCTTTATTATTTTCTGGGTGAGGAGTAAGAGGA |
| | GCAGGCTCCTGCACAGTGACTACATGAACATGACTCCCCGCCGCCCCGGGCCCACC |
| | CGCAAGCATTACCAGCCCTATGCCCCACCACGCGACTTCGCAGCCTATCGCTCCAG |
| | AGTGAAGTTCAGCAGGAGCGCAGACGCCCCCGCGTACCAGCAGGGCCAGAACCAG |
| | CTCTATAACGAGCTCAATCTAGGACGAAGAGAGGAGTACGATGTTTTGGACAAGA |
| | GACGTGGCCGGGACCCTGAGATGGGGGGAAAGCCGAGAAGGAAGAACCCTCAGGA |
| | AGGCCTGTACAATGAACTGCAGAAAGATAAGATGGCGGAGGCCTACAGTGAGATT |
| | GGGATGAAAGGCGAGCGCCGGAGGGGCAAGGGGCACGATGGCCTTTACCAGGGTC |
| | TCAGTACAGCCACCAAGGACACCTACGACGCCCTTCACATGCAGGCCCTGCCCCCT |
| | CGCCGGCGGAAACGGTCCGGATCTGGGGCTACAAACTTCTCTCTCTTGAAGCAGG |
| | CCGGAGATGTCGAAGAAAATCCAGGCCCTATGCAGCCCATCCTGTTGCTCCTGGCC |
| | TTCCTCTTGCTGCCTCGGGCCGACGCCGGCGAGATCATCGGCGGACACGAGGCCAA |
| | GCCCCACAGCAGGCCCTACATGGCCTACCTGATGATCTGGGACCAGAAGAGCCTG |
| | AAGCGGTGCGGAGGCTTCCTGATCCGGGACGACTTCGTGCTGACCGCCGCCCACTG |
| | CTGGGGAAGCAGCATCAACGTGACCCTGGGCGCTCACAACATCAAGGAGCAGGAG |
| | CCCACCCAGCAGTTCATCCCTGTGAAGCGGCCCATCCCTCACCCCGCCTACAACCC |
| | CAAGAACTTCAGCAACGACATCATGCTGCTGCAGCTGGAGCGGAAGGCCAAGCGG |
| | ACCCGGGCCGTGCAGCCCCTGCGGCTGCCCAGCAACAAGGCCCAGGTGAAGCCTGG |
| | CCAGACCTGCAGCGTGGCCGGCTGGGGCCAGACCGCTCCCCTGGGCAAGCACAGCC |
| | ACACCCTGCAGGAGGTGAAGATGACCGTGCAGGAGGACCGGAAGTGCGAGAGCGA |
| | CCTGCGGCACTACTACGACAGCACCATCGAGCTGTGCGTGGGAGACCCCGAGATC |
| | AAGAAGACCAGCTTCAAGGGCGACAGCGGCGGACCCCTGGTGTGCAACAAGGTGG |
| | CCCAGGGCATCGTGAGCTACGGCAGGAACAACGGCATGCCCCCTCGGGCCTGCACC |
| | AAGGTGAGCAGCTTCGTGCACTGGATCAAGAAGACCATGAAGCGGTACCGCCGCA |
| | AACGCTCTGGGTCCGGAGAAGGGCGGGGATCCTTGCTCACATGTGGGGATGTTGA |
| | AGAGAATCCTGGGCCAATGGCCGCCGAGCCCGTGGAGGACAACTGCATCAACTTC |
| | GTGGCCATGAAGTTCATCGACAACACCCTGTACTTCATCGCCGAGGACGACGAGA |
| | ACATCGAGCCCGACTACTTCGGCAAGCTGGAGAGCAAGCTGAGCGTGATCCGGAA |
| | CCTGAACGACCAGGTGCTGTTCATCGACCAGGGCAACCGGCCTCTGTTCGAGGAC |
| | ATGACCGACAGCGACTGCCGGGACAACGCTCCCCGGACCATCTTCATCATCAGCAT |
| | GTACAAGGACAGCCAGCCCCGGGGAATGGCCGTGACCATCAGCGTGAAGTGCGAG |
| | AAGATCAGCACCCTGAGCTGCGAGAACAAGATCATCAGCTTCAAGGAGATGAACC |
| | CTCCCGACAACATCAAGGACACCAAGAGCGACATCATCTTCTTCCAGCGGAGCGT |
| | GCCTGGCCACGACAACAAGATGCAGTTCGAGAGCAGCAGCTACGAGGGCTACTTC |
| | CTGGCCTGCGAGAAGGAGCGGGACCTGTTCAAGCTGATCCTGAAGAAGGAGGACG |
| | AGCTGGGCGACCGGAGCATCATGTTCACCGTGCAGAACGAGGACTAACTCGAGGG |
| | ATCCGGATTAGTCCAATTTGTTAAAGACAGGATATCAGTGGTCCAGGCTCTAGTT |
| | TTGACTCAACAATATCACCAGCTGAAGCCTATAGAGTACGAGCCATAGATAAAAT |
| | AAAAGATTTTATTTAGTCTCCAGAAAAAGGGGGGAATGAAAGACCCCACCTGTAG |
| | GTTTGGCAAGCTAGCTTAAGTAACGCCATTTTGCAAGGCATGGAAAAATACATAA |
| | CTGAGAATAGAGAAGTTCAGATCAAGGTCAGGAACAGATGGAACAGCTGAATAT |
| | GGGCCAAACAGGATATCTGTGGTAAGCAGTTCCTGCCCCGGCTCAGGGCCAAGAA |
| | CAGATGGAACAGCTGAATATGGGCCAAACAGGATATCTGTGGTAAGCAGTTCCTG |
| | CCCCGGCTCAGGGCCAAGAACAGATGGTCCCCAGATGCGGTCCAGCCCTCAGCAGT |
| | TTCTAGAGAACCATCAGATGTTTCCAGGGTGCCCCAAGGACCTGAAATGACCCTG |
| | TGCCTTATTTGAACTAACCAATCAGTTCGCTTCTCGCTTCTGTTCGCGCGCTTCTG |
| | CTCCCCGAGCTCAATAAAAGAGCCCACAACCCCTCACTCGGGGCGCCAGTCCTCCG |
| | ATTGACTGAGTCGCCCGGGTACCCGTGTATCCAATAAACCCTCTTGCAGTTGCATC |
| | CGACTTGTGGTCTCGCTGTTCCTTGGGAGGGTCTCCTCTGAGTGATTGACTACCCG |
| | TCAGCGGGGGTCTTTCACACATGCAGCATGTATCAAAATTAATTTGGTTTTTTTT |
| | CTTAAGTATTTACATTAAATGGCCATAGTACTTAAAGTTACATTGGCTTCCTTGA |
| | AATAAACATGGAGTATTCAGAATGTGTCATAAATATTTCTAATTTTAAGATAGT |
| | ATCTCCATTGGCTTTCTACTTTTTCTTTTATTTTTTTTTGTCCTCTGTCTTCCATT |
| | TGTTGTTGTTGTTGTTTGTTTGTTTGTTTGTTGGTTGGTTGGTTAATTTTTTTTT |
| | AAAGATCCTACACTATAGTTCAAGCTAGACTATTAGCTACTCTGTAACCCAGGGT |
| | GACCTTGAAGTCATGGGTAGCCTGCTGTTTTAGCCTTCCCACATCTAAGATTACA |
| | GGTATGAGCTATCATTTTTGGTATATTGATTGATTGATTGATTGATGTGTGTGTG |
| | TGTGATTGTGTTTGTGTGTGTGACTGTGAAAATGTGTGTATGGGTGTGTGTGAAT |
| | GTGTGTATGTATGTGTGTGTGTGAGTGTGTGTGTGTGTGTGTGCATGTGTGTGTG |
| | TGTGACTGTGTCTATGTGTATGACTGTGTGTGTGTGTGTGTGTGTGTGTGTGTGT |
| | GTGTGTGTGTGTGTGTTGTGAAAAAATATTCTATGGTAGTGAGAGCCAACGCTCC |
| | GGCTCAGGTGTCAGGTTGGTTTTTGAGACAGAGTCTTTCACTTAGCTTGGAATTC |
| | ACTGGCCGTCGTTTTACAACGTCGTGACTGGGAAAACCCTGGCGTTACCCAACTT |
| | AATCGCCTTGCAGCACATCCCCCTTTCGCCAGCTGGCGTAATAGCGAAGAGGCCCG |
| | CACCGATCGCCCTTCCCAACAGTTGCGCAGCCTGAATGGCGAATGGCGCCTGATGC |
| | GGTATTTTCTCCTTACGCATCTGTGCGGTATTTCACACCGCATATGGTGCACTCTC |
| | AGTACAATCTGCTCTGATGCCGCATAGTTAAGCCAGCCCCGACACCCGCCAACACC |
| | CGCTGACGCGCCCTGACGGGCTTGTCTGCTCCCGGCATCCGCTTACAGACAAGCTG |
| | TGACCGTCTCCGGGAGCTGCATGTGTCAGAGGTTTTCACCGTCATCACCGAAACGC |
| | GCGATGACGAAAGGGCCTCGTGATACGCCTATTTTTATAGGTTAATGTCATGATA |
| | ATAATGGTTTCTTAGACGTCAGGTGGCACTTTTCGGGGAAATGTGCGCGGAACCC |
| | CTATTTGTTTATTTTTCTAAATACATTCAAATATGTATCCGCTCATGAGACAATA |
| | ACCCTGATAAATGCTTCAATAATATTGAAAAAGGAAGAGTATGAGTATTCAACAT |
| | TTCCGTGTCGCCCTTATTCCCTTTTTTGCGGCATTTTGCCTTCCTGTTTTTGCTCA |
| | CCCAGAAACGCTGGTGAAAGTAAAAGATGCTGAAGATCAGTTGGGTGCACGAGTG |
| | GGTTACATCGAACTGGATCTCAACAGCGGTAAGATCCTTGAGAGTTTTCGCCCCG |
| | AAGAACGTTTTCCAATGATGAGCACTTTTAAAGTTCTGCTATGTGGCGCGGTATT |
| | ATCCCGTATTGACGCCGGGCAAGAGCAACTCGGTCGCCGCATACACTATTCTCAG |
| | AATGACTTGGTTGAGTACTCACCAGTCACAGAAAAGCATCTTACGGATGGCATGA |
| | CAGTAAGAGAATTATGCAGTGCTGCCATAACCATGAGTGATAACACTGCGGCCAA |
| | CTTACTTCTGACAACGATCGGAGGACCGAAGGAGCTAACCGCTTTTTTGCACAAC |
| | ATGGGGGATCATGTAACTCGCCTTGATCGTTGGGAACCGGAGCTGAATGAAGCCA |
| | TACCAAACGACGAGCGTGACACCACGATGCCTGTAGCAATGGCAACAACGTTGCG |
| | CAAACTATTAACTGGCGAACTACTTACTCTAGCTTCCCGGCAACAATTAATAGAC |
| | TGGATGGAGGCGGATAAAGTTGCAGGACCACTTCTGCGCTCGGCCCTTCCGGCTG |
| | GCTGGTTTATTGCTGATAAATCTGGAGCCGGTGAGCGTGGGTCTCGCGGTATCAT |
| | TGCAGCACTGGGGCCAGATGGTAAGCCCTCCCGTATCGTAGTTATCTACACGACG |
| | GGGAGTCAGGCAACTATGGATGAACGAAATAGACAGATCGCTGAGATAGGTGCCT |
| | CACTGATTAAGCATTGGTAACTGTCAGACCAAGTTTACTCATATATACTTTAGAT |
| | TGATTTAAAACTTCATTTTTAATTTAAAAGGATCTAGGTGAAGATCCTTTTTGAT |
| | AATCTCATGACCAAAATCCCTTAACGTGAGTTTTCGTTCCACTGAGCGTCAGACCC |
| | CGTAGAAAAGATCAAAGGATCTTCTTGAGATCCTTTTTTTCTGCGCGTAATCTGC |
| | TGCTTGCAAACAAAAAAACCACCGCTACCAGCGGTGGTTTGTTTGCCGGATCAAG |
| | AGCTACCAACTCTTTTTCCGAAGGTAACTGGCTTCAGCAGAGCGCAGATACCAAA |
| | TACTGTCCTTCTAGTGTAGCCGTAGTTAGGCCACCACTTCAAGAACTCTGTAGCA |
| | CCGCCTACATACCTCGCTCTGCTAATCCTGTTACCAGTGGCTGCTGCCAGTGGCGA |
| | TAAGTCGTGTCTTACCGGGTTGGACTCAAGACGATAGTTACCGGATAAGGCGCAG |
| | CGGTCGGGCTGAACGGGGGGTTCGTGCACACAGCCCAGCTTGGAGCGAACGACCT |
| | ACACCGAACTGAGATACCTACAGCGTGAGCATTGAGAAAGCGCCACGCTTCCCGA |
| | AGGGAGAAAGGCGGACAGGTATCCGGTAAGCGGCAGGGTCGGAACAGGAGAGCGC |
| | ACGAGGGAGCTTCCAGGGGGAAACGCCTGGTATCTTTATAGTCCTGTCGGGTTTC |
| | GCCACCTCTGACTTGAGCGTCGATTTTTGTGATGCTCGTCAGGGGGGCGGAGCCT |
| | ATGGAAAAACGCCAGCAACGCGGCCTTTTTACGGTTCCTGGCCTTTTGCTGGCCTT |
| | TTGCTCACATGTTCTTTCCTGCGTTATCCCCTGATTCTGTGGATAACCGTATTACC |
| | GCCTTTGAGTGAGCTGATACCGCTCGCCGCAGCCGAACGACCGAGCGCAGCGAGTC |
| | AGTGAGCGAGGAAGCGGAAGAGCGCCCAATACGCAAACCGCCTCTCCCCGCGCGT |
| | TGGCCGATTCATTAATGCAGCTGGCACGACAGGTTTCCCGACTGGAAAGCGGGCA |
| | GTGAGCGCAACGCAATTAATGTGAGTTAGCTCACTCATTAGGCACCCCAGGCTTT |
| | ACACTTTATGCTTCCGGCTCGTATGTTGTGTGGAATTGTGAGCGGATAACAATTT |
| | CACACAGGAAACAGCTATGACCATGATTACGCCAAGCTTTGCTCTTAGGAGTTTC |
| | CTAATACATCCCAAACTCAAATATATAAAGCATTTGACTTGTTCTATGCCCTAGG |
| | GGGCGGGGGGAAGCTAAGCCAGCTTTTTTTAACATTTAAAATGTTAATTCCATTT |
| | TAAATGCACAGATGTTTTTATTTCATAAGGGTTTCAATGTGCATGAATGCTGCAA |
| | TATTCCTGTTACCAAAGCTAGTATAAATAAAAATAGATAAACGTGGAAATTACTT |
| | AGAGTTTCTGTCATTAACGTTTCCTTCCTCAGTTGACAACATAAATGCGCTGCTG |
| | AGCAAGCCAGTTTGCATCTGTCAGGATCAATTTCCCATTATGCCAGTCATATTAA |
| | TTACTAGTCAATTAGTTGATTTTTATTTTTGACATATACATGTGAATGAAAGACC |
| | CCACCTGTAGGTTTGGCAAGCTAGCTTAAGTAACGCCATTTTGCAAGGCATGGAA |
| | AAATACATAACTGAGAATAGAAAAGTTCAGATCAAGGTCAGGAACAGATGGAAC |
| | AGCTGAATATGGGCCAAACAGGATATCTGTGGTAAGCAGTTCCTGCCCCGGCTCA |
| | GGGCCAAGAACAGATGGAACAGCTGAATATGGGCCAAACAGGATATCTGTGGTAA |
| | GCAGTTCCTGCCCCGGCTCAGGGCCAAGAACAGATGGTCCCCAGATGCGGTCCAGC |
| | CCTCAGCAGTTTCTAGAGAACCATCAGATGTTTCCAGGGTGCCCCAAGGACCTGA |
| | AATGACCCTGTGCCTTATTTGAACTAACCAATCAGTTCGCTTCTCGCTTCTGTTCG |
| | CGCGCTTATGCTCCCCGAGCTCAATAAAAGAGCCCACAACCCCTCACTCGGGGCGC |
| | CAGTCCTCCGATTGACTGAGTCGCCCGGGTACCCGTGTATCCAATAAACCCTCTTG |
| | CAGTTGCATCCGACTTGTGGTCTCGCTGTTCCTTGGGAGGGTCTCCTCTGAGTGAT |
| | TGACTACCCGTCAGCGGGGGTCTTTCATTTGGGGGCTCGTCCGGGATCGGGAGAC |
| | CCCTGCCCAGGGACCACCGACCCACCACCGGGAGGTAAGCTGGCCAGCAACTTATC |
| | TGTGTCTGTCCGATTGTCTAGTGTCTATGACTGATTTTATGCGCCTGCGTCGGTA |
| | CTAGTTAGCTAACTAGCTCTGTATCTGGCGGACCCGTGGTGGAACTGACGAGTTC |
| | GGAACACCCGGCCGCAACCCTGGGAGACGTCCCAGGGACTTCGGGGGCCGTTTTTG |
| | TGGCCCGACCTGAGTCCTAAAATCCCGATCGTTTAGGACTCTTTGGTGCACCCCCC |
| | TTAGAGGAGGGATATGTGGTTCTGGTAGGAGACGAGAACCTAAAACAGTTCCCGC |
| | CTCCGTCTGAATTTTTGCTTTCGGTTTGGGACCGAAGCCGCGCCGCGCGTCTTGTC |
| | TGCTGCAGCATCGTTCTGTGTTGTCTCTGTCTGACTGTGTTTCTGTATTTGTCTGA |
| | AAATATGGGCCCGGGCTAGACTGTTACCACTCCCTTAAGTTTGACCTTAGGTCAC |
| | TGGAAAGATGTCGAGCGGATCGCTCACAACCAGTCGGTAGATGTCAAGAAGAGAC |
| | GTTGGGTTACCTTCTGCTCTGCAGAATGGCCAACCTTTAACGTCGGATGGCCGCG |
| | AGACGGCACCTTTAACCGAGACCTCATCACCCAGGTTAAGATCAAGGTCTTTTCA |
| | CCTGGCCCGCATGGACACCCAGACCAGGTCCCCTACATCGTGACCTGGGAAGCCTT |
| | GGCTTTTGACCCCCCTCCCTGGGTCAAGCCCTTTGTACACCCTAAGCCTCCGCCTC |
| | CTCTTCCTCCATCCGCCCCGTCTCTCCCCCTTGAACCTCCTCGTTCGACCCCGCCTC |
| | GATCCTCCCTTTATCCAGCCCTCACTCCTTCTCTAGGCGCCCCCATATGGCCATAT |
| | GAGATCTTATATGGGGCACCCCCGCCCCTTGTAAACTTCCCTGACCCTGACATGAC |
| | AAGAGTTACTAACAGCCCCTCTCTCCAAGCTCACTTACAGGCTCTCTACTTAGTCC |
| | AGCACGAAGTCTGGAGACCTCTGGCGGCAGCCTACCAAGAACAACTGGACCGACC |
| | GGTGGTACCTCACCCTTACCGAGTCGGCGACACAGTGTGGGTCCGCCGACACCAGA |
| | CTAAGAACCTAGAACCTCGCTGGAAAGGACCTTACACAGTCCTGCTGACCACCCCC |
| | ACCGCCCTCAAAGTAGACGGCATCGCAGCTTGGATACACGCCGCCCACGTGAAGGC |
| | TGCCGACCCCGGGGGTGGACCATCCTCTAGACTG |
| SEQ ID NO: | CCATGGGCCCCGGCGTGCTGCTGCTGCTGCTGGTGGCCACCGCCTGGCACGGCCAG |
| 112 | GGCGGCGTGGTGAGCCACTTCAACGACTGCCCTCTGAGCCACGACGGCTACTGCCT |
| TBB/H GzB | GCACGACGGCGTGTGCATGTACATCGAGGCCCTGGACAAGTACGCCTGCAACTGC |
| Pfn | GTGGTGGGCTACATCGGCGAGAGATGCCAGTACAGAGACCTGAAGTGGTGGGAGC |
| | TGAGAGCCGCCGCCCCCACCACCACACCCGCTCCCAGACCCCCTACCCCTGCCCCCA |
| | CCATCGCCAGCCAGCCCCTGAGCCTGAGACCCGAGGCCTGCAGACCTGCCGCCGGC |
| | GGAGCCGTGCACACCAGAGGCCTGGACTTCGCCTGCGACATCTACATCTGGGCTCC |
| | CCTGGCCGGCACCTGCGGCGTGCTGCTGCTGAGCCTGGTGATCACCCTGTACTGCA |
| | ACCACAAGAGAGGCAGAAAGAAGCTGCTGTACATCTTCAAGCAGCCCTTCATGAG |
| | ACCCGTGCAGACCACCCAGGAGGAGGACGGCTGCAGCTGCAGATTCCCCGAGGAG |
| | GAGGAGGGCGGCTGCGAGCTGAGAAGAAAGAGAAGCGGCAGCGGCGAGGGCAGA |
| | GGCAGCCTGCTGACCTGCGGCGACGTGGAGGAGAACCCCGGACCCATGGCTCTCCC |
| | AGTGACTGCCCTACTGCTTCCCCTAGCGCTTCTCCTGCATGCAGAGGTGCAGCTGC |
| | AGCAGTCTGGAGGAGGCTTGGTGCAACCTGGAGGATCCATGAAACTCTCCTGTGT |
| | TGCCTCTGGATTCACTTTCAGTAACTACTGGATGAACTGGGTCCGCCAGTCTCCAG |
| | AGAAGGGGCTTGAGTGGGTTGCTGAAATTAGATTGAAATCTAATAATTATGCAAC |
| | ACATTATGCGGAGTCTGTGAAAGGGAGGTTCACCATCTCAAGAGATGATTCCAAA |
| | AGTAGTGTCTACCTGCAAATGAACAACTTAAGAGCTGAAGACACTGGCATTTATT |
| | ACTGTACCTTTGGTAACTCCTTTGCTTACTGGGGCCAAGGGACCACGGTCACCGTC |
| | TCCTCAGGTGGAGGCGGTTCAGGCGGAGGTGGCTCTGGCGGTGGCGGATCGCAGG |
| | CCGTGGTCACTCAGGAATCTGCACTCACCACATCACCTGGTGAAACAGTCACACTC |
| | ACTTGTCGCTCAAGTACTGGGGCTGTTACAACTAGTAACTATGCCAACTGGGTCC |
| | AAGAAAAACCAGATCATTTATTCACTGGTCTAATAGGTGGTACCAACAACCGAGC |
| | ACCAGGTGTTCCTGCCAGATTCTCAGGCTCCCTGATTGGAGACAAGGCTGCCCTCA |
| | CCATCACAGGGGCACAGACTGAGGATGAGGCAATATATTTCTGTGCTCTATGGTA |
| | CAGCAACCATTGGGTGTTCGGTGGAGGAACCAAACTGACTGTCCTAGGATCAGAG |
| | GCGGCCGCAATTGAAGTTATGTATCCTCCTCCTTACCTAGACAATGAGAAGAGCA |
| | ATGGAACCATTATCCATGTGAAAGGGAAACACCTTTGTCCAAGTCCCCTATTTCC |
| | CGGACCTTCTAAGCCCTTTTGGGTGCTGGTGGTGGTTGGTGGAGTCCTGGCTTGC |
| | TATAGCTTGCTAGTAACAGTGGCCTTTATTATTTTCTGGGTGAGGAGTAAGAGGA |
| | GCAGGCTCCTGCACAGTGACTACATGAACATGACTCCCCGCCGCCCCGGGCCCACC |
| | CGCAAGCATTACCAGCCCTATGCCCCACCACGCGACTTCGCAGCCTATCGCTCCAG |
| | AGTGAAGTTCAGCAGGAGCGCAGACGCCCCCGCGTACCAGCAGGGCCAGAACCAG |
| | CTCTATAACGAGCTCAATCTAGGACGAAGAGAGGAGTACGATGTTTTGGACAAGA |
| | GACGTGGCCGGGACCCTGAGATGGGGGGAAAGCCGAGAAGGAAGAACCCTCAGGA |
| | AGGCCTGTACAATGAACTGCAGAAAGATAAGATGGCGGAGGCCTACAGTGAGATT |
| | GGGATGAAAGGCGAGCGCCGGAGGGGCAAGGGGCACGATGGCCTTTACCAGGGTC |
| | TCAGTACAGCCACCAAGGACACCTACGACGCCCTTCACATGCAGGCCCTGCCCCCT |
| | CGCCGGCGGAAACGGTCCGGATCTGGGGCTACAAACTTCTCTCTCTTGAAGCAGG |
| | CCGGAGATGTCGAAGAAAATCCAGGCCCTATGCAGCCCATCCTGTTGCTCCTGGCC |
| | TTCCTCTTGCTGCCTCGGGCCGACGCCGGCGAGATCATCGGCGGACACGAGGCCAA |
| | GCCCCACAGCAGGCCCTACATGGCCTACCTGATGATCTGGGACCAGAAGAGCCTG |
| | AAGCGGTGCGGAGGCTTCCTGATCCGGGACGACTTCGTGCTGACCGCCGCCCACTG |
| | CTGGGGAAGCAGCATCAACGTGACCCTGGGCGCTCACAACATCAAGGAGCAGGAG |
| | CCCACCCAGCAGTTCATCCCTGTGAAGCGGCCCATCCCTCACCCCGCCTACAACCC |
| | CAAGAACTTCAGCAACGACATCATGCTGCTGCAGCTGGAGCGGAAGGCCAAGCGG |
| | ACCCGGGCCGTGCAGCCCCTGCGGCTGCCCAGCAACAAGGCCCAGGTGAAGCCTGG |
| | CCAGACCTGCAGCGTGGCCGGCTGGGGCCAGACCGCTCCCCTGGGCAAGCACAGCC |
| | ACACCCTGCAGGAGGTGAAGATGACCGTGCAGGAGGACCGGAAGTGCGAGAGCGA |
| | CCTGCGGCACTACTACGACAGCACCATCGAGCTGTGCGTGGGAGACCCCGAGATC |
| | AAGAAGACCAGCTTCAAGGGCGACAGCGGCGGACCCCTGGTGTGCAACAAGGTGG |
| | CCCAGGGCATCGTGAGCTACGGCAGGAACAACGGCATGCCCCCTCGGGCCTGCACC |
| | AAGGTGAGCAGCTTCGTGCACTGGATCAAGAAGACCATGAAGCGGTACCGCCGCA |
| | AACGCTCTGGGTCCGGAGAAGGGCGGGGATCCTTGCTCACATGTGGGGATGTTGA |
| | AGAGAATCCTGGGCCAATGGCCGCTCGGCTGCTGCTGCTGGGCATCCTGTTGCTCC |
| | TGCTCCCTCTGCCCGTGCCCGCCCCCTGCCACACCGCCGCCCGGAGCGAGTGCAAG |
| | CGGAGCCACAAGTTCGTGCCTGGCGCCTGGCTGGCCGGAGAGGGCGTGGACGTGA |
| | CCAGCCTGCGGAGGAGCGGCAGCTTCCCTGTGGACACCCAGCGGTTCCTGCGGCCT |
| | GACGGCACCTGCACCCTGTGCGAGAACGCCCTGCAGGAGGGCACCCTGCAGAGGCT |
| | GCCCCTGGCCCTGACCAACTGGAGGGCCCAGGGAAGCGGCTGCCAGCGGCACGTGA |
| | CCAGGGCCAAGGTGAGCAGCACCGAGGCCGTGGCCCGGGACGCCGCCCGGAGCATC |
| | CGGAACGACTGGAAGGTGGGCCTGGACGTGACCCCCAAGCCCACCAGCAACGTGC |
| | ACGTGAGCGTGGCAGGCAGCCACAGCCAGGCCGCCAACTTCGCCGCCCAGAAGACC |
| | CACCAGGACCAGTACAGCTTCAGCACCGACACCGTGGAGTGCCGGTTCTACAGCTT |
| | CCACGTGGTGCACACACCCCCTCTGCACCCCGACTTCAAGCGGGCCCTGGGCGACC |
| | TGCCCCACCACTTCAACGCCAGCACCCAGCCTGCCTACCTGCGGCTGATCAGCAAC |
| | TACGGCACCCACTTCATCCGGGCTGTGGAGCTGGGAGGCAGGATCAGCGCCCTGAC |
| | CGCCCTGCGGACCTGCGAGCTGGCCCTGGAGGGCCTGACCGACAACGAGGTGGAG |
| | GACTGCCTGACCGTGGAGGCCCAGGTGAACATCGGCATCCACGGCAGCATCAGCG |
| | CCGAGGCCAAGGCCTGCGAGGAGAAGAAGAAGAAGCACAAGATGACCGCCAGCTT |
| | CCACCAGACCTACCGGGAGCGGCACAGCGAGGTGGTGGGAGGCCACCACACCAGC |
| | ATCAACGACCTGCTGTTCGGCATCCAGGCTGGACCCGAGCAGTACAGCGCCTGGGT |
| | GAACAGCCTGCCTGGCAGCCCCGGACTGGTGGACTACACCCTGGAGCCCCTGCACG |
| | TGCTGCTGGACAGCCAGGACCCCAGGCGGGAGGCCCTGCGGAGGGCCCTGAGCCA |
| | GTACCTGACCGACCGGGCTCGGTGGCGGGACTGCAGCAGACCCTGCCCCCCTGGCA |
| | GGCAGAAGAGCCCCAGGGACCCCTGCCAGTGCGTGTGCCACGGCAGCGCTGTGACC |
| | ACCCAGGACTGCTGCCCCAGGCAGCGGGGACTGGCCCAGCTGGAGGTGACCTTCAT |
| | CCAGGCCTGGGGCCTGTGGGGCGACTGGTTCACCGCCACCGACGCCTACGTGAAGC |
| | TGTTCTTCGGAGGCCAGGAGCTGCGGACCAGCACCGTGTGGGACAACAACAACCC |
| | CATCTGGAGCGTGCGGCTGGACTTCGGCGACGTGCTGCTGGCCACCGGCGGACCCC |
| | TGCGGCTGCAGGTGTGGGACCAGGACAGCGGCAGGGACGACGACCTGCTGGGCAC |
| | CTGCGACCAGGCTCCCAAGAGCGGCAGCCACGAGGTGCGGTGCAACCTGAACCAC |
| | GGCCACCTGAAGTTCCGGTACCACGCCAGGTGCCTGCCCCACCTGGGCGGAGGCAC |
| | CTGCCTGGACTACGTGCCCCAGATGCTGCTGGGCGAGCCCCCTGGCAACCGGAGCG |
| | GCGCTGTGTGGTAACTCGAGGGATCCGGATTAGTCCAATTTGTTAAAGACAGGAT |
| | ATCAGTGGTCCAGGCTCTAGTTTTGACTCAACAATATCACCAGCTGAAGCCTATA |
| | GAGTACGAGCCATAGATAAAATAAAAGATTTTATTTAGTCTCCAGAAAAAGGGG |
| | GGAATGAAAGACCCCACCTGTAGGTTTGGCAAGCTAGCTTAAGTAACGCCATTTT |
| | GCAAGGCATGGAAAAATACATAACTGAGAATAGAGAAGTTCAGATCAAGGTCAG |
| | GAACAGATGGAACAGCTGAATATGGGCCAAACAGGATATCTGTGGTAAGCAGTTC |
| | CTGCCCCGGCTCAGGGCCAAGAACAGATGGAACAGCTGAATATGGGCCAAACAGG |
| | ATATCTGTGGTAAGCAGTTCCTGCCCCGGCTCAGGGCCAAGAACAGATGGTCCCC |
| | AGATGCGGTCCAGCCCTCAGCAGTTTCTAGAGAACCATCAGATGTTTCCAGGGTG |
| | CCCCAAGGACCTGAAATGACCCTGTGCCTTATTTGAACTAACCAATCAGTTCGCTT |
| | CTCGCTTCTGTTCGCGCGCTTCTGCTCCCCGAGCTCAATAAAAGAGCCCACAACCC |
| | CTCACTCGGGGCGCCAGTCCTCCGATTGACTGAGTCGCCCGGGTACCCGTGTATCC |
| | AATAAACCCTCTTGCAGTTGCATCCGACTTGTGGTCTCGCTGTTCCTTGGGAGGG |
| | TCTCCTCTGAGTGATTGACTACCCGTCAGCGGGGGTCTTTCACACATGCAGCATGT |
| | ATCAAAATTAATTTGGTTTTTTTTCTTAAGTATTTACATTAAATGGCCATAGTAC |
| | TTAAAGTTACATTGGCTTCCTTGAAATAAACATGGAGTATTCAGAATGTGTCATA |
| | AATATTTCTAATTTTAAGATAGTATCTCCATTGGCTTTCTACTTTTTCTTTTATT |
| | TTTTTTTGTCCTCTGTCTTCCATTTGTTGTTGTTGTTGTTTGTTTGTTTGTTTGTT |
| | GGTTGGTTGGTTAATTTTTTTTTAAAGATCCTACACTATAGTTCAAGCTAGACTA |
| | TTAGCTACTCTGTAACCCAGGGTGACCTTGAAGTCATGGGTAGCCTGCTGTTTTA |
| | GCCTTCCCACATCTAAGATTACAGGTATGAGCTATCATTTTTGGTATATTGATTG |
| | ATTGATTGATTGATGTGTGTGTGTGTGATTGTGTTTGTGTGTGTGACTGTGAAAA |
| | TGTGTGTATGGGTGTGTGTGAATGTGTGTATGTATGTGTGTGTGTGAGTGTGTGT |
| | GTGTGTGTGTGCATGTGTGTGTGTGTGACTGTGTCTATGTGTATGACTGTGTGTG |
| | TGTGTGTGTGTGTGTGTGTGTGTGTGTGTGTGTGTGTGTTGTGAAAAAATATTCT |
| | ATGGTAGTGAGAGCCAACGCTCCGGCTCAGGTGTCAGGTTGGTTTTTGAGACAGA |
| | GTCTTTCACTTAGCTTGGAATTCACTGGCCGTCGTTTTACAACGTCGTGACTGGG |
| | AAAACCCTGGCGTTACCCAACTTAATCGCCTTGCAGCACATCCCCCTTTCGCCAGC |
| | TGGCGTAATAGCGAAGAGGCCCGCACCGATCGCCCTTCCCAACAGTTGCGCAGCCT |
| | GAATGGCGAATGGCGCCTGATGCGGTATTTTCTCCTTACGCATCTGTGCGGTATT |
| | TCACACCGCATATGGTGCACTCTCAGTACAATCTGCTCTGATGCCGCATAGTTAA |
| | GCCAGCCCCGACACCCGCCAACACCCGCTGACGCGCCCTGACGGGCTTGTCTGCTC |
| | CCGGCATCCGCTTACAGACAAGCTGTGACCGTCTCCGGGAGCTGCATGTGTCAGA |
| | GGTTTTCACCGTCATCACCGAAACGCGCGATGACGAAAGGGCCTCGTGATACGCC |
| | TATTTTTATAGGTTAATGTCATGATAATAATGGTTTCTTAGACGTCAGGTGGCAC |
| | TTTTCGGGGAAATGTGCGCGGAACCCCTATTTGTTTATTTTTCTAAATACATTCA |
| | AATATGTATCCGCTCATGAGACAATAACCCTGATAAATGCTTCAATAATATTGAA |
| | AAAGGAAGAGTATGAGTATTCAACATTTCCGTGTCGCCCTTATTCCCTTTTTTGC |
| | GGCATTTTGCCTTCCTGTTTTTGCTCACCCAGAAACGCTGGTGAAAGTAAAAGAT |
| | GCTGAAGATCAGTTGGGTGCACGAGTGGGTTACATCGAACTGGATCTCAACAGCG |
| | GTAAGATCCTTGAGAGTTTTCGCCCCGAAGAACGTTTTCCAATGATGAGCACTTT |
| | TAAAGTTCTGCTATGTGGCGCGGTATTATCCCGTATTGACGCCGGGCAAGAGCAA |
| | CTCGGTCGCCGCATACACTATTCTCAGAATGACTTGGTTGAGTACTCACCAGTCAC |
| | AGAAAAGCATCTTACGGATGGCATGACAGTAAGAGAATTATGCAGTGCTGCCATA |
| | ACCATGAGTGATAACACTGCGGCCAACTTACTTCTGACAACGATCGGAGGACCGA |
| | AGGAGCTAACCGCTTTTTTGCACAACATGGGGGATCATGTAACTCGCCTTGATCG |
| | TTGGGAACCGGAGCTGAATGAAGCCATACCAAACGACGAGCGTGACACCACGATG |
| | CCTGTAGCAATGGCAACAACGTTGCGCAAACTATTAACTGGCGAACTACTTACTC |
| | TAGCTTCCCGGCAACAATTAATAGACTGGATGGAGGCGGATAAAGTTGCAGGACC |
| | ACTTCTGCGCTCGGCCCTTCCGGCTGGCTGGTTTATTGCTGATAAATCTGGAGCCG |
| | GTGAGCGTGGGTCTCGCGGTATCATTGCAGCACTGGGGCCAGATGGTAAGCCCTC |
| | CCGTATCGTAGTTATCTACACGACGGGGAGTCAGGCAACTATGGATGAACGAAAT |
| | AGACAGATCGCTGAGATAGGTGCCTCACTGATTAAGCATTGGTAACTGTCAGACC |
| | AAGTTTACTCATATATACTTTAGATTGATTTAAAACTTCATTTTTAATTTAAAAG |
| | GATCTAGGTGAAGATCCTTTTTGATAATCTCATGACCAAAATCCCTTAACGTGAG |
| | TTTTCGTTCCACTGAGCGTCAGACCCCGTAGAAAAGATCAAAGGATCTTCTTGAG |
| | ATCCTTTTTTTCTGCGCGTAATCTGCTGCTTGCAAACAAAAAAACCACCGCTACCA |
| | GCGGTGGTTTGTTTGCCGGATCAAGAGCTACCAACTCTTTTTCCGAAGGTAACTG |
| | GCTTCAGCAGAGCGCAGATACCAAATACTGTCCTTCTAGTGTAGCCGTAGTTAGG |
| | CCACCACTTCAAGAACTCTGTAGCACCGCCTACATACCTCGCTCTGCTAATCCTGT |
| | TACCAGTGGCTGCTGCCAGTGGCGATAAGTCGTGTCTTACCGGGTTGGACTCAAG |
| | ACGATAGTTACCGGATAAGGCGCAGCGGTCGGGCTGAACGGGGGGTTCGTGCACA |
| | CAGCCCAGCTTGGAGCGAACGACCTACACCGAACTGAGATACCTACAGCGTGAGC |
| | ATTGAGAAAGCGCCACGCTTCCCGAAGGGAGAAAGGCGGACAGGTATCCGGTAAG |
| | CGGCAGGGTCGGAACAGGAGAGCGCACGAGGGAGCTTCCAGGGGGAAACGCCTGG |
| | TATCTTTATAGTCCTGTCGGGTTTCGCCACCTCTGACTTGAGCGTCGATTTTTGTG |
| | ATGCTCGTCAGGGGGGCGGAGCCTATGGAAAAACGCCAGCAACGCGGCCTTTTTA |
| | CGGTTCCTGGCCTTTTGCTGGCCTTTTGCTCACATGTTCTTTCCTGCGTTATCCCC |
| | TGATTCTGTGGATAACCGTATTACCGCCTTTGAGTGAGCTGATACCGCTCGCCGC |
| | AGCCGAACGACCGAGCGCAGCGAGTCAGTGAGCGAGGAAGCGGAAGAGCGCCCAA |
| | TACGCAAACCGCCTCTCCCCGCGCGTTGGCCGATTCATTAATGCAGCTGGCACGAC |
| | AGGTTTCCCGACTGGAAAGCGGGCAGTGAGCGCAACGCAATTAATGTGAGTTAGC |
| | TCACTCATTAGGCACCCCAGGCTTTACACTTTATGCTTCCGGCTCGTATGTTGTGT |
| | GGAATTGTGAGCGGATAACAATTTCACACAGGAAACAGCTATGACCATGATTACG |
| | CCAAGCTTTGCTCTTAGGAGTTTCCTAATACATCCCAAACTCAAATATATAAAGC |
| | ATTTGACTTGTTCTATGCCCTAGGGGGCGGGGGGAAGCTAAGCCAGCTTTTTTTA |
| | ACATTTAAAATGTTAATTCCATTTTAAATGCACAGATGTTTTTATTTCATAAGGG |
| | TTTCAATGTGCATGAATGCTGCAATATTCCTGTTACCAAAGCTAGTATAAATAAA |
| | AATAGATAAACGTGGAAATTACTTAGAGTTTCTGTCATTAACGTTTCCTTCCTCA |
| | GTTGACAACATAAATGCGCTGCTGAGCAAGCCAGTTTGCATCTGTCAGGATCAAT |
| | TTCCCATTATGCCAGTCATATTAATTACTAGTCAATTAGTTGATTTTTATTTTTG |
| | ACATATACATGTGAATGAAAGACCCCACCTGTAGGTTTGGCAAGCTAGCTTAAGT |
| | AACGCCATTTTGCAAGGCATGGAAAAATACATAACTGAGAATAGAAAAGTTCAG |
| | ATCAAGGTCAGGAACAGATGGAACAGCTGAATATGGGCCAAACAGGATATCTGTG |
| | GTAAGCAGTTCCTGCCCCGGCTCAGGGCCAAGAACAGATGGAACAGCTGAATATG |
| | GGCCAAACAGGATATCTGTGGTAAGCAGTTCCTGCCCCGGCTCAGGGCCAAGAAC |
| | AGATGGTCCCCAGATGCGGTCCAGCCCTCAGCAGTTTCTAGAGAACCATCAGATG |
| | TTTCCAGGGTGCCCCAAGGACCTGAAATGACCCTGTGCCTTATTTGAACTAACCA |
| | ATCAGTTCGCTTCTCGCTTCTGTTCGCGCGCTTATGCTCCCCGAGCTCAATAAAAG |
| | AGCCCACAACCCCTCACTCGGGGCGCCAGTCCTCCGATTGACTGAGTCGCCCGGGT |
| | ACCCGTGTATCCAATAAACCCTCTTGCAGTTGCATCCGACTTGTGGTCTCGCTGTT |
| | CCTTGGGAGGGTCTCCTCTGAGTGATTGACTACCCGTCAGCGGGGGTCTTTCATTT |
| | GGGGGCTCGTCCGGGATCGGGAGACCCCTGCCCAGGGACCACCGACCCACCACCGG |
| | GAGGTAAGCTGGCCAGCAACTTATCTGTGTCTGTCCGATTGTCTAGTGTCTATGA |
| | CTGATTTTATGCGCCTGCGTCGGTACTAGTTAGCTAACTAGCTCTGTATCTGGCG |
| | GACCCGTGGTGGAACTGACGAGTTCGGAACACCCGGCCGCAACCCTGGGAGACGT |
| | CCCAGGGACTTCGGGGGCCGTTTTTGTGGCCCGACCTGAGTCCTAAAATCCCGATC |
| | GTTTAGGACTCTTTGGTGCACCCCCCTTAGAGGAGGGATATGTGGTTCTGGTAGG |
| | AGACGAGAACCTAAAACAGTTCCCGCCTCCGTCTGAATTTTTGCTTTCGGTTTGG |
| | GACCGAAGCCGCGCCGCGCGTCTTGTCTGCTGCAGCATCGTTCTGTGTTGTCTCTG |
| | TCTGACTGTGTTTCTGTATTTGTCTGAAAATATGGGCCCGGGCTAGACTGTTACC |
| | ACTCCCTTAAGTTTGACCTTAGGTCACTGGAAAGATGTCGAGCGGATCGCTCACA |
| | ACCAGTCGGTAGATGTCAAGAAGAGACGTTGGGTTACCTTCTGCTCTGCAGAATG |
| | GCCAACCTTTAACGTCGGATGGCCGCGAGACGGCACCTTTAACCGAGACCTCATCA |
| | CCCAGGTTAAGATCAAGGTCTTTTCACCTGGCCCGCATGGACACCCAGACCAGGTC |
| | CCCTACATCGTGACCTGGGAAGCCTTGGCTTTTGACCCCCCTCCCTGGGTCAAGCC |
| | CTTTGTACACCCTAAGCCTCCGCCTCCTCTTCCTCCATCCGCCCCGTCTCTCCCCCT |
| | TGAACCTCCTCGTTCGACCCCGCCTCGATCCTCCCTTTATCCAGCCCTCACTCCTTC |
| | TCTAGGCGCCCCCATATGGCCATATGAGATCTTATATGGGGCACCCCCGCCCCTTG |
| | TAAACTTCCCTGACCCTGACATGACAAGAGTTACTAACAGCCCCTCTCTCCAAGCT |
| | CACTTACAGGCTCTCTACTTAGTCCAGCACGAAGTCTGGAGACCTCTGGCGGCAGC |
| | CTACCAAGAACAACTGGACCGACCGGTGGTACCTCACCCTTACCGAGTCGGCGACA |
| | CAGTGTGGGTCCGCCGACACCAGACTAAGAACCTAGAACCTCGCTGGAAAGGACC |
| | TTACACAGTCCTGCTGACCACCCCCACCGCCCTCAAAGTAGACGGCATCGCAGCTT |
| | GGATACACGCCGCCCACGTGAAGGCTGCCGACCCCGGGGGTGGACCATCCTCTAGA |
| | CTG |
| SEQ ID NO: | GGATCCGGATTAGTCCAATTTGTTAAAGACAGGATATCAGTGGTCCAGGCTCTAG |
| 113 | TTTTGACTCAACAATATCACCAGCTGAAGCCTATAGAGTACGAGCCATAGATAAA |
| T4 + pro-IL- | ATAAAAGATTTTATTTAGTCTCCAGAAAAAGGGGGGAATGAAAGACCCCACCTGT |
| 18 (MT1- | AGGTTTGGCAAGCTAGCTTAAGTAACGCCATTTTGCAAGGCATGGAAAAATACAT |
| MMP) | AACTGAGAATAGAGAAGTTCAGATCAAGGTCAGGAACAGATGGAACAGCTGAAT |
| | ATGGGCCAAACAGGATATCTGTGGTAAGCAGTTCCTGCCCCGGCTCAGGGCCAAG |
| | AACAGATGGAACAGCTGAATATGGGCCAAACAGGATATCTGTGGTAAGCAGTTCC |
| | TGCCCCGGCTCAGGGCCAAGAACAGATGGTCCCCAGATGCGGTCCAGCCCTCAGCA |
| | GTTTCTAGAGAACCATCAGATGTTTCCAGGGTGCCCCAAGGACCTGAAATGACCC |
| | TGTGCCTTATTTGAACTAACCAATCAGTTCGCTTCTCGCTTCTGTTCGCGCGCTTC |
| | TGCTCCCCGAGCTCAATAAAAGAGCCCACAACCCCTCACTCGGGGCGCCAGTCCTC |
| | CGATTGACTGAGTCGCCCGGGTACCCGTGTATCCAATAAACCCTCTTGCAGTTGCA |
| | TCCGACTTGTGGTCTCGCTGTTCCTTGGGAGGGTCTCCTCTGAGTGATTGACTACC |
| | CGTCAGCGGGGGTCTTTCACACATGCAGCATGTATCAAAATTAATTTGGTTTTTT |
| | TTCTTAAGTATTTACATTAAATGGCCATAGTACTTAAAGTTACATTGGCTTCCTT |
| | GAAATAAACATGGAGTATTCAGAATGTGTCATAAATATTTCTAATTTTAAGATA |
| | GTATCTCCATTGGCTTTCTACTTTTTCTTTTATTTTTTTTTGTCCTCTGTCTTCCA |
| | TTTGTTGTTGTTGTTGTTTGTTTGTTTGTTTGTTGGTTGGTTGGTTAATTTTTTT |
| | TTAAAGATCCTACACTATAGTTCAAGCTAGACTATTAGCTACTCTGTAACCCAGG |
| | GTGACCTTGAAGTCATGGGTAGCCTGCTGTTTTAGCCTTCCCACATCTAAGATTA |
| | CAGGTATGAGCTATCATTTTTGGTATATTGATTGATTGATTGATTGATGTGTGTG |
| | TGTGTGATTGTGTTTGTGTGTGTGACTGTGAAAATGTGTGTATGGGTGTGTGTGA |
| | ATGTGTGTATGTATGTGTGTGTGTGAGTGTGTGTGTGTGTGTGTGCATGTGTGTG |
| | TGTGTGACTGTGTCTATGTGTATGACTGTGTGTGTGTGTGTGTGTGTGTGTGTGT |
| | GTGTGTGTGTGTGTGTGTTGTGAAAAAATATTCTATGGTAGTGAGAGCCAACGCT |
| | CCGGCTCAGGTGTCAGGTTGGTTTTTGAGACAGAGTCTTTCACTTAGCTTGGAAT |
| | TCACTGGCCGTCGTTTTACAACGTCGTGACTGGGAAAACCCTGGCGTTACCCAACT |
| | TAATCGCCTTGCAGCACATCCCCCTTTCGCCAGCTGGCGTAATAGCGAAGAGGCCC |
| | GCACCGATCGCCCTTCCCAACAGTTGCGCAGCCTGAATGGCGAATGGCGCCTGATG |
| | CGGTATTTTCTCCTTACGCATCTGTGCGGTATTTCACACCGCATATGGTGCACTCT |
| | CAGTACAATCTGCTCTGATGCCGCATAGTTAAGCCAGCCCCGACACCCGCCAACAC |
| | CCGCTGACGCGCCCTGACGGGCTTGTCTGCTCCCGGCATCCGCTTACAGACAAGCT |
| | GTGACCGTCTCCGGGAGCTGCATGTGTCAGAGGTTTTCACCGTCATCACCGAAAC |
| | GCGCGATGACGAAAGGGCCTCGTGATACGCCTATTTTTATAGGTTAATGTCATGA |
| | TAATAATGGTTTCTTAGACGTCAGGTGGCACTTTTCGGGGAAATGTGCGCGGAAC |
| | CCCTATTTGTTTATTTTTCTAAATACATTCAAATATGTATCCGCTCATGAGACAA |
| | TAACCCTGATAAATGCTTCAATAATATTGAAAAAGGAAGAGTATGAGTATTCAAC |
| | ATTTCCGTGTCGCCCTTATTCCCTTTTTTGCGGCATTTTGCCTTCCTGTTTTTGCT |
| | CACCCAGAAACGCTGGTGAAAGTAAAAGATGCTGAAGATCAGTTGGGTGCACGAG |
| | TGGGTTACATCGAACTGGATCTCAACAGCGGTAAGATCCTTGAGAGTTTTCGCCC |
| | CGAAGAACGTTTTCCAATGATGAGCACTTTTAAAGTTCTGCTATGTGGCGCGGTA |
| | TTATCCCGTATTGACGCCGGGCAAGAGCAACTCGGTCGCCGCATACACTATTCTCA |
| | GAATGACTTGGTTGAGTACTCACCAGTCACAGAAAAGCATCTTACGGATGGCATG |
| | ACAGTAAGAGAATTATGCAGTGCTGCCATAACCATGAGTGATAACACTGCGGCCA |
| | ACTTACTTCTGACAACGATCGGAGGACCGAAGGAGCTAACCGCTTTTTTGCACAA |
| | CATGGGGGATCATGTAACTCGCCTTGATCGTTGGGAACCGGAGCTGAATGAAGCC |
| | ATACCAAACGACGAGCGTGACACCACGATGCCTGTAGCAATGGCAACAACGTTGC |
| | GCAAACTATTAACTGGCGAACTACTTACTCTAGCTTCCCGGCAACAATTAATAGA |
| | CTGGATGGAGGCGGATAAAGTTGCAGGACCACTTCTGCGCTCGGCCCTTCCGGCT |
| | GGCTGGTTTATTGCTGATAAATCTGGAGCCGGTGAGCGTGGGTCTCGCGGTATCA |
| | TTGCAGCACTGGGGCCAGATGGTAAGCCCTCCCGTATCGTAGTTATCTACACGAC |
| | GGGGAGTCAGGCAACTATGGATGAACGAAATAGACAGATCGCTGAGATAGGTGCC |
| | TCACTGATTAAGCATTGGTAACTGTCAGACCAAGTTTACTCATATATACTTTAGA |
| | TTGATTTAAAACTTCATTTTTAATTTAAAAGGATCTAGGTGAAGATCCTTTTTGA |
| | TAATCTCATGACCAAAATCCCTTAACGTGAGTTTTCGTTCCACTGAGCGTCAGAC |
| | CCCGTAGAAAAGATCAAAGGATCTTCTTGAGATCCTTTTTTTCTGCGCGTAATCT |
| | GCTGCTTGCAAACAAAAAAACCACCGCTACCAGCGGTGGTTTGTTTGCCGGATCA |
| | AGAGCTACCAACTCTTTTTCCGAAGGTAACTGGCTTCAGCAGAGCGCAGATACCA |
| | AATACTGTCCTTCTAGTGTAGCCGTAGTTAGGCCACCACTTCAAGAACTCTGTAG |
| | CACCGCCTACATACCTCGCTCTGCTAATCCTGTTACCAGTGGCTGCTGCCAGTGGC |
| | GATAAGTCGTGTCTTACCGGGTTGGACTCAAGACGATAGTTACCGGATAAGGCGC |
| | AGCGGTCGGGCTGAACGGGGGGTTCGTGCACACAGCCCAGCTTGGAGCGAACGAC |
| | CTACACCGAACTGAGATACCTACAGCGTGAGCATTGAGAAAGCGCCACGCTTCCC |
| | GAAGGGAGAAAGGCGGACAGGTATCCGGTAAGCGGCAGGGTCGGAACAGGAGAGC |
| | GCACGAGGGAGCTTCCAGGGGGAAACGCCTGGTATCTTTATAGTCCTGTCGGGTT |
| | TCGCCACCTCTGACTTGAGCGTCGATTTTTGTGATGCTCGTCAGGGGGGCGGAGCC |
| | TATGGAAAAACGCCAGCAACGCGGCCTTTTTACGGTTCCTGGCCTTTTGCTGGCCT |
| | TTTGCTCACATGTTCTTTCCTGCGTTATCCCCTGATTCTGTGGATAACCGTATTAC |
| | CGCCTTTGAGTGAGCTGATACCGCTCGCCGCAGCCGAACGACCGAGCGCAGCGAGT |
| | CAGTGAGCGAGGAAGCGGAAGAGCGCCCAATACGCAAACCGCCTCTCCCCGCGCG |
| | TTGGCCGATTCATTAATGCAGCTGGCACGACAGGTTTCCCGACTGGAAAGCGGGC |
| | AGTGAGCGCAACGCAATTAATGTGAGTTAGCTCACTCATTAGGCACCCCAGGCTT |
| | TACACTTTATGCTTCCGGCTCGTATGTTGTGTGGAATTGTGAGCGGATAACAATT |
| | TCACACAGGAAACAGCTATGACCATGATTACGCCAAGCTTTGCTCTTAGGAGTTT |
| | CCTAATACATCCCAAACTCAAATATATAAAGCATTTGACTTGTTCTATGCCCTAG |
| | GGGGCGGGGGGAAGCTAAGCCAGCTTTTTTTAACATTTAAAATGTTAATTCCATT |
| | TTAAATGCACAGATGTTTTTATTTCATAAGGGTTTCAATGTGCATGAATGCTGCA |
| | ATATTCCTGTTACCAAAGCTAGTATAAATAAAAATAGATAAACGTGGAAATTACT |
| | TAGAGTTTCTGTCATTAACGTTTCCTTCCTCAGTTGACAACATAAATGCGCTGCT |
| | GAGCAAGCCAGTTTGCATCTGTCAGGATCAATTTCCCATTATGCCAGTCATATTA |
| | ATTACTAGTCAATTAGTTGATTTTTATTTTTGACATATACATGTGAATGAAAGAC |
| | CCCACCTGTAGGTTTGGCAAGCTAGCTTAAGTAACGCCATTTTGCAAGGCATGGA |
| | AAAATACATAACTGAGAATAGAAAAGTTCAGATCAAGGTCAGGAACAGATGGAA |
| | CAGCTGAATATGGGCCAAACAGGATATCTGTGGTAAGCAGTTCCTGCCCCGGCTC |
| | AGGGCCAAGAACAGATGGAACAGCTGAATATGGGCCAAACAGGATATCTGTGGTA |
| | AGCAGTTCCTGCCCCGGCTCAGGGCCAAGAACAGATGGTCCCCAGATGCGGTCCAG |
| | CCCTCAGCAGTTTCTAGAGAACCATCAGATGTTTCCAGGGTGCCCCAAGGACCTG |
| | AAATGACCCTGTGCCTTATTTGAACTAACCAATCAGTTCGCTTCTCGCTTCTGTTC |
| | GCGCGCTTATGCTCCCCGAGCTCAATAAAAGAGCCCACAACCCCTCACTCGGGGCG |
| | CCAGTCCTCCGATTGACTGAGTCGCCCGGGTACCCGTGTATCCAATAAACCCTCTT |
| | GCAGTTGCATCCGACTTGTGGTCTCGCTGTTCCTTGGGAGGGTCTCCTCTGAGTGA |
| | TTGACTACCCGTCAGCGGGGGTCTTTCATTTGGGGGCTCGTCCGGGATCGGGAGA |
| | CCCCTGCCCAGGGACCACCGACCCACCACCGGGAGGTAAGCTGGCCAGCAACTTAT |
| | CTGTGTCTGTCCGATTGTCTAGTGTCTATGACTGATTTTATGCGCCTGCGTCGGT |
| | ACTAGTTAGCTAACTAGCTCTGTATCTGGCGGACCCGTGGTGGAACTGACGAGTT |
| | CGGAACACCCGGCCGCAACCCTGGGAGACGTCCCAGGGACTTCGGGGGCCGTTTTT |
| | GTGGCCCGACCTGAGTCCTAAAATCCCGATCGTTTAGGACTCTTTGGTGCACCCCC |
| | CTTAGAGGAGGGATATGTGGTTCTGGTAGGAGACGAGAACCTAAAACAGTTCCCG |
| | CCTCCGTCTGAATTTTTGCTTTCGGTTTGGGACCGAAGCCGCGCCGCGCGTCTTGT |
| | CTGCTGCAGCATCGTTCTGTGTTGTCTCTGTCTGACTGTGTTTCTGTATTTGTCTG |
| | AAAATATGGGCCCGGGCTAGACTGTTACCACTCCCTTAAGTTTGACCTTAGGTCA |
| | CTGGAAAGATGTCGAGCGGATCGCTCACAACCAGTCGGTAGATGTCAAGAAGAGA |
| | CGTTGGGTTACCTTCTGCTCTGCAGAATGGCCAACCTTTAACGTCGGATGGCCGCG |
| | AGACGGCACCTTTAACCGAGACCTCATCACCCAGGTTAAGATCAAGGTCTTTTCA |
| | CCTGGCCCGCATGGACACCCAGACCAGGTCCCCTACATCGTGACCTGGGAAGCCTT |
| | GGCTTTTGACCCCCCTCCCTGGGTCAAGCCCTTTGTACACCCTAAGCCTCCGCCTC |
| | CTCTTCCTCCATCCGCCCCGTCTCTCCCCCTTGAACCTCCTCGTTCGACCCCGCCTC |
| | GATCCTCCCTTTATCCAGCCCTCACTCCTTCTCTAGGCGCCCCCATATGGCCATAT |
| | GAGATCTTATATGGGGCACCCCCGCCCCTTGTAAACTTCCCTGACCCTGACATGAC |
| | AAGAGTTACTAACAGCCCCTCTCTCCAAGCTCACTTACAGGCTCTCTACTTAGTCC |
| | AGCACGAAGTCTGGAGACCTCTGGCGGCAGCCTACCAAGAACAACTGGACCGACC |
| | GGTGGTACCTCACCCTTACCGAGTCGGCGACACAGTGTGGGTCCGCCGACACCAGA |
| | CTAAGAACCTAGAACCTCGCTGGAAAGGACCTTACACAGTCCTGCTGACCACCCCC |
| | ACCGCCCTCAAAGTAGACGGCATCGCAGCTTGGATACACGCCGCCCACGTGAAGGC |
| | TGCCGACCCCGGGGGTGGACCATCCTCTAGACTGCCATGGCAGCTGAGCCTGTGGA |
| | GGACAACTGCATCAACTTCGTGGCCATGAAGTTCATCGACAACACCCTGTACTTC |
| | ATCGCTGAGGACGACGAGAACGGAGGCGGGGGTAGCATCCCTGAGAGCCTGAGAG |
| | CTGGTGGGGGAGGTGGAAGCGCTGCCGCTTACTTCGGAAAGCTGGAGAGCAAGCT |
| | GAGCGTGATCAGAAACCTGAACGACCAGGTGCTGTTCATCGACCAGGGCAACAGA |
| | CCTCTGTTCGAGGACATGACCGACAGCGACTGCAGAGACAACGCTCCCAGAACCA |
| | TCTTCATCATCAGCATGTACAAGGACAGCCAGCCTAGAGGCATGGCCGTGACCAT |
| | CAGCGTGAAGTGCGAGAAGATCAGCACCCTGAGCTGCGAGAACAAGATCATCAGC |
| | TTCAAGGAGATGAACCCTCCTGACAACATCAAGGACACCAAGAGCGACATCATCT |
| | TCTTCCAGAGAAGCGTGCCTGGACACGACAACAAGATGCAGTTCGAGAGCAGCAG |
| | CTACGAGGGCTACTTCCTGGCTTGCGAGAAGGAGAGAGACCTGTTCAAGCTGATC |
| | CTGAAGAAGGAGGACGAGCTGGGAGACAGAAGCATCATGTTCACCGTGCAGAACG |
| | AGGACAGAGCTAAGAGAAGCGGATCTGGAGCTACCAACTTCAGCCTGCTGAAGCA |
| | GGCTGGAGATGTGGAGGAGAACCCTGGACCCATGGGCTGGCTGTGTTCCGGCCTG |
| | CTGTTTCCTGTGTCCTGTCTGGTGCTGCTGCAGGTGGCCAGCTCCGGGAACATGAA |
| | AGTGCTGCAGGAGCCCACATGTGTGTCCGACTACATGTCCATCTCTACATGTGAG |
| | TGGAAGATGAACGGCCCCACAAACTGCTCTACCGAGCTGCGGCTGCTGTACCAGC |
| | TGGTGTTTCTGCTGAGCGAGGCCCACACCTGTATCCCAGAAAATAATGGCGGGGC |
| | CGGGTGTGTGTGCCACCTGCTGATGGATGACGTGGTGTCTGCCGACAATTACACC |
| | CTGGACCTGTGGGCCGGACAGCAGCTGCTGTGGAAGGGGTCCTTCAAACCCTCTG |
| | AGCACGTGAAGCCAAGGGCCCCCGGCAACCTGACAGTGCACACCAACGTGTCTGA |
| | TACACTGCTGCTGACATGGAGCAATCCATACCCTCCTGACAACTACCTGTACAACC |
| | ACCTGACCTACGCCGTGAATATCTGGAGCGAAAATGATCCTGCCGACTTTCGGAT |
| | TTACAATGTGACCTATCTGGAGCCCTCCCTGAGAATTGCCGCCTCTACCCTGAAAT |
| | CTGGAATCTCCTACCGCGCCAGGGTGCGGGCCTGGGCCCAGTGTTACAACACCACC |
| | TGGTCTGAGTGGAGCCCAAGCACCAAGTGGCACAATTCTTATCGGGAGCCTTTTG |
| | AGCAGCACCTGATCCCCTGGCTGGGACACCTGCTGGTGGGGCTGTCTGGCGCCTTT |
| | GGCTTCATCATTCTGGTGTACCTGCTGATCAACTGTAGGAATACAGGCCCTTGGC |
| | TGAAGAAGGTGCTGAAGTGTAACACCCCCGACCCCTCTAAGTTCTTCAGCCAGCT |
| | GTCCTCTGAACACGGGGGAGATGTGCAGAAGTGGCTGTCCAGCCCTTTCCCATCC |
| | AGCTCCTTTAGCCCCGGGGGCCTGGCCCCTGAGATCTCTCCACTGGAAGTGCTGGA |
| | GCGGGACAAGGTGACCCAGCTGCTGCTGCAGCAGGACAAGGTGCCAGAACCCGCC |
| | TCCCTGAGCTCCAACCACAGCCTGACATCTTGCTTTACAAATCAGGGATACTTCTT |
| | CTTCCACCTGCCCGATGCCCTGGAGATCGAGGCCTGCCAGGTGTACTTCACCTACG |
| | ATCCCTACTCTGAGGAAGACCCAGATGAGGGCGTGGCCGGGGCCCCAACCGGGTCC |
| | AGCCCACAGCCACTGCAGCCACTGTCCGGCGAAGATGACGCCTACTGCACATTCCC |
| | TTCCAGGGATGACCTGCTGCTGTTCAGCCCATCTCTGCTGGGCGGACCCTCTCCTC |
| | CAAGCACAGCCCCAGGGGGATCCGGCGCCGGGGAAGAGAGGATGCCCCCTAGCCT |
| | GCAGGAGCGCGTGCCCAGAGACTGGGACCCCCAGCCCCTGGGCCCTCCAACCCCTG |
| | GGGTGCCCGACCTGGTGGACTTCCAGCCTCCACCCGAGCTGGTGCTGAGGGAGGCC |
| | GGCGAAGAGGTGCCCGACGCCGGCCCCCGGGAGGGCGTGTCCTTCCCTTGGTCCAG |
| | ACCTCCAGGACAGGGCGAGTTCCGCGCCCTGAACGCCAGGCTGCCTCTGAACACCG |
| | ATGCCTACCTGTCTCTGCAGGAACTGCAGGGCCAGGACCCAACCCACCTGGTGCGG |
| | AGAAAGCGCAGCGGCTCCGGCGAGGGCCGGGGCAGCCTGCTGACCTGCGGCGACG |
| | TGGAAGAGAACCCCGGACCCATGGGCCCAGGAGTTCTGCTGCTCCTGCTGGTGGCC |
| | ACAGCTTGGCATGGTCAGGGAGGTGTGGTGTCGCACTTCAATGACTGTCCACTGT |
| | CGCACGATGGATACTGCCTCCATGATGGTGTGTGCATGTACATCGAGGCATTGGA |
| | CAAGTATGCATGCAACTGTGTCGTCGGCTACATCGGAGAGCGATGTCAGTACCGA |
| | GACCTGAAGTGGTGGGAACTGAGAGCGGCCGCAATTGAAGTTATGTATCCTCCTC |
| | CTTACCTAGACAATGAGAAGAGCAATGGAACCATTATCCATGTGAAAGGGAAACA |
| | CCTTTGTCCAAGTCCCCTATTTCCCGGACCTTCTAAGCCCTTTTGGGTGCTGGTGG |
| | TGGTTGGTGGAGTCCTGGCTTGCTATAGCTTGCTAGTAACAGTGGCCTTTATTAT |
| | TTTCTGGGTGAGGAGTAAGAGGAGCAGGCTCCTGCACAGTGACTACATGAACATG |
| | ACTCCCCGCCGCCCCGGGCCCACCCGCAAGCATTACCAGCCCTATGCCCCACCACG |
| | CGACTTCGCAGCCTATCGCTCCAGAGTGAAGTTCAGCAGGAGCGCAGACGCCCCCG |
| | CGTACCAGCAGGGCCAGAACCAGCTCTATAACGAGCTCAATCTAGGACGAAGAGA |
| | GGAGTACGATGTTTTGGACAAGAGACGTGGCCGGGACCCTGAGATGGGGGGAAAG |
| | CCGAGAAGGAAGAACCCTCAGGAAGGCCTGTACAATGAACTGCAGAAAGATAAGA |
| | TGGCGGAGGCCTACAGTGAGATTGGGATGAAAGGCGAGCGCCGGAGGGGCAAGGG |
| | GCACGATGGCCTTTACCAGGGTCTCAGTACAGCCACCAAGGACACCTACGACGCCC |
| | TTCACATGCAGGCCCTGCCCCCTCGCTAACAGCCACTCGAG |

### 7. EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

What is claimed is:

1. An immunoresponsive cell expressing a modified pro-cytokine of the IL-1 superfamily, wherein the modified pro-cytokine comprises, from N-terminus to C-terminus:
(a) a pro-peptide;
(b) a cleavage site recognized by a protease other than caspase-1, cathepsin G, elastase or proteinase 3; and
(c) a fragment of a cytokine of the IL-1 superfamily.

2. The immunoresponsive cell of claim 1, wherein the protease is granzyme B (GzB).

3. The immunoresponsive cell of claim 2, wherein the cleavage site has a sequence of SEQ ID NO: 26.

4. The immunoresponsive cell of claim 3, wherein the modified pro-cytokine is a modified pro-IL-18 and has a sequence of SEQ ID NO: 27.

5. The immunoresponsive cell of claim 4, wherein the modified pro-IL-18 was expressed from a polynucleotide of SEQ ID NO: 103 or 111.

6. The immunoresponsive cell of claim 1, wherein the protease is caspase-3.

7. The immunoresponsive cell of claim 6, wherein the cleavage site has a sequence of SEQ ID NO: 28.

8. The immunoresponsive cell of claim 7, wherein the modified pro-cytokine is a modified pro-IL-18 and has a sequence of SEQ ID NO: 29.

9. The immunoresponsive cell of claim 8, wherein the modified pro-IL-18 was expressed from a polynucleotide of SEQ ID NO: 109.

10. The immunoresponsive cell of claim 1, wherein the protease is caspase-8.

11. The immunoresponsive cell of claim 10, wherein the cleavage site has a sequence of SEQ ID NO: 30.

12. The immunoresponsive cell of claim 11, wherein the modified pro-cytokine is a modified pro-IL-18 and has a sequence of SEQ ID NO: 31.

13. The immunoresponsive cell of claim 12, wherein the modified pro-IL-18 was expressed from a polynucleotide of SEQ ID NO: 107.

14. The immunoresponsive cell of claim 1, wherein the protease is MT1-MMP.

15. The immunoresponsive cell of claim 14, wherein the cleavage site has a sequence of SEQ ID NO: 32.

16. The immunoresponsive cell of claim 15, wherein the modified pro-cytokine is a modified pro-IL-18 and has a sequence of SEQ ID NO: 33.

17. The immunoresponsive cell of claim 16, wherein the modified pro-IL-18 was expressed from a polynucleotide of SEQ ID NO: 113.

18. The immunoresponsive cell of any of the preceding claims, wherein the cytokine fragment is a polypeptide having at least 85%, 90%, 95%, 97%, 98%, 99% or 100% sequence identity to SEQ ID: 24.

19. The immunoresponsive cell of any of the preceding claims, wherein the pro-peptide is a polypeptide having at least 85%, 90%, 95%, 97%, 98%, 99% or 100% sequence identity to SEQ ID: 25.

20. The immunoresponsive cell of claim 1, wherein the modified pro-cytokine is a modified pro-IL-36a and has a sequence of SEQ ID NO: 37.

21. The immunoresponsive cell of claim 20, wherein the cytokine fragment is a polypeptide having at least 85%, 90%, 95%, 97%, 98%, 99% or 100% sequence identity to SEQ ID: 42.

22. The immunoresponsive cell of claim 1, wherein the modified pro-cytokine is a modified pro-IL-36β and has a sequence of SEQ ID NO: 39.

23. The immunoresponsive cell of claim 22, wherein the cytokine fragment is a polypeptide having at least 85%, 90%, 95%, 97%, 98%, 99% or 100% sequence identity to SEQ ID: 43.

24. The immunoresponsive cell of claim 1, wherein the modified pro-cytokine is a modified pro-IL-36γ and has a sequence of SEQ ID NO: 41.

25. The immunoresponsive cell of claim 24, wherein the cytokine fragment is a polypeptide having at least 85%, 90%, 95%, 97%, 98%, 99% or 100% sequence identity to SEQ ID: 44.

26. The immunoresponsive cell of any of the preceding claims, further comprising an exogenous polynucleotide encoding the protease.

27. The immunoresponsive cell of any of the preceding claims, wherein said immunoresponsive cell is an αβ T cell, γδ T cell, or a Natural Killer (NK) cell.

28. The immunoresponsive cell of claim 27, wherein said T cell is an αβ T cell.

29. The immunoresponsive cell of claim 27, wherein said T cell is a γδ T-cell.

30. The immunoresponsive cell of any of the preceding claims, further comprising a chimeric antigen receptor (CAR).

31. The immunoresponsive cell of claim 30, wherein the CAR is a second-generation chimeric antigen receptor (CAR) comprising:
a signalling region;
a first co-stimulatory signalling region;
a transmembrane domain; and
a first binding element that specifically interacts with a first epitope on a first target antigen.

32. The immunoresponsive cell of claim 31, wherein the first epitope is an epitope on a MUC1 target antigen.

33. The immunoresponsive cell of claim 32, wherein said first binding element comprises the CDRs of the HMFG2 antibody.

34. The immunoresponsive cell of claim 32, wherein said first binding element comprises the V_{H} and V_{L} domains of the HMFG2 antibody.

35. The immunoresponsive cell of claim 32, wherein said first binding element comprises an HMFG2 single-chain variable fragment (scFv).

36. The immunoresponsive cell of any of the preceding claims, further comprising a chimeric co-stimulatory receptor (CCR), wherein the CCR comprises:
a second co-stimulatory signalling region;
transmembrane domain; and
a second binding element that specifically interacts with a second epitope on a second target antigen.

37. The immunoresponsive cell of claim 36, wherein the second co-stimulatory domain is different from the first co-stimulatory domain.

38. The immunoresponsive cell of any of claims 36-37, wherein the second target antigen comprising said second epitope is selected from the group consisting of ErbB homodimers and heterodimers.

39. The immunoresponsive cell of claim 35, wherein said second target antigen is HER2.

40. The immunoresponsive cell of claim 35, wherein said second target antigen is the EGF receptor.

41. The immunoresponsive cell of any of claims 36-40, wherein said second binding element comprises T1E, the binding moiety of ICR12, or the binding moiety of ICR62.

42. The immunoresponsive cell of any of claims 1-41, wherein the cell expresses a modified pro-IL-18, wherein the modified pro-IL-18 is a polypeptide of SEQ ID NO: 27, and wherein the cell further expresses:
GzB, expressed from an exogenous polynucleotide;
a chimeric antigen receptor (CAR) comprising:
a signalling region;
i. a first co-stimulatory signalling region;
ii. a transmembrane domain; and
iii. a first binding element that specifically interacts with a first epitope on a MUC1 target antigen; and
a chimeric co-stimulatory receptor (CCR) comprising:
iv. a second co-stimulatory signalling region;
v. transmembrane domain; and
vi. a second binding element that specifically interacts with a second epitope on a second target antigen.

43. A polynucleotide or set of polynucleotides comprising a first nucleic acid encoding a modified pro-cytokine, wherein the modified pro-cytokine comprises, from N-terminus to C-terminus:
(a) a pro-peptide;
(b) a cleavage site recognized by a protease other than caspase-1, cathepsin G, elastase or proteinase 3; and
(c) a cytokine fragment of the IL-1 superfamily.

44. The polynucleotide or set of polynucleotides of claim 43, wherein the protease is granzyme B (GzB).

45. The polynucleotide or set of polynucleotides of claim 44, wherein the cleavage site has a sequence of SEQ ID NO: 26.

46. The polynucleotide or set of polynucleotides of claim 45, wherein the modified pro-cytokine is a modified pro-IL-18 and comprises a sequence of SEQ ID NO: 27.

47. The polynucleotide or set of polynucleotides of claim 46, comprising a sequence of SEQ ID NO: 103 or 111.

48. The polynucleotide or set of polynucleotides of claim 43, wherein the protease is caspase-3.

49. The polynucleotide or set of polynucleotides of claim 48, wherein the cleavage site has a sequence of SEQ ID NO: 28.

50. The polynucleotide or set of polynucleotides of claim 49, wherein the modified cytokine is a modified pro-IL-18 and comprises a sequence of SEQ ID NO: 29.

51. The polynucleotide or set of polynucleotides of claim 50, comprising a sequence of SEQ ID NO: 109.

52. The polynucleotide or set of polynucleotides of claim 43, wherein the protease is caspase-8.

53. The polynucleotide or set of polynucleotides of claim 52, wherein the cleavage site has a sequence of SEQ ID NO: 30.

54. The polynucleotide or set of polynucleotides of claim 53, wherein the modified cytokine is a modified pro-IL-18 and comprises a sequence of SEQ ID NO: 31.

55. The polynucleotide or set of polynucleotides of claim 54, comprising a sequence of SEQ ID NO: 107.

56. The polynucleotide or set of polynucleotides of claim 43, wherein the protease is MT1-MMP.

57. The polynucleotide or set of polynucleotides of claim 56, wherein the cleavage site has a sequence of SEQ ID NO: 32.

58. The polynucleotide or set of polynucleotides of claim 57, wherein the modified cytokine is a modified pro-IL-18 and comprises a sequence of SEQ ID NO: 33.

59. The polynucleotide or set of polynucleotides of claim 58, comprising a sequence of SEQ ID NO: 113.

60. The polynucleotide or set of polynucleotides of any of claims 43-59, further comprising a second nucleic acid encoding the protease.

61. The polynucleotide or set of polynucleotides of claim 60, wherein the first nucleic acid and the second nucleic acid are in a single vector.

62. The polynucleotide or set of polynucleotides of any one of claims 43-61, wherein the cytokine fragment is a polypeptide having at least 85%, 90%, 95%, 97%, 98%, 99% or 100% sequence identity to SEQ ID: 24.

63. The polynucleotide or set of polynucleotides of any of claims 43-62, wherein the cytokine fragment can bind and activate an IL-18 receptor when the cleavage site is cleaved.

64. The polynucleotide or set of polynucleotides of any of claims 43-63, wherein the pro-peptide is a polypeptide having at least 85%, 90%, 95%, 97%, 98%, 99% or 100% sequence identity to SEQ ID: 25.

65. The polynucleotide or set of polynucleotides of claim 43, wherein the modified pro-cytokine is a modified pro-IL-36a and has a sequence of SEQ ID NO: 37.

66. The polynucleotide or set of polynucleotides of claim 65, wherein the cytokine fragment is a polypeptide having at least 85%, 90%, 95%, 97%, 98%, 99% or 100% sequence identity to SEQ ID: 42.

67. The polynucleotide or set of polynucleotides of claim 43, wherein the modified pro-cytokine is a modified pro-IL-36β and has a sequence of SEQ ID NO: 39.

68. The polynucleotide or set of polynucleotides of claim 67, wherein the cytokine fragment is a polypeptide having at least 85%, 90%, 95%, 97%, 98%, 99% or 100% sequence identity to SEQ ID: 43.

69. The polynucleotide or set of polynucleotides of claim 43, wherein the modified pro-cytokine is a modified pro-IL-36γ and has a sequence of SEQ ID NO: 41.

70. The polynucleotide or set of polynucleotides of claim 69, wherein the cytokine fragment is a polypeptide having at least 85%, 90%, 95%, 97%, 98%, 99% or 100% sequence identity to SEQ ID: 44.

71. A polynucleotide or set of polynucleotides comprising a first nucleic acid encoding a modified pro-IL-36α, β or γ, wherein the modified pro-IL-36 α, p or γ comprises, from N-terminus to C-terminus:
(a) a pro-peptide;
(b) a cleavage site recognized by a protease other than cathepsin G, elastase or proteinase 3; and
(c) an IL-36 fragment.

72. The polynucleotide or set of polynucleotides of claim 71, wherein the protease is granzyme B (GzB).

73. The polynucleotide or set of polynucleotides of claim 72, wherein the cleavage site has a sequence of SEQ ID NO: 26.

74. The polynucleotide or set of polynucleotides of claim 72, wherein the modified pro-IL-36 α, β or γ comprises a sequence of SEQ ID NO: 37, 39 or 41.

75. The polynucleotide or set of polynucleotides of any of claims 71-74, further comprising a second nucleic acid encoding the protease.

76. The polynucleotide or set of polynucleotides of claim 75, wherein the first nucleic acid and the second nucleic acid are in a single vector.

77. The polynucleotide or set of polynucleotides of any one of claims 71-76, wherein the IL-36 fragment is a polypeptide having at least 85%, 90%, 95%, 97%, 98%, 99% or 100% sequence identity to SEQ ID: 42, 43 or 44.

78. The polynucleotide or set of polynucleotides of any of claims 65-71, wherein the IL-36 fragment can bind and activate an IL-36 receptor when the cleavage site is cleaved.

79. The polynucleotide or set of polynucleotides of any of claims 43-78, further comprising a third nucleic acid encoding a chimeric antigen receptor (CAR).

80. The polynucleotide or set of polynucleotides of claim 79, wherein the CAR is a second-generation chimeric antigen receptor (CAR), comprising:
a signalling region;
a first co-stimulatory signalling region;
a transmembrane domain; and
a first binding element that specifically interacts with a first epitope on a first target antigen.

81. The polynucleotide or set of polynucleotides of claim 80, wherein the first epitope is an epitope on a MUC1 target antigen.

82. The polynucleotide or set of polynucleotides of claim 80, wherein said first binding element comprises the CDRs of the HMFG2 antibody.

83. The polynucleotide or set of polynucleotides of claim 80, wherein said first binding element comprises the V_{H} and V_{L} domains of HMFG2 antibody.

84. The polynucleotide or set of polynucleotides of claim 80, wherein said first binding element comprises HMFG2 single-chain variable fragment (scFv).

85. The polynucleotide or set of polynucleotides of any of claims 43-84, further comprising a fourth nucleic acid encoding a chimeric co-stimulatory receptor (CCR), wherein the CCR comprises:
a second co-stimulatory signalling region;
a transmembrane domain; and
a second binding element that specifically interacts with a second epitope on a second target antigen.

86. The polynucleotide or set of polynucleotides of claim 85, wherein the second target antigen comprising said second epitope is selected from the group consisting of ErbB homodimers and heterodimers.

87. The polynucleotide or set of polynucleotides of claim 85, wherein said second target antigen is HER2.

88. The polynucleotide or set of polynucleotides of claim 85, wherein said second target antigen is EGF receptor.

89. The polynucleotide or set of polynucleotides of any of claims 43-88, wherein said second binding element comprises T1E, the binding moiety of ICR12, or the binding moiety of ICR62.

90. The polynucleotide or set of polynucleotides of any of claims 85-89, wherein the third nucleic acid and the fourth nucleic acid are in a single vector.

91. The polynucleotide or set of polynucleotides of any of claims 43-90, comprising:
a first nucleic acid encoding a modified pro-IL-18, wherein the modified pro-IL-18 is a polypeptide of SEQ ID NO: 27;
second nucleic acid encoding GzB;
a third nucleic acid encoding a chimeric antigen receptor (CAR), wherein the CAR comprises:
i. a signalling region;
ii. a first co-stimulatory signalling region;
iii. a transmembrane domain; and
iv. a first binding element that specifically interacts with a first epitope on a MUC1 target antigen;
a fourth nucleic acid encoding a chimeric co-stimulatory receptor (CCR), wherein the CCR comprises:
v. a second co-stimulatory signalling region;
vi. transmembrane domain; and
vii. a second binding element that specifically interacts with a second epitope on a second target antigen.

92. The polynucleotide or set of polynucleotides of claim 91, comprising the polynucleotide of SEQ ID NO: 103.

93. The polynucleotide or set of polynucleotides of any of claims 43-92, wherein said first nucleic acid and said third nucleic acid are in a single vector.

94. The polynucleotide or set of polynucleotides of any of claims 43-92, wherein said first nucleic acid and said fourth nucleic acid are expressed from a single vector.

95. The polynucleotide or set of polynucleotides of any of claims 43-92, wherein said first nucleic acid, said second nucleic acid, said third nucleic acid, and said fourth nucleic acid are expressed from a single vector.

96. The polynucleotide or set of polynucleotides of any of claims 43-95, comprising:
a first nucleic acid encoding a modified pro-IL-36, wherein the modified pro-IL-36 is a polypeptide of SEQ ID NO: 37, 39 or 41;
second nucleic acid encoding GzB;
a third nucleic acid encoding a chimeric antigen receptor (CAR), wherein the CAR comprises:
i. a signalling region;
ii. a first co-stimulatory signalling region;
iii. a transmembrane domain; and
iv. a first binding element that specifically interacts with a first epitope on a MUC1 target antigen;
a fourth nucleic acid encoding a chimeric co-stimulatory receptor (CCR), wherein the CCR comprises:
v. a second co-stimulatory signalling region;
vi. transmembrane domain; and
vii. a second binding element that specifically interacts with a second epitope on a second target antigen.

97. A γδ T cell expressing:
(a) a second generation chimeric antigen receptor (CAR) comprising
i. a signalling region;
ii. a co-stimulatory signalling region;
iii. a transmembrane domain; and
iv. a first binding element that specifically interacts with a first epitope on a first target antigen; and
(b) a chimeric co-stimulatory receptor (CCR) comprising
v. a co-stimulatory signalling region which is different from that of (ii);
vi. a transmembrane domain; and
vii. a second binding element that specifically interacts with a second epitope on a second target antigen.

98. The γδ T cell of claim 97, wherein said first target antigen is the same as said second target antigen.

99. The γδ T cell of claim 97, wherein said first target antigen is a MUC antigen.

100. The γδ T cell of claim 97, wherein said first binding element comprises the CDRs of the HMFG2 antibody.

101. The γδ T cell of claim 99, wherein said first binding element comprises the V_{H} and V_{L} domains of HMFG2 antibody.

102. The γδ T cell of any one of claims 97-101, wherein said first binding element comprises HMFG2 single-chain variable fragment (scFv).

103. The γδ T cell of any one of claims 97-102, wherein said second target antigen comprising said second epitope is selected from the group consisting of ErbB homodimers and heterodimers.

104. The γδ T cell of any one of claims 97-103, wherein said second target antigen is HER2.

105. The γδ T cell of claim 104, wherein said second target antigen is EGF receptor.

106. The γδ T cell of any one of claims 97 to 105, wherein said second binding element comprises T1E, ICR12, or ICR62.

107. The γδ T cell of claim 106, wherein said second binding element is T1E.

108. The γδ T cell of any one of claims 97 to 107, wherein said second target antigen is αvβ6 integrin.

109. The γδ T cell of claim 108, wherein said second binding element is A20 peptide.

110. A method of preparing the immunoresponsive cell of any one of claims 1 to 42, said method comprising transfecting or transducing the polynucleotide or set of polynucleotides of any one of claims 43 to 96 into an immunoresponsive cell.

111. A method for directing a T cell-mediated immune response to a target cell in a patient in need thereof, said method comprising:
administering to the patient a therapeutically effective number of the immunoresponsive cells of any one of claims 1 to 42 or the γδ T cell of any one of claims 97 to 109.

112. The method of claim 111, wherein the target cell expresses MUC1.

113. A method of treating cancer, said method comprising:
administering to the patient an effective amount of the immunoresponsive cell of any one of claims 1 to 42 or the γδ T cell of any one of claims 97 to 109.

114. An immunoresponsive cell of any one of claims 1 to 42, polynucleotide of any one of claims 43 to 96, or the γδ T cell of any one of claims 97 to 109 for use (i) in a therapy or as a medicament or (ii) in the treatment of a cancer patient.

115. The method of claim 113 or the immunoresponsive cell, polynucleotide, or γδ T cell of claim 114, wherein the patient's cancer cell expresses MUC 1.

116. The method of claim 113 or the immunoresponsive cell, polynucleotide, or γδ T cell of claim 114, wherein the patient has a cancer selected from the group consisting of breast cancer, ovarian cancer, pancreatic cancer, colorectal cancer, lung cancer, gastric cancer, bladder cancer, prostate cancer, esophageal cancer, endometrial cancer, hepatobiliary cancer, duodenal carcinoma, thyroid carcinoma, renal cell carcinoma, multiple myeloma, and non-Hodgkin's lymphoma.

117. The method or the immunoresponsive cell, polynucleotide, or γδ T cell of claim 116, wherein the patient has breast cancer.

118. The method or the immunoresponsive cell, polynucleotide, or γδ T cell of claim 116, wherein the patient has ovarian cancer.

119. Use of an immunoresponsive cell of any one of claims 1 to 42, polynucleotide of any one of claims 43 to 96, or the γδ T cell of any one of claims 97 to 109 in the manufacture of a medicament for the treatment of a pathological disorder.

120. A method of making an immunoresponsive cell, comprising a step of introducing a transgene.

121. The method of claim 120, wherein the transgene encodes a CAR or pCAR.

122. The method of claim 120, wherein the transgene encodes a modified pro-cytokine of IL-1 superfamily, wherein the modified pro-cytokine comprises, from N-terminus to C-terminus:
(a) a pro-peptide;
(b) a cleavage site recognized by a protease other than caspase-1, cathepsin G, elastase or proteinase 3; and
(c) a cytokine fragment of the IL-1 superfamily.

123. The method of any one of claims 120-122, further comprising a preceding step of activating the γδ T cell with an anti-γδ TCR antibody.

124. The method of claim 123, wherein the anti-γδ TCR antibody is immobilised.
